(19) United States
(12) Patent Application Publication
(10) Pub. No.: US 2009/0281138 A1
(43) Pub. Date: Nov. 12, 2009

(54) Bis-(Sulfonylamino) Derivatives in Therapy 066

(75) Inventors: Johan Bylund, Sodertalje (SE); Marie E. Ek, Sodertalje (SE); Annika Kers, Sodertalje (SE); Gunnar Nordvall, Sodertalje (SE); Liselotte Ohberg, Sodertalje (SE); Jenny Viklund, Sodertalje (SE); Stefan Von Berg, Sodertalje (SE); Jorg Holenz, Sodertalje (SE); Katja Narhi, Sodertalje (SE); Daniel Sohn, Sodertalje (SE); Martin H. Johansson, Sodertalje (SE)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodertalje (SE)

(21) Appl. No.: 12/271,552

(22) Filed: Nov. 14, 2008

Related U.S. Application Data
(60) Provisional application No. 60/988,164, filed on Nov. 15, 2007.

(51) Int. Cl. A61K 31/437; C07D 405/02; C07D 471/04; A61K 31/426; A61P 29/00; C07D 277/12; A61K 31/4427

(52) U.S. Cl. 514/302; 546/284.1; 546/115; 548/200; 514/336; 514/365

(57) ABSTRACT

The invention provides compounds of formula

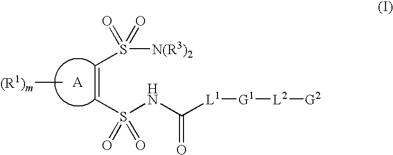

wherein R¹, R³, L¹, L², G¹, G², A and m are as defined in the specification and optical isomers, racemates and tautomers thereof, and pharmaceutically acceptable salts thereof; together with processes for their preparation, pharmaceutical compositions containing them and their use in therapy. The compounds are inhibitors of microsomal prostaglandin E synthase-1.

### CROSS-REFERENCE TO RELATED APPLICATIONS

This application claims benefit under 35 U.S.C. §119(e) of U.S. Provisional Application 60/988,164, filed Nov. 15, 2007, which is incorporated herein by reference in its entirety.

### FIELD OF THE INVENTION

The present invention relates to bis-(sulfonylamino) derivatives, processes for their preparation, pharmaceutical compositions containing them and their use in therapy.

### BACKGROUND OF THE INVENTION

Modulation of prostaglandin metabolism is at the center of current anti-inflammatory therapies. NSAIDs and COX-2 inhibitors block the activity of cyclooxygenases and their ability to convert arachidonic acid into prostaglandin H2 (PGH2). PGH2 can be subsequently metabolized by terminal prostaglandin synthases to the corresponding biologically active PGs, namely, PGI2, thromboxane (Tx) A2, PGD2, PGF2α, and PGE2. A combination of pharmacological, genetic and neutralizing antibody approaches demonstrates the importance of PGE2 in inflammation. The conversion of PGH2 to PGE2 by prostaglandin E synthases (PGES) may therefore represent a pivotal step in the propagation of inflammatory stimuli.

Microsomal prostaglandin E synthase-1 (mPGES-1) is an inducible PGES after exposure to pro-inflammatory stimuli. mPGES-1 is induced in the periphery and in the CNS by inflammation and represents therefore a target for acute and chronic inflammatory disorders.

PGE2 is a major prostanoid driving inflammatory processes. The Prostanoid is produced from arachidonic acid liberated by Phospholipases (PLAs). Arachidonic acid is transformed by the action of Prostaglandin H Synthase (PGH Synthase, cycloxygenase) into PGH2 which is a substrate for mPGES-1, that is the terminal enzyme transforming PGH2 to the pro-inflammatory PGE2.

NSAIDs reduce PGE2 by inhibiting cyclooxygenase, but at the same time reducing other prostanoids, giving side effects such as ulcerations in the GI tract. mPGES-1 inhibition gives a similar effect on PGE2 production without affecting the formation of other prostanoids, and hence a more favourable profile.

By blocking the formation of PGE2 in animal models of inflammatory pain a reduced inflammation, pain and fever response has been demonstrated, Kojima et al., The Journal of Immunology 2008, 180, 8361-6, Xu et al., The Journal of Pharmacology and Experimental Therapeutics 2008, 326, 754-63.

In abdominal aortic aneurism, inflammation leads to connective tissue degradation and smooth muscle apoptosis ultimately leading to aortic dilation and rupture. In animals lacking mPGES-1a slower disease progression and disease severity has been demonstrated Wang et al. Circulation, 2008, 117, 1302-1309.

Several lines of evidence indicate that PGE2 is involved in malignant growth. PGE2 facilitates tumour progression by stimulation of cellular proliferation and angiogenesis and by modulation of immunosuppression. In support of a role for PGE2 in carcinogenesis genetic deletion of mPGES-1 in mice suppresses the intestinal tumorigenesis Nakanishi et al. Cancer Research 2008, 68(9), 3251-9. In man, mPGES-1 is also upregulated in cancers such as colorectal cancer Schröder Journal of Lipid Research 2006, 47, 1071-80.

Myositis is chronic muscle disorder characterized by muscle weakness and fatigue. Proinflammatory cytokines and prostanoids have been implicated in the development of myositis. In skeletal muscle tissue from patients suffering from myositis an increase in cyclooxygenases and mPGES-1 has been demonstrated, implicating mPGES-1 as a target for treating this condition. Korotkova Annals of the Rheumatic Diseases 2008, 67, 1596-1602.

In atherosclerosis inflammation of the vasculature leads to atheroma formation that eventually may progress into infarction. In patients with carotid atherosclerosis an increase in mPGES-1 in plaque regions have been found Gómez-Hernández Atherosclerosis 2006, 187, 139-49. In an animal model of atherosclerosis, mice lacking the mPGES-1 receptor was found to show a retarded atherogenesis and a concomitant reduction in macrophage-derived foam cells together with an increase in vascular smooth muscle cells. Wang Proceedings of National Academy of Sciences 2006, 103(39), 14507-12.

The present invention is directed to novel compounds that are selective inhibitors of the microsomal prostaglandin E synthase-1 enzyme and would therefore be useful for the treatment of pain and inflammation in a variety of diseases or conditions.

### DISCLOSURE OF THE INVENTION

In one aspect we disclose a compound of formula (I) or a pharmaceutically acceptable salt thereof

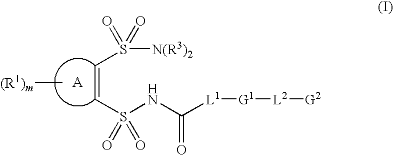

wherein:
A is selected from phenyl or a 5- or 6-membered heteroaryl moiety; said phenyl or a 5- or 6-membered heteroaryl moiety in group A being optionally fused to a phenyl, a 5- or 6-membered heteroaryl, C₅₋₆-carbocyclyl or C₅₋₆heterocyclyl ring;
R¹ is independently selected from halogen, nitro, SF₅, OH, CHO, CO₂R⁴, CONR⁵R⁶, C₁₋₄alkyl, C₁₋₄alkoxy, G³, OG³ or OCH₂G³; said C₁₋₄alkyl or C₁₋₄alkoxy being optionally substituted by OH or by one or more F atoms;
m represents an integer 0, 1 or 2;
R³ is hydrogen;
L¹ represents a direct bond, C₁₋₄alkylene, C₂₋₄alkenylene or C₂₋₄alkynylene;
L² represents a direct bond, —O—, —OCH₂—, C₁₋₂alkylene or —C≡C—;
G¹ represents phenyl, 5- or 6-membered heteroaryl, C₃₋₁₀carbocyclyl or C₅₋₈heterocyclyl;
G² represents H, C₁₋₆alkyl, C₁₋₆alkenylene, phenyl, 5- or 6-membered heteroaryl, C₃₋₁₀carbocyclyl or C₅₋₈heterocyclyl; said C₁₋₆alkyl being optionally further substituted by one or more groups selected from OH, C₁₋₆alkoxy and halogen;
The phenyl, heteroaryl, carbocyclyl or heterocyclyl moieties in G¹ and G² being optionally fused to one or two further rings independently selected from phenyl, a 5- or 6-membered heteroaryl, C₅₋₆-carbocyclyl or C₅₋₆heterocyclyl ring;
Any phenyl, heteroaryl, carbocyclyl or heterocyclyl moieties in G¹ and G² being optionally substituted by one or more substituents independently selected from halogen, OH, CN, NO₂, CO₂R⁹, C₁₋₆alkyl, C₁₋₆alkoxy, C₁₋₄thioalkoxy, SO₂NR¹⁰R¹¹,NR¹²R¹³, —O(CH₂)₂—O—(CH₂)₂—C₁₋₆alkoxy, —NHCOC(OH)(CH₃)CF₃, —CH₂OCH₂CF₂CHF₂ or —CH₂OCH₂CH₂CF₃; said C₁₋₆alkyl or C₁₋₆alkoxy being optionally substituted by OH, C₁₋₆alkoxy, phenyl or by one or more F atoms;
G³ represents phenyl or 5- or 6-membered heteroaryl; and
Each R⁴, R⁵, R⁶, R⁹, R¹⁰, R¹¹, R¹² and R¹³ is independently selected from H or C₁₋₄alkyl, provided that the compounds
1,2-Benzenedisulfonamide, N1-[[(4,6-dimethyl-2-pyrimidinyl)amino]carbonyl];
1,2-Benzenedisulfonamide, N1-[[(4,6-dimethoxy-1,3,5-triazin-2-yl)amino]carbonyl];
1,2-Benzenedisulfonamide, N1-[[(4-methoxy-6-methyl-2-pyrimidinyl)amino]carbonyl];
1,2-Benzenedisulfonamide, N1-[[(4,6-dimethoxy-2-pyrimidinyl)amino]carbonyl] are excluded.

As used herein, a C₁-C₆ alkyl moiety is a linear or branched alkyl moiety containing from 1 to 6 carbon atoms, such as a C₁-C₄ or C₁-C₂ alkyl moiety. Examples of C₁-C₆ alkyl moieties include methyl, ethyl, n-propyl, i-propyl, n-butyl, s-butyl and t-butyl, pentyl and hexyl. For the avoidance of doubt, where two alkyl moieties are present in a substituent, the alkyl moieties may be the same or different.

As used herein, a C₁-C₄ alkylene or C₁-C₂ alkylene group is any divalent linear or branched C₁-C₄ or C₁-C₂ alkyl moiety. Linear C₁-C₄ alkylene groups are methylene, ethylene, n-propylene and n-butylene groups. Branched C₁-C₄ alkylene groups include —CH(CH₃)—, —CH(CH₃)—CH₂— and —CH₂—CH(CH₃)—.

As used herein, a C₂-C₄ alkenylene group is any divalent linear or branched C₂-C₄ alkylene moiety that includes a carbon-carbon double bond.

As used herein, a C₂-C₄ alkynylene group is any divalent linear or branched C₂-C₄ alkylene moiety that includes a carbon-carbon triple bond.

As used herein, a halogen is chlorine, fluorine, bromine or iodine. A halogen is typically fluorine, chlorine or bromine.

As used herein, a C₁-C₆ alkoxy moiety is a said C₁-C₆ alkyl moiety attached to an oxygen atom. Examples include methoxy and ethoxy.

As used herein, a C₁-C₄ thioalkoxy moiety is a said C₁-C₄ alkyl moiety attached to a sulphur atom. Examples include methylthio and ethylthio.

As used herein, a 5- or 6-membered heteroaryl moiety is a monocyclic 5- or 6-membered aromatic ring, containing at least one heteroatom, for example 1, 2 or 3 heteroatoms, selected from O, S and N. Examples include imidazolyl, isoxazolyl, pyrrolyl, thienyl, thiazolyl, furanyl, pyridyl, pyrazinyl, pyrimidinyl, pyridazinyl, oxadiazolyl, oxazolyl, isoxazolyl, thiazolyl, isothiazolyl, thiadiazolyl, pyrazolyl and triazolyl moieties.

In one embodiment, a 5- or 6-membered heteroaryl moiety is pyrrolyl, thienyl, furanyl, pyridyl, pyrimidinyl, oxazolyl, thiazolyl or pyrazolyl moiety.

As used herein, a 5- to 8-membered heterocyclyl moiety is a monocyclic non-aromatic, saturated or unsaturated C₅-C₈ carbocyclic ring, in which at least one, for example, 1, 2 or 3, carbon atoms in the ring are replaced with a moiety selected independently from O, S, SO, SO₂ and N and optionally incorporating one or more carbonyl (C═O) groups. Typically, it is a saturated C₅-C₈ ring such as a C₅-C₆ ring in which 1, 2 or 3 of the carbon atoms in the ring are replaced with a moiety selected from O, S, SO₂ and NH and optionally incorporating one or two CO moieties. Examples include azetidinyl, pyrazolidinyl, piperidyl, piperidin-2,6-dionyl, piperidin-2-onyl, perhydroazepinyl (hexamethylene iminyl), piperazinyl, morpholinyl, thiomorpholinyl, S-oxothiomorpholinyl, S,S-dioxothiomorpholinyl, 1,3-dioxolanyl, 1,4-dioxanyl, pyrrolidinyl, imidazolidinyl, imidazol-2-onyl, pyrrolidin-2-onyl, tetrahydrofuranyl, tetrahydrothienyl, S,S-dioxotetrahydrothienyl (tetramethylenesulfonyl), dithiolanyl, thiazolidinyl, oxazolidinyl, tetrahydropyranyl and pyrazolinyl moieties. In one embodiment, a 5- to 8-membered heterocyclyl moiety is morpholinyl, tetrahydrofuranyl or S,S-dioxotetrahydrothienyl.

For the avoidance of doubt, although the above definitions of heteroaryl and heterocyclyl groups refer to an “N” moiety which can be present in the ring, as will be evident to a skilled chemist the N atom will carry a hydrogen atom (or will carry a substituent as defined above) if it is attached to each of the adjacent ring atoms via a single bond.

As used herein, a C₃-C₁₀ carbocyclyl moiety is a monocyclic or polycyclic non-aromatic saturated or unsaturated hydrocarbon ring having from 3 to 10 carbon atoms. In one embodiment, it is a saturated ring system (i.e. a cycloalkyl moiety) having from 3 to 7 carbon atoms. Examples include adamantyl, cyclopropyl, cyclobutyl, cyclopentyl, cyclohexyl and cycloheptyl and bicycloheptyl. In one embodiment, a C₃-C₁₀ carbocyclyl moiety is adamantyl, cyclopentyl, cyclohexyl or bicycloheptyl moiety. In another embodiment, it is a C₅-C₆ cycloalkyl moiety.

Examples of bicyclic ring systems in which the two rings are fused together include naphthyl, indanyl, quinolyl, tetrahydroquinolyl, benzofuranyl, indolyl, isoindolyl, indolinyl, benzofuranyl, benzothienyl, indazolyl, benzimidazolyl, benzthiazolyl, benzmorpholinyl, isoquinolyl, chromanyl, indenyl, quinazolyl, quinoxalyl, isocromanyl, tetrahydronaphthyl, pyrido-oxazolyl, pyridothiazolyl, dihydrobenzofuranyl, 1,3-benzodioxolyl, 2,3-dihydro-1,4-benzodioxinyl, 1,3-benzodioxinyl and 3,4-dihydro-isochromenyl.

In one embodiment, a bicyclic fused ring system is a naphthyl, indanyl, indolyl, benzofuranyl, benzothienyl, benzthiazolyl, benzmorpholinyl, pyrido-oxazolyl, pyridothiazolyl or dihydrobenzofuranyl moiety.

In one embodiment, a bicyclic fused ring system is a naphthyl, indolyl, benzofuranyl, benzothienyl or quinolyl moiety.

Examples of tricyclic ring systems in which the three rings are fused together include xanthenyl, carbazolyl, acridinyl, phenothiazinyl, phenoxazinyl, dibenzofuranyl, dibenzothienyl, S,S,-dioxodibenzothienyl, fluorenyl, phenanthrenyl and anthracenyl. In one embodiment, a tricyclic fused ring system is a dibenzofuranyl or S,S,-dioxodibenzothienyl moiety.

For the avoidance of doubt, when the phenyl, heteroaryl, carbocyclyl or heterocyclyl moieties in G¹ and G² are fused to one or two further rings, said fused rings may be substituted at one or more ring positions with such substituents as described above.

As used herein, the term “aryl” refers to an aromatic ring structure made up of from 5 to 14 carbon atoms. Ring structures containing 5, 6, 7 and 8 carbon atoms would be single-ring (monocyclic) aromatic groups, for example, phenyl. Ring structures containing 8, 9, 10, 11, 12, 13, or 14 would be polycyclic, for example naphthyl. The aromatic ring can be substituted at one or more ring positions with such substituents as described above. The term “aryl” also includes—unless stated to the contrary—polycyclic ring systems having two or more cyclic rings in which two or more carbons are common to two adjoining rings (the rings are “fused rings”) wherein at least one of the rings is aromatic, for example, the other cyclic rings can be cycloalkyls, cycloalkenyls, cycloalkynyls, aryls and/or heterocyclyls. The terms ortho, meta and para apply to 1,2-, 1,3- and 1,4-disubstituted benzenes, respectively. For example, the names 1,2-dimethylbenzene and ortho-dimethylbenzene are synonymous.

In one embodiment, A is selected from phenyl or pyridyl; said phenyl or pyridyl being optionally fused to a phenyl, a 5- or 6-membered heteroaryl, C₅₋₆-carbocyclyl or C₅₋₆heterocyclyl ring. Examples of fused ring systems for A include naphthyl, indanyl, quinolyl, tetrahydroquinolyl, benzofuranyl, indolyl, benzothienyl, indazolyl, benzimidazolyl, benzthiazolyl, indenyl, tetrahydronaphthyl, pyrido-oxazolyl, pyridothiazolyl, dihydrobenzofuranyl, 1,3-benzodioxolyl and 2,3-dihydro-1,4-benzodioxinyl. In another embodiment, A is phenyl or pyridyl. In another embodiment, A is phenyl. In another embodiment, A is pyridyl.

In one embodiment, R¹ is independently selected from halogen, nitro, SF₅, OH, CHO, C₁₋₄alkyl or C₁₋₄alkoxy; said C₁₋₄alkyl or C₁₋₄alkoxy being optionally substituted by OH or by one or more F atoms.

In another embodiment, R¹ is independently selected from halogen, C₁₋₄alkyl or C₁₋₄alkoxy; said C₁₋₄alkyl or C₁₋₄alkoxy being optionally substituted by OH or by one or more F atoms.

In one embodiment, m represents an integer 0 or 1. In another embodiment, m represents an integer 0.

In one embodiment, each R³ is independently selected from hydrogen, CN and C₁₋₄alkyl. In another embodiment, each R³ represents hydrogen.

In one embodiment, L¹ represents a direct bond, C₁₋₂alkylene or C₂alkenylene. In one embodiment L¹ represents a direct bond or C₁₋₄alkylene.

In another embodiment, L¹ represents a direct bond.

In one embodiment L² represents a direct bond, —OCH₂— or —C≡C—;

In one embodiment, L² represents a direct bond or —C≡C—. In another embodiment, L² represents a direct bond. In another embodiment, L² represents —C≡C—.

In one embodiment, G¹ represents phenyl or 5- or 6-membered heteroaryl; optionally fused to one further ring independently selected from phenyl and 5- or 6-membered heteroaryl.

In another embodiment, G¹ represents phenyl; optionally fused to one further ring independently selected from phenyl and 5- or 6-membered heteroaryl.

In one embodiment G¹ represents phenyl, pyridyl, thiazolyl, thienyl, furanyl, pyrimidinyl, cyclohexyl, adamantyl or bicycloheptyl.

In another embodiment, G¹ represents phenyl.

In one embodiment, G² represents H, C₁₋₆alkyl, phenyl or 5- or 6-membered heteroaryl; said phenyl or 5- or 6-membered heteroaryl being optionally fused to one further ring independently selected from phenyl, a 5- or 6-membered heteroaryl, C₅₋₆-carbocyclyl or C₅₋₆heterocyclyl ring.

In one embodiment G² represents phenyl, benzofuranyl, benzothienyl, benzthiazolyl, [1,3]oxazolo[4,5-c]pyridyl, [1,3]oxazolo[5,4-c]pyridyl, benzoxazolyl, 2,3-dihydro-1-benzofuranyl, indolyl, pyridyl, quinolyl, cyclopropyl, cyclopentyl, cyclohexyl, cycloheptyl.

In one embodiment G² represents C₂₋₄alkenylene;

Any phenyl, heteroaryl, carbocyclyl or heterocyclyl moieties in G¹ and G² being optionally substituted by one or more substituents independently selected from halogen, OH, CN, NO₂, CO₂R⁹, C₁₋₆alkyl, C₁₋₆alkoxy, C₁₋₄thioalkoxy, SO₂NR¹⁰R¹¹,NR¹²R¹³, —O(CH₂)₂—O—(CH₂)₂—C₁₋₆alkoxy, —NHCOC(OH)(CH₃)CF₃, —CH₂OCH₂CF₂CHF₂ or —CH₂OCH₂CH₂CF₃; said C₁₋₆alkyl or C₁₋₆alkoxy being optionally substituted by OH, C₁₋₆alkoxy, phenyl or by one or more F atoms;

In one embodiment any phenyl, heteroaryl, carbocyclyl or heterocyclyl moieties in G¹ and G² being optionally substituted by one or more substituents independently selected from halogen, CO₂R⁹, C₁₋₆alkyl, C₁₋₆alkoxy, —O(CH₂)₂—O—(CH₂)₂—C₁₋₆alkoxy, CH₂OCH₂CF₂CHF₂ or —CH₂OCH₂CH₂CF₃; said C₁₋₆alkyl or C₁₋₆alkoxy being optionally substituted by OH, C₁₋₆alkoxy, phenyl or by one or more F atoms;

In one embodiment, any phenyl, heteroaryl, carbocyclyl or heterocyclyl moieties in G¹ and G² are optionally substituted by one or more substituents independently selected from halogen, CN, NO₂, C₁₋₆alkyl and C₁₋₆alkoxy; said C₁₋₆alkyl or C₁₋₆alkoxy being optionally substituted by OH or by one or more F atoms. In another embodiment, any phenyl, heteroaryl, carbocyclyl or heterocyclyl moieties in G¹ and G² are optionally substituted by one or more substituents independently selected from halogen, C₁₋₆alkyl and C₁₋₆alkoxy; said C₁₋₆alkyl being optionally substituted by OH or by one or more F atoms.

In one embodiment, A is phenyl or pyridyl; R¹ is independently selected from halogen, C₁₋₄alkyl or C₁₋₄alkoxy; said C₁₋₄alkyl or C₁₋₄alkoxy being optionally substituted by OH or by one or more F atoms; m represents an integer 0 or 1; each R³ represents hydrogen; L¹ represents a direct bond; L² represents a direct bond; G¹ represents phenyl; optionally fused to one further ring independently selected from phenyl and 5- or 6-membered heteroaryl; G² represents H, phenyl or 5- or 6-membered heteroaryl; optionally fused to one further ring independently selected from phenyl, a 5- or 6-membered heteroaryl, C₅₋₆-carbocyclyl or C₅₋₆heterocyclyl ring; and any phenyl or heteroaryl moieties in G¹ and G² are optionally substituted by one or more substituents independently selected from halogen, C₁₋₆alkyl and C₁₋₆alkoxy; said C₁₋₆alkyl being optionally substituted by OH or by one or more F atoms.

In one embodiment, A is phenyl; m represents an integer 0; each R³ represents hydrogen; L¹ represents a direct bond; L² represents a direct bond; G¹ represents phenyl; optionally fused to one further ring independently selected from phenyl and 5- or 6-membered heteroaryl; G² represents H, phenyl or 5- or 6-membered heteroaryl; optionally fused to one further ring independently selected from phenyl, a 5- or 6-membered heteroaryl, C₅₋₆-carbocyclyl or C₅₋₆heterocyclyl ring; and any phenyl or heteroaryl moieties in G¹ and G² are optionally substituted by one or more substituents independently selected from halogen, C₁₋₆alkyl and C₁₋₆alkoxy; said C₁₋₆alkyl being optionally substituted by OH or by one or more F atoms.

In one embodiment, A is phenyl; m represents an integer 0; each R³ represents hydrogen; L¹ represents a direct bond; L² represents —C≡C—; G¹ represents phenyl; optionally fused to one further ring independently selected from phenyl and 5- or 6-membered heteroaryl; G² represents C₁₋₆alkyl optionally substituted by one or more groups selected from OH, C₁₋₆alkoxy and halogen; and any phenyl or heteroaryl moieties in G¹ is optionally substituted by one or more substituents independently selected from halogen, C₁₋₆alkyl and C₁₋₆alkoxy; said C₁₋₆alkyl being optionally substituted by OH or by one or more F atoms.

Examples of compounds of the invention include:
5-Benzofuran-2-yl-N-(2-sulfamoylphenyl)sulfonyl-pyridine-2-carboxamide
5-(2,3-Dichlorophenyl)-N-(2-sulfamoylphenyl)sulfonyl-pyridine-2-carboxamide
4-Benzofuran-2-yl-N-(2-sulfamoylphenyl)sulfonyl-benzamide
4-Benzothiophen-2-yl-N-(2-sulfamoylphenyl)sulfonyl-benzamide
4-Benzothiazol-2-yl-N-(2-sulfamoylphenyl)sulfonyl-benzamide
4-(7-Oxa-3,9-diazabicyclo[4.3.0]nona-2,4,8,10-tetraen-8-yl)-N-(2-sulfamoylphenyl)sulfonyl-benzamide
4-(7-Oxa-5,9-diazabicyclo[4.3.0]nona-2,4,8,10-tetraen-8-yl)-N-(2-sulfamoylphenyl)sulfonyl-benzamide
4-Benzooxazol-2-yl-N-(2-sulfamoylphenyl)sulfonyl-benzamide
2-Phenyl-N-(2-sulfamoylphenyl)sulfonyl-benzofuran-6-carboxamide
4-Bromo-N-(2-sulfamoylphenyl)sulfonyl-benzamide
4-Bromo-2-chloro-N-(2-sulfamoylphenyl)sulfonyl-benzamide
4-Bromo-3-methyl-N-(2-sulfamoylphenyl)sulfonyl-benzamide
4-Bromo-3-fluoro-N-(2-sulfamoylphenyl)sulfonyl-benzamide
4-Bromo-2-fluoro-N-(2-sulfamoylphenyl)sulfonyl-benzamide
4-Bromo-2-methyl-N-(2-sulfamoylphenyl)sulfonyl-benzamide
2-(1-Adamantyl)-N-(2-sulfamoylphenyl)sulfonyl-acetamide
N-(2-Sulfamoylphenyl)sulfonylnorbornane-2-carboxamide
1-Phenyl-N-(2-sulfamoylphenyl)sulfonyl-cyclohexane-1-carboxamide
3-(Difluoromethoxy)-N-(2-sulfamoylphenyl)sulfonyl-benzamide
3-Bromo-4-fluoro-N-(2-sulfamoylphenyl)sulfonyl-benzamide
N-(2-sulfamoylphenyl)sulfonyl-3-(2,2,3,3-tetrafluoropropoxymethyl)benzamide
4-Methyl-N-(2-sulfamoylphenyl)sulfonyl-2-[3-(trifluoromethyl)phenyl]1,3-thiazole-5-carboxamide
4-Chloro-2-fluoro-N-(2-sulfamoylphenyl)sulfonyl-benzamide
2-Benzyl-4-chloro-N-(2-sulfamoylphenyl)sulfonyl-benzamide
2-Phenyl-N-(2-sulfamoylphenyl)sulfonyl-benzofuran-5-carboxamide
4-Methyl-N-(2-sulfamoylphenyl)sulfonyl-2-[4-(trifluoromethyl)phenyl]1,3-thiazole-5-carboxamide
2-(2,3-Dihydrobenzofuran-5-yl)-4-methyl-N-(2-sulfamoylphenyl)sulfonyl-1,3-thiazole-5-carboxamide
2-(4-Chlorophenyl)-4-methyl-N-(2-sulfamoylphenyl)sulfonyl-1,3-thiazole-5-carboxamide
4-Methyl-2-phenyl-N-(2-sulfamoylphenyl)sulfonyl-1,3-thiazole-5-carboxamide
4-Phenylmethoxy-N-(2-sulfamoylphenyl)sulfonyl-benzamide
4-Phenyl-N-(2-sulfamoylphenyl)sulfonyl-benzamide
N-(2-Sulfamoylphenyl)sulfonyl-4-tert-butyl-benzamide
1-Methyl-N-(2-sulfamoylphenyl)sulfonyl-indole-2-carboxamide
5-Pyridin-2-yl-N-(2-sulfamoylphenyl)sulfonyl-thiophene-2-carboxamide
5-Phenyl-N-(2-sulfamoylphenyl)sulfonyl-thiophene-2-carboxamide
5-(3,4-Dichlorophenyl)-N-(2-sulfamoylphenyl)sulfonyl-furan-2-carboxamide
N-(2-Sulfamoylphenyl)sulfonyl-5-[3-(trifluoromethyl)phenyl]furan-2-carboxamide
1-(3,5-Dichlorophenyl)-5-propyl-N-(2-sulfamoylphenyl)sulfonyl-pyrazole-4-carboxamide
3,6-Dichloro-N-(2-sulfamoylphenyl)sulfonyl-benzothiophene-2-carboxamide
N-(2-Sulfamoylphenyl)sulfonylbenzothiophene-3-carboxamide
Ethyl 4-[5-[(2-Sulfamoylphenyl)sulfonylcarbamoyl]-2-furyl]benzoate
2-(3-Chlorophenyl)-4-methyl-N-(2-sulfamoylphenyl)sulfonyl-1,3-thiazole-5-carboxamide
4-(3,3-Dimethylbut-1-ynyl)-N-(2-sulfamoylphenylsulfonyl)benzamide
4-(3-Hydroxy-3-methylbut-1-ynyl)-N-(2-sulfamoylphenylsulfonyl)benzamide
4-(Benzofuran-2-yl)-2-methyl-N-(2-sulfamoylphenylsulfonyl)benzamide;
4-(Benzofuran-2-yl)-2-methyl-N-(2-sulfamoylphenylsulfonyl)benzamide;
4-(Benzofuran-2-yl)-3,5-dimethoxy-N-(2-sulfamoylphenylsulfonyl)benzamide;
4-(Benzofuran-2-yl)-2-methoxy-N-(2-sulfamoylphenylsulfonyl)benzamide;
4-(Benzofuran-2-yl)-2-hydroxy-N-(2-sulfamoylphenylsulfonyl)benzamide;
4-(Benzofuran-2-yl)-3-methoxy-N-(2-sulfamoylphenylsulfonyl)benzamide;
4-(Benzofuran-2-yl)-3-hydroxy-N-(2-sulfamoylphenylsulfonyl)benzamide;
4-(Benzofuran-2-yl)-2,6-dimethyl-N-(2-sulfamoylphenylsulfonyl)benzamide;
4-(3-Methoxyprop-1-ynyl)-N-(2-sulfamoylphenylsulfonyl)benzamide;
4-(3-Methylbut-3-en-1-ynyl)-N-(2-sulfamoylphenylsulfonyl)benzamide;
6-(Phenylethynyl)-N-(2-sulfamoylphenylsulfonyl)nicotinamide;
4-(3-Ethyl-3-hydroxypent-1-ynyl)-N-(2-sulfamoylphenylsulfonyl)benzamide;
4-(3-Hydroxy-3-methylpent-1-ynyl)-N-(2-sulfamoylphenylsulfonyl)benzamide;
4-((1-Hydroxycyclopentyl)ethynyl)-N-(2-sulfamoylphenylsulfonyl)benzamide;
3-(3-Hydroxy-3-methylbut-1-ynyl)-N-(2-sulfamoylphenylsulfonyl)benzamide;
3-(3,3-Dimethylbut-1-ynyl)-N-(2-sulfamoylphenylsulfonyl)benzamide;
4-(3,3-Dimethylbut-1-ynyl)-N-(2-sulfamoylphenylsulfonyl)-1-naphthamide;
4-(Benzofuran-2-yl)-N-(2-sulfamoylphenylsulfonyl)-1-naphthamide;
2-(Benzofuran-2-yl)-4-methyl-N-(2-sulfamoylphenylsulfonyl)thiazole-5-carboxamide;
3′-(3-Hydroxy-3-methylbut-1-ynyl)-N-(2-sulfamoylphenylsulfonyl)biphenyl-2-carboxamide;
4-(Cyclopentylethynyl)-N-(2-sulfamoylphenylsulfonyl)benzamide;
3-(Cyclopentylethynyl)-N-(2-sulfamoylphenylsulfonyl)benzamide;
4-(Cyclopentylethynyl)-2-methyl-N-(2-sulfamoylphenylsulfonyl)benzamide;
4-(3,3-Dimethylbut-1-ynyl)-3-methoxy-2-methyl-N-(2-sulfamoylphenylsulfonyl)benzamide;
4-(Benzofuran-2-yl)-3-methoxy-2-methyl-N-(2-sulfamoylphenylsulfonyl)benzamide;
4-(Pyridin-3-ylethynyl)-N-(2-sulfamoylphenylsulfonyl)benzamide;
4-(Pyridin-2-ylethynyl)-N-(2-sulfamoylphenylsulfonyl)benzamide;
4-(Phenylethynyl)-N-(2-sulfamoylphenylsulfonyl)benzamide;
4-(3,3-Dimethylbut-1-ynyl)-3-fluoro-N-(2-sulfamoylphenylsulfonyl)benzamide;
2-(3-Methoxyphenyl)-N-(2-sulfamoylphenylsulfonyl)benzofuran-5-carboxamide;
2-(4-Methoxyphenyl)-N-(2-sulfamoylphenylsulfonyl)benzofuran-5-carboxamide;
2-tert-Butyl-N-(2-sulfamoylphenylsulfonyl)benzofuran-5-carboxamide;
2-(1-Hydroxycyclopentyl)-N-(2-sulfamoylphenylsulfonyl)benzofuran-5-carboxamide;
2-Cyclopentyl-N-(2-sulfamoylphenylsulfonyl)benzofuran-5-carboxamide;
3-Cyano-4-(3,3-dimethylbut-1-ynyl)-N-(2-sulfamoylphenylsulfonyl)benzamide;
4-(Benzofuran-2-yl)-3-cyano-N-(2-sulfamoylphenylsulfonyl)benzamide;
4-Chloro-2-hydroxy-N-(2-sulfamoylphenylsulfonyl)benzamide;
4-Bromo-2-hydroxy-N-(2-sulfamoylphenylsulfonyl)benzamide;
4-(Benzofuran-2-yl)-2-fluoro-N-(2-sulfamoylphenylsulfonyl)benzamide;
4-(3,3-Dimethylbut-1-ynyl)-2-fluoro-N-(2-sulfamoylphenylsulfonyl)benzamide;
4-(Cyclopentylethynyl)-2-fluoro-N-(2-sulfamoylphenylsulfonyl)benzamide;
4-(Cyclopentylethynyl)-2-fluoro-3-methoxy-N-(2-sulfamoylphenylsulfonyl)benzamide;
4-(Benzofuran-2-yl)-2-fluoro-3-methoxy-N-(2-sulfamoylphenylsulfonyl)benzamide;
5-(Cyclohexylethynyl)-N-(2-sulfamoylphenylsulfonyl)picolinamide;
5-(3,3-Dimethylbut-1-ynyl)-N-(2-sulfamoylphenylsulfonyl)picolinamide;
4-(3,3-Dimethylbut-1-ynyl)-2-fluoro-3-methoxy-N-(2-sulfamoylphenylsulfonyl)benzamide;
4-(Benzofuran-2-yl)-2-chloro-N-(2-sulfamoylphenylsulfonyl)benzamide;
4-(Cyclopentylethynyl)-2-hydroxy-N-(2-sulfamoylphenylsulfonyl)benzamide;
6-(Cyclopentylethynyl)-N-(2-sulfamoylphenylsulfonyl)nicotinamide;
6-(Pyridin-2-ylethynyl)-N-(2-sulfamoylphenylsulfonyl)nicotinamide;
6-(Pyridin-3-ylethynyl)-N-(2-sulfamoylphenylsulfonyl)nicotinamide;
2-(3,3-Dimethylbut-1-ynyl)-N-(2-sulfamoylphenylsulfonyl)pyrimidine-5-carboxamide;
N-(2-Sulfamoylphenylsulfonyl)-4-((3,3,3-trifluoropropoxy)methyl)benzamide;
4-(Cyclopentylethynyl)-3-(hydroxymethyl)-N-(2-sulfamoylphenylsulfonyl)benzamide;
6-(3-Methylbut-1-ynyl)-N-(2-sulfamoylphenylsulfonyl)nicotinamide;
3-(Hydroxymethyl)-4-(phenylethynyl)-N-(2-sulfamoylphenylsulfonyl)benzamide;
4-(Cyclohexylethynyl)-3-(hydroxymethyl)-N-(2-sulfamoylphenylsulfonyl)benzamide;
2-((4-chlorophenyl)ethynyl)-N-(2-sulfamoylphenylsulfonyl)pyrimidine-5-carboxamide;
4-(Benzofuran-2-yl)-3-(hydroxymethyl)-N-(2-sulfamoylphenylsulfonyl)benzamide;
4-(Benzofuran-2-yl)-N-(2-sulfamoylphenylsulfonyl)cyclohexanecarboxamide;
(1S,4S)-4-(Benzofuran-2-yl)-N-(2-sulfamoylphenylsulfonyl)cyclohexanecarboxamide;
(1R,4R)-4-(Benzofuran-2-yl)-N-(2-sulfamoylphenylsulfonyl)cyclohexanecarboxamide;
4-(Benzofuran-2-yl)-1-methyl-N-(2-sulfamoylphenylsulfonyl)cyclohexanecarboxamide;
(1R,4R)-4-(Benzofuran-2-yl)-1-methyl-N-(2-sulfamoylphenylsulfonyl)cyclohexanecarboxamide;
(1S,4S)-4-(Benzofuran-2-yl)-1-methyl-N-(2-sulfamoylphenylsulfonyl)cyclohexanecarboxamide;
4-(3,3-Dimethylbut-1-ynyl)-3-methoxy-N-(2-sulfamoylphenylsulfonyl)benzamide;
4-(Cyclopropylethynyl)-3-methoxy-N-(2-sulfamoylphenylsulfonyl)benzamide;
4-(3-Methoxy-3-methylbut-1-ynyl)-N-(2-sulfamoylphenylsulfonyl)benzamide;
4-(3-Methylbut-1-ynyl)-N-(2-sulfamoylphenylsulfonyl)benzamide;
3-Methoxy-4-(3-methoxy-3-methylbut-1-ynyl)-N-(2-sulfamoylphenylsulfonyl)-benzamide;
3-Hydroxy-4-(3-methoxy-3-methylbut-1-ynyl)-N-(2-sulfamoylphenylsulfonyl)-benzamide;
6-(3,3-Dimethylbut-1-ynyl)-N-(2-sulfamoylphenylsulfonyl)nicotinamide;
6-(Benzofuran-2-yl)-N-(2-sulfamoylphenylsulfonyl)nicotinamide;
4-(3,3-Dimethylbut-1-ynyl)-3-(2-(2-methoxyethoxy)ethoxy)-N-(2-sulfamoylphenylsulfonyl)benzamide;
4-(Benzofuran-2-yl)-3-(2-(2-methoxyethoxy)ethoxy)-N-(2-sulfamoylphenylsulfonyl)benzamide;
2-(2-Methoxyphenyl)-N-(2-sulfamoylphenylsulfonyl)benzofuran-5-carboxamide;
2-(1-tert-Butoxyethyl)-N-(2-sulfamoylphenylsulfonyl)benzofuran-5-carboxamide;
2-(Pyridin-2-yl)-N-(2-sulfamoylphenylsulfonyl)benzofuran-5-carboxamide;
2-(Pyridin-3-yl)-N-(2-sulfamoylphenylsulfonyl)benzo furan-5-carboxamide;
2-(2-Hydroxypropan-2-yl)-N-(2-sulfamoylphenylsulfonyl)benzofuran-5-carboxamide;
2-(2-Methoxypropan-2-yl)-N-(2-sulfamoylphenylsulfonyl)benzofuran-5-carboxamide;
2-Cyclopropyl-N-(2-sulfamoylphenylsulfonyl)benzofuran-5-carboxamide;
4-(Benzofuran-2-yl)-3-isopropoxy-N-(2-sulfamoylphenylsulfonyl)benzamide;
4-(3,3-Dimethylbut-1-ynyl)-3-isopropoxy-N-(2-sulfamoylphenylsulfonyl)benzamide;
4-(3-Hydroxy-3-methylbut-1-ynyl)-3-isopropoxy-N-(2-sulfamoylphenylsulfonyl)benzamide;
4-(Cyclopentylethynyl)-3-isopropoxy-N-(2-sulfamoylphenylsulfonyl)benzamide;
4-(Cyclohexylethynyl)-3-isopropoxy-N-(2-sulfamoylphenylsulfonyl)benzamide;
4-(Cyclopropylethynyl)-3-isopropoxy-N-(2-sulfamoylphenylsulfonyl)benzamide;
4-((1-Hydroxycycloheptyl)ethynyl)-3-isopropoxy-N-(2-sulfamoylphenylsulfonyl)benzamide;
6-(3,3-Dimethylbut-1-ynyl)-5-(2-(2-methoxyethoxy)ethoxy)-N-(2-sulfamoylphenylsulfonyl)nicotinamide;
6-(Benzofuran-2-yl)-5-(2-(2-methoxyethoxy)ethoxy)-N-(2-sulfamoylphenylsulfonyl)nicotinamide;
6-(Cyclopentylethynyl)-5-(2-(2-methoxyethoxy)ethoxy)-N-(2-sulfamoylphenylsulfonyl)nicotinamide;
6-(Cyclopentylethynyl)-5-methoxy-N-(2-sulfamoylphenylsulfonyl)nicotinamide;
6-(Cyclohexylethynyl)-5-methoxy-N-(2-sulfamoylphenylsulfonyl)nicotinamide;
5-Methoxy-N-(2-sulfamoylphenylsulfonyl)-6-((4-(trifluoromethyl)phenyl)ethynyl)nicotinamide;
N-(3-Dimethylaminopropyl)-N′-ethylcarbodiimide hydrochloride;
1-(2-Methoxyethyl)-2-phenyl-N-(2-sulfamoylphenylsulfonyl)-1H-indole-5-carboxamide;
6-(Cyclopropylethynyl)-5-isopropoxy-N-(2-sulfamoylphenylsulfonyl)nicotinamide;
6-(Cyclopentylethynyl)-5-isopropoxy-N-(2-sulfamoylphenylsulfonyl)nicotinamide;
6-(Cyclohexylethynyl)-5-isopropoxy-N-(2-sulfamoylphenylsulfonyl)nicotinamide-4-(Benzofuran-2-yl)-3-(3-methoxy-3-methylbutoxy)-N-(2-sulfamoylphenylsulfonyl)benzamide;
4-(Cyclopentylethynyl)-3-fluoro-N-(2-sulfamoylphenylsulfonyl)benzamide;
6-(Benzofuran-2-yl)-5-chloro-N-(2-sulfamoylphenylsulfonyl)nicotinamide;
5-Chloro-6-(cyclopentylethynyl)-N-(2-sulfamoylphenylsulfonyl)nicotinamide;
5-Chloro-6-(3,3-dimethylbut-1-ynyl)-N-(2-sulfamoylphenylsulfonyl)nicotinamide;
4-(Benzofuran-2-yl)-N-(2-sulfamoylphenylsulfonyl)-2-(trifluoromethyl)benzamide;
4-(3,3-Dimethylbut-1-ynyl)-N-(2-sulfamoylphenylsulfonyl)-2-(trifluoromethyl)benzamide;
4-(Benzofuran-2-yl)-2,6-difluoro-N-(2-sulfamoylphenylsulfonyl)benzamide;
4-(Cyclopentylethynyl)-2,6-difluoro-N-(2-sulfamoylphenylsulfonyl)benzamide;
4-(Benzofuran-2-yl)-3-(3-hydroxy-3-methylbut-1-ynyl)-N-(2-sulfamoylphenylsulfonyl)benzamide;
4-(Benzofuran-2-yl)-3-bromo-N-(2-sulfamoylphenylsulfonyl)benzamide;
4-(Benzyloxy)-3-(3-hydroxy-3-methylbut-1-ynyl)-N-(2-sulfamoylphenylsulfonyl)benzamide;
4-(Benzyloxy)-3-iodo-N-(2-sulfamoylphenylsulfonyl)benzamide;
2-Benzyl-N-(2-sulfamoylphenylsulfonyl)-1H-indole-5-carboxamide;
7-(Cyclopropylethynyl)-2,2-difluoro-N-(2-sulfamoylphenylsulfonyl)benzo[d][1,3]dioxole-4-carboxamide;
4-(Cyclopropylethynyl)-N-(2-sulfamoylphenylsulfonyl)-3-(3,3,3-trifluoropropoxy)benzamide;
4-(Benzofuran-2-yl)-N-(4-(hydroxymethyl)-2-sulfamoylphenylsulfonyl)benzamide;
Benzene-1,2-disulfonic acid 1-amide 2[(quinoline-3-carbonyl)-amide]
   and pharmaceutically acceptable salts of any one thereof.

The present invention further provides a process for the preparation of a compound of formula (I) or a pharmaceutically acceptable salt thereof as defined above which comprises,

(a) reacting a compound of formula (II)

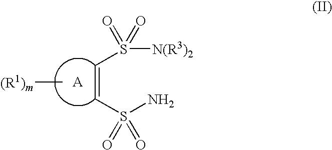

wherein R¹, R³, A and m are as defined in formula (I),
with a compound of formula (III)

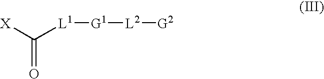

wherein L¹, L², G¹ and G² are as defined in formula (I) and X represents a leaving group such as OH or halogen; or
(b) when L² represents a direct bond and G¹ and G² are both aromatic moieties, reacting a compound of formula (IV)

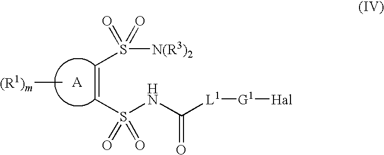

wherein Hal represents a halogen atom and R¹, R³, A, m and L¹ are as defined in formula (I),
with a nucleophile G²-M wherein M represents an organo-tin or organo boronic acid group;
and optionally after (a) or (b) carrying out one or more of the following:
converting the compound obtained to a further compound of the invention
forming a pharmaceutically acceptable salt of the compound.

In process (a), the reaction may conveniently be carried out in an organic solvent such as acetonitrile, dichloromethane, N,N-dimethylformamide or N-methylpyrrolidinone at a temperature, for example, in the range from 0° C. to the boiling point of the solvent. If necessary or desired, a base and/or a coupling reagent such as 4-(dimethylamino)pyridine (DMAP), 1-ethyl-3-(3-dimethylaminopropyl)carbodiimide hydrochloride (EDC), HATU (O-(7-Azabenzotriazol-1-yl)-N,N,N′,N′-tetramethyluronium hexafluorophosphate), O-(1H-benzotriazol-1-yl)-N,N,N′,N′-tetramethyluronium (HBTU), HOAT (1-Hydroxy-7-azabenzotriazole), HOBT (1-Hydroxybenzotriazole hydrate), triethylamine or DIEA (N,N-Diisopropylethylamine), and any combinations of the above, may be added. In one embodiment, the solvent is N,N-dimethylformamide and 4-(dimethylamino)pyridine (DMAP) and 1-ethyl-3-(3-dimethylaminopropyl)carbodiimide hydrochloride (EDC) are used as reagents.

In process (b), the reaction may conveniently be carried out by reaction with an appropriate aryl boronic acid or an aryl boronic ester. The reaction may be carried out using a suitable palladium catalyst such as Pd(PPh₃)₄, Pd(dppf)Cl₂, or Pd(OAc)₂ or Pd₂(dba)₃ together with a suitable ligand such as P(tert-butyl)₃, 2-(dicyclohexylphosphino)biphenyl, or 2-(2′,6′-dimethoxybiphenyl)-dicyclohexylphosphine, or a nickel catalyst such as nickel on charcoal or Ni(dppe)Cl₂ together with zinc and sodium triphenylphosphinetrimetasulfonate. A suitable base such as an alkyl amine, e.g. triethylamine, or potassium carbonate, sodium carbonate, cesium carbonate, sodium hydroxide or cesium fluoride may be used in the reaction, which can be performed in the temperature range of +20° C. to +160° C., using an oil bath or a microwave oven, in a suitable solvent or solvent mixture such as toluene, tetrahydrofuran, dimethoxyethane/water, N,N-dimethylformamide or dioxane. The boronic acid or boronic ester may be formed in situ, by reaction of the corresponding aryl halide (e.g., the aryl bromide) with an alkyllithium reagent such as butyllithium to form an intermediate aryl lithium species, which then is reacted with a suitable boron compound, e.g., trimethyl borate, tributyl borate or triisopropyl borate.

Alternatively, the reaction may be carried out by reaction with an appropriate alkyne. The reaction may be carried out using a suitable palladium catalyst such as Pd(PPh₃)₄, PdCl₂(PPh₃)₂, [PdCl₂(CH₃CN)₂] or Pd(PPh₃)₂(OAc)₂. The reaction may be preformed in the presence of a suitable ligand such as Xphos. The reaction may be preformed in the presence of a suitable copper catalyst such as copper(I) iodide. A suitable base such as triethylamine, buthylamine, diisopropylamine or cesium carbonate may be used in the reaction, which can be performed in the temperature range of +20° C. to +160° C., using an oil bath or a microwave oven, in a suitable solvent or a mixture of solvents such as N,N-dimethylformamide, dimethyl sulfoxide, acetonitrile, toluene, tetrahydrofuran, dimethoxyethane/water or dioxane.

Specific processes for the preparation of compounds of Formula (I) are disclosed within the Examples section of the present specification. Such processes form an aspect of the present invention.

The necessary starting materials are either commercially available, are known in the literature or may be prepared using known techniques. Specific processes for the preparation of certain key starting materials are disclosed within the Examples section of the present specification and such processes form an aspect of the present invention. Certain intermediates are novel. Such novel intermediates form another aspect of the invention.

Compounds of formula (I) can be converted into further compounds of formula (I) using standard procedures.

It will be appreciated by those skilled in the art that in the processes of the present invention certain functional groups such as hydroxyl or amino groups may need to be protected by protecting groups. Thus, the preparation of the compounds of formula (I) may involve, at an appropriate stage, the addition and/or removal of one or more protecting groups.

The protection and deprotection of functional groups is described in ‘Protective Groups in Organic Chemistry’, edited by J. W. F. McOmie, Plenum Press (1973) and ‘Protective Groups in Organic Synthesis’, 3^{rd} edition, T. W. Greene and P. G. M. Wuts, Wiley-Interscience (1999).

As used herein, a pharmaceutically acceptable salt is a salt with a pharmaceutically acceptable acid or base. Pharmaceutically acceptable acids include both inorganic acids such as hydrochloric, sulphuric, phosphoric, diphosphoric, hydrobromic or nitric acid and organic acids such as citric, fumaric, maleic, malic, ascorbic, succinic, tartaric, benzoic, acetic, methanesulphonic, ethanesulphonic, benzenesulphonic or p-toluenesulphonic acid. Pharmaceutically acceptable bases include alkali metal (e.g. sodium or potassium) and alkali earth metal (e.g. calcium or magnesium) hydroxides and organic bases such as alkyl amines, aralkyl amines and heterocyclic amines. Compounds of formula (I) are capable of existing in stereoisomeric forms. It will be understood that the invention encompasses the use of all geometric and optical isomers (including atropisomers) of the compounds of formula (I) and mixtures thereof including racemates. The use of tautomers and mixtures thereof also form an aspect of the present invention. Enantiomerically pure forms are particularly desired.

The compounds of formula (I) and their pharmaceutically acceptable salts have activity as pharmaceuticals, in particular as selective inhibitors of the microsomal prostaglandin E synthase-1 enzyme, and may therefore be beneficial in the treatment or prophylaxis of pain and of inflammatory diseases and conditions. Furthermore, by selectively inhibiting the pro-inflammatory PGE2, it is believed that compounds of the invention would have a reduced potential for side effects associated with the inhibition of other prostaglandins by conventional non-steroidal anti-inflammatory drugs, such as gastrointestinal and renal toxicity.

More particularly, the compounds of formula (I) and their pharmaceutically acceptable salts may be used in the treatment of osteoarthritis, rheumatoid arthritis, acute or chronic pain, neuropathic pain, apnea, sudden infant death (SID), wound healing, cancer, benign or malignant neoplasias, stroke, atherosclerosis and Alzheimer's disease.

Even more particularly, the compounds of formula (I) and their pharmaceutically acceptable salts may be used in the treatment of osteoarthritis, rheumatoid arthritis, benign or malignant neoplasias or acute or chronic pain.

Thus, the present invention provides a compound of formula (I) or a pharmaceutically-acceptable salt thereof as hereinbefore defined for use in therapy.

In a further aspect, the present invention provides the use of a compound of formula (I) or a pharmaceutically acceptable salt thereof as hereinbefore defined in the manufacture of a medicament for use in therapy.

One aspect of the invention provides compound of formula (I) or a pharmaceutically acceptable salt thereof

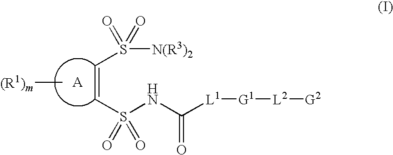

wherein:
A is selected from phenyl or a 5- or 6-membered heteroaryl moiety; said phenyl or a 5- or 6-membered heteroaryl moiety in group A being optionally fused to a phenyl, a 5- or 6-membered heteroaryl, C₅₋₆-carbocyclyl or C₅₋₆heterocyclyl ring;
R¹ is independently selected from halogen, nitro, SF₅, OH, CHO, CO₂R⁴, CONR⁵R⁶, C₁₋₄alkyl, C₁₋₄alkoxy, G³, OG³ or OCH₂G³; said C₁₋₄alkyl or C₁₋₄alkoxy being optionally substituted by OH or by one or more F atoms;
m represents an integer 0, 1 or 2;
Each R³ is independently selected from hydrogen, CN and C₁₋₄alkyl; said C₁₋₄alkyl being optionally substituted with OH, CN, C₁₋₄alkoxy, NR⁷R⁸ or one or more F atoms;
L¹ represents a direct bond, C₁₋₄alkylene, C₂₋₄alkenylene or C₂₋₄alkynylene;
L² represents a direct bond, —O—, —OCH₂—, C₁₋₂alkylene or —C≡C—;
G¹ represents phenyl, 5- or 6-membered heteroaryl, C₃₋₁₀carbocyclyl or C₅₋₈heterocyclyl;
G² represents H, C₁₋₆alkyl, C₁₋₆alkenyl, phenyl, 5- or 6-membered heteroaryl, C₃₋₁₀carbocyclyl or
C₅₋₈heterocyclyl; said C₁₋₆alkyl being optionally further substituted by one or more groups selected from OH, C₁₋₆alkoxy and halogen;
The phenyl, heteroaryl, carbocyclyl or heterocyclyl moieties in G¹ and G² being optionally fused to one or two further rings independently selected from phenyl, a 5- or 6-membered heteroaryl, C₅₋₆-carbocyclyl or C₅₋₆heterocyclyl ring;
Any phenyl, heteroaryl, carbocyclyl or heterocyclyl moieties in G¹ and G² being optionally substituted by one or more substituents independently selected from halogen, OH, CN, NO₂, CO₂R⁹, C₁₋₆alkyl, C₁₋₆alkoxy, C₁₋₄thioalkoxy, SO₂NR¹⁰R¹¹, NR¹²R¹³, —O(CH₂)₂—O—(CH₂)₂—C₁₋₆alkoxy, —NHCOC(OH)(CH₃)CF₃, —CH₂OCH₂CF₂CHF₂ or —CH₂OCH₂CH₂CF₃; said C₁₋₆alkyl or C₁₋₆alkoxy being optionally substituted by OH, C₁₋₆alkoxy, phenyl or by one or more F atoms;
G³ represents phenyl or 5- or 6-membered heteroaryl; and
Each R⁴, R⁵, R⁶, R⁷, R⁸, R⁹, R¹⁰, R¹¹, R¹² and R¹³ is independently selected from H or C₁₋₄alkyl,
for use in therapy.

In a further aspect, the present invention provides the use of a compound of formula (I) or a pharmaceutically acceptable salt thereof as hereinbefore defined in the manufacture of a medicament for the treatment of human diseases or conditions in which modulation of microsomal prostaglandin E synthase-1 activity is beneficial.

In a further aspect, the present invention provides the use of a compound of formula (I) or a pharmaceutically acceptable salt thereof as hereinbefore defined in the manufacture of a medicament for use in the treatment of an inflammatory disease or condition.

In a further aspect, the present invention provides the use of a compound of formula (I) or a pharmaceutically acceptable salt thereof as hereinbefore defined in the manufacture of a medicament for use in treating osteoarthritis, rheumatoid arthritis, acute or chronic pain, neuropathic pain, apnea, SID, wound healing, cancer, benign or malignant neoplasias, stroke, atherosclerosis or Alzheimer's disease.

In a further aspect, the present invention provides the use of a compound of formula (I) or a pharmaceutically acceptable salt thereof as hereinbefore defined in the manufacture of a medicament for use in treating acute or chronic pain, nociceptive pain, neuropathic pain, apnea, sudden infant death (SID), atherosclerosis, cancer, aneurysm, hyperthermia, myositis, Alzheimer's disease or arthritis.

In a further aspect, the present invention provides the use of a compound of formula (I) or a pharmaceutically acceptable salt thereof as hereinbefore defined in the manufacture of a medicament for use in treating osteoarthritis, rheumatoid arthritis, benign or malignant neoplasias or acute or chronic pain.

In another aspect, the invention provides a compound of formula (I) or a pharmaceutically acceptable salt thereof as hereinbefore defined for use as a medicament.

In another aspect, the invention provides a compound of formula (I) or a pharmaceutically acceptable salt thereof as hereinbefore defined for the treatment of diseases or conditions in which modulation of microsomal prostaglandin E synthase-1 activity is beneficial.

In another aspect, the invention provides a compound of formula (I) or a pharmaceutically acceptable salt thereof as hereinbefore defined for the treatment of an inflammatory disease or condition.

In another aspect, the invention provides a compound of formula (I) or a pharmaceutically acceptable salt thereof as hereinbefore defined for the treatment of osteoarthritis, rheumatoid arthritis, acute or chronic pain, neuropathic pain, apnea, SID, wound healing, cancer, benign or malignant neoplasias, stroke, atherosclerosis or Alzheimer's disease. In another aspect, the invention provides a compound of formula (I) or a pharmaceutically acceptable salt thereof as hereinbefore defined for the treatment of osteoarthritis, rheumatoid arthritis, benign or malignant neoplasias or acute or chronic pain. In the context of the present specification, the term “therapy” also includes “prophylaxis” unless there are specific indications to the contrary. The terms “therapeutic” and “therapeutically” should be construed accordingly.

Prophylaxis is expected to be particularly relevant to the treatment of persons who have suffered a previous episode of, or are otherwise considered to be at increased risk of, the disease or condition in question. Persons at risk of developing a particular disease or condition generally include those having a family history of the disease or condition, or those who have been identified by genetic testing or screening to be particularly susceptible to developing the disease or condition.

The invention also provides a method of treating, or reducing the risk of, a disease or condition in which modulation of microsomal prostaglandin E synthase-1 activity is beneficial which comprises administering to a patient in need thereof a therapeutically effective amount of a compound of formula (I) or a pharmaceutically acceptable salt thereof as hereinbefore defined.

The invention still further provides a method of treating, or reducing the risk of, an inflammatory disease or condition which comprises administering to a patient in need thereof a therapeutically effective amount of a compound of formula (I) or a pharmaceutically acceptable salt thereof as hereinbefore defined.

The invention still further provides a method of treating, or reducing the risk of, osteoarthritis, rheumatoid arthritis, acute or chronic pain, neuropathic pain, apnea, SID, wound healing, cancer, benign or malignant neoplasias, stroke, atherosclerosis or Alzheimer's disease which comprises administering to a patient in need thereof a therapeutically effective amount of a compound of formula (I) or a pharmaceutically acceptable salt thereof as hereinbefore defined.

The invention still further provides a method of treating, or reducing the risk of, osteoarthritis, rheumatoid arthritis, benign or malignant neoplasias or acute or chronic pain which comprises administering to a patient in need thereof a therapeutically effective amount of a compound of formula (I) or a pharmaceutically acceptable salt thereof as hereinbefore defined.

For the above-mentioned therapeutic uses the dosage administered will, of course, vary with the compound employed, the mode of administration, the treatment desired and the disorder indicated. The daily dosage of the compound of the invention may be in the range from 0.05 mg/kg to 100 mg/kg.

The compounds of formula (I) and pharmaceutically acceptable salts thereof may be used on their own but will generally be administered in the form of a pharmaceutical composition in which the formula (I) compound/salt (active ingredient) is in association with a pharmaceutically acceptable adjuvant, diluent or carrier. Conventional procedures for the selection and preparation of suitable pharmaceutical formulations are described in, for example, “Pharmaceuticals—The Science of Dosage Form Designs”, M. E. Aulton, Churchill Livingstone, 1988.

Depending on the mode of administration, the pharmaceutical composition will preferably comprise from 0.05 to 99% w (percent by weight), more preferably from 0.05 to 80% w, still more preferably from 0.10 to 70% w, and even more preferably from 0.10 to 50% w, of active ingredient, all percentages by weight being based on total composition.

The present invention also provides a pharmaceutical composition comprising a compound of formula (I) or a pharmaceutically acceptable salt thereof as hereinbefore defined, in association with a pharmaceutically acceptable adjuvant, diluent or carrier.

The invention further provides a process for the preparation of a pharmaceutical composition of the invention which comprises mixing a compound of formula (I) or a pharmaceutically acceptable salt thereof as hereinbefore defined with a pharmaceutically acceptable adjuvant, diluent or carrier.

The pharmaceutical compositions may be administered topically (e.g. to the skin) in the form, e.g., of creams, solutions or suspensions; or systemically, e.g. by oral administration in the form of tablets, capsules, syrups, powders or granules; or by parenteral administration in the form of solutions or suspensions; or by subcutaneous administration; or by rectal administration in the form of suppositories; or transdermally.

For oral administration the compound of the invention may be admixed with an adjuvant or a carrier, for example, lactose, saccharose, sorbitol, mannitol; a starch, for example, potato starch, corn starch or amylopectin; a cellulose derivative; a binder, for example, gelatine or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatine, talcum and titanium dioxide. Alternatively, the tablet may be coated with a suitable polymer dissolved in a readily volatile organic solvent.

For the preparation of soft gelatine capsules, the compound of the invention may be admixed with, for example, a vegetable oil or polyethylene glycol. Hard gelatine capsules may contain granules of the compound using either the above-mentioned excipients for tablets. Also liquid or semisolid formulations of the compound of the invention may be filled into hard gelatine capsules.

Liquid preparations for oral application may be in the form of syrups or suspensions, for example, solutions containing the compound of the invention, the balance being sugar and a mixture of ethanol, water, glycerol and propylene glycol. Optionally such liquid preparations may contain colouring agents, flavouring agents, saccharine and/or carboxymethylcellulose as a thickening agent or other excipients known to those skilled in art.

The compounds of the invention may also be administered in conjunction with other compounds used for the treatment of the above conditions.

Thus, the invention further relates to combination therapies wherein a compound of formula (I) or a pharmaceutically acceptable salt thereof, or a pharmaceutical composition or formulation comprising a compound of formula (I) is administered concurrently, simultaneously, sequentially or separately with another pharmaceutically active compound or compounds selected from the following:

(i) neuropathic pain therapies including for example gabapentin, lidoderm, pregablin and equivalents and pharmaceutically active isomer(s) and metabolite(s) thereof.
(ii) nociceptive pain therapies such as celecoxib, etoricoxib, lumiracoxib, rofecoxib, valdecoxib, diclofenac, loxoprofen, naproxen, paracetamol and equivalents and pharmaceutically active isomer(s) and metabolite(s) thereof.
(iii) migraine therapies including for example almotriptan, amantadine, bromocriptine, butalbital, cabergoline, dichloralphenazone, eletriptan, frovatriptan, lisuride, naratriptan, pergolide, pramipexole, rizatriptan, ropinirole, sumatriptan, zolmitriptan, zomitriptan, and equivalents and pharmaceutically active isomer(s) and metabolite(s) thereof.

Such combination products employ the compounds of this invention within the dosage range described herein and the other pharmaceutically active compound or compounds within approved dosage ranges and/or the dosage described in their respective publication reference(s).

Chemical names were generated by CambridgeSoft MedChem ELN v2.1.

### SPECIFIC EMBODIMENTS

1. A compound of formula (I) or a pharmaceutically acceptable salt thereof

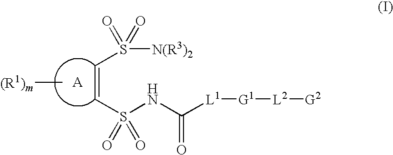

wherein:
A is selected from phenyl or a 5- or 6-membered heteroaryl moiety; said phenyl or a 5- or 6-membered heteroaryl moiety in group A being optionally fused to a phenyl, a 5- or 6-membered heteroaryl, C₅₋₆-carbocyclyl or C₅₋₆heterocyclyl ring;
R¹ is independently selected from halogen, nitro, SF₅, OH, CHO, CO₂R⁴, CONR⁵R⁶, C₁₋₄alkyl, C₁₋₄alkoxy, G³, OG³ or OCH₂G³; said C₁₋₄alkyl or C₁₋₄alkoxy being optionally substituted by OH or by one or more F atoms;
m represents an integer 0, 1 or 2;
R³ is hydrogen;
L¹ represents a direct bond, C₁₋₄alkylene, C₂₋₄alkenylene or C₂₋₄alkynylene;
L² represents a direct bond, —O—, —OCH₂—, C₁₋₂alkylene or —C≡C—;
G¹ represents phenyl, 5- or 6-membered heteroaryl, C₃₋₁₀carbocyclyl or C₅₋₈heterocyclyl;
G² represents H, C₁₋₆alkyl, C₁₋₆alkenyl, phenyl, 5- or 6-membered heteroaryl, C₃₋₁₀carbocyclyl or
C₅₋₈heterocyclyl; said C₁₋₆alkyl being optionally further substituted by one or more groups selected from OH, C₁₋₆alkoxy and halogen;
the phenyl, heteroaryl, carbocyclyl or heterocyclyl moieties in G¹ and G² being optionally fused to one or two further rings independently selected from phenyl, a 5- or 6-membered heteroaryl, C₅₋₆-carbocyclyl or C₅₋₆heterocyclyl ring;
any phenyl, heteroaryl, carbocyclyl or heterocyclyl moieties in G¹ and G² being optionally substituted by one or more substituents independently selected from halogen, OH, CN, NO₂, CO₂R⁹, C₁₋₆alkyl, C₁₋₆alkoxy, C₁₋₄thioalkoxy, SO₂NR¹⁰R¹¹,NR¹²R¹³, —O(CH₂)₂—O—(CH₂)₂—C₁₋₆alkoxy, —NHCOC(OH)(CH₃)CF₃, —CH₂OCH₂CF₂CHF₂ or —CH₂OCH₂CH₂CF₃; said C₁₋₆alkyl or C₁₋₆alkoxy being optionally substituted by OH, C₁₋₆alkoxy, phenyl or by one or more F atoms;
G³ represents phenyl or 5- or 6-membered heteroaryl; and
each R⁴, R⁵, R⁶, R⁹, R¹⁰, R¹¹, R¹² and R¹³ is independently selected from H or C₁₋₄alkyl;
provided that the compounds
1,2-Benzenedisulfonamide, N1-[[(4,6-dimethyl-2-pyrimidinyl)amino]carbonyl];
1,2-Benzenedisulfonamide, N1-[[(4,6-dimethoxy-1,3,5-triazin-2-yl)amino]carbonyl];
1,2-Benzenedisulfonamide, N1-[[(4-methoxy-6-methyl-2-pyrimidinyl)amino]carbonyl];
1,2-Benzenedisulfonamide, N1-[[(4,6-dimethoxy-2-pyrimidinyl)amino]carbonyl] are excluded.
   2. A compound according to embodiment 1 wherein
   G¹ represents phenyl, 5- or 6-membered heteroaryl, C₃₋₁₀carbocyclyl or C₅₋₈heterocyclyl;
   G² represents H, C₁₋₆alkyl, phenyl, 5- or 6-membered heteroaryl, C₃₋₁₀carbocyclyl or C₅₋₈heterocyclyl; said C₁₋₆alkyl being optionally further substituted by one or more groups selected from OH, C₁₋₆alkoxy and halogen;
   any phenyl, heteroaryl, carbocyclyl or heterocyclyl moieties in G¹ and G² being optionally substituted by one or more substituents independently selected from halogen, OH, CN, NO₂, CO₂R⁹, C₁₋₆alkyl, C₁₋₆alkoxy, C₁₋₄thioalkoxy, SO₂NR¹⁰R¹¹,NR¹²R¹³, —NHCOC(OH)(CH₃)CF₃ or —CH₂OCH₂CF₂CHF₂; said C₁₋₆alkyl or C₁₋₆alkoxy being optionally substituted by OH or by one or more F atoms.
   3. A compound according to embodiments 1 or 2 wherein A represents phenyl.
   4. A compound according to any one of embodiments 1-3 wherein R¹ is independently selected from halogen, C₁₋₄alkyl or C₁₋₄alkoxy; said C₁₋₄alkyl or C₁₋₄alkoxy being optionally substituted by OH or by one or more F atoms;
   5. A compound according to any one of embodiments 1-4 wherein m is 0 or 1.
   6. A compound according to any one of embodiments 1-4 wherein m is 0.
   7. A compound according to any one of embodiments 1-6 wherein L¹ is a direct bond or C₁₋₄alkylene.
   8. A compound according to any one of embodiments 1-7 wherein L² is a direct bond, —OCH₂— or —C≡C—.
   9. A compound according to embodiment 1 wherein any phenyl, heteroaryl, carbocyclyl or heterocyclyl moieties in G¹ and G² is optionally substituted by one or more substituents independently selected from halogen, CO₂R⁹, C₁₋₆alkyl, C₁₋₆alkoxy, —O(CH₂)₂—O—(CH₂)₂—C₁₋₆alkoxy, —CH₂OCH₂CF₂CHF₂ or —CH₂OCH₂CH₂CF₃; said C₁₋₆alkyl or C₁₋₆alkoxy being optionally substituted by OH, C₁₋₆alkoxy, phenyl or by one or more F atoms.
   10. A compound according to any one of embodiments 1-9 wherein any phenyl, heteroaryl, carbocyclyl or heterocyclyl moieties in G¹ and G² is optionally substituted by one or more substituents independently selected from halogen, CO₂R⁹, C₁₋₆alkyl, C₁₋₆alkoxy or
   —CH₂OCH₂CF₂CHF₂; said C₁₋₆alkyl or C₁₋₆alkoxy being optionally substituted by OH or by one or more F atoms.
   11. A compound according to any one of embodiments 1-10 wherein G¹ is phenyl, pyridyl, thiazolyl, thienyl, furanyl, pyrimidinyl, cyclohexyl, adamantyl or bicycloheptyl.
   12. A compound according to any one of embodiments 1-11 wherein G² is phenyl, benzofuranyl, benzothienyl, benzthiazolyl, [1,3]oxazolo[4,5-c]pyridyl, [1,3]oxazolo[5,4-c]pyridyl, benzoxazolyl, 2,3-dihydro-1-benzofuranyl, indolyl, pyridyl, quinolyl, cyclopropyl, cyclopentyl, cyclohexyl, or cycloheptyl.
   13. A compound according to embodiment 1 wherein G² represents C₂₋₄alkenylene.
   14. A compound according to embodiment 1 wherein A is selected from phenyl or pyridyl;
   R¹ is independently selected from halogen, C₁₋₄alkyl or C₁₋₄alkoxy; said C₁₋₄alkyl or C₁₋₄alkoxy being optionally substituted by OH or by one or more F atoms;
   m represents an integer 0 or 1;
   R³ is hydrogen;
   L¹ represents a direct bond or C₁₋₄alkylene;
   L² represents a direct bond, —OCH₂—, C₁₋₂alkylene or —C≡C—;
   G¹ represents phenyl, 5- or 6-membered heteroaryl or C₃₋₁₀carbocyclyl; optionally fused to one further ring selected from phenyl or 5- or 6-membered heteroaryl;
   G² represents H, C₁₋₆alkyl, C₂₋₄alkenylene, phenyl, 5- or 6-membered heteroaryl, C₃₋₁₀carbocyclyl or C₅₋₈heterocyclyl; said C₁₋₆alkyl being optionally further substituted by one or more groups selected from OH, C₁₋₆alkoxy or halogen;
   the phenyl, heteroaryl, carbocyclyl or heterocyclyl moieties in G¹ and G² being optionally fused to one or two further rings independently selected from phenyl, a 5- or 6-membered heteroaryl, C₅₋₆-carbocyclyl or C₅₋₆heterocyclyl ring;
   any phenyl, heteroaryl, carbocyclyl or heterocyclyl moieties in G¹ and G² being optionally substituted by one or more substituents independently selected from halogen, OH, CN, NO₂, CO₂R⁹, C₁₋₆alkyl, C₁₋₆alkoxy, C₁₋₄thioalkoxy, SO₂NR¹⁰R¹¹,NR¹²R¹³, —O(CH₂)₂—O—(CH₂)₂—C₁₋₆alkoxy, —NHCOC(OH)(CH₃)CF₃ or —CH₂OCH₂CF₂CHF₂; said C₁₋₆alkyl or C₁₋₆alkoxy being optionally substituted by OH, C₁₋₆alkoxy, phenyl or by one or more F atoms;
   G³ represents phenyl or 5- or 6-membered heteroaryl; and
   each R⁴, R⁵, R⁶, R⁹, R¹⁰, R¹¹, R¹² and R¹³ is independently selected from H or C₁₋₄alkyl.
   15. A compound according to embodiment 1 wherein
   A is selected from phenyl;
   R¹ is independently selected from halogen, C₁₋₄alkyl or C₁₋₄alkoxy; said C₁₋₄alkyl or C₁₋₄alkoxy being optionally substituted by OH or by one or more F atoms;
   m represents an integer 0 or 1;
   R³ is hydrogen;
   L¹ represents a direct bond or C₁₋₄alkylene;
   L² represents a direct bond, —OCH₂—, C₁₋₂alkylene or —C≡C—;
   G¹ represents phenyl or 5- or 6-membered heteroaryl; optionally fused to one further ring selected from phenyl or 5- or 6-membered heteroaryl;
   G² represents H, C₁₋₆alkyl, C₁₋₆alkenylene, phenyl, 5- or 6-membered heteroaryl, C₃₋₁₀carbocyclyl or C₅₋₈heterocyclyl; said C₁₋₆alkyl being optionally further substituted by one or more groups selected from OH, C₁₋₆alkoxy or halogen;
   the phenyl, heteroaryl, carbocyclyl or heterocyclyl moieties in G¹ and G² being optionally fused to one or two further rings independently selected from phenyl, a 5- or 6-membered heteroaryl, C₅₋₆-carbocyclyl or C₅₋₆heterocyclyl ring; any phenyl, heteroaryl, carbocyclyl or heterocyclyl moieties in G¹ and G² being optionally substituted by one or more substituents independently selected from halogen, OH, CN, NO₂, CO₂R⁹, C₁₋₆alkyl, C₁₋₆alkoxy, C₁₋₄thioalkoxy, SO₂NR¹⁰R¹¹,NR¹²R¹³, —O(CH₂)₂—O—(CH₂)₂—C₁₋₆alkoxy, —NHCOC(OH)(CH₃)CF₃, —CH₂OCH₂CF₂CHF₂ or —CH₂OCH₂CH₂CF₃; said C₁₋₆alkyl or C₁₋₆alkoxy being optionally substituted by OH, C₁₋₆alkoxy, phenyl or by one or more F atoms.
   16. A compound according to any preceding embodiment being an entity selected from:
5-Benzofuran-2-yl-N-(2-sulfamoylphenyl)sulfonyl-pyridine-2-carboxamide
5-(2,3-Dichlorophenyl)-N-(2-sulfamoylphenyl)sulfonyl-pyridine-2-carboxamide
4-Benzofuran-2-yl-N-(2-sulfamoylphenyl)sulfonyl-benzamide
4-Benzothiophen-2-yl-N-(2-sulfamoylphenyl)sulfonyl-benzamide
4-Benzothiazol-2-yl-N-(2-sulfamoylphenyl)sulfonyl-benzamide
4-(7-Oxa-3,9-diazabicyclo[4.3.0]nona-2,4,8,10-tetraen-8-yl)-N-(2-sulfamoylphenyl)sulfonyl-benzamide
4-(7-Oxa-5,9-diazabicyclo[4.3.0]nona-2,4,8,10-tetraen-8-yl)-N-(2-sulfamoylphenyl)sulfonyl-benzamide
4-Benzooxazol-2-yl-N-(2-sulfamoylphenyl)sulfonyl-benzamide
2-Phenyl-N-(2-sulfamoylphenyl)sulfonyl-benzofuran-6-carboxamide
4-Bromo-N-(2-sulfamoylphenyl)sulfonyl-benzamide
4-Bromo-2-chloro-N-(2-sulfamoylphenyl)sulfonyl-benzamide
4-Bromo-3-methyl-N-(2-sulfamoylphenyl)sulfonyl-benzamide
4-Bromo-3-fluoro-N-(2-sulfamoylphenyl)sulfonyl-benzamide
4-Bromo-2-fluoro-N-(2-sulfamoylphenyl)sulfonyl-benzamide
4-Bromo-2-methyl-N-(2-sulfamoylphenyl)sulfonyl-benzamide
2-(1-Adamantyl)-N-(2-sulfamoylphenyl)sulfonyl-acetamide
N-(2-Sulfamoylphenyl)sulfonylnorbornane-2-carboxamide
1-Phenyl-N-(2-sulfamoylphenyl)sulfonyl-cyclohexane-1-carboxamide
3-(Difluoromethoxy)-N-(2-sulfamoylphenyl)sulfonyl-benzamide
3-Bromo-4-fluoro-N-(2-sulfamoylphenyl)sulfonyl-benzamide
N-(2-Sulfamoylphenyl)sulfonyl-3-(2,2,3,3-tetrafluoropropoxymethyl)benzamide
4-Methyl-N-(2-sulfamoylphenyl)sulfonyl-2-[3-(trifluoromethyl)phenyl]1,3-thiazole-5-carboxamide
4-Chloro-2-fluoro-N-(2-sulfamoylphenyl)sulfonyl-benzamide
2-Benzyl-4-chloro-N-(2-sulfamoylphenyl)sulfonyl-benzamide
2-Phenyl-N-(2-sulfamoylphenyl)sulfonyl-benzofuran-5-carboxamide
4-Methyl-N-(2-sulfamoylphenyl)sulfonyl-2-[4-(trifluoromethyl)phenyl]1,3-thiazole-5-carboxamide
2-(2,3-Dihydrobenzofuran-5-yl)-4-methyl-N-(2-sulfamoylphenyl)sulfonyl-1,3-thiazole-5-carboxamide
2-(4-Chlorophenyl)-4-methyl-N-(2-sulfamoylphenyl)sulfonyl-1,3-thiazole-5-carboxamide
4-Methyl-2-phenyl-N-(2-sulfamoylphenyl)sulfonyl-1,3-thiazole-5-carboxamide
4-Phenylmethoxy-N-(2-sulfamoylphenyl)sulfonyl-benzamide
4-Phenyl-N-(2-sulfamoylphenyl)sulfonyl-benzamide
N-(2-Sulfamoylphenyl)sulfonyl-4-tert-butyl-benzamide
1-Methyl-N-(2-sulfamoylphenyl)sulfonyl-indole-2-carboxamide
5-Pyridin-2-yl-N-(2-sulfamoylphenyl)sulfonyl-thiophene-2-carboxamide
5-Phenyl-N-(2-sulfamoylphenyl)sulfonyl-thiophene-2-carboxamide
5-(3,4-Dichlorophenyl)-N-(2-sulfamoylphenyl)sulfonyl-furan-2-carboxamide
N-(2-Sulfamoylphenyl)sulfonyl-5-[3-(trifluoromethyl)phenyl]furan-2-carboxamide
1-(3,5-Dichlorophenyl)-5-propyl-N-(2-sulfamoylphenyl)sulfonyl-pyrazole-4-carboxamide
3,6-Dichloro-N-(2-sulfamoylphenyl)sulfonyl-benzothiophene-2-carboxamide
N-(2-Sulfamoylphenyl)sulfonylbenzothiophene-3-carboxamide
Ethyl 4-[5-[(2-Sulfamoylphenyl)sulfonylcarbamoyl]-2-furyl]benzoate
2-(3-Chlorophenyl)-4-methyl-N-(2-sulfamoylphenyl)sulfonyl-1,3-thiazole-5-carboxamide
4-(3,3-Dimethylbut-1-ynyl)-N-(2-sulfamoylphenylsulfonyl)benzamide
4-(3-Hydroxy-3-methylbut-1-ynyl)-N-(2-sulfamoylphenylsulfonyl)benzamide
4-(Benzofuran-2-yl)-2-methyl-N-(2-sulfamoylphenylsulfonyl)benzamide;
4-(Benzofuran-2-yl)-2-methyl-N-(2-sulfamoylphenylsulfonyl)benzamide;
4-(Benzofuran-2-yl)-3,5-dimethoxy-N-(2-sulfamoylphenylsulfonyl)benzamide;
4-(Benzofuran-2-yl)-2-methoxy-N-(2-sulfamoylphenylsulfonyl)benzamide;
4-(Benzofuran-2-yl)-2-hydroxy-N-(2-sulfamoylphenylsulfonyl)benzamide;
4-(Benzofuran-2-yl)-3-methoxy-N-(2-sulfamoylphenylsulfonyl)benzamide;
4-(Benzofuran-2-yl)-3-hydroxy-N-(2-sulfamoylphenylsulfonyl)benzamide;
4-(Benzofuran-2-yl)-2,6-dimethyl-N-(2-sulfamoylphenylsulfonyl)benzamide;
4-(3-Methoxyprop-1-ynyl)-N-(2-sulfamoylphenylsulfonyl)benzamide;
4-(3-Methylbut-3-en-1-ynyl)-N-(2-sulfamoylphenylsulfonyl)benzamide;
6-(Phenylethynyl)-N-(2-sulfamoylphenylsulfonyl)nicotinamide;
4-(3-Ethyl-3-hydroxypent-1-ynyl)-N-(2-sulfamoylphenylsulfonyl)benzamide;
4-(3-Hydroxy-3-methylpent-1-ynyl)-N-(2-sulfamoylphenylsulfonyl)benzamide;
4-((1-Hydroxycyclopentyl)ethynyl)-N-(2-sulfamoylphenylsulfonyl)benzamide;
3-(3-Hydroxy-3-methylbut-1-ynyl)-N-(2-sulfamoylphenylsulfonyl)benzamide;
3-(3,3-Dimethylbut-1-ynyl)-N-(2-sulfamoylphenylsulfonyl)benzamide;
4-(3,3-Dimethylbut-1-ynyl)-N-(2-sulfamoylphenylsulfonyl)-1-naphthamide;
4-(Benzofuran-2-yl)-N-(2-sulfamoylphenylsulfonyl)-1-naphthamide;
2-(Benzofuran-2-yl)-4-methyl-N-(2-sulfamoylphenylsulfonyl)thiazole-5-carboxamide;
3′-(3-Hydroxy-3-methylbut-1-ynyl)-N-(2-sulfamoylphenylsulfonyl)biphenyl-2-carboxamide;
4-(Cyclopentylethynyl)-N-(2-sulfamoylphenylsulfonyl)benzamide;
3-(Cyclopentylethynyl)-N-(2-sulfamoylphenylsulfonyl)benzamide;
4-(Cyclopentylethynyl)-2-methyl-N-(2-sulfamoylphenylsulfonyl)benzamide;
4-(3,3-Dimethylbut-1-ynyl)-3-methoxy-2-methyl-N-(2-sulfamoylphenylsulfonyl)benzamide;
4-(Benzofuran-2-yl)-3-methoxy-2-methyl-N-(2-sulfamoylphenylsulfonyl)benzamide;
4-(Pyridin-3-ylethynyl)-N-(2-sulfamoylphenylsulfonyl)benzamide;
4-(Pyridin-2-ylethynyl)-N-(2-sulfamoylphenylsulfonyl)benzamide;
4-(Phenylethynyl)-N-(2-sulfamoylphenylsulfonyl)benzamide;
4-(3,3-Dimethylbut-1-ynyl)-3-fluoro-N-(2-sulfamoylphenylsulfonyl)benzamide;
2-(3-Methoxyphenyl)-N-(2-sulfamoylphenylsulfonyl)benzofuran-5-carboxamide;
2-(4-Methoxyphenyl)-N-(2-sulfamoylphenylsulfonyl)benzofuran-5-carboxamide;
2-tert-Butyl-N-(2-sulfamoylphenylsulfonyl)benzofuran-5-carboxamide;
2-(1-Hydroxycyclopentyl)-N-(2-sulfamoylphenylsulfonyl)benzofuran-5-carboxamide;
2-Cyclopentyl-N-(2-sulfamoylphenylsulfonyl)benzofuran-5-carboxamide;
3-Cyano-4-(3,3-dimethylbut-1-ynyl)-N-(2-sulfamoylphenylsulfonyl)benzamide;
4-(Benzofuran-2-yl)-3-cyano-N-(2-sulfamoylphenylsulfonyl)benzamide;
4-Chloro-2-hydroxy-N-(2-sulfamoylphenylsulfonyl)benzamide;
4-Bromo-2-hydroxy-N-(2-sulfamoylphenylsulfonyl)benzamide;
4-(Benzofuran-2-yl)-2-fluoro-N-(2-sulfamoylphenylsulfonyl)benzamide;
4-(3,3-Dimethylbut-1-ynyl)-2-fluoro-N-(2-sulfamoylphenylsulfonyl)benzamide;
4-(Cyclopentylethynyl)-2-fluoro-N-(2-sulfamoylphenylsulfonyl)benzamide;
4-(Cyclopentylethynyl)-2-fluoro-3-methoxy-N-(2-sulfamoylphenylsulfonyl)benzamide;
4-(Benzofuran-2-yl)-2-fluoro-3-methoxy-N-(2-sulfamoylphenylsulfonyl)benzamide;
5-(Cyclohexylethynyl)-N-(2-sulfamoylphenylsulfonyl)picolinamide;
5-(3,3-Dimethylbut-1-ynyl)-N-(2-sulfamoylphenylsulfonyl)picolinamide;
4-(3,3-Dimethylbut-1-ynyl)-2-fluoro-3-methoxy-N-(2-sulfamoylphenylsulfonyl)benzamide;
4-(Benzofuran-2-yl)-2-chloro-N-(2-sulfamoylphenylsulfonyl)benzamide;
4-(Cyclopentylethynyl)-2-hydroxy-N-(2-sulfamoylphenylsulfonyl)benzamide;
6-(Cyclopentylethynyl)-N-(2-sulfamoylphenylsulfonyl)nicotinamide;
6-(Pyridin-2-ylethynyl)-N-(2-sulfamoylphenylsulfonyl)nicotinamide;
6-(Pyridin-3-ylethynyl)-N-(2-sulfamoylphenylsulfonyl)nicotinamide;
2-(3,3-Dimethylbut-1-ynyl)-N-(2-sulfamoylphenylsulfonyl)pyrimidine-5-carboxamide;
N-(2-Sulfamoylphenylsulfonyl)-4-((3,3,3-trifluoropropoxy)methyl)benzamide;
4-(Cyclopentylethynyl)-3-(hydroxymethyl)-N-(2-sulfamoylphenylsulfonyl)benzamide;
6-(3-Methylbut-1-ynyl)-N-(2-sulfamoylphenylsulfonyl)nicotinamide;
3-(Hydroxymethyl)-4-(phenylethynyl)-N-(2-sulfamoylphenylsulfonyl)benzamide;
4-(Cyclohexylethynyl)-3-(hydroxymethyl)-N-(2-sulfamoylphenylsulfonyl)benzamide;
2-((4-chlorophenyl)ethynyl)-N-(2-sulfamoylphenylsulfonyl)pyrimidine-5-carboxamide;
4-(Benzofuran-2-yl)-3-(hydroxymethyl)-N-(2-sulfamoylphenylsulfonyl)benzamide;
4-(Benzofuran-2-yl)-N-(2-sulfamoylphenylsulfonyl)cyclohexanecarboxamide;
(1S,4S)-4-(Benzofuran-2-yl)-N-(2-sulfamoylphenylsulfonyl)cyclohexanecarboxamide;
(1R,4R)-4-(Benzofuran-2-yl)-N-(2-sulfamoylphenylsulfonyl)cyclohexanecarboxamide;
4-(Benzofuran-2-yl)-1-methyl-N-(2-sulfamoylphenylsulfonyl)cyclohexanecarboxamide;
(1R,4R)-4-(Benzofuran-2-yl)-1-methyl-N-(2-sulfamoylphenylsulfonyl)cyclohexanecarboxamide;
(1S,4S)-4-(Benzofuran-2-yl)-1-methyl-N-(2-sulfamoylphenylsulfonyl)cyclohexanecarboxamide;
4-(3,3-Dimethylbut-1-ynyl)-3-methoxy-N-(2-sulfamoylphenylsulfonyl)benzamide;
4-(Cyclopropylethynyl)-3-methoxy-N-(2-sulfamoylphenylsulfonyl)benzamide;
4-(3-Methoxy-3-methylbut-1-ynyl)-N-(2-sulfamoylphenylsulfonyl)benzamide;
4-(3-Methylbut-1-ynyl)-N-(2-sulfamoylphenylsulfonyl)benzamide;
3-Methoxy-4-(3-methoxy-3-methylbut-1-ynyl)-N-(2-sulfamoylphenylsulfonyl)-benzamide;
3-Hydroxy-4-(3-methoxy-3-methylbut-1-ynyl)-N-(2-sulfamoylphenylsulfonyl)-benzamide;
6-(3,3-Dimethylbut-1-ynyl)-N-(2-sulfamoylphenylsulfonyl)nicotinamide;
6-(Benzofuran-2-yl)-N-(2-sulfamoylphenylsulfonyl)nicotinamide;
4-(3,3-Dimethylbut-1-ynyl)-3-(2-(2-methoxyethoxy)ethoxy)-N-(2-sulfamoylphenylsulfonyl)benzamide;
4-(Benzofuran-2-yl)-3-(2-(2-methoxyethoxy)ethoxy)-N-(2-sulfamoylphenylsulfonyl)benzamide;
2-(2-Methoxyphenyl)-N-(2-sulfamoylphenylsulfonyl)benzofuran-5-carboxamide;
2-(1-tert-Butoxyethyl)-N-(2-sulfamoylphenylsulfonyl)benzofuran-5-carboxamide;
2-(Pyridin-2-yl)-N-(2-sulfamoylphenylsulfonyl)benzofuran-5-carboxamide;
2-(Pyridin-3-yl)-N-(2-sulfamoylphenylsulfonyl)benzo furan-5-carboxamide;
2-(2-Hydroxypropan-2-yl)-N-(2-sulfamoylphenylsulfonyl)benzofuran-5-carboxamide;
2-(2-Methoxypropan-2-yl)-N-(2-sulfamoylphenylsulfonyl)benzofuran-5-carboxamide;
2-Cyclopropyl-N-(2-sulfamoylphenylsulfonyl)benzofuran-5-carboxamide;
4-(Benzofuran-2-yl)-3-isopropoxy-N-(2-sulfamoylphenylsulfonyl)benzamide;
4-(3,3-Dimethylbut-1-ynyl)-3-isopropoxy-N-(2-sulfamoylphenylsulfonyl)benzamide;
4-(3-Hydroxy-3-methylbut-1-ynyl)-3-isopropoxy-N-(2-sulfamoylphenylsulfonyl)benzamide;
4-(Cyclopentylethynyl)-3-isopropoxy-N-(2-sulfamoylphenylsulfonyl)benzamide;
4-(Cyclohexylethynyl)-3-isopropoxy-N-(2-sulfamoylphenylsulfonyl)benzamide;
4-(Cyclopropylethynyl)-3-isopropoxy-N-(2-sulfamoylphenylsulfonyl)benzamide;
4-((1-Hydroxycycloheptyl)ethynyl)-3-isopropoxy-N-(2-sulfamoylphenylsulfonyl)benzamide;
6-(3,3-Dimethylbut-1-ynyl)-5-(2-(2-methoxyethoxy)ethoxy)-N-(2-sulfamoylphenylsulfonyl)nicotinamide;
6-(Benzofuran-2-yl)-5-(2-(2-methoxyethoxy)ethoxy)-N-(2-sulfamoylphenylsulfonyl)nicotinamide;
6-(Cyclopentylethynyl)-5-(2-(2-methoxyethoxy)ethoxy)-N-(2-sulfamoylphenylsulfonyl)nicotinamide;
6-(Cyclopentylethynyl)-5-methoxy-N-(2-sulfamoylphenylsulfonyl)nicotinamide;
6-(Cyclohexylethynyl)-5-methoxy-N-(2-sulfamoylphenylsulfonyl)nicotinamide;
5-Methoxy-N-(2-sulfamoylphenylsulfonyl)-6-((4-(trifluoromethyl)phenyl)ethynyl)nicotinamide;
N-(3-Dimethylaminopropyl)-N′-ethylcarbodiimide hydrochloride;
1-(2-Methoxyethyl)-2-phenyl-N-(2-sulfamoylphenylsulfonyl)-1H-indole-5-carboxamide;
6-(Cyclopropylethynyl)-5-isopropoxy-N-(2-sulfamoylphenylsulfonyl)nicotinamide;
6-(Cyclopentylethynyl)-5-isopropoxy-N-(2-sulfamoylphenylsulfonyl)nicotinamide;
6-(Cyclohexylethynyl)-5-isopropoxy-N-(2-sulfamoylphenylsulfonyl)nicotinamide-4-(Benzofuran-2-yl)-3-(3-methoxy-3-methylbutoxy)-N-(2-sulfamoylphenylsulfonyl)benzamide;
4-(Cyclopentylethynyl)-3-fluoro-N-(2-sulfamoylphenylsulfonyl)benzamide;
6-(Benzofuran-2-yl)-5-chloro-N-(2-sulfamoylphenylsulfonyl)nicotinamide;
5-Chloro-6-(cyclopentylethynyl)-N-(2-sulfamoylphenylsulfonyl)nicotinamide;
5-Chloro-6-(3,3-dimethylbut-1-ynyl)-N-(2-sulfamoylphenylsulfonyl)nicotinamide;
4-(Benzofuran-2-yl)-N-(2-sulfamoylphenylsulfonyl)-2-(trifluoromethyl)benzamide;
4-(3,3-Dimethylbut-1-ynyl)-N-(2-sulfamoylphenylsulfonyl)-2-(trifluoromethyl)benzamide;
4-(Benzofuran-2-yl)-2,6-difluoro-N-(2-sulfamoylphenylsulfonyl)benzamide;
4-(Cyclopentylethynyl)-2,6-difluoro-N-(2-sulfamoylphenylsulfonyl)benzamide;
4-(Benzofuran-2-yl)-3-(3-hydroxy-3-methylbut-1-ynyl)-N-(2-sulfamoylphenylsulfonyl)benzamide;
4-(Benzofuran-2-yl)-3-bromo-N-(2-sulfamoylphenylsulfonyl)benzamide;
4-(Benzyloxy)-3-(3-hydroxy-3-methylbut-1-ynyl)-N-(2-sulfamoylphenylsulfonyl)benzamide;
4-(Benzyloxy)-3-iodo-N-(2-sulfamoylphenylsulfonyl)benzamide;
2-Benzyl-N-(2-sulfamoylphenylsulfonyl)-1H-indole-5-carboxamide;
7-(Cyclopropylethynyl)-2,2-difluoro-N-(2-sulfamoylphenylsulfonyl)benzo[d][1,3]dioxole-4-carboxamide;
4-(Cyclopropylethynyl)-N-(2-sulfamoylphenylsulfonyl)-3-(3,3,3-trifluoropropoxy)benzamide;
4-(Benzofuran-2-yl)-N-(4-(hydroxymethyl)-2-sulfamoylphenylsulfonyl)benzamide; and
Benzene-1,2-disulfonic acid 1-amide 2[(quinoline-3-carbonyl)-amide] or a pharmaceutically acceptable salt thereof.
   17. A process for the preparation of a compound of formula (I) or a pharmaceutically acceptable salt thereof as defined in embodiment 1 which comprises,
   (a) reacting a compound of formula (II)

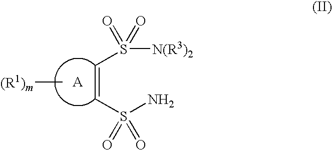

wherein R¹, R³, A and m are as defined in formula (I),
with a compound of formula (III)

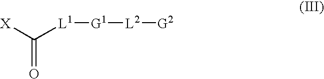

wherein L¹, L², G¹ and G² are as defined in formula (I) and X represents a leaving group such as OH or halogen; or
(b) when L² represents a direct bond and G¹ and G² are both aromatic moieties, reacting a compound of formula (IV)

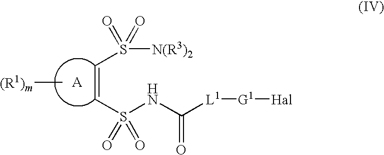

wherein Hal represents a halogen atom and R¹, R³, A, m and L¹ are as defined in formula (I),
with a nucleophile G²-M wherein M represents an organo-tin or organo boronic acid group;
and optionally after (a) or (b) carrying out one or more of the following:
converting the compound obtained to a further compound of the invention
forming a pharmaceutically acceptable salt of the compound.
   18. A pharmaceutical composition comprising a compound of formula (I) or a pharmaceutically acceptable salt thereof as in any one of embodiments 1 to 16 in association with a pharmaceutically acceptable adjuvant, diluent or carrier.
   19. A process for the preparation of a pharmaceutical composition as in embodiment 18 which comprises mixing a compound of formula (I) or a pharmaceutically acceptable salt thereof as in any one of embodiments 1 to 16 with a pharmaceutically acceptable adjuvant, diluent or carrier.
   20. A compound of formula (I) or a pharmaceutically acceptable salt thereof

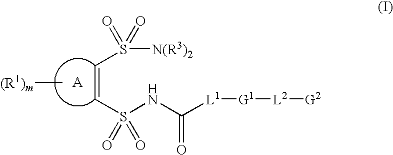

wherein:
A is selected from phenyl or a 5- or 6-membered heteroaryl moiety; said phenyl or a 5- or 6-membered heteroaryl moiety in group A being optionally fused to a phenyl, a 5- or 6-membered heteroaryl, C₅₋₆-carbocyclyl or C₅₋₆heterocyclyl ring;
R¹ is independently selected from halogen, nitro, SF₅, OH, CHO, CO₂R⁴, CONR⁵R⁶, C₁₋₄alkyl, C₁₋₄alkoxy, G³, OG³ or OCH₂G³; said C₁₋₄alkyl or C₁₋₄alkoxy being optionally substituted by OH or by one or more F atoms;
m represents an integer 0, 1 or 2;

each R³ is independently selected from hydrogen, CN and C₁₋₄alkyl; said C₁₋₄alkyl being optionally substituted with OH, CN, C₁₋₄alkoxy, NR⁷R⁸, or one or more F atoms;

L¹ represents a direct bond, C₁₋₄alkylene, C₂₋₄alkenylene or C₂₋₄alkynylene;
L² represents a direct bond, —O—, —OCH₂—, C₁₋₂alkylene or —C≡C—;
G¹ represents phenyl, 5- or 6-membered heteroaryl, C₃₋₁₀carbocyclyl or
C₅₋₈heterocyclyl;
G² represents H, C₁₋₆alkyl, C₁₋₆alkenyl, phenyl, 5- or 6-membered heteroaryl, C₃₋₁₀carbocyclyl or
C₅₋₈heterocyclyl; said C₁₋₆alkyl being optionally further substituted by one or more groups selected from OH, C₁₋₆alkoxy and halogen;
the phenyl, heteroaryl, carbocyclyl or heterocyclyl moieties in G¹ and G² being optionally fused to one or two further rings independently selected from phenyl, a 5- or 6-membered heteroaryl, C₅₋₆-carbocyclyl or C₅₋₆heterocyclyl ring;
any phenyl, heteroaryl, carbocyclyl or heterocyclyl moieties in G¹ and G² being optionally substituted by one or more substituents independently selected from halogen, OH, CN, NO₂, CO₂R⁹, C₁₋₆alkyl, C₁₋₆alkoxy, C₁₋₄thioalkoxy, SO₂NR¹⁰R¹¹,NR¹²R¹³, —O(CH₂)₂—O—(CH₂)₂—C₁₋₆alkoxy, —NHCOC(OH)(CH₃)CF₃, —CH₂OCH₂CF₂CHF₂ or —CH₂OCH₂CH₂CF₃; said C₁₋₆alkyl or C₁₋₆alkoxy being optionally substituted by OH, C₁₋₆alkoxy, phenyl or by one or more F atoms;
G³ represents phenyl or 5- or 6-membered heteroaryl; and
each R⁴, R⁵, R⁶, R⁷, R⁸, R⁹, R¹⁰, R¹¹, R¹² and R¹³ is independently selected from H or C₁₋₄alkyl.
for use in therapy.
21. A compound according to embodiment 1 wherein
A is selected from phenyl or a 5- or 6-membered heteroaryl moiety; said phenyl or a 5- or 6-membered heteroaryl moiety in group A being optionally fused to a phenyl, a 5- or 6-membered heteroaryl, C₅₋₆-carbocyclyl or C₅₋₆heterocyclyl ring;
R¹ is independently selected from halogen, nitro, SF₅, OH, CHO, CO₂R⁴, CONR⁵R⁶, C₁₋₄alkyl, C₁₋₄alkoxy, G³, OG³ or OCH₂G³; said C₁₋₄alkyl or C₁₋₄alkoxy being optionally substituted by OH or by one or more F atoms;
m represents an integer 0, 1 or 2;
each R³ is independently selected from hydrogen, CN and C₁₋₄alkyl; said C₁₋₄alkyl being optionally substituted with OH, CN, C₁₋₄alkoxy, NR⁷R⁸ or one or more F atoms;
L¹ represents a direct bond, C₁₋₄alkylene, C₂₋₄alkenylene or C₂₋₄alkynylene;
L² represents a direct bond, —O—, —OCH₂—, C₁₋₂alkylene or —C≡C—;
G¹ represents phenyl, 5- or 6-membered heteroaryl, C₃₋₁₀carbocyclyl or C₅₋₈heterocyclyl;
G² represents H, C₁₋₆alkyl, phenyl, 5- or 6-membered heteroaryl, C₃₋₁₀carbocyclyl or C₅₋₈heterocyclyl; said C₁₋₆alkyl being optionally further substituted by one or more groups selected from OH, C₁₋₆alkoxy and halogen;
the phenyl, heteroaryl, carbocyclyl or heterocyclyl moieties in G¹ and G² being optionally fused to one or two further rings independently selected from phenyl, a 5- or 6-membered heteroaryl, C₅₋₆-carbocyclyl or C₅₋₆heterocyclyl ring;
any phenyl, heteroaryl, carbocyclyl or heterocyclyl moieties in G¹ and G² being optionally substituted by one or more substituents independently selected from halogen, OH, CN, NO₂, CO₂R⁹, C₁₋₆alkyl, C₁₋₆alkoxy, C₁₋₄thioalkoxy, SO₂NR¹⁰R¹¹,NR¹²R¹³, —NHCOC(OH)(CH₃)CF₃ or —CH₂OCH₂CF₂CHF₂; said C₁₋₆alkyl or C₁₋₆alkoxy being optionally substituted by OH or by one or more F atoms;
G³ represents phenyl or 5- or 6-membered heteroaryl; and
each R⁴, R⁵, R⁶, R⁷, R⁸, R⁹, R¹⁰, R¹¹, R¹² and R¹³ is independently selected from H or C₁₋₄alkyl;
for use in therapy.
22. Use of a compound of formula (I) or a pharmaceutically acceptable salt thereof as in embodiment 20 in the manufacture of a medicament for the treatment of human diseases or conditions in which modulation of microsomal prostaglandin E synthase-1 activity is beneficial.
23. Use of a compound of formula (I) or a pharmaceutically acceptable salt thereof as in embodiment 20 in the manufacture of a medicament for use in treating osteoarthritis, rheumatoid arthritis, benign or malignant neoplasias or acute or chronic pain.
24. Use of a compound of formula (I) or a pharmaceutically acceptable salt thereof as in embodiment 20 in the manufacture of a medicament for use in treating acute or chronic pain, nociceptive pain, neuropathic pain, apnea, sudden infant death (SID), atherosclerosis, cancer, aneurysm, hyperthermia, myositis, Alzheimer's disease or arthritis.
25. A method of treating, or reducing the risk of, an inflammatory disease or condition which comprises administering to a patient in need thereof a therapeutically effective amount of a compound of formula (I) or a pharmaceutically acceptable salt thereof as defined in embodiment 20.

The present invention will now be further explained by reference to the following illustrative examples.

### General Methods

All solvents used were analytical grade and commercially available anhydrous solvents were routinely used for reactions. Reactions were typically run under an inert atmosphere of nitrogen or argon.

¹H, ¹⁹F and ¹³C NMR spectra were recorded on a Varian Unity+400 NMR Spectrometer equipped with a 5 mm BBO probehead with Z-gradients, or a Varian Gemini 300 NMR spectrometer equipped with a 5 mm BBI probehead, or a Bruker Avance 400 NMR spectrometer equipped with a 60 μl dual inverse flow probehead with Z-gradients, or a Varian Mercury Plus 400 NMR Spectrometer equipped with a Varian 400 ATB PFG probe, or a Bruker DPX400 NMR spectrometer equipped with a 4-nucleus probehead equipped with Z-gradients, or a Bruker Avance 600 NMR spectrometer equipped with a 5 mm BBI probehead with Z-gradients, or Bruker 500 MHz Avance III NMR spectrometer, operating at 500 MHz for ¹H, 125 MHz for ¹³C, and 50 MHz for ¹⁵N equipped with a 5 mm TXI probehead with Z-gradients.

Unless specifically noted in the examples, spectra were recorded at 400 MHz for proton, 376 MHz for fluorine-19 and 100 MHz for carbon-13.

The following reference signals were used: the middle line of DMSO-d₆ δ 2.50 (1H), δ 39.51 (13C); the middle line of CD₃OD δ 3.31 (1H) or δ 49.15 (13C); CDCl₃ δ 7.26 (1H) and the middle line of CDCl₃ δ 77.16 (13C) (unless otherwise indicated). NMR spectra are either reported from high to low field or from low to high field.

Mass spectra were recorded on a Waters LCMS consisting of an Alliance 2795 (LC), Waters PDA 2996 and a ZQ single quadrupole mass spectrometer. The mass spectrometer was equipped with an electrospray ion source (ESI) operated in a positive or negative ion mode. The capillary voltage was 3 kV and cone voltage was 30 V. The mass spectrometer was scanned between m/z 100-700 with a scan time of 0.3 s. Separations were performed on either Waters X-Terra MS C8 (3.5 μm, 50 or 100 mm×2.1 mm i.d.) or an ACE 3 AQ (100 mm×2.1 mm i.d.) obtained from ScantecLab. Flow rates were regulated to 1.0 or 0.3 mL/min, respectively. The column temperature was set to 40° C. A linear gradient was applied using a neutral or acidic mobile phase system, starting at 100% A (A: 95:5 10 mM NH₄OAc:MeCN, or 95:5 8 mM HCOOH:MeCN) ending at 100% B (MeCN).

Alternatively, mass spectra were recorded on a Waters LCMS consisting of an Alliance 2690 Separations Module, Waters 2487 Dual 1 Absorbance Detector (220 and 254 nm) and a Waters ZQ single quadrupole mass spectrometer. The mass spectrometer was equipped with an electrospray ion source (ESI) operated in a positive or negative ion mode. The capillary voltage was 3 kV and cone voltage was 30 V. The mass spectrometer was scanned between m/z 97-800 with a scan time of 0.3 or 0.8 s. Separations were performed on a Chromolith Performance RP-18e (100×4.6 mm). A linear gradient was applied starting at 95% A (A: 0.1% HCOOH (aq.)) ending at 100% B (MeCN) in 5 minutes. Flow rate: 2.0 mL/min.

Alternatively, LC-MS analyses were performed on a LC-MS system consisting of a Waters Alliance 2795 HPLC, a Waters PDA 2996 diode array detector, a Sedex 85 ELS detector and a ZQ single quadrupole mass spectrometer. The mass spectrometer was equipped with an electrospray ion source (ES) operated in positive and negative ion mode. The capillary voltage was set to 3.3 kV and the cone voltage to 28 V, respectively. The mass spectrometer scanned between m/z 100-800 with a scan time of 0.3s. The diode array detector scanned from 200-400 nm. The temperature of the ELS detector was adjusted to 40° C. and the pressure was set to 1.9 bar. Separation was performed on an Gemini C18, 3.0 mm×50 mm, 3 μm, (Phenomenex) run at a flow rate of 1 ml/min. A linear gradient was applied starting at 100% A (A: 10 mM NH₄OAc in 5% CH3CN) ending at 100% B (B: CH3CN) in 4.0 min followed by 100% B until 5.5 min. The column oven temperature was set to 40° C.

Alternatively, LC-MS analyses were performed on a LC-MS consisting of a Waters sample manager 2777C, a Waters 1525μ binary pump, a Waters 1500 column oven, a Waters ZQ single quadrupole mass spectrometer, a Waters PDA2996 diode array detector and a Sedex 85 ELS detector. The mass spectrometer was configured with an atmospheric pressure chemical ionisation (APCI) ion source which was further equipped with atmospheric pressure photo ionisation (APPI) device. The mass spectrometer scanned in the positive mode, switching between APCI and APPI mode. The mass range was set to m/z 100-800 using a scan time of 0.1 s. The APPI repeller and the APCI corona were set to 0.58 kV and 0.70μ, respectively. In addition, the desolvation temperature (350° C.), desolvation gas (450 L/Hr) and cone gas (0 L/Hr) were constant for both APCI and APPI mode. Separation was performed using a Gemini column C18, 3.0 mm×50 mm, 3 μm, (Phenomenex) and run at a flow rate of 0.8 ml/min. A linear gradient was used starting at 100% A (A: 10 mM NH4OAc in 5% MeOH) and ending at 100% B (MeOH) in 4.0 min followed by 100% B until 5.5 min. The column oven temperature was set to 55° C.

Microwave irradiation was performed in a Creator™, Initiator™ or Smith Synthesizer™ Single-mode microwave cavity producing continuous irradiation at 2450 MHz. HPLC analyses were performed on an Agilent HP 1000 system consisting of G1379A Micro Vacuum Degasser, G1312A Binary Pump, G1367A Well plate auto-sampler, G1316A Thermostatted Column Compartment and G1315B Diode Array Detector.

Column: X-Terra MS, Waters, 3.0×100 mm, 3.5 μm. The column temperature was set to 40° C. and the flow rate to 1.0 ml/min. The Diode Array Detector was scanned from 210-300 nm, step and peak width were set to 2 nm and 0.05 min, respectively. A linear gradient was applied, starting at 100% A (A: 95:5 10 mM NH₄OAc:MeCN) and ending at 100% B (B: MeCN), in 4 min.

Alternatively, HPLC analyses were performed on a Gynkotek P580 HPG consisting of gradient pump with a Gynkotek UVD 170S UV-vis.-detector equipped with a Chromolith Performance RP column (C18, 100 mm×4.6 mm). The column temperature was set to 25° C. A linear gradient was applied using MeCN/0.1 trifluoroacetic acid in MilliQ water, run from 10% to 100% MeCN in 5 minutes. Flow rate: 3 ml/min.

Thin layer chromatography (TLC) was performed on Merck TLC-plates (Silica gel 60 F₂₅₄) and UV visualized the spots. Flash chromatography was performed on a Combi Flash® Companion™ using RediSep™ normal-phase flash columns or using Merck Silica gel 60 (0.040-0.063 mm). Typical solvents used for flash chromatography were mixtures of chloroform/methanol, dichloromethane/methanol, heptane/ethyl acetate, chloroform/methanol/ammonia (aq.) and dichlorormethane/methanol/NH₃ (aq.). SCX ion exchange columns were performed on Isolute® columns. Chromatography through ion exchange columns were typically performed in solvents such a methanol.

Preparative chromatography was run on a Waters autopurification HPLC with a diode array detector. Column: XTerra MS C8, 19×300 mm, 10 μm. Narrow gradients with MeCN/(95:5 0.1M NH₄OAc:MeCN) were used at a flow rate of 20 ml/min. Alternatively, purification was achieved on a semi preparative Shimadzu LC-8A HPLC with a Shimadzu SPD-10A UV-vis.-detector equipped with a Waters Symmetry® column (C18, 5 μm, 100 mm×19 mm). Narrow gradients with MeCN/0.1% trifluoroacetic acid in MilliQ Water were used at a flow rate of 10 ml/min.

GCMS compound identification was performed on a GC/DIP-MS system supplied by Agilent Technologies consisting of a GC 6890N, G1530N, a G2614A Autosampler, G2613A injector and a G2589N mass spectrometer. The mass spectrometer was equipped with a Direct Inlet Probe (DIP) interface manufactured by SIM GmbH. The mass spectrometer was equipped with an electron impact (EI) ion source and the electron voltage was set to 70 eV. The mass spectrometer scanned between m/z 50-550 and the scan speed was set to 2.91 scan/s. Solvent delay was set from 0 min to 2.3 min. The column used was a VF-5 MS, ID 0.25 mm×15m, 0.25 μm (Varian Inc.). When introduced by GC, a linear temperature gradient was applied starting at 40-110° C. (hold 1 min) and ending at 200-300° C. (hold 1 min), 25° C./minute, depending on method used.

Preparative chromatography was run on a Waters FractionLynx system with a Autosampler combined Automated Fraction Collector (Waters 2767), Gradient Pump (Waters 2525), Column Switch (Waters CFO) and PDA (Waters 2996). Column; XTerra® Prep MS C8 10 μm OBD™ 19×300 mm, with guard column; XTerra® Prep MS C8 10 μm 19×10 mm Cartridge. A gradient from 100% A (95% 0.1M NH₄OAc in MilliQ water and 5% MeCN) to 100% B (100% MeCN) was applied for LC-separation at flow rate 20 mL/min. The PDA was scanned from 210-350 nm. UV triggering determined the fraction collection.

Alternatively, preparative chromatography was run on a Waters FractionLynx system with a Autosampler combined Automated Fraction Collector (Waters 2767), Gradient Pump (Waters 2425), Make Up Pump (Waters 515), Waters Passive Splitter, Column Switch (Waters SFO), PDA (Waters 2996) and Waters ZQ mass spectrometer. Column; XBridge™ Prep C8 5 μm OBD™ 19×250 mm, with guard column; XTerra® Prep MS C8 10 μm 19×10 mm Cartridge. A gradient from within 100% A (95% 0.1 M NH₄OAc in MilliQ water and 5% MeCN) to 100% B (100% MeCN) was applied for LC-separation at flow rate 20 mL/min. The PDA was scanned from 210-350 nm. The ZQ mass spectrometer was run with ESI in positive or negative mode. The Capillary Voltage was 3 kV and the Cone Voltage was 30V. Mixed triggering, UV and MS signal, determined the fraction collection.

### ABBREVIATIONS

PPSE trimethylsilylpolyphosphate ester
DMAP 4-(dimethylamino)pyridine

### DMF N,N-dimethylformamide

DMSO dimethyl sulfoxide
EDC 1-ethyl-3-(3-dimethylaminopropyl)carbodiimide hydrochloride
RT room temperature
Rt retention time
tert tertiary
DCM dichloromethane
THF tetrahyrofuran

### EXAMPLE 1

### 5-Benzofuran-2-yl-N-(2-sulfamoylphenyl)sulfonyl-pyridine-2-carboxamide

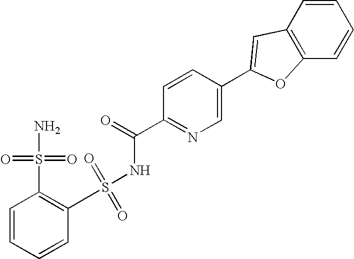

5-Bromo-N-(2-sulfamoylphenyl)sulfonyl-pyridine-2-carboxamide (57 mg, 0.14 mmol) was dissolved in DMF (800 μl), then benzofuran-2-boronic acid (24 mg, 0.15 mmol) was added followed by the addition of 2M sodium carbonate solution (400 μl). The mixture was subjected to vacuum/argon (×3); tetrakis(triphenylphosphine)palladium (8 mg, 0.05 mol %) was added and the reaction was allowed to stir at 90° C. overnight. Water was added to the cooled mixture that was then acidified (HCl). The resulting solid was filtered off, washed with water and was then purified by preparative HPLC (XTerra MS C8 column, acetonitrile/ammonium acetate buffer) to give the title compound as a solid (15 mg, 24% yield).

¹H NMR (400 MHz, MeOH) □ ppm 9.08 (d, 1H), 8.38 (dd, 1H), 8.33 (dd, 1H), 8.17-8.24 (m, 2H), 7.62-7.74 (m, 3H), 7.58 (d, 1H), 7.44 (s, 1H), 7.31-7.39 (m, 1H), 7.27 (t, 1H).

MS m/z M−H 455.7, M+H 457.7.

### a) 5-Bromo-N-(2-sulfamoylphenyl)sulfonyl-pyridine-2-carboxamide

Benzene-1,2-disulfonamide (1.0 g, 4.2 mmol), 5-bromopicolinic acid (1.3 g, 6.3 mmol), EDC (1.22 g, 6.3 mmol) and DMAP (1.3 g, 10.5 mmol) were mixed in DMF (25 ml) and the reaction mixture was stirred for 3 hours. The reaction mixture was diluted with water and washed twice with ethyl acetate. The aqueous layer was acidified (HCl) and the resulting solid was filtered off, washed with water then dried (high vacuum over P₂O₅) to give the title compound as a solid (1.4 g, 79% yield).

¹H NMR (400 MHz, DMSO-d₆) δ ppm 8.87 (dd, 1H), 8.36 (dd, 1H), 8.30 (dd, 1H), 8.16 (dd, 1H), 7.87-7.97 (m, 3H), 7.57 (br. s., 2H); MS m/z M−H 417.6, 419.6, M+H 419.6, 421.6.

### EXAMPLE 2

### 5-(2,3-Dichlorophenyl)-N-(2-sulfamoylphenyl)sulfonyl-pyridine-2-carboxamide

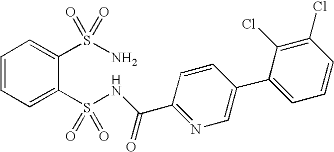

The title compound was synthesized using 2,3-dichlorophenylboronic acid and following an analogous preparation to that described for Example 1 (4 mg, 6% yield).

¹H NMR (400 MHz, MeOH) δ ppm 8.60 (d, 1H), 8.39 (dd, 1H), 8.17-8.23 (m, 2H), 7.95 (dd, 1H), 7.65-7.76 (m, 2H), 7.62 (dd, 1H), 7.33-7.46 (m, 2H); MS m/z M−H 483.7, 485.7, M+H 485.9, 487.9.

### EXAMPLE 3

### 4-Benzofuran-2-yl-N-(2-sulfamoylphenyl)sulfonyl-benzamide

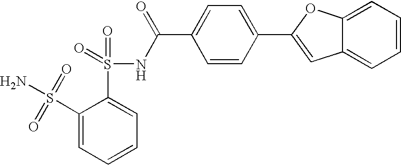

Benzene-1,2-disulfonamide (118 mg, 0.5 mmol), 4-benzofuran-2-ylbenzoic acid (153 mg, 0.65 mmol), EDC (124 mg, 0.65 mmol) and DMAP (183 mg, 1.5 mmol) were mixed in DMF (3 ml) and the reaction mixture was stirred for 3 hours. The reaction mixture was diluted with water (0.5 ml) and filtered. The filtrate was purified by HPLC to give the product as a solid (70 mg, 15% yield).

¹H NMR (400 MHz, DMSO-d₆) δ ppm 8.35-8.39 (m, 1H), 8.13-8.19 (m, 1H), 8.02 (s, 4H), 7.85-7.96 (m, 2H), 7.71 (dd, 1H), 7.66 (dd, 1H), 7.65 (s, 1H), 7.45 (s, 2H), 7.37 (ddd, 1H), 7.26-7.32 (m, 1H).

MS m/z M−H 455.4.

### EXAMPLE 4

### 4-Benzothiophen-2-yl-N-(2-sulfamoylphenyl)sulfonyl-benzamide

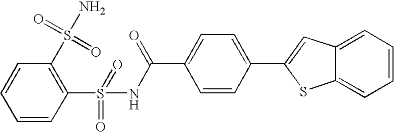

The title compound was synthesized using the appropriate benzoic acid derivative and following an analogous preparation to that described for Example 3 (7 mg, 30% yield).

¹H NMR (400 MHz, MeOH) δ ppm 8.48 (br. s., 1H) 8.28 (dd, 1H) 7.96 (d, 2H) 7.79-7.89 (m, 7H) 7.31-7.40 (m, 2H).

MS m/z M−H 471.2.

### EXAMPLE 5

### 4-Benzothiazol-2-yl-N-(2-sulfamoylphenyl)sulfonyl-benzamide

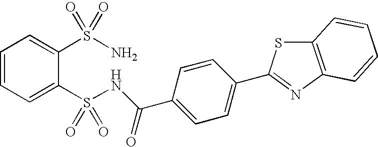

Benzene-1,2-disulfonamide (50 mg, 0.21 mmol), 4-benzothiazol-2-ylbenzoic acid (81 mg, 0.32 mmol), DMAP (65 mg, 0.53 mmol) and EDC (61 mg, 0.32 mmol) were mixed in DMF (1.8 ml) and the reaction mixture was stirred until a clear solution was obtained (2 h). The crude product was purified by preparative HPLC (XTerra MS C8 column, acetonitrile/ammonium acetate buffer) to give the title compound as a solid (28 mg, 28% yield).

¹H NMR (400 MHz, MeOH) δ ppm 8.34 (dd, 1H), 8.19 (dd, 1H), 8.14-8.17 (m, 2H), 8.08-8.12 (m, 2H), 8.01-8.06 (m, 2H), 7.67-7.72 (m, 1H), 7.62-7.67 (m, 1H), 7.52-7.57 (m, 1H), 7.42-7.48 (m, 1H).

MS m/z M−H 472.0, M+H 473.7.

### EXAMPLE 6

### 4-(7-Oxa-3,9-diazabicyclo[4.3.0]nona-2,4,8,10-tetraen-8-yl)-N-(2-sulfamoylphenyl)sulfonyl-benzamide

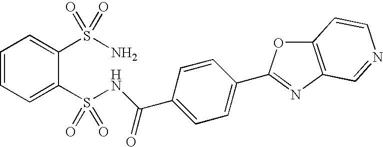

The title compound was obtained as a solid (40 mg, 21% yield) using the appropriate benzoic acid derivative and following an analogous procedure to that described for Example 5 except the reaction was heated to 50° C. for 2 h to give a clear solution.

¹H NMR (400 MHz, DMSO-d₆) δ ppm 9.21 (s, 1H), 8.65 (d, 1H), 8.25-8.33 (m, 3H), 8.07-8.15 (m, 3H), 8.00 (d, 1H), 7.75-7.86 (m, 2H), 7.47 (br. s., 2H).

MS m/z M−H 457.0, M+H 459.0.

### a) 4-([1,3]Oxazolo[4,5-c]pyridin-2-yl)benzoic Acid

To a solution of methyl 4-(oxazolo[4,5-c]pyridin-2-yl)benzoate (1.27 g, 5.0 mmol) in MeOH (20 ml) and THF (20 ml), was added an 2N aqueous solution of LiOH (5 ml, 10.0 mmol).The reaction mixture was stirred at RT for 20 h and then concentrated to one third volume. The solid was filtered off, washed with CH₃CN (3×) and diethyl ether, and dried over P₂O₅ at 50° C. under reduced pressure to give lithium 4-([1,3]oxazolo[4,5-c]pyridin-2-yl)benzoate (0.98 g, 80%).

¹H NMR (DMSO-d₆ AcOH) δ 7.93 (d, 1H), 8.17 (d, 2H), 8.33 (d, 2H), 8.63 (d, 1H), 9.17 (s, 1H).

LCMS (ESI) for C₁₃H₈N₂O₃ (M=240.22): 241 [MH]⁺.

### b) Methyl 4-(oxazolo[4,5-c]pyridin-2-yl)benzoate

A solution of PPSE was prepared by heating to reflux a mixture of P₂O₅ (4.26 g, 15 mmol) and hexamethyldisiloxane (12.75 ml, 60 mmol) in 1,2-dichlorobenzene (30 ml) under an argon atmosphere until the solution becomes clear (˜5 min.).

Methyl 4-(4-hydroxypyridin-3-ylcarbamoyl)benzoate (2.91 g, 10 mmol) was added to PPSE at 180° C. (oil bath temperature) and the mixture was refluxed with vigorous stirring for 2 h. After cooling, a precipitate appeared. Diethyl ether was added to the reaction mixture, the solid was collected by filtration and washed with diethyl ether. The solid was then suspended in DCM-MeOH and the mixture was neutralised with aqueous saturated NaHCO₃ solution. The aqueous layer was back extracted with DCM, the organic layers were combined and washed with brine, dried over MgSO₄ and concentrated. The remaining solid was triturated with diethyl ether, filtered, washed with diethyl ether and dried under vacuo at 50° C. to afford methyl 4-(oxazolo[4,5-c]pyridin-2-yl)benzoate (1.00 g, 79%).

¹H NMR (DMSO-d₆): δ 3.94 (s, 3H), 7.97 (dd, 1H), 8.22 (d, 2H), 8.35 (d, 2H), 8.66 (d, 1H), 9.20 (s 1H).

LCMS (EIC) for C₁₄H₁₀N₂O₃ (M=254.25): 254 [M]⁺.

### c) Methyl 4-(hydroxypyridin-3-ylcarbamoyl)benzoate

A mixture of terephthalic acid monomethyl ester (7.20 g, 40 mmol), SOCl₂ (60 ml) and DMF (50 μl) was stirred at reflux for 1 h. After removal of the excess SOCl₂, the residue was azeotroped with toluene (3×) to remove the residual SOCl₂. The crude acid chloride was dissolved in DCM (10 ml) and added dropwise at 0° C. to a solution of 3-amino-4-hydroxypyridine (7.32 g, 40 mmol) in pyridine (40 ml). The reaction mixture was stirred at RT during 2.5 days. Pyridine was evaporated and water was added to the residue.

The solid was filtered off, washed with water (3×), a mixture 1:3 of CH₃CN—diethyl ether, diethyl ether and dried under vacuo at 60° C. to afford methyl 4-(4-hydroxypyridin-3-ylcarbamoyl)benzoate (9.70 g, 89%) which was used without further purification.

¹H NMR (DMSO-d₆): δ 3.88 (s, 3H), 6.3 1 (d, 1H), 7.71 (d, 1H), 8.01 (d, 2H), 8.09 (d, 2H), 8.75 (s, 1H), 9.43 (s, 1H).

### EXAMPLE 7

### 4-(7-Oxa-5,9-diazabicyclo[4.3.0]nona-2,4,8,10-tetraen-8-yl)-N-(2-sulfamoylphenyl)sulfonyl-benzamide

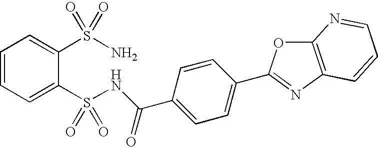

The title compound was obtained as a solid (12 mg, 11% yield) using the appropriate benzoic acid derivative and following an analogous procedure to that described for Example 5 except the reaction was heated to 50° C. for 2 h to give a clear solution.

¹H NMR (400 MHz, MeOH) δ ppm 8.32-8.40 (m, 2H), 8.28 (d, 2H), 8.15-8.24 (m, 4H), 7.60-7.74 (m, 2H), 7.49 (dd, 1H).

MS m/z M−H 457.0, M+H 458.7.

### a) 4-(Oxazolo[5,4-b]pyridin-2-yl)benzoic Acid

To a solution of methyl 4-(oxazolo[5,4-b]pyridin-2-yl)benzoate (1.016 g, 4.0 mmol) in MeOH (12 ml) and THF (12 ml), was added an 2N aqueous solution of LiOH (4 ml, 8.0 mmol).The reaction mixture was stirred at RT for 15 h. The solvents were evaporated off, the residue diluted with CH₃CN to afford a solid, which was filtered off, washed with CH₃CN and diethyl ether. The solid was then added to 6M HCl (15 ml) giving a white precipitate which was filtered off, washed with water and dried over P₂O₅ at 50° C. under reduced pressure to give 4-(oxazolo[5,4-b]pyridin-2-yl)benzoic acid (0.60 g, 63%).

¹H NMR (DMSO-d₆): δ 7.53 (m, 1H), 8.15 (d, 2H), 8.32 (m, 3H), 8.41 (d, 1H).

LCMS (EIC) for C₁₃H₈N₂O₃ (M=240.22): 240 [M].⁺.

### b) Methyl 4-(oxazolo[5,4-b]pyridin-2-yl)benzoate

A solution of PPSE (trimethylsilylpolyphosphate ester) was prepared according to the literature (Aizpurua, J. M., Paloma, C. Bull Soc. Chim. Fr. 1984, 142) by heating to reflux a mixture of P₂O₅ (3.124 g, 11 mmol) and hexamethyldisiloxane (9 ml, 42.3 mmol) in 1,2-dichlorobenzene (20 ml) under an argon atmosphere until the solution became clear (˜5 min.).

After cooling, methyl 4-(2-chloropyridin-3-ylcarbamoyl)benzoate (2.91 g, 10 mmol) was added to PPSE and the mixture was refluxed with vigorous stirring for 24 h. After cooling, diethyl ether was added to the reaction mixture, the precipitate was collected by filtration and washed with petroleum ether. The solid was then dissolved in DCM, the solution was washed with an aqueous saturated NaHCO₃ solution, dried over MgSO₄ and concentrated. A crystalline solid precipitate which was collected, washed with petroleum ether and dried under vacuo to afford methyl 4-(oxazolo[5,4-b]pyridin-2-yl)benzoate (2.06 g, 81%).

¹H NMR (CDCl₃): δ 3.98 (s, 3H), 7.39 (dd, 1H), 8.12 (d, 1H), 8.22 (d, 2H), 8.22 (d, 2H), 8.39 (dd, 1H).

LCMS (ESI) for C₁₄H₁₀N₂O₃ (M=254.25): 255 [MH]⁺.

### c) Methyl 4-(2-chloropyridin-3-ylcarbamoyl)benzoate

A mixture of terephthalic acid monomethyl ester (2.70 g, 1.5 mmol), SOCl₂ (25 ml) and 5 drops of DMF was stirred at RT overnight. After removal of the excess SOCl₂, the residue was azeotroped with toluene (3×) to remove the residual SOCl₂. The crude acid chloride was dissolved in THF (10 ml) and added dropwise to a solution of 2-chloropyridin-3-amine (1.93 g, 1.5 mmol) and triethylamine (2.8 ml, 2.0 mmol) in THF (30 ml) at 0° C.

The reaction mixture was stirred at RT overnight; the precipitate was filtered off and the filtrate was concentrated. The crude solid was triturated with diethyl ether, filtered, washed with diethyl ether and dried under vacuo to afford methyl 4-(2-chloropyridin-3-ylcarbamoyl)benzoate (2.68 g, 61%) as a white solid. The filtrate was evaporated and the residue was purified by flash chromatography (DCM/EtOAc 95:5) to afford a second batch of methyl 4-(2-chloropyridin-3-ylcarbamoyl)benzoate (0.63 g, 14%).

¹H NMR (CDCl₃): δ 4.02 (s, 3H), 7.35 (dd, 1H), 7.98 (d, 2H), 8.18 (dd, 1H), 8.21 (d, 2H), 8.45 (s, 1H), 8.91 (dd, 1H).

LCMS (ESI) for C₁₄H₁₁C1N₂O₃ (M=290.71): 291 [MH]⁺.

### EXAMPLE 8

### 4-Benzooxazol-2-yl-N-(2-sulfamoylphenyl)sulfonyl-benzamide

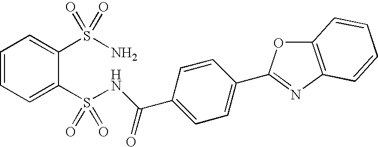

Benzene-1,2-disulfonamide (50 mg, 0.21 mmol), 4-benzooxazol-2-ylbenzoic acid (51 mg, 0.21 mmol), DMAP (65 mg, 0.53 mmol) and EDC (57 mg, 0.29 mmol) were mixed in DMF (1.8 ml) and the reaction mixture was stirred for 1 h at RT, then at 50° C. until a clear solution was obtained (30 min). The crude material was purified by preparative HPLC (XTerra MS C8 column, acetonitrile/ammonium acetate buffer) to give the title compound as a solid (40 mg, 42% yield).

¹H NMR (400 MHz, MeOH) δ ppm 8.51 (dd, 1H), 8.33 (d, 2H), 8.25-8.30 (m, 1H), 8.07 (d, 2H), 7.82-7.89 (m, 2H), 7.75-7.80 (m, 1H), 7.71 (dd, 1H), 7.38-7.51 (m, 2H).

MS m/z M−H 456.0, M+H 457.8.

### EXAMPLE 9

### 2-Phenyl-N-(2-sulfamoylphenyl)sulfonyl-benzofuran-6-carboxamide

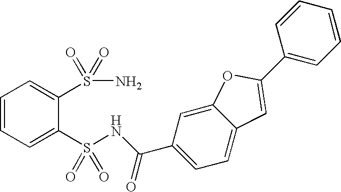

Benzene-1,2-disulfonamide (50 mg, 0.21 mmol), 2-phenylbenzofuran-6-carboxylic acid (Example 29a) (53 mg, 0.21 mmol), DMAP (57 mg, 0.46 mmol) and EDC (45 mg, 0.23 mmol) were mixed in DMF (1.8 ml) and the reaction mixture was stirred at RT until a clear solution was obtained (2 h). The crude material was purified by preparative HPLC (XTerra MS C8 column, acetonitrile/ammonium acetate buffer) to give the title compound as a film (82 mg, 61% yield).

¹H NMR (400 MHz, MeOH) δ ppm 8.34 (dd, 1H), 8.16-8.22 (m, 2H), 7.89-7.97 (m, 3H), 7.66-7.71 (m, 1H), 7.61-7.66 (m, 1H), 7.56 (d, 1H), 7.44-7.51 (m, 2H), 7.35-7.41 (m, 1H), 7.22 (s, 1H).

MS m/z M−H 455.0.

### a) 2-Phenyl-benzofuran-6-carboxylic Acid

A mixture of 2-phenyl-benzofuran-6-carboxylic acid methyl ester (490 mg, 1.94 mmol) and LiOH.H₂O (326 mg, 7.26 mmol) in ethanol (20 mL) was heated at reflux for 1 hour. The ethanol was removed under reduced pressure and the residue was partitioned between ethyl acetate and water. The aqueous layer was then separated and acidified to pH 4 using citric acid. The precipitated solid was isolated by filtration and dried under high vacuum to give 2-phenyl-benzofuran-6-carboxylic acid (240 mg, 52% yield).

¹H NMR (400 MHz, DMSO-d₆): δ (ppm) 12.8 (br s, 1H), 8.14 (s, 1H), 8.02-7.96 (d, 2H), 7.92-7.86 (d, 1H), 7.80-7.74 (dd, 1H), 7.60-7.52 (m, 3H), 7.50-7.44 (m, 1H); ¹⁹F NMR (400 MHz, DMSO-d₆): δ (ppm) 57.5.

ESMS: m/z [M⁺+1] 238.89.

### b) 2-Phenyl-benzofuran-6-carboxylic Acid Methyl Ester

A mixture of 3-hydroxy-4-iodo-benzoic acid methyl ester (2 g, 7.20 mmol), phenylacetylene (3.68 g, 36.02 mmol), CuI (68 mg, 0.35 mmol), Pd(PPh₃)₂Cl₂ (253 mg, 36.04 mmol) and tetramethylguanidine (8.3 g, 72.06 mmol) in DMF was heated at 60° C. for 10 minutes and then at RT overnight. The reaction mixture was poured into aqueous 2N HCl (70 mL) and the product was extracted with ethyl acetate. The combined extracts were washed with water, dried over Na₂SO₄, and concentrated under reduced pressure. Purification of the crude product by flash column chromatography using 10-30% ethyl acetate/hexane as eluent afforded 2-phenyl-benzofuran-6-carboxylic acid methyl ester (430 mg, 24% yield).

¹H NMR (400 MHz, CDCl₃): δ (ppm) 8.22 (s, 1H), 7.98-7.94 (m, 1H), 7.93-7.88 (m, 2H), 7.64-7.6 (m, 1H), 7.52-7.46 (m, 2H), 7.44-7.38 (s, 1H), 7.08-7.06 (s, 1H), 3.97 (s, 3H).

ESMS: m/z [M⁺+1] 253.07.

### EXAMPLE 10

### 4-Bromo-N-(2-sulfamoylphenyl)sulfonyl-benzamide

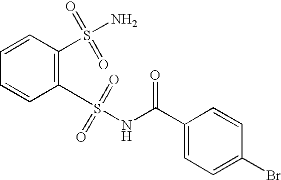

Benzene-1,2-disulfonamide (118 mg, 0.5 mmol), 4-bromobenzoic acid (131 mg, 0.65 mmol), EDC (124 mg, 0.65 mmol) and DMAP (183 mg, 1.5 mmol) were mixed in DMF (3 ml) and the reaction mixture was stirred for 3 hours. The reaction mixture was diluted with water (0.5 ml) and filtered. The filtrate was purified by HPLC to give the product as a solid (91 mg, 43%).

¹H NMR (400 MHz, DMSO-d₆) δ ppm 8.14 (d, 1H), 7.98 (d, 1H), 7.80 (d, 2H), 7.54-7.67 (m, 2H), 7.51 (d, 2H), 7.42 (s, 2H).

MS m/z M+H 419, 421, M−H 417, 419.

### EXAMPLE 11

### 4-Bromo-2-chloro-N-(2-sulfamoylphenyl)sulfonyl-benzamide

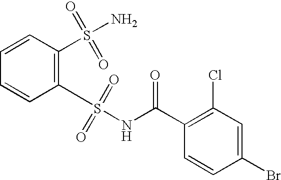

Benzene-1,2-disulfonamide (42 mg, 0.18 mmol), 2-chloro-4-bromobenzoic acid (131 mg, 0.65 mmol), EDC (48 mg, 0.25 mmol) and DMAP (76 mg, 0.63 mmol) were mixed in DMF (1 ml) and the reaction mixture was stirred for 3 hours. The reaction mixture was diluted with water (0.2 ml) and filtered. The filtrate was purified by HPLC to give the product as a solid (42 mg, 51%).

¹H NMR (400 MHz, DMSO-d₆) δ ppm 8.20 (dd, 1H), 8.03 (d, 1H), 7.59-7.72 (m, 3H), 7.56 (d, 1H), 7.48 (dd, 1H), 7.36 (s, 2H).

MS m/z, M−H 451, 453.

The compounds of Examples 12 to 21 and 23 were prepared using the appropriate carboxylic acid derivative and following an analogous procedure to that described for Example 11.

### EXAMPLE 12

### 4-Bromo-3-methyl-N-(2-sulfamoylphenyl)sulfonyl-benzamide

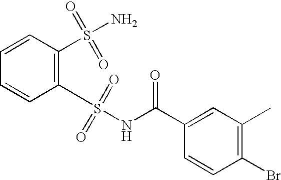

46 mg, 59%

¹H NMR (400 MHz, DMSO-d₆) δ ppm 8.18 (d, 1H), 8.03 (d, 1H), 7.84 (s, 1H), 7.62-7.74 (m, 2H), 7.51-7.62 (m, 2H), 7.42 (s, 2H), 2.35 (s, 3H).

MS m/z M+H 433, 435, M−H 431, 433.

### EXAMPLE 13

### 4-Bromo-3-fluoro-N-(2-sulfamoylphenyl)sulfonyl-benzamide

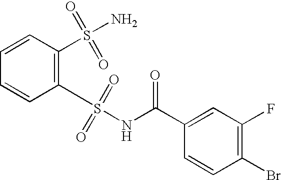

44 mg, 56%.

¹H NMR (400 MHz, DMSO-d₆) δ ppm 8.15 (dd, 1H), 7.99 (dd, 1H), 7.54-7.71 (m, 5H), 7.40 (s, 1H).

MS m/z M−H 435, 437.

### EXAMPLE 14

### 4-Bromo-2-fluoro-N-(2-sulfamoylphenyl)sulfonyl-benzamide

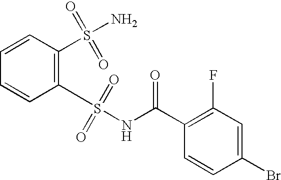

40 mg, 51%.

¹H NMR (400 MHz, MeOH) δ ppm 8.34 (dd, 1H), 8.19 (dd, 1H), 7.75 (t, 1H), 7.67-7.72 (m, 1H), 7.62-7.67 (m, 1H), 7.28-7.34 (m, 2H).

MS m/z M−H 435, 437

### EXAMPLE 15

### 4-Bromo-2-methyl-N-(2-sulfamoylphenyl)sulfonyl-benzamide

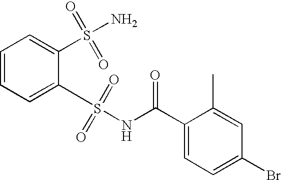

48 mg, 62%.

¹H NMR (400 MHz, MeOH) δ ppm 8.40-8.44 (m, 1H), 8.22-8.27 (m, 1H), 7.74-7.82 (m, 2H), 7.50 (d, 1H), 7.33-7.40 (m, 2H), 2.33 (s, 3H).

MS m/z M−H 431, 433.

### EXAMPLE 16

### 2-(1-Adamantyl)-N-(2-sulfamoylphenyl)sulfonyl-acetamide

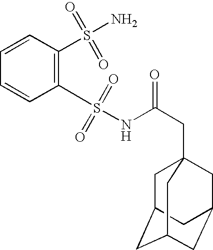

35 mg, 47%.

¹H NMR (400 MHz, DMSO-d₆) δ ppm 11.93 (br. s., 1H), 8.25 (d, 1H), 8.14 (d, 1H), 7.79-7.96 (m, 2H), 7.27 (s, 2H), 1.98 (s, 2H), 1.85 (br. s., 3H), 1.56-1.66 (m, 3H), 1.40-1.54 (m, 9H).

MS m/z M+H 413, M−H 411.

### EXAMPLE 17

### N-(2-Sulfamoylphenyl)sulfonylnorbornane-2-carboxamide

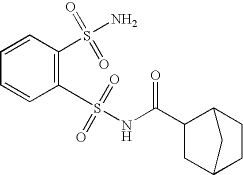

22 mg, 34%.

MS m/z, M−H 357; Rt HPLC (XTerra) 1.85 min.

### EXAMPLE 18

### 1-Phenyl-N-(2-sulfamoylphenyl)sulfonyl-cyclohexane-1-carboxamide

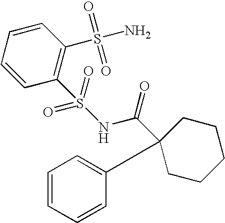

12 mg, 16%.

H NMR (400 MHz, MeOH) δ ppm 8.08-8.27 (m, 2H), 7.65-7.86 (m, 2H), 7.16-7.28 (m, 5H), 2.24-2.35 (m, 2H), 1.69-1.79 (m, 2H), 1.48-1.62 (m, 3H), 1.35-1.48 (m, 2H), 1.20-1.34 (m, 1H).

MS m/z M−H 421.

### EXAMPLE 19

### 3-(Difluoromethoxy)-N-(2-sulfamoylphenyl)sulfonyl-benzamide

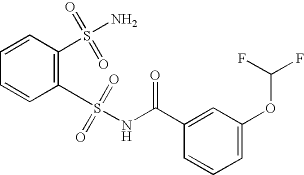

40 mg, 55%

¹H NMR (400 MHz, MeOH) δ ppm 8.34-8.40 (m, 1H), 8.19-8.24 (m, 1H), 7.81 (d, 1H), 7.68-7.78 (m, 3H), 7.43 (t, 1H), 7.27 (dd, 1H), 6.85 (t, 1H).

MS m/z M+H 407, M−H 405.

### EXAMPLE 20

### 3-Bromo-4-fluoro-N-(2-sulfamoylphenyl)sulfonyl-benzamide

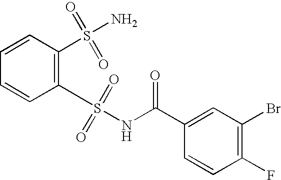

27 mg, 34%.

¹H NMR (400 MHz, MeOH) δ ppm 8.33-8.38 (m, 1H), 8.19-8.24 (m, 2H), 7.93-7.98 (m, 1H), 7.68-7.77 (m, 2H), 7.22 (t, 1H).

MS m/z M−H 335, 337.

### EXAMPLE 21

### N-(2-Sulfamoylphenyl)sulfonyl-3-(2,2,3,3-tetrafluoropropoxymethyl)benzamide

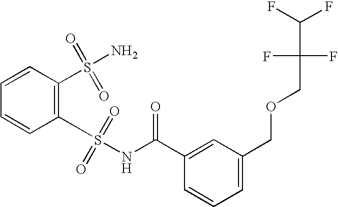

56 mg, 64%.

¹H NMR (400 MHz, DMSO-d₆) δ ppm 8.31-8.35 (m, 1H), 8.12-8.16 (m, 1H), 7.82-7.93 (m, 4H), 7.57 (d, 1H), 7.48 (t, 1H), 7.40 (s, 2H), 6.54 (tt, 1H), 4.66 (s, 2H), 3.98 (t, 2H).

MS m/z M+H 485, M−H 483.

### EXAMPLE 22

### 4-Methyl-N-(2-sulfamoylphenyl)sulfonyl-2-[3-(trifluoromethyl)phenyl]1,3-thiazole-5-carboxamide

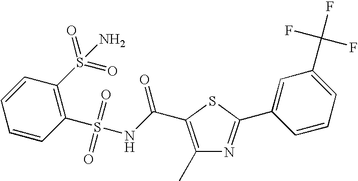

Benzene-1,2-disulfonamide (84 mg, 0.36 mmol), 4-methyl-2-[3-(trifluoromethyl)phenyl]1,3-thiazole-5-carboxylic acid (142 mg, 0.5 mmol), EDC (96 mg, 0.5 mmol) and DMAP (152 mg, 1.26 mmol) were mixed in DMF (2 ml) and the reaction mixture was stirred for 3 hours. The reaction mixture was diluted with water (0.5 ml) and filtered. The filtrate was purified by HPLC to give the product as a solid (77 mg, 42%).

¹H NMR (400 MHz, MeOH) δ ppm 8.35 (dd, 1H), 8.26 (s, 1H), 8.15-8.23 (m, 2H), 7.77 (d, 1H), 7.64-7.75 (m, 3H), 2.67 (s, 3H).

MS m/z M+H 506.6, M−H 504.6.

### EXAMPLE 23

### 4-Chloro-2-fluoro-N-(2-sulfamoylphenyl)sulfonyl-benzamide

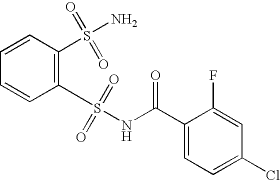

33 mg, 46%.

¹H NMR (400 MHz, DMSO-d₆) δ ppm 8.27-8.36 (m, 1H), 8.13-8.19 (m, 1H), 7.83-7.94 (m, 2H), 7.68 (t, 1H), 7.51-7.58 (m, 1H), 7.33-7.47 (m, 3H).

MS m/z M−H 391.

### General Procedure for Examples 24-25

To a solution of the appropriate carboxylic acid (1 mmol) in dry DMF (15 mL), benzene-1,2-disulfonamide (0.9 mmol), EDC (1 mmol) and DMAP (1 mmol) were added. The reaction mixture was heated at 40-45° C. for 4 to 17 hours. Most of the DMF was then removed under reduced pressure and the crude product was purified without further work-up using preparative HPLC. Alternatively, after removal of DMF, the residue was partitioned between ethyl acetate and aqueous 1N HCl. The organic layer was separated, washed with water, dried over sodium sulfate and concentrated in vacuo. The crude product was then purified by flash column chromatography or recrystallization.

### EXAMPLE 24

### 2-Benzyl-4-chloro-N-(2-sulfamoylphenyl)sulfonyl-benzamide

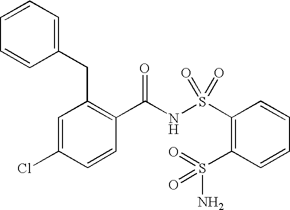

Following the general procedure, 2-benzyl-4-chlorobenzoic acid (330 mg, 1.34 mmol) was reacted with benzene-1,2-disulfonamide (285 mg, 1.21 mmol), EDC (257 mg, 1.34 mmol) and DMAP (164 mg, 1.34 mmol) for 17 hours. Purification of the crude product by preparative HPLC afforded the title compound (60 mg, 11%).

¹H NMR (400 MHz, MeOH-d₄): δ (ppm) 8.48 (dd, 1H), 8.28 (dd, 1H), 7.76-7.95 (m, 2H), 7.56 (d, 1H), 7.08-7.34 (m, 5H), 7.03 (d, 2H), 4.03 (s, 2H).

ESMS: m/z [M−1]: 463 and 465.

### EXAMPLE 25

### 2-Phenyl-N-(2-sulfamoylphenyl)sulfonyl-benzofuran-5-carboxamide

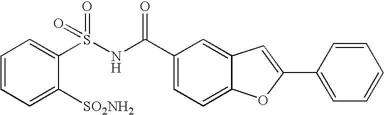

Following the general procedure, 2-phenyl-benzofuran-5-carboxylic acid (200 mg, 0.83 mmol) was reacted with benzene-1,2-disulfonamide (179 mg, 0.75 mmol), EDC (161 mg, 0.84 mmol) and DMAP (103 mg, 0.84 mmol) for 4 hours. The crude product was purified by preparative HPLC to afford the title compound (62 mg, 16%).

¹H NMR (400 MHz, CDCl₃): δ (ppm) 9.53 (br s, 1H), 8.60 (d, 1H), 8.28 (d, 1H) 8.07 (br s, 1H), 7.92-7.79 (m, 4H), 7.76 (d, 1H), 7.56 (d, 1H), 7.47 (t, 2H), 7.4 (d, 1H), 7.07 (s, 1H), 5.73 (br s, 2H).

ESMS: m/z [M−1] 454.92.

### a) 2-Phenyl-benzofuran-5-carboxylic acid

A mixture of 2-phenyl-benzofuran-5-carboxylic acid methyl ester (1.7 g, 6.73 mmol) and LiOH.H₂O (1.14 g, 27.16 mmol) in ethanol (50 mL) was heated to reflux for 45 minutes. Most of the ethanol was then removed under reduced pressure and the residue was partitioned between ethyl acetate and water. The aqueous layer was separated and acidified with citric acid to pH 4. The precipitated white solid was filtered off, washed with water and dried to afford 2-phenyl-benzofuran-5-carboxylic acid (710 mg, 44%).

¹H NMR (400 MHz, DMSO-d₆): δ (ppm) 12.95 (br s, 1H), 8.29 (s, 1H), 7.96 (d, 2H), 7.74 (d, 1H), 7.60-7.50 (m, 3H), 7.50-7.41 (m, 2H).

ESMS: m/z [M⁺+1] 238.96.

### b) 2-Phenyl-benzofuran-5-carboxylic Acid Methyl Ester

A mixture of methyl 4-hydroxy-3-iodobenzoate (1 g, 3.59 mmol), CuI (35 mg, 0.183 mmol), Pd(PPh₃)₂Cl₂ (127 mg, 0.180 mmol), tetramethylguanidine (4.14 g, 35.9 mmol) in DMF (20 mL) was stirred at RT for 10 minutes. Phenylacetylene (1.83 g, 17.98 mmol) was then added and the mixture was stirred for 2 hours at 60° C. and then at RT overnight. The reaction mixture was poured into 2N HCl (100 mL) and the product was extracted with ethyl acetate. The organic layer was washed with water, dried over Na₂SO₄, filtered and concentrated under reduced pressure. The crude product was purified by flash column chromatography using 30% ethyl acetate/hexane to afford 2-phenyl-benzofuran-5-carboxylic acid methyl ester as a yellow solid (700 mg, 77%).

¹H NMR (400 MHz, CDCl₃): δ (ppm) 8.33 (s, 1H) 8.03 (d, 1H) 7.89 (d, 2H) 7.56 (d, 1H) 7.48 (t, 2H) 7.41 (d, 1H) 7.09 (s, 1H) 3.96 (s, 3H).

### EXAMPLE 26

### 4-Methyl-N-(2-sulfamoylphenyl)sulfonyl-2-[4-(trifluoromethyl)phenyl]1,3-thiazole-5-carboxamide

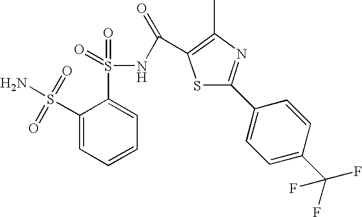

4-Methyl-2-[4-(trifluoromethyl)phenyl]1,3-thiazole-5-carboxylic acid (122 mg, 0.42 mmol), triethylamine (42 mg, 0.42 mmol) and O-(1H-benzotriazol-1-yl)-N,N,N′,N′-tetramethyluronium (HBTU) (160 mg, 0.42 mmol) were mixed in MeCN/DMF (3 ml, 2:1).

After 10 minutes, benzene-1,2-disulfonamide (100 mg, 0.42 mmol) was added and the reaction mixture was stirred for 12-14 hours. The reaction mixture was filtered and purified by HPLC (XTerra MS C8 column, acetonitrile/ammonium acetate buffer) (138 mg, 65%).

¹H NMR (400 MHz, DMSO-d₆) δ ppm 8.09-8.21 (m, 3H), 7.99-8.06 (d, 1H), 7.80-7.89 (d, 2H), 7.57-7.74 (m, 2H), 7.35 (br s, 2H), 2.56 (s, 3H).

MS (ES−) 504, 505.

### EXAMPLE 27

### 2-(2,3-Dihydrobenzofuran-5-yl)-4-methyl-N-(2-sulfamoylphenyl)sulfonyl-1,3-thiazole-5-carboxamide

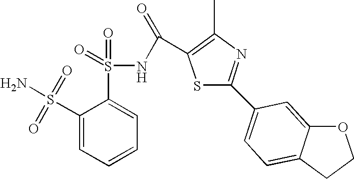

Benzene-1,2-disulfonamide (100 mg, 0.42 mmol), the carboxylic acid (110 mg, 0.42 mmol), EDC (80 mg, 0.42 mmol) and DMAP (103 mg, 0.84 mmol) were mixed in DMF (3 ml) and the reaction mixture was stirred for 12-15 hours. The reaction mixture was filtered and purified by HPLC (XTerra MS C8 column, acetonitrile/ammonium acetate buffer) to give the product as a solid (19 mg, 15%).

¹H NMR (400 MHz, DMSO-d₆) δ ppm 8.09 (d, 1H), 7.88 (d, 1H), 7.67-7.79 (m, 2H), 7.47-7.65 (m, 3H), 7.38 (s, 2H), 6.86 (dd, 2H), 3.07 (m, 2H), 2.54 (s, 3H).

MS (ES−) 478, 479.

The compounds of Examples 28 to 30 were prepared using the appropriate carboxylic acid derivative and following an analogous procedure to that described for Example 27.

### EXAMPLE 28

### 2-(4-Chlorophenyl)-4-methyl-N-(2-sulfamoylphenyl)sulfonyl-1,3-thiazole-5-carboxamide

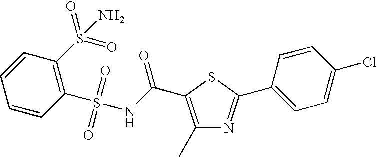

22 mg, 11%.

¹H NMR (400 MHz, DMSO-d₆) δ ppm 8.15 (dd, 1H), 8.01 (dd, 1H), 7.90-7.96 (m, 2H), 7.64-7.70 (m, 1H), 7.58-7.64 (m, 1H), 7.51-7.56 (m, 2H), 7.39 (s., 2H), 2.57 (s, 3H).

### EXAMPLE 29

### 4-Methyl-2-phenyl-N-(2-sulfamoylphenyl)sulfonyl-1,3-thiazole-5-carboxamide

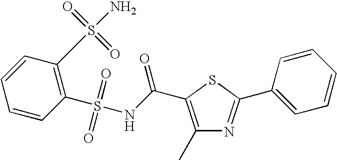

20 mg, 11%.

¹H NMR (400 MHz, DMSO-d₆) δ ppm 8.15 (dd, 1H), 8.01 (dd, 1H), 7.88-7.95 (m, 2H), 7.64-7.71 (m, 1H), 7.57-7.64 (m, 1H), 7.44-7.52 (m, 3H), 7.39 (br. s., 2H), 2.57 (s, 3H).

### EXAMPLE 30

### 4-Phenylmethoxy-N-(2-sulfamoylphenyl)sulfonyl-benzamide

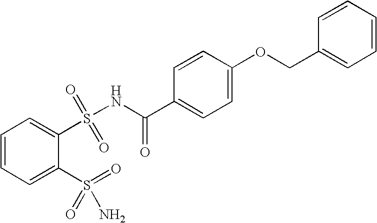

13 mg, 14%.

¹H NMR (400 MHz, DMSO-d₆) δ ppm 8.28 (dd, 1H), 8.18 (dd, 1H), 7.92-7.98 (m, 2H), 7.58-7.69 (m, 2H), 7.40-7.45 (m, 2H), 7.34-7.39 (m, 2H), 7.27-7.33 (m, 1H), 6.92-6.98 (m, 2H), 5.11 (s, 2H).

### General Procedure for EXAMPLES 31-41

Stock solutions of carboxylic acids/acid chlorides in DMF were treated with EDC and DMAP. To these were added stock solutions of benzene-1,2-disulfonamide in DMF in 48 wells and the reaction was put on a shaker overnight. The solvent was removed (centrifuge) and preparative chromatography was run on a Waters FractionLynx system with a Autosampler combined Automated Fraction Collector (Waters 2767), Gradient Pump (Waters 2525), Regeneration Pump (Waters 600), Make Up Pump (Waters 515), Waters Active Splitter, Column Switch (Waters CFO), PDA (Waters 2996) and Waters ZQ mass spectrometer. Column; XBridge™ Prep C8 5 μm OBD™ 19×100 mm, with guard column; XTerra Prep MS C8 10 μm 19×10 mm Cartridge. A gradient from 100% A (95% 0.1M NH₄OAc in MilliQ water and 5% MeCN) to 100% B (100% MeCN) was applied for LC-separation at flow rate 25 ml/min. The PDA was scanned from 210-350 nm. The ZQ mass spectrometer was run with ESI in positive mode. The Capillary Voltage was 3 kV and the Cone Voltage was 30V. Mixed triggering, UV and MS signal, determined the fraction collection.

Purity analysis was run on a Water Acquity system with PDA (Waters 2996) and Waters ZQ mass spectrometer. Column; Acquity UPLC™ BEH Cₛ 1.7 μm 2.1×50 mm. The column temperature was set to 65° C. A linear 2 min 15sec gradient from 100% A (A: 95% 0.01 M NH₄OAc in MilliQ water and 5% MeCN) to 100% B (5% 0.01M NH₄OAc in MilliQ water and 95% MeCN) was applied for LC-separation at flow rate 1.0 ml/min. The PDA was scanned from 210-350 nm and 254 nm was extracted for purity determination. The ZQ mass spectrometer was run with ESI in pos/neg switching mode. The Capillary Voltage was 3 kV and the Cone Voltage was 30V.

Alternatively, preparative chromatography was carried out on an HPLC (XTerra MS C8 column, acetonitrile/ammonium acetate buffer).

### EXAMPLE 31

### 4-Phenyl-N-(2-sulfamoylphenyl)sulfonyl-benzamide

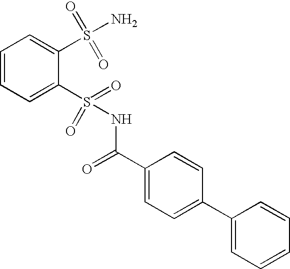

¹H NMR (400 MHz, DMSO-d₆) δ ppm 8.2 (d, 1H), 7.93-8.03 (m, 3H), 7.55-7.73 (m, 6H), 7.45-7.54 (m, 2H), 7.33-7.43 (m, 1H), 6.97-7.32 (s, 3 NH).

MS (ES−) 415, 416 Rt HPLC (XTerra) 4.23 min.

### EXAMPLE 32

### N-(2-Sulfamoylphenyl)sulfonyl-4-tert-butyl-benzamide

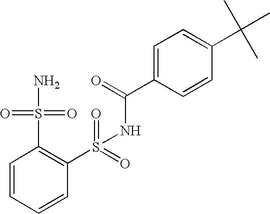

MS (ES−) 395 Rt HPLC (Xterra) 3.54 min.

### EXAMPLE 33

### 1-Methyl-N-(2-sulfamoylphenyl)sulfonyl-indole-2-carboxamide

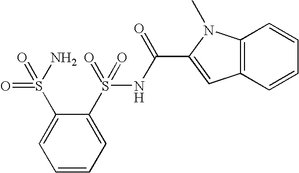

MS (ES−) 392 HPLC Rt (Xterra) 3.54 min.

### EXAMPLE 34

### 5-Pyridin-2-yl-N-(2-sulfamoylphenyl)sulfonyl-thiophene-2-carboxamide

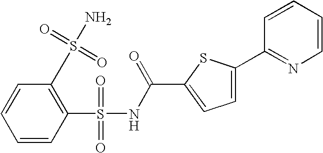

MS (ES−) 422, Rt HPLC (XTerra) 5.37 min.

### EXAMPLE 35

### 5-Phenyl-N-(2-sulfamoylphenyl)sulfonyl-thiophene-2-carboxamide

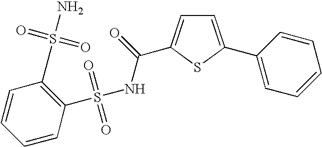

MS (ES−) 421 HPLC Rt (Xterra) 4.12 min.

### EXAMPLE 36

### 5-(3,4-Dichlorophenyl)-N-(2-sulfamoylphenyl)sulfonyl-furan-2-carboxamide

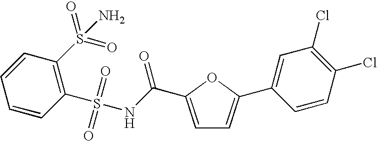

MS (ES−) 474, 475 Rt HPLC (Xterra) 4.13 min.

### EXAMPLE 37

### N-(2-Sulfamoylphenyl)sulfonyl-5-[3-(trifluoromethyl)phenyl]furan-2-carboxamide

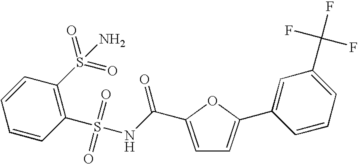

MS (ES−) 474, Rt HPLC 4.77 min.

### EXAMPLE 38

### 1-(3,5-Dichlorophenyl)-5-propyl-N-(2-sulfamoylphenyl)sulfonyl-pyrazole-4-carboxamide

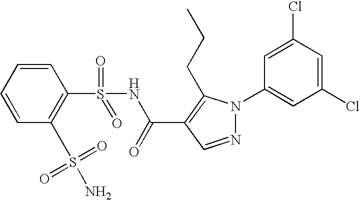

MS m/z, M+H 516.8, 518.8, M−H 515.0, 517.1; Rt HPLC (FractionLynx) 0.62 min.

### EXAMPLE 39

### 3,6-Dichloro-N-(2-sulfamoylphenyl)sulfonyl-benzothiophene-2-carboxamide

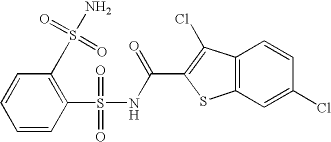

MS m/z, M+H 464.7, 466.7; Rt HPLC (FractionLynx) 0.86 min.

### EXAMPLE 40

### N-(2-Sulfamoylphenyl)sulfonylbenzothiophene-3-carboxamide

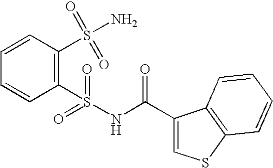

MS (ES−) 395 Rt HPLC (FractionLynx) 0.65 min.

### EXAMPLE 41

### Ethyl 4-[5-[(2-Sulfamoylphenyl)sulfonylcarbamoyl]-2-furyl]benzoate

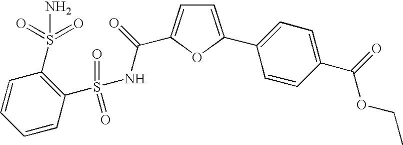

MS (ES−) 477 Rt HPLC (FractionLynx) 0.76 min.

### EXAMPLE 42

### 2-(3-Chlorophenyl)-4-methyl-N-(2-sulfamoylphenyl)sulfonyl-1,3-thiazole-5-carboxamide

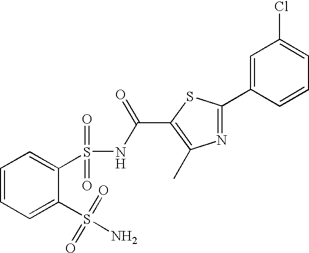

The title compound (57 mg, 42%) was synthesized by a procedure analogous to that described for Example 27.

¹H NMR (400 MHz, MeOH-d₄) δ ppm 8.32 (dd, 1H), 8.19 (dd, 1H), 7.96-7.98 (m, 1H), 7.85 (dt, 1H), 7.63-7.72 (m, 2H), 7.43-7.49 (m, 2H), 2.66 (s, 3H).

### EXAMPLE 43

### 4-(3,3-Dimethylbut-1-ynyl)-N-(2-sulfamoylphenylsulfonyl)benzamide

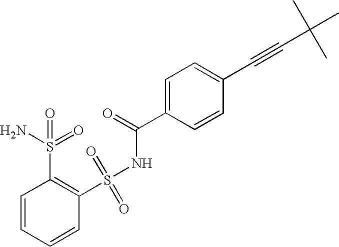

4-Bromo-N-(2-sulfamoylphenylsulfonyl)benzamide (80 mg, 0.19 mmol), (2-tert-butyl-1-ethynyl)diisopropoxyborane (100 mg, 0.48 mmol), sodium carbonate (81 mg, 0.76 mmol) and (1,1′-bis(diphenylphosphino)ferrocene)-dichloropalladium(II) (15.70 mg, 0.02 mmol) were suspended in DMF (2.5 mL) and water (0.2 mL) and the reaction mixture was stirred for 3 hours at 90° C. under an atmosphere of argon. The reaction mixture was filtered and purified by HPLC to give the product as a solid (40 mg, 49%).

¹H NMR (DMSO-d₆) δ ppm 8.27-8.38 (m, 1H), 8.10-8.18 (m, 1H), 7.80-7.94 (m, 4H), 7.37-7.47 (m, 3H), 1.29 (s, 9H).

MS m/z M−H 419, M+H 421.

### EXAMPLE 44

### 4-(3-Hydroxy-3-methylbut-1-ynyl)-N-(2-sulfamoylphenylsulfonyl)benzamide

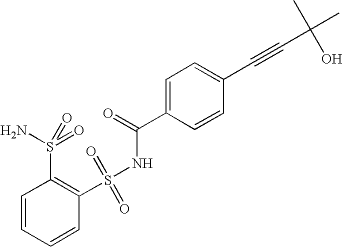

4-Bromo-N-(2-sulfamoylphenylsulfonyl)benzamide (80 mg, 0.19 mmol), 2-methyl-3-butyn-2-ol (0.018 mL, 0.19 mmol), copper(I) iodide (9.08 mg, 0.05 mmol), tetrakis(triphenylphosphine)palladium(0) (28.7 mg, 0.02 mmol) and triethylamine (0.080 mL, 0.57 mmol) were dissolved in THF (2 mL) and stirred under an atmosphere of argon at 50° C. for 3 hours and then stirred at RT for another 10 hours. The reaction mixture was filtered and purified by HPLC. The fractions containing the product were collected and the solvent was removed in vacuum. The residue was again purified by HPLC to yield the product as a solid (16 mg, 20%).

¹H NMR (MeOH) δ ppm 8.31 (dd, 1H), 8.18 (dd, 1H), 7.93 (d, 2H), 7.60-7.72 (m, 2H), 7.37 (d, 2H), 1.55 (s, 6H).

MS m/z M−H 421, M+H 423.

### EXAMPLE 45

### 4-(Benzofuran-2-yl)-3-methyl-N-(2-sulfamoylphenylsulfonyl)benzamide

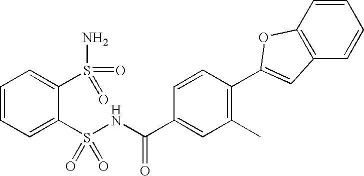

4-Bromo-3-methyl-N-(2-sulfamoylphenyl)sulfonyl-benzamide (198 mg, 0.46 mmol), benzofuran-2-ylboronic acid (111 mg, 0.69 mmol) and (1,1′-bis(diphenylphosphino)ferrocene)-dichloropalladium(II) (18.80 mg, 0.02 mmol) were dissolved in N,N-dimethylformamide (2.5 mL) (solvent was bubbled with argon). To this was added 2 M aqueous sodium carbonate (0.685 mL) and the resulting mixture was heated to 120° C. for 1 hour in a microwave. The reaction mixture was filtered through a pad of celite which was rinsed with ethyl acetate. The filtrate was concentrated in vacuo. The residue was dissolved in dimethyl sulfoxide (1.5 mL) and purified by preparative HPLC to give 89 mg (41% yield) of the title compound.

¹H NMR (400 MHz, DMSO-d₆) δ ppm 8.16 (d, 1H), 8.01 (d, 1H), 7.80-7.90 (m, 3H), 7.55-7.73 (m, 4H), 7.23-7.38 (m, 3H), 2.57 (s, 3H), 1.89 (s, 2H); MS (ESI) m/z 471 [M+H]⁺

### EXAMPLE 46

### 4-(Benzofuran-2-yl)-2-methyl-N-(2-sulfamoylphenylsulfonyl)benzamide

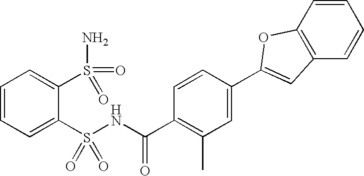

The title compound was synthesized as described for Example 45 in 6% yield, starting from 4-bromo-2-methyl-N-(2-sulfamoylphenyl)sulfonyl-benzamide.

¹H NMR (400 MHz, CD₃OD) δ ppm 8.38 (dd, 1H), 8.22 (dd, 1.39 Hz, 1H), 7.65-7.76 (m, 5H), 7.60 (d, 1H), 7.52 (d, 1H), 7.18-7.32 (m, 3H), 2.48 (s, 3H), 1.97 (s, 2H); MS (ESI) m/z 471 [M+H]⁺

### EXAMPLE 47

### 4-(Benzofuran-2-yl)-3,5-dimethoxy-N-(2-sulfamoylphenylsulfonyl)benzamide

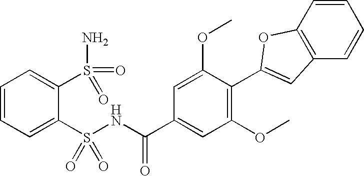

The title compound was synthesized as described for Example 45 in 39% yield, starting from 4-bromo-3,5-dimethoxy-N-(2-sulfamoylphenylsulfonyl)benzamide.

¹H NMR (400 MHz, DMSO-d₆) δ ppm 8.33 (br. s., 1H), 8.14 (dd, 1H), 7.84 (br. s., 2H), 7.66 (d, 1H), 7.57 (d, 1H), 7.46 (s, 2H), 7.33 (s, 1H), 7.20-7.34 (m, 4H), 6.96 (d, 1H), 3.81 (s, 6H); MS (ESI) m/z 517 [M+H]⁺

### a) 4-Bromo-3,5-dimethoxy-N-(2-sulfamoylphenylsulfonyl)benzamide

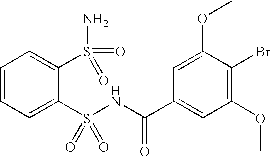

Benzene-1,2-disulfonamide (0.2 g, 0.85 mmol), 4-bromo-3,5-dimethoxybenzoic acid (0.221 g, 0.85 mmol), N-(3-dimethylaminopropyl)-N′-ethylcarbodiimide hydrochloride (0.227 g, 1.19 mmol) and 4-dimethylaminopyridine (0.259 g, 2.12 mmol) were dissolved in N,N-dimethylformamide (3 mL) and the reaction mixture was stirred at room temperature for 1.5 hour. Water was added and the solution was washed with ethyl acetate. The aqueous phase was acidified with 2 M hydrochloric acid and the product precipitated. The aqueous phase was extracted with ethyl acetate. The combined organic phases were dried over magnesium sulfate and concentrated to give 0.225 g (56% yield) of the title compound.

¹H NMR (400 MHz, DMSO-d₆) δ ppm 8.33-8.40 (m, 1H), 8.17 (dd, 1H), 7.84-8.00 (m, 3H), 7.42 (br. s., 1H), 7.24 (s, 2H), 2.89 (s, 3H), 2.73 (s, 3H); MS (ESI) m/z 479, 481 [M+H]⁺

### EXAMPLE 48

### 4-(Benzofuran-2-yl)-2-methoxy-N-(2-sulfamoylphenylsulfonyl)benzamide

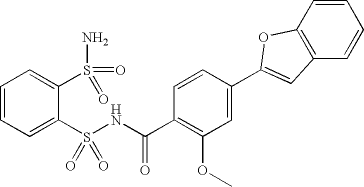

The title compound was synthesized as described for Example 45 in 4% yield, starting from 4-bromo-3,5-dimethoxy-N-(2-sulfamoylphenylsulfonyl)benzamide.

¹H NMR (400 MHz, DMSO-d₆) δ ppm 8.17 (d, 1H), 7.81 (s, 1H), 7.55-7.74 (m, 6H), 7.48 (s, 1H), 7.38 (t, 1H), 7.29 (t, 1H), 4.06 (s, 3H); MS (ESI) m/z 487.2 [M+H]+

### a) 4-Bromo-2-methoxy-N-(2-sulfamoylphenylsulfonyl)benzamide

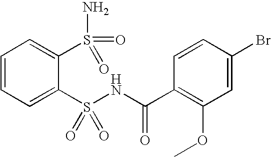

The title compound was synthesized as described for Example 47a) in 26.5% yield, starting from 4-bromo-2-methoxybenzoic acid.

MS (ESI) m/z 449, 451 [M+H]⁺

### EXAMPLE 49

### 4-(Benzofuran-2-yl)-2-hydroxy-N-(2-sulfamoylphenylsulfonyl)benzamide

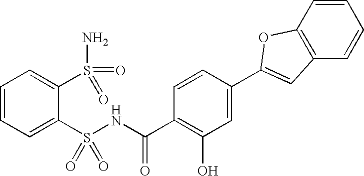

The title compound was synthesized as described for Example 45 in 73% yield, starting from 4-bromo-2-hydroxy-N-(2-sulfamoylphenylsulfonyl)benzamide.

¹H NMR (400 MHz, DMSO-d₆) δ ppm 8.24 (dd, 1.39 Hz, 1H), 8.05 (dd, 1.39 Hz, 1H), 7.83 (d, 1H), 7.61-7.77 (m, 4H), 7.50 (s, 1H), 7.24-7.37 (m, 6H); MS (ESI) m/z 473.1 [M+H]⁺

### a) 4-Bromo-2-hydroxy-N-(2-sulfamoylphenylsulfonyl)benzamide

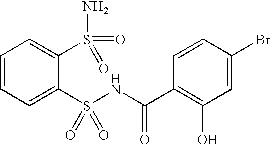

The title compound was synthesized as described for Example 47a) in 4.3% yield, starting from 4-bromo-2-hydroxybenzoic acid.

¹H NMR (400 MHz, CD₃OD) δ ppm 8.33 (dd, 1H), 8.21 (dd, 1H) 7.64-7.76 (m, 3H), 7.00 (d, 1H); MS (ESI) m/z 433.2, 435.2 [M−H]

### EXAMPLE 50

### 4-(Benzofuran-2-yl)-3-methoxy-N-(2-sulfamoylphenylsulfonyl)benzamide

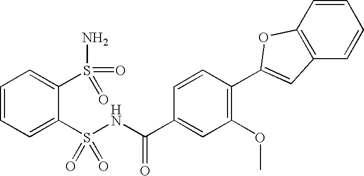

The title compound was synthesized as described for Example 45 in 34% yield, starting from 4-bromo-3-methoxy-N-(2-sulfamoylphenylsulfonyl)benzamide.

¹H NMR (400 MHz, DMSO-d₆) δ ppm 8.32-8.40 (m, 1H), 8.12-8.19 (m, 1H), 8.02 (d, 1H), 7.84-7.93 (m, 2H), 7.60-7.67 (m, 2H), 7.57 (s, 1H), 7.45 (s, 2H), 7.32-7.39 (m, 1H), 7.27 (t, 1H), 4.05 (s, 3H); MS (ESI) m/z 487.1 [M+H]+

### a) 4-Bromo-3-methoxy-N-(2-sulfamoylphenylsulfonyl)benzamide

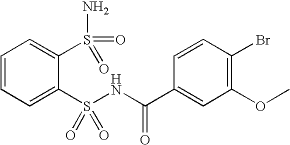

The title compound was synthesized as described for Example 47a) in 80% yield, starting from 4-bromo-3-methoxybenzoic acid.

¹H NMR (400 MHz, DMSO-d₆) δ ppm 8.36 (dd, 1.64 Hz, 1H), 8.17 (dd, 1H), 7.86-7.97 (m, 4H), 7.70 (d, 1H), 7.59 (d, 1H), 7.44 (s, 1H), 7.40 (dd, 1H), 3.90 (s, 3H); MS (ESI) m/z 449, 451 [M+H]⁺

### EXAMPLE 51

### 4-(Benzofuran-2-yl)-3-hydroxy-N-(2-sulfamoylphenylsulfonyl)benzamide

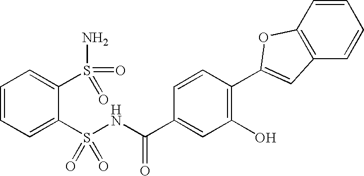

The title compound was synthesized as described for Example 45 in 9% yield, starting from 4-bromo-3-hydroxy-N-(2-sulfamoylphenylsulfonyl)benzamide.

¹H NMR (400 MHz, DMSO-d₆) δ ppm 8.17 (d, 1H), 8.03 (d, 1H), 7.84 (d, 1H), 7.67 (d, 2H), 7.60 (d, 2H), 7.46-7.50 (m, 2H), 7.44 (s, 1H), 7.30 (t, 1H), 7.24 (t, 1H); MS (ESI) m/z 473.1 [M+H]⁺

### a) 4-Bromo-3-hydroxy-N-(2-sulfamoylphenylsulfonyl)benzamide

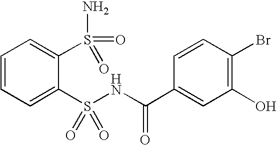

4-Bromo-3-methoxy-N-(2-sulfamoylphenylsulfonyl)benzamide (200 mg, 0.45 mmol) was dissolved in dichloromethane (3 mL) and cooled to 0° C. Boron tribromide (0.210 mL, 2.23 mmol) was added and mixture was stirred at 0° C. for 2 hours. The reaction mixture was allowed to reach room temperature and was stirred over night. The reaction mixture was washed with water and the combined aqueous phases were extracted with ethyl acetate. The combined organic phases were dried over magnesium sulfate and concentrated to give 190 mg (98% yield) of the title compound.

¹H NMR (400 MHz, DMSO-d₆) δ ppm 10.70 (br. s., 1H), 8.33 (dd, 1H), 8.16 (dd, 1H), 7.85-7.96 (m, 2H), 7.61 (d, 1H), 7.41 (s, 2H), 7.35 (d, 1H), 7.30 (dd, 1H); MS (ESI) m/z 435, 437 [M+H]⁺

### EXAMPLE 52

### 4-(Benzofuran-2-yl)-2,6-dimethyl-N-(2-sulfamoylphenylsulfonyl)benzamide

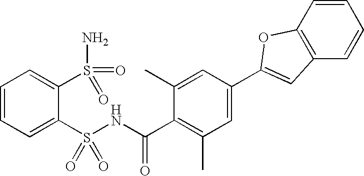

The title compound was synthesized as described for Example 45 in 14% yield, starting from 4-bromo-2,6-dimethyl-N-(2-sulfamoylphenylsulfonyl)benzamide.

¹H NMR (400 MHz, CD₃OD) δ ppm 8.46-8.52 (m, 1H), 8.28 (dd, 1H), 7.82 (dd, 2H), 7.47-7.62 (m, 4H), 7.28 (t, 1H), 7.21 (t, 1H), 7.18 (s, 1H), 2.27 (s, 5H); MS (ESI) m/z 483.4 [M−H]⁻

### a) 4-Bromo-2,6-dimethyl-N-(2-sulfamoylphenylsulfonyl)benzamide

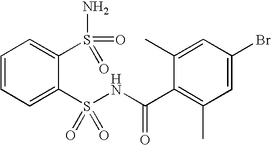

4-Bromo-2,6-dimethylbenzoic acid (0.2 g, 0.87 mmol), fluoro-N,N,N′,N′-tetramethylformamidinium hexafluorophosphate (0.254 g, 0.96 mmol) and triethylamine (0.487 mL, 3.49 mmol) were dissolved in N,N-dimethylformamide (4.5 mL). Benzene-1,2-disulfonamide (0.248 g, 1.05 mmol) was added and the reaction mixture was stirred at room temperature over night. The reaction mixture was diluted with water and washed with ethyl acetate. The aqueous phase was acidified using 2 M hydrochloric acid and extracted with ethyl acetate. The combined organic phases were dried over magnesium sulfate and concentrated to give 450 mg of the title compound, used in next step without further purification.

MS (ESI) m/z 445.2, 447.2 [M−H]⁻

### EXAMPLE 53

### 4-(3-Methoxyprop-1-ynyl)-N-(2-sulfamoylphenylsulfonyl)benzamide

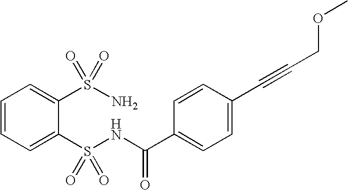

Copper(I) iodide (2.85 μL, 0.08 mmol) was added to a stirred solution of 3-methoxyprop-1-yne (0.035 mL, 0.41 mmol), 4-iodo-N-(2-sulfamoylphenylsulfonyl)benzamide (0.1723 g, 0.37 mmol), tetrakis(triphenylphosphine)palladium(0) (0.0305 g, 0.03 mmol) and triethylamine (0.50 mL, 3.59 mmol) in N,N-dimethylformamide (5 mL) under an atmosphere of nitrogen. The resulting mixture was heated at 65° C. over night. Water and ethyl acetate was added, the aqueous phase was acidified (pH ˜1) with 2 M hydrochloric acid and extracted with ethyl acetate. The organic phase was washed with water, water/brine (1:1) and brine, dried over magnesium sulfate and the solvent was evaporated. Purification by preparative HPLC gave 0.079 g (52% yield) of the title compound,

¹H NMR (400 MHz, DMSO-d₆) δ ppm 8.27-8.37 (m, 1H) 8.09-8.19 (m, 1H) 7.81-7.94 (m, 4H) 7.54 (d, 2H) 7.42 (s, 2H) 4.35 (s, 2H) 3.33 (s, 3H); MS (ESI) m/z 407.0 [M−H]⁻

### EXAMPLE 54

### 4-(3-Methylbut-3-en-1-ynyl)-N-(2-sulfamoylphenylsulfonyl)benzamide

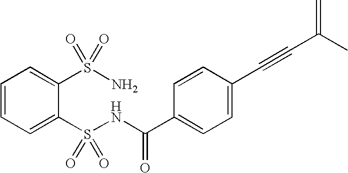

The title compound was synthesized as described for Example 53 in 60% yield, starting from 2-methylbut-1-en-3-yne.

¹H NMR (400 MHz, DMSO-d₆) δ ppm 8.28-8.36 (m, 1H) 8.13 (d, 1H) 7.80-7.92 (m, 4H) 7.52 (d, 2H) 7.42 (s, 2H) 5.29-5.53 (m, 2H) 1.96 (s, 3H); MS (ESI) m/z 403.0 [M−H]⁻

### EXAMPLE 55

### 6-(Phenylethynyl)-N-(2-sulfamoylphenylsulfonyl)nicotinamide

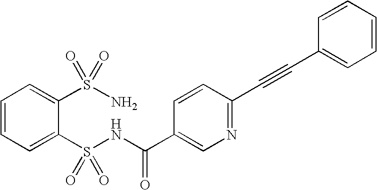

The title compound was synthesized as described for Example 53 in 99% yield, starting from 6-bromo-N-(2-sulfamoylphenylsulfonyl)nicotinamide and phenylacetylene. Purification by column chromatography, using 0-10% methanol in dichloromethane as the eluent. The residue was washed with dichloromethane.

¹H NMR (400 MHz, DMSO-d₆) δ ppm 9.01 (d, 1H) 8.29-8.39 (m, 1H) 8.24 (dd, 1H) 8.12 (dd, 1H) 7.84 (d, 2H) 7.73 (d, 1H) 7.57-7.70 (m, 2H) 7.38-7.55 (m, 5H); MS (ESI) m/z 403.0 [M−H]

### EXAMPLE 56

### 4-(3-Ethyl-3-hydroxypent-1-ynyl)-N-(2-sulfamoylphenylsulfonyl)benzamide

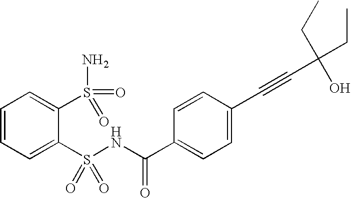

Bis(triphenylphosphine)palladium(II) chloride (50.2 mg, 0.07 mmol) and copper(I) iodide (13.63 mg, 0.07 mmol) were added to a solution of 4-bromo-N-(2-sulfamoylphenyl)sulfonyl-benzamide (300 mg, 0.72 mmol), 3-ethylpent-1-yn-3-ol (0.184 mL, 1.43 mmol) and diisopropylamine (0.306 mL, 2.15 mmol) in degassed N,N-dimethylformamide (1.5 mL). The reaction mixture was heated at 100° C. in a microwave for 1 hour. The reaction mixture was filtered through a pad of celite which was rinsed with ethyl acetate. The filtrate was concentrated in vacuo. The residue was dissolved in dimethyl sulfoxide (1.5 mL) and purified by preparative HPLC to give 88 mg (27% yield) of the title compound.

¹H NMR (400 MHz, DMSO-d₆) δ ppm 8.13 (dd, 1H), 7.99 (dd, 1H), 7.85 (d, 2H), 7.54-7.67 (m, 2H), 7.34 (d, 2H), 1.54-1.70 (m, 4H), 0.99 (t, 6H); MS (ESI) m/z 451.2 [M+H]⁺

### EXAMPLE 57

### 4-(3-Hydroxy-3-methylpent-1-ynyl)-N-(2-sulfamoylphenylsulfonyl)benzamide

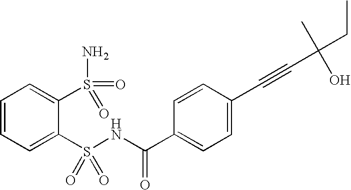

The title compound was synthesized as described for Example 56 in 10% yield, starting from 3-methylpent-1-yn-3-ol.

¹H NMR (400 MHz, DMSO-d₆) δ ppm 8.32-8.37 (m, 1H), 8.15 (dd, 1H), 7.86 (d, 2H), 7.84-7.94 (m, 2H), 7.48 (d, 2H), 7.42 (br. s., 2H), 1.56-1.73 (m, 2H), 1.41 (s, 3H), 0.99 (t, 3H); MS (ESI) m/z 435.1 [M−H]

### EXAMPLE 58

### 4-((1-Hydroxycyclopentyl)ethynyl)-N-(2-sulfamoylphenylsulfonyl)benzamide

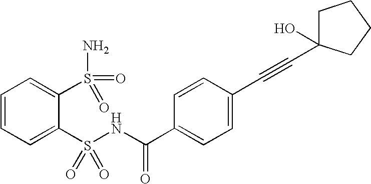

The title compound was synthesized as described for Example 45 in 34% yield, starting from 1-ethynylcyclopentanol.

¹H NMR (400 MHz, DMSO-d₆) δ ppm 8.13 (dd, 1H), 7.99 (dd, 1H), 7.84 (d, 2H), 7.55-7.66 (m, 2H), 7.44 (br. s., 2H), 7.33 (d, 2H), 5.36 (br. s., 1H), 1.82-1.95 (m, 4H), 1.61-1.79 (m, 4H); MS (ESI) m/z 449.1 [M+H]⁺

### EXAMPLE 59

### 3-(3-Hydroxy-3-methylbut-1-ynyl)-N-(2-sulfamoylphenylsulfonyl)benzamide

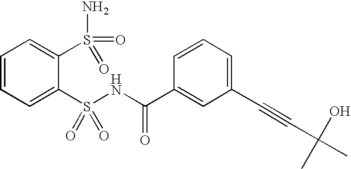

The title compound was synthesized as described for Example 56 in 14% yield, starting from 3-bromo-N-(2-sulfamoylphenylsulfonyl)benzamide.

¹H NMR (400 MHz, DMSO-d₆) δ ppm 8.14 (dd, 1H), 7.99 (dd, 1H), 7.93 (s, 1H), 7.80 (d, 1H), 7.55-7.67 (m, 2H), 7.36-7.41 (m, 1H), 7.31 (t, 1H), 2.19 (br. s., 1H), 1.46 (s, 6H); MS (ESI) m/z 421.3 [M−H]⁻

### a) 3-Bromo-N-(2-sulfamoylphenylsulfonyl)benzamide

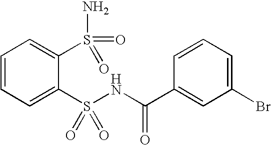

The title compound was synthesized as described for Example 47a) in 86% yield, starting from 3-bromobenzoic acid.

¹H NMR (400 MHz, DMSO-d₆) δ ppm 8.36 (dd, 1H), 8.17 (dd, 1H), 8.10 (s, 1H), 7.77-7.98 (m, 5H), 7.39-7.48 (m, 3H); MS (ESI) m/z 417, 419 [M−H]⁻

### EXAMPLE 60

### 3-(3,3-Dimethylbut-1-ynyl)-N-(2-sulfamoylphenylsulfonyl)benzamide

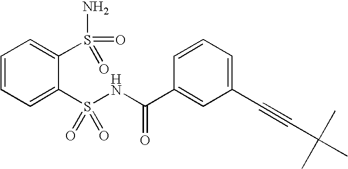

3-Bromo-N-(2-sulfamoylphenylsulfonyl)benzamide (200 mg, 0.48 mmol), (2-tert-butyl-1-ethynyl)diisopropoxyborane (0.135 mL, 0.57 mmol) and (1,1′-bis(diphenylphosphino)ferrocene)-dichloropalladium(II) (19.62 mg, 0.02 mmol) were dissolved in N,N-dimethylformamide (2.0 mL) (the solvent was bubbled with argon). Aqueous 2 M sodium carbonate (0.685 mL) was added and the resulting mixture was heated at 120° C. for 40 min in a microwave. The reaction mixture was filtered through a pad of celite which was rinsed with ethyl acetate. The filtrate was concentrated in vacuo. The residue was dissolved in dimethyl sulfoxide (1.5 mL) and purified by preparative HPLC to give 9 mg (4% yield) of the title compound.

¹H NMR (400 MHz, CD₃OD) δ ppm 8.38-8.44 (m, 1H), 8.22-8.27 (m, 1H), 7.89 (s, 1H), 7.75-7.85 (m, 3H), 7.49 (d, 1H), 7.36 (t, 1H), 1.32 (s, 9H); MS (ESI) m/z 421.1 [M+H]⁺

### EXAMPLE 61

### 4-(3,3-Dimethylbut-1-ynyl)-N-(2-sulfamoylphenylsulfonyl)-1-naphthamide

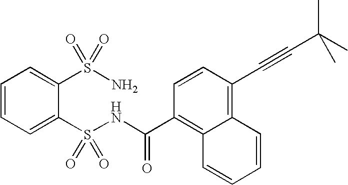

Diisopropyl 3,3-dimethylbut-1-ynylboronate (0.100 mL, 0.43 mmol), 4-bromo-N-(2-sulfamoylphenylsulfonyl)-1-naphthamide (200 mg, 0.43 mmol), [1,1′-bis(diphenylphosphino)ferrocene]dichloropalladium (35 mg, 0.04 mmol) and potassium carbonate (353 mg, 2.56 mmol) were dissolved in tetrahydrofurane (5 mL) and water (1 mL) in a microwave vial. The reaction was irradiated for 60 minutes at 150° C. in a microwave oven, filtered through a plug of celite and concentrated in vacuo. Purification by preparative HPLC gave 19 mg (9% yield) of the title compound.

¹H NMR (CD₃OD) δ ppm 8.45-8.40 (m, 2H) 8.29-8.22 (m, 2H) 7.80 (d, 1H) 7.74 (dd, 1H) 7.72-7.68 (m, 1H) 7.55-7.47 (m, 3H) 1.42 (s, 9H); MS (ESI) m/z 469 [M−1]⁻

### a) 4-Bromo-N-(2-sulfamoylphenylsulfonyl)-1-naphthamide

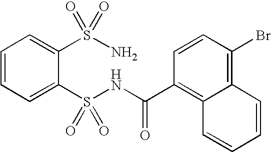

Benzene-1,2-disulfonamide (750 mg, 3.17 mmol), 4-bromo-1-naphthoic acid (797 mg, 3.17 mmol), N1-((ethylimino)methylene)-N3,N3-dimethylpropane-1,3-diamine hydrochloride (852 mg, 4.44 mmol) and 4-dimethylaminopyridine (970 mg, 7.94 mmol) were dissolved in anhydrous N,N-dimethylformamide (15 mL) and the reaction was stirred at room temperature over night. Water (100 mL) was added and the solution was extracted with ethyl acetate. The aqueous phase was acidified with hydrochloric acid (2 M) and extracted with ethyl acetate. The combined organic phases were washed with water, dried over magnesium sulfate and concentrated in vacuo, to give 1.515 g (80% yield) of the title compound.

MS (ESI) m/z 469, 467 [M−1]⁻

### EXAMPLE 62

### 4-(Benzofuran-2-yl)-N-(2-sulfamoylphenylsulfonyl)-1-naphthamide

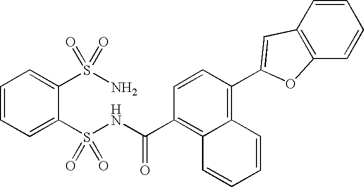

The title compound was synthesized as described for Example 61 in 31% yield, starting from benzofuran-2-ylboronic acid.

¹H NMR (DMSO-d₆) δ ppm 13.19 (br. s., 2H) 8.51 (d, 1H) 8.48-8.44 (m, 1H) 8.25-8.22 (m, 1H) 8.18 (br. s., 1H) 8.01-7.93 (m, 4H) 7.78 (d, 1H) 7.74 (d, 1H) 7.72-7.64 (m, 2H) 7.51 (s, 1H) 7.46 (s, 2H) 7.44-7.39 (m, 1H) 7.35 (t, 1H); MS (ESI) m/z 505 [M−1]⁻

### EXAMPLE 63

### 2-(Benzofuran-2-yl)-4-methyl-N-(2-sulfamoylphenylsulfonyl)thiazole-5-carboxamide

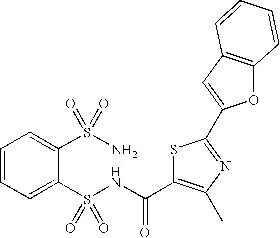

The title compound was synthesized as described for Example 61 in 6% yield, starting from 2-bromo-4-methyl-N-(2-sulfamoylphenylsulfonyl)thiazole-5-carboxamide and benzofuran-2-ylboronic acid.

¹H NMR (CD₃OD) δ 8.14-8.11 (m, 1H) 8.0-7.9 (m, 1H) 7.52-7.42 (m, 3H) 7.37 (d, 1H) 7.27 (s, 1H) 7.22-7.17 (m, 1H) 7.09 (t, 1H) 2.47 (s, 3H); MS (ESI) m/z 476 [M−1]⁻

### a) 2-Bromo-4-methyl-N-(2-sulfamoylphenylsulfonyl)thiazole-5-carboxamide

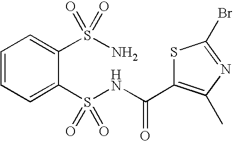

The title compound was synthesized as described for Example 61 a) in 90% yield, starting from 2-bromo-4-methylthiazole-5-carboxylic acid.

MS (ESI) m/z 440, 438 [M−1]⁻

### EXAMPLE 64

### 3′-(3-Hydroxy-3-methylbut-1-ynyl)-N-(2-sulfamoylphenylsulfonyl)biphenyl-2-carboxamide

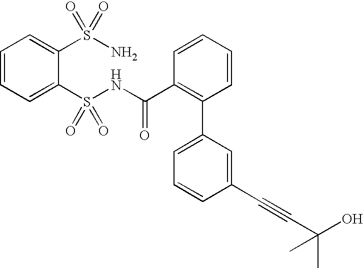

2-Bromo-N-(2-sulfamoylphenylsulfonyl)benzamide (370 mg, 0.88 mmol), [1,1′-bis(diphenylphosphino)ferrocene]dichloropalladium (71 mg, 0.09 mmol) and potassium carbonate (732 mg, 5.29 mmol) and 2-methyl-4-(3-(4,4,5,5-tetramethyl-1,3,2-dioxaborolan-2-yl)phenyl)but-3-yn-2-ol (328 mg, 1.15 mmol) were dissolved in tetrahydrofurane (4 mL) and water (1 mL) in a microwave vial. The reaction was heated for 120 min at 150° C. in a microwave, filtered through a plug of celite and concentrated in vacuo. Purification by preparative HPLC gave 7 mg (2% yield) of the title compound:

¹H NMR (CD₃OD) δ ppm 8.23-8.18 (m, 2H) 7.79-7.72 (m, 1H) 7.68-7.63 (m, 1H) 7.53-7.43 (m, 2H) 7.38-7.32 (m, 1H) 7.27 (d, 1H) 7.21 (t, 1H) 7.16-7.12 (m, 1H) 7.10-7.040 (m, 1H) 6.94 (t, 1H) 1.47 (s, 6H); MS (ESI) m/z 497 [M−1]⁻

### a) 2-Methyl-4-(3-(4,4,5,5-tetramethyl-1,3,2-dioxaborolan-2-yl)phenyl) but-3-yn-2-ol

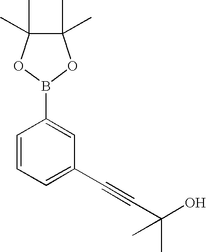

Bis(dibenzylideneacetone)palladium (186 mg, 0.32 mmol) and tricyclohexylphosphine (212 mg, 0.76 mmol) were dissolved in anhydrous dioxane (10 mL) and stirred for 30 min. A solution of bis(pinacolato)diboron (2.877 g, 11.33 mmol), potassium acetate (1.588 g, 16.19 mmol) and 4-(3-bromophenyl)-2-methylbut-3-yn-2-ol (2.580 g, 10.79 mmol) in anhydrous dioxane (10 mL), was added and the reaction was heated at 130° C. for 60 min in a microwave. Purification by column chromatography, using 0 to 100% ethyl acetate in heptane as the eluent, gave 2.72 g (88% yield) of the title compound:

¹H NMR (CD₃OD) δ ppm 7.76 (s, 1H) 7.71-7.66 (m, 1H) 7.52-7.47 (m, 1H) 7.34 (t, 1H) 1.58 (s, 6H) 1.37 (s, 12H)

### EXAMPLE 65

### 4-(Cyclopentylethynyl)-N-(2-sulfamoylphenylsulfonyl)benzamide

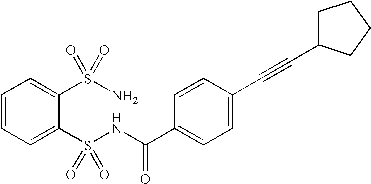

4-Bromo-N-(2-sulfamoylphenyl)sulfonyl-benzamide (200 mg, 0.48 mmol), copper(I) iodide (5 g, 0.02 mmol), bis(triphenylphosphine)palladium(II) chloride (17 mg, 0.02 mmol), ethynylcyclopentane (0.055 mL, 0.48 mmol) and diisopropylamine (0.202 mL, 1.43 mmol) were slurried in anhydrous N,N-dimethylformamide (3 mL) in a microwave vial. The reaction was heated for 90 min at 100° C. in a microwave, filtered through a plug of celite and concentrated in vacuo. Purification by preparative HPLC gave 34 mg (16% yield) of the title compound:

¹H NMR (CD₃OD) δ ppm 8.29 (d, 1H) 8.18 (d, 1H) 7.90 (d, 2H) 7.71-7.56 (m, 2H) 7.32 (d, 2H) 2.91-2.79 (m, 1H) 2.06-1.93 (m, 2H) 1.83-1.73 (m, 2H) 1.73-1.57 (m, 4H); MS (ESI) m/z 431 [M−1]⁻

### EXAMPLE 66

### 3-(Cyclopentylethynyl)-N-(2-sulfamoylphenylsulfonyl)benzamide

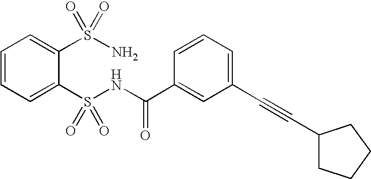

The title compound was synthesized as described for Example 65 in 6% yield, starting from 3-bromo-N-(2-sulfamoylphenylsulfonyl)benzamide.

¹H NMR (CD₃OD) δ ppm 8.28 (dd, 1H) 8.18 (dd, 1H) 7.97 (s, 1H) 7.90 (d, 1H) 7.71-7.59 (m, 2H) 7.41-7.37 (m, 1H) 7.28 (t, 1H) 2.89-2.80 (m, 1H) 2.05-1.96 (m, 4H) 1.83-1.59 (m, 4H)); MS (ESI) m/z 431 [M−1]⁻

### EXAMPLE 67

### 4-(Cyclopentylethynyl)-2-methyl-N-(2-sulfamoylphenylsulfonyl)benzamide

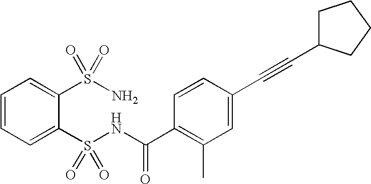

The title compound was synthesized as described for Example 65 in 10% yield, starting from 4-bromo-2-methyl-N-(2-sulfamoylphenylsulfonyl)benzamide.

¹H NMR (CD₃OD) δ ppm 8.33 (d, 1H) 8.20 (d, 1H) 7.73-7.51 (m, 3H) 7.12-7.08 (m, 2H) 2.88-2.77 (m, 1H) 2.34 (s, 3H) 2.03-1.94 (m, 2H) 1.81-1.721 (m, 2H) 1.72-1.58 (m, 4H); MS (ESI) m/z 445 [M−1]⁻

### EXAMPLE 68

### 4-(3,3-Dimethylbut-1-ynyl)-3-methoxy-2-methyl-N-(2-sulfamoylphenylsulfonyl)benzamide

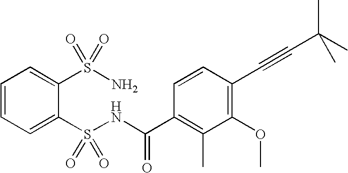

The title compound was synthesized as described for Example 61 in 14% yield, starting from diisopropyl 3,3-dimethylbut-1-ynylboronate and 4-bromo-3-methoxy-2-methyl-N-(2-sulfamoylphenylsulfonyl)benzamide.

¹H NMR (CD₃OD) δ ppm 8.39-8.31 (m, 1H) 8.24-8.15 (m, 1H) 7.77-7.64 (m, 4H) 7.24 (d, 1H) 7.11 (d, 1H) 3.83 (s, 3H) 2.25 (s, 3H) 1.33 (s, 9H); MS (ESI) m/z 465 [M+1]⁺

### a) 4-Bromo-3-methoxy-2-methyl-N-(2-sulfamoylphenylsulfonyl)benzamide

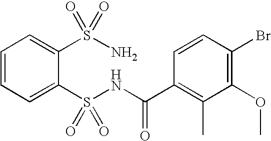

The title compound was synthesized as described for Example 61a) in 87% yield, starting from 4-bromo-3-methoxy-2-methylbenzoic acid.

MS (ESI) m/z 463, 461 [M−1]⁻

### b) 4-Bromo-3-methoxy-2-methylbenzoic acid

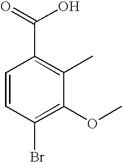

Methyl 4-bromo-3-methoxy-2-methylbenzoate (1.3 g, 5.02 mmol) was dissolved in 15% sodium hydroxide (20 mL) and heated at 100° C. for 1 hour. The mixture was allowed to cool to room temperature, acidified using hydrochloric acid (4 M) and was extracted with dichloromethane. The combined organic phases were dried over magnesium sulfate and concentrated in vacuo to give 1.15 g (94% yield) of the title compound:

MS (ESI) m/z 245, 243 [M−1]⁻

### c) Methyl 4-bromo-3-methoxy-2-methylbenzoate

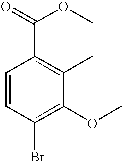

Methyl 4-bromo-3-hydroxy-2-methylbenzoate (1.51 g, 6.16 mmol), iodomethane (1.161 mL, 18.48 mmol) and potassium carbonate (2.55 g, 18.48 mmol) were dissolved in N,N-dimethylformamide (10 mL) and acetone (10 mL) and stirred at room temperature over night. Water was added and the aqueous phase was extracted with ethyl acetate and dichloromethane. The combined organic phases were washed with water, dried over magnesium sulfate and concentrated in vacuo to gave 1.3 g (81% yield) of the title compound.

¹H NMR (CDCl₃) δ ppm 7.56-7.49 (m, 1H) 7.47-7.38 (m, 1H) 3.90 (s, 3H) 3.81 (s, 3H) 2.57 (s, 3H)

### d) Methyl 4-bromo-3-hydroxy-2-methylbenzoate

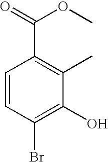

A solution of bromine (1.608 mL, 31.29 mmol) in dichloromethane (20 mL) was added was added dropwise over 30 min to a solution of 2-methylpropan-2-amine (3.30 mL, 31.29 mmol) in dichloromethane (100 mL) at −78° C. The solution was stirred for 30 min at −78° C. A solution of methyl 3-hydroxy-2-methylbenzoate (5.2 g, 31.29 mmol) in dichloromethane (30 mL) was added over 30 min. The reaction was allowed to reach room temperature, stirred over night and water was added. The aqueous phase was extracted with dichloromethane and the combined organic phases were washed with water, dried over magnesium sulphate and concentrated in vacuo. Purification by column chromatography, using a gradient of 0 to 10% ethyl acetate in heptane as the eluent, gave 1.51 g (20% yield) of the title compound:

MS (ESI) m/z 245, 243 [M−1]⁻

### EXAMPLE 69

### 4-(Benzofuran-2-yl)-3-methoxy-2-methyl-N-(2-sulfamoylphenylsulfonyl)benzamide

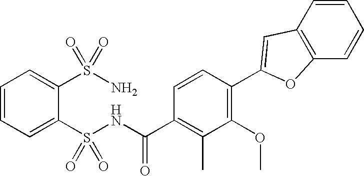

The title compound was synthesized as described for Example 61 in 48% yield, starting from benzofuran-2-ylboronic acid and 4-bromo-3-methoxy-2-methyl-N-(2-sulfamoylphenylsulfonyl)benzamide.

¹H NMR (DMSO-d₆) δ ppm 8.19 (dd, 1H) 8.02 (dd, 1H) 7.73-7.57 (m, 5H) 7.50 (d, 1H) 7.42 (d, 1H) 7.36-7.29 (m, 1H) 7.30-7.22 (m, 1H) 3.69 (s, 3H) 2.33 (s, 3H); MS (ESI) m/z 499 [M−1]⁻

### EXAMPLE 70

### 4-(Pyridin-3-ylethynyl)-N-(2-sulfamoylphenylsulfonyl)benzamide

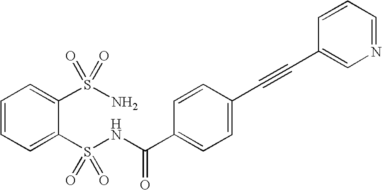

Copper(I) iodide (3.56 μL, 0.11 mmol) was added to a stirred solution of 4-iodo-N-(2-sulfamoylphenylsulfonyl)benzamide (0.2129 g, 0.46 mmol), 3-ethynylpyridine (0.0545 g, 0.53 mmol), tetrakis(triphenylphosphine)palladium(0) (0.0346 g, 0.03 mmol) and triethylamine (1 mL, 7.17 mmol) in N,N-dimethylformamide (5 mL) under an atmosphere of nitrogen. The resulting mixture was heated at 65° C. over night. Water was added and the mixture was acidified (pH-1) using 2 M hydrochloric acid. The formed solid was removed by filtration, stirred with warm methanol, filtered and dried. Dissolved in boiling acetonitrile, allowed to cool down to room temperature, filtered, washed with acetonitrile and dried in vacuo to give 0.066 g (33% yield) of the title compound.

¹H NMR (400 MHz, DMSO-d₆) δ ppm 8.82 (s, 1H) 8.64 (d, 1H) 8.37 (dd, 1H) 8.17 (dd, 1H) 8.04-8.10 (m, 1H) 7.86-7.98 (m, 4H) 7.70 (d, 2H) 7.53 (dd, 1H) 7.43 (br. s., 2H); MS (ESI) m/z 442.0 [M+H]⁺, MS (ESI) m/z 440.2 [M−H]⁻

### EXAMPLE 71

### 4-(Pyridin-2-ylethynyl)-N-(2-sulfamoylphenylsulfonyl)benzamide

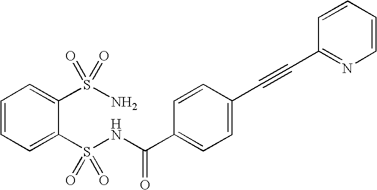

Synthesized as described for Example 70 in 31% yield, starting from 2-ethynylpyridine. The aqueous phase was acidified using hydrochloric acid 2M, extracted with ethyl acetate and the combined organic phases were dried over magnesium sulfate and concentrated. The residue was washed with dichloromethane/methanol (9:1), filtered and dried in vacuo.

¹H NMR (400 MHz, DMSO-d₆) δ ppm 8.63 (d, 1H) 8.35 (dd, 1H) 7.94 (d, 2H) 7.84-7.91 (m, 3H) 7.70 (t, 3H) 7.39-7.48 (m, 3H); MS (ESI) m/z 442.0 [M+H]⁺, MS (ESI) m/z 440.2 [M−H]⁻

### EXAMPLE 72

### 4-(Phenylethynyl)-N-(2-sulfamoylphenylsulfonyl)benzamide

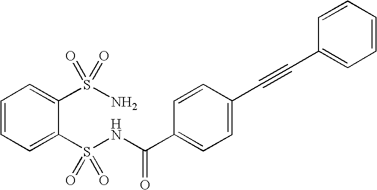

Copper(I) iodide (2.349 μL, 0.07 mmol) was added to a stirred solution of 4-iodo-N-(2-sulfamoylphenylsulfonyl)benzamide (0.200 g, 0.43 mmol), phenylacetylene (0.060 mL, 0.55 mmol), tetrakis(triphenylphosphine)palladium(0) (0.0283 g, 0.02 mmol) and triethylamine (1.5 mL, 10.76 mmol) in N,N-dimethylformamide (5 mL) under an atmosphere of nitrogen. The resulting mixture was heated at 65° C. for 3.5 h. Ethyl acetate and water was added. The aqueous phase was extracted with ethyl acetate and the combined organic phases were washed with water and brine, dried over magnesium sulfate and concentrated. Dichloromethane was added and the precipitated product was filtered off to give 0.035 g. The residue was purified by column chromatography, using a gradient of 0-10% methanol in dichloromethane as the eluent, to give 0.024g. The two fractions were combined to give 0.059 g (31% yield) of the title compound.

¹H NMR (400 MHz, DMSO-d₆) δ ppm 8.20 (d, 1H) 8.03 (d, 1H) 7.92 (d, 2H) 7.63-7.73 (m, 2H) 7.52-7.60 (m, 4H) 7.41-7.47 (m, 5H); MS (ESI) m/z 439.2 [M−H]⁻

### EXAMPLE 73

### 4-(3,3-Dimethylbut-1-ynyl)-3-fluoro-N-(2-sulfamoylphenylsulfonyl)benzamide

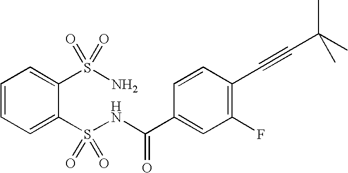

A mixture of 4-bromo-3-fluoro-N-(2-sulfamoylphenylsulfonyl)benzamide (0.10 g, 0.23 mmol), diisopropyl 3,3-dimethylbut-1-ynylboronate (0.11 mL, 0.46 mmol), 1,1′-bis(diphenylphosphino)ferrocene-palladium dichloride (0.019 g, 0.020 mmol), N,N-dimethylformamide (2 mL) and 2 M sodium carbonate (0.34 mL, 0.69 mmol) under an atmosphere of argon was heated at 120° C. for 1 hour in a microwave. The reaction mixture was partitioned between ethyl acetate and water, the organic phase was dried over magnesium sulfate and evaporated. Purification by preparative HPLC, gave 0.023 g (23% yield) of the title compound.

¹H NMR (DMSO-d₆) δ ppm 8.19-8.27 (m, 1H) 8.00-8.07 (m, 1H) 7.72-7.82 (m, 2H) 7.63 (dd, 1H) 7.57 (dd, 1H) 7.41 (t, 1H) 7.33 (br. s., 2H) 1.20 (s, 9H); MS (ESI) m/z 437 [M−1]⁻.

### a) 4-Bromo-3-fluoro-N-(2-sulfamoylphenylsulfonyl)benzamide

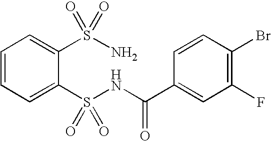

To a solution of benzene-1,2-disulfonamide (0.47 g, 2.00 mmol) and 4-bromo-3-fluorobenzoic acid (0.44 g, 2.00 mmol) in N,N-dimethylformamide (20 mL) was N-(3-dimethylaminopropyl)-N′-ethylcarbodiimide hydrochloride (0.58 g, 3.00 mmol) and 4-(dimethylamino)pyridine (0.37 g, 3.00 mmol) added, the resulting mixture was stirred at room temperature over night. Water was added and the mixture was washed with ethyl acetate. The aqueous phase was acidified by addition of 1 M hydrochloric acid and extracted with ethyl acetate. The organic phase was dried over magnesium sulfate and evaporated to give 0.77 g (88% yield) of the title compound.

¹H NMR (DMSO-d₆) δ ppm 8.30-8.37 (m, 1H) 8.14 (d, 1H) 7.78-7.94 (m, 4H) 7.66 (dd, 1H) 7.45 (br. s., 2H); MS (ESI) m/z 435, 437 [M−1]⁻.

### EXAMPLE 74

### 2-(3-Methoxyphenyl)-N-(2-sulfamoylphenylsulfonyl)benzofuran-5-carboxamide

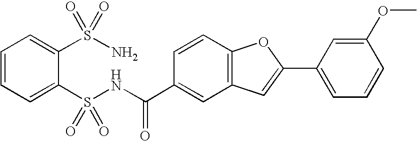

The title compound was synthesized as described for Example 73a) in 11% yield, starting from 2-(3-methoxyphenyl)benzofuran-5-carboxylic acid. Purification by preparative HPLC.

¹H NMR (DMSO-d₆) δ ppm 8.34-8.44 (m, 1H) 8.29 (d, 1H) 8.13-8.21 (m, 1H) 7.81-7.97 (m, 3H) 7.72 (d, 1H) 7.63 (s, 1H) 7.52-7.58 (m, 1H) 7.48-7.51 (m, 1H) 7.39-7.48 (m, 3H) 7.02 (dd, 1H) 3.86 (s, 3H); MS (ESI) m/z 485 [M−1]⁻.

### a) 2-(3-Methoxyphenyl)benzofuran-5-carboxylic acid

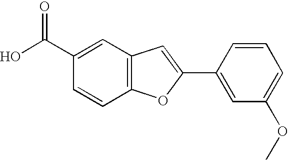

A solution of lithium hydroxide (0.066 g, 2.74 mmol) in water (1 mL) was added to a solution of methyl 2-(3-methoxyphenyl)benzofuran-5-carboxylate (0.13 g, 0.46 mmol) in tetrahydrofuran (3 mL). The resulting mixture was stirred at room temperature over night, water was added, the mixture was acidified by the addition of 1 M hydrochloric acid and extracted with ethyl acetate. The organic phase was dried over magnesium sulfate and evaporated to give 0.12 g (95% yield) of the title compound.

¹H NMR (DMSO-d₆) δ ppm 8.28 (d, 1H) 7.94 (dd, 1H) 7.74 (d, 1H) 7.60 (d, 1H) 7.51-7.56 (m, 1H) 7.42-7.50 (m, 2H) 7.00-7.06 (m, 1H) 3.87 (s, 3H); MS (ESI) m/z 267 [M−1]⁻.

### b) Methyl 2-(3-methoxyphenyl)benzofuran-5-carboxylate

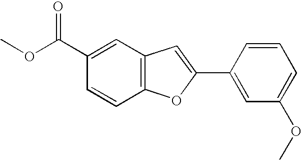

A mixture of methyl 4-hydroxy-3-iodobenzoate (0.14 g, 0.50 mmol), 3-ethynylanisole (0.19 mL, 1.50 mmol), bis(triphenylphosphine)palladium(II) chloride (0.035 g, 0.050 mmol), copper(I) iodide (9.5 mg, 0.050 mmol) and 1,1,3,3-tetramethylguanidine (0.63 mL, 5.00 mmol) in N,N-dimethylformamide (5 mL) under an atmosphere of argon was heated at 70° C. for 3 days. The reaction mixture was diluted with ethyl acetate and washed with water. The organic phase was dried over magnesium sulfate and the solvent was evaporated. Purification by column chromatography, using heptane:ethyl acetate (9:1) as the eluent, gave 0.13 g (91% yield) of the title compound.

¹H NMR (CDCl₃) δ ppm 8.35 (dd, 1H) 8.04 (dd, 1H) 7.57 (d, 1H) 7.49 (ddd, 1H) 7.37-7.45 (m, 2H) 7.10 (d, 1H) 6.96 (ddd, 1H) 3.98 (s, 3H) 3.93 (s, 3H); MS (EI) m/z 282 [M]⁺.

### EXAMPLE 75

### 2-(4-Methoxyphenyl)-N-(2-sulfamoylphenylsulfonyl)benzofuran-5-carboxamide

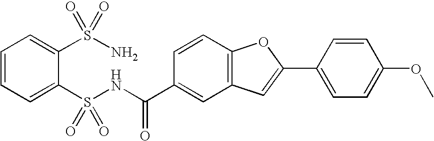

The title compound was synthesized as described for Example 74 in 27% yield, starting from 2-(4-methoxyphenyl)benzofuran-5-carboxylic acid.

¹H NMR (DMSO-d₆) δ ppm 8.36-8.41 (m, 1H) 8.25 (d, 1H) 8.15-8.20 (m, 1H) 7.87-7.97 (m, 4H) 7.81 (dd, 1H) 7.69 (d, 1H) 7.42 (d, 3H) 7.07-7.13 (m, 2H) 3.84 (s, 3H);

MS (ESI) m/z 485 [M−1]⁻.

### a) 2-(4-Methoxyphenyl)benzofuran-5-carboxylic acid

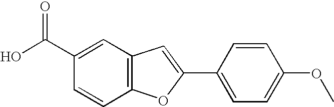

The title compound was synthesized as described for Example 74a) in 94% yield, starting from methyl 2-(4-methoxyphenyl)benzofuran-5-carboxylate.

¹H NMR (DMSO-d₆) δ ppm 12.81 (br. s., 1H) 8.18 (d, 1H) 7.80-7.88 (m, 3H) 7.64 (d, 1H) 7.34 (d, 1H) 7.02-7.09 (m, 2H) 3.78 (s, 3H); MS (ESI) m/z 267 [M−1]⁻.

### b) Methyl 2-(4-methoxyphenyl)benzofuran-5-carboxylate

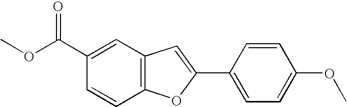

The title compound was synthesized as described for Example 74b) in 98% yield, starting from 1-ethynyl-4-methoxybenzene.

¹H NMR (CDCl₃) δ ppm 8.31 (d, 1H) 8.01 (dd, 1H) 7.79-7.87 (m, 2H) 7.54 (d, 1H) 6.99-7.06 (m, 2H) 6.96 (d, 1H) 3.97 (s, 3H) 3.90 (s, 3H); MS (EI) m/z 282 [M]⁺.

### EXAMPLE 76

### 2-tert-Butyl-N-(2-sulfamoylphenylsulfonyl)benzofuran-5-carboxamide

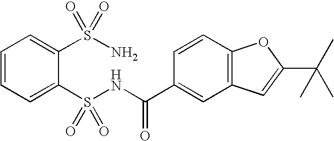

The title compound was synthesized as described for Example 74 in 46% yield, starting from 2-tert-butylbenzofuran-5-carboxylic acid.

¹H NMR (DMSO-d₆) δ ppm 8.36 (d, 1H) 8.12-8.21 (m, 2H) 7.86-7.97 (m, 2H) 7.77 (dd, 1H) 7.60 (d, 1H) 7.43 (s, 2H) 6.69 (s, 1H) 1.35 (s, 9H); MS (ESI) m/z 435 [M−1]⁻.

### a) 2-tert-Butylbenzofuran-5-carboxylic Acid

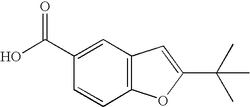

The title compound was synthesized as described for Example 74a) in 94% yield, starting from methyl 2-tert-butylbenzofuran-5-carboxylate.

¹H NMR (DMSO-d₆) δ ppm 12.77 (br. s., 1H) 8.08-8.15 (m, 1H) 7.78 (dd, 1H) 7.54 (d, 1H) 6.63 (d, 1H) 1.30 (s, 9H); MS (ESI) m/z 217 [M−1]⁻.

### b) Methyl 2-tert-butylbenzofuran-5-carboxylate

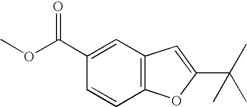

The title compound was synthesized as described for Example 76b) in 95% yield, starting from 3,3-Dimethyl-1-butyne as described.

¹H NMR (CDCl₃) δ ppm 8.09 (d, 1H) 7.81 (dd, 1H) 7.30 (d, 1H) 6.28 (d, 1H) 3.80 (s, 3H) 1.26 (s, 9H); MS (EI) m/z 232 [M]⁺.

### EXAMPLE 77

### 2-(1-Hydroxycyclopentyl)-N-(2-sulfamoylphenylsulfonyl)benzofuran-5-carboxamide

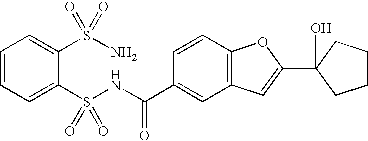

The title compound was synthesized as described for Example 74 in 29% yield, starting from 2-(1-hydroxycyclopentyl)benzofuran-5-carboxylic acid.

¹H NMR (DMSO-d₆) δ ppm 8.30-8.40 (m, 1H) 8.12-8.23 (m, 2H) 7.84-7.96 (m, 2H) 7.75-7.81 (m, 1H) 7.60 (d, 1H) 7.42 (s, 2H) 6.82 (s, 1H) 5.40 (br. s., 1H) 1.94-2.05 (m, 2H) 1.80-1.94 (m, 4H) 1.65-1.78 (m, 2H); MS (ESI) m/z 463 [M−1]⁻.

### a) 2-(1-Hydroxycyclopentyl)benzofuran-5-carboxylic Acid

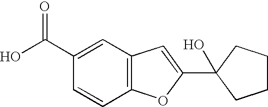

The title compound was synthesized as described for Example 74a) in 99% yield, starting from methyl 2-(1-hydroxycyclopentyl)benzofuran-5-carboxylate.

¹H NMR (DMSO-d₆) δ ppm 12.84 (br. s., 1H) 8.21 (d, 1H) 7.85 (dd, 1H) 7.61 (d, 1H) 6.84 (s, 1H) 5.38 (s, 1H) 1.95-2.07 (m, 2H) 1.66-1.95 (m, 6H); MS (ESI) m/z 245 [M−1]⁻.

### b) Methyl 2-(1-hydroxycyclopentyl)benzofuran-5-carboxylate

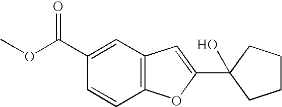

The title compound was synthesized as described for Example 74b) in 95% yield, starting from 1-ethynylcyclopentanol.

¹H NMR (CDCl₃) δ ppm 8.19 (dd, 1H) 7.92 (dd, 1H) 7.39 (d, 1H) 6.61 (d, 1H) 3.87 (s, 3H) 2.06-2.20 (m, 2H) 1.73-2.00 (m, 6H); MS (EI) m/z 260 [M]⁺.

### EXAMPLE 78

### 2-Cyclopentyl-N-(2-sulfamoylphenylsulfonyl)benzofuran-5-carboxamide

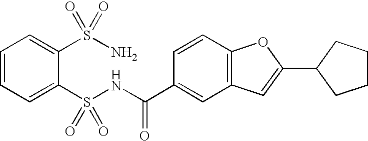

The title compound was synthesized as described for Example 74 in 38% yield, starting from 2-cyclopentylbenzofuran-5-carboxylic acid.

¹H NMR (DMSO-d₆) δ 8.15-8.28 (m, 1H) 7.97-8.06 (m, 2H) 7.75 (br. s., 2H) 7.62 (dt, 1H) 7.39-7.49 (m, 1H) 7.29 (s, 2H) 6.59 (s, 1H) 3.07-3.17 (m, 1H) 1.86-1.96 (m, 2H) 1.47-1.67 (m, 6H); MS (ESI) m/z 447 [M−1]⁻.

### a) 2-Cyclopentylbenzofuran-5-carboxylic Acid

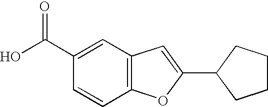

The title compound was synthesized as described for Example 74a) in 83% yield, starting from methyl 2-cyclopentylbenzofuran-5-carboxylate.

¹H NMR (DMSO-d₆) δ ppm 12.82 (br. s., 1H) 8.15 (d, 1H) 7.83 (dd, 1H) 7.58 (d, 1H) 6.72 (s, 1H) 3.23-3.31 (m, 1H) 2.01-2.10 (m, 2H) 1.64-1.78 (m, 6H); MS (ESI) m/z 229 [M−1]⁻.

### b) Methyl 2-cyclopentylbenzofuran-5-carboxylate

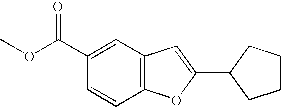

The title compound was synthesized as described for Example 74b) in 97% yield, starting from cyclopentylacetylene.

¹H NMR (CDCl₃) δ ppm 8.07 (dd, 1H) 7.80 (dd, 1H) 7.28 (dt, 1H) 6.30 (t, 1H) 3.80 (s, 3H) 3.05-3.15 (m, 1H) 1.91-2.02 (m, 2H) 1.52-1.74 (m, 6H); MS (EI) m/z 244 [M]⁺.

### EXAMPLE 79

### 3-Cyano-4-(3,3-dimethylbut-1-ynyl)-N-(2-sulfamoylphenylsulfonyl)benzamide

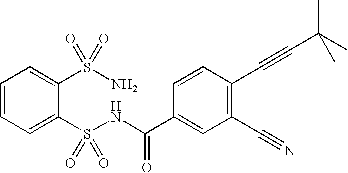

A mixture of 4-bromo-3-cyano-N-(2-sulfamoylphenylsulfonyl)benzamide (0.11 g, 0.25 mmol), 3,3-dimethyl-1-butyne (0.046 mL, 0.37 mmol), copper(I) iodide (4.72 mg, 0.020 mmol), bis(triphenylphosphine)palladium(II) chloride (0.017 g, 0.020 mmol), and diisopropylamine (0.11 mL, 0.74 mmol) in N,N-dimethylformamide (2 mL) under an atmosphere of argon was heated at 100° C. for 2 hours in a microwave. The reaction mixture was partitioned between ethyl acetate and diluted hydrochloric acid, the organic phase was dried over magnesium sulfate and the solvent was evaporated. Purification by preparative HPLC followed by column chromatography, using 5% methanol in chloroform as the eluent, gave 0.020g (18% yield) of the title compound.

¹H NMR (DMSO-d₆) δ ppm 8.00-8.06 (m, 2H) 7.94 (dd, 1H) 7.86 (dd, 1H) 7.50-7.56 (m, 1H) 7.45-7.50 (m, 1H) 7.41 (d, 1H) 7.28 (s, 2H) 1.19 (s, 9H); MS (ESI) m/z 444 [M−1]⁻.

### a) 4-Bromo-3-cyano-N-(2-sulfamoylphenylsulfonyl)benzamide

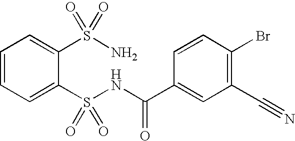

The title compound was synthesized as described for Example 73a) in 25% yield, starting from 4-bromo-3-cyanobenzoic acid. Purification by column chromatography using a step-wise gradient of methanol (10-20%) in chloroform as the eluent.

MS (ESI) m/z 442, 444 [M−1]⁻.

### EXAMPLE 80

### 4-(Benzofuran-2-yl)-3-cyano-N-(2-sulfamoylphenylsulfonyl)benzamide

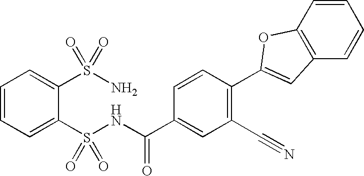

A mixture of 4-bromo-3-cyano-N-(2-sulfamoylphenylsulfonyl)benzamide (0.24 g, 0.54 mmol), 2-benzofuranboronic acid (0.11 g, 0.70 mmol), 1,1′-bis(diphenylphosphino)ferrocene-palladium dichloride (0.044 g, 0.050 mmol), N,N-dimethylformamide (4 mL) and 2 M sodium carbonate (0.81 mL, 1.62 mmol) under an atmosphere of argon was heated at 120° C. for 0.5 hour in a microwave. The reaction mixture was partitioned between ethyl acetate and diluted hydrochloric acid, the organic phase was dried over magnesium sulfate and evaporated. Purification by preparative HPLC gave 0.07 1g (27% yield) of the title compound.

¹H NMR (DMSO-d₆) δ ppm 8.31 (br. s., 1H) 8.16-8.21 (m, 1H) 8.09-8.13 (m, 1H) 8.05-8.08 (m, 1H) 7.96-8.00 (m, 1H) 7.66-7.74 (m, 4H) 7.55-7.59 (m, 1H) 7.29-7.39 (m, 3H) 7.19-7.24 (m, 1H); MS (ESI) m/z 480 [M−1]⁻.

### EXAMPLE 81

### 4-Chloro-2-hydroxy-N-(2-sulfamoylphenylsulfonyl)benzamide

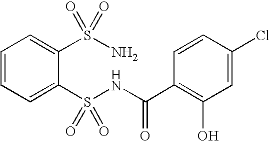

The title compound was synthesized as described for Example 61a) in 1% yield, starting from 4-chloro-2-hydroxybenzoic acid.

¹H NMR (CD₃OD) δ ppm 8.21 (dd, 1H) 8.09 (dd, 1H) 7.69 (d, 1H) 7.63-7.50 (m, 2H) 6.71 (d, 1H) 6.64 (dd, 1H); MS (ESI) m/z 389 [M−1]⁻

### EXAMPLE 82

### 4-Bromo-2-hydroxy-N-(2-sulfamoylphenylsulfonyl)benzamide

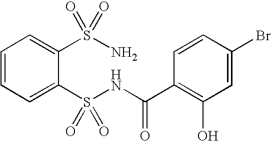

The title compound was synthesized as described for Example 61a) in 1% yield, starting from 4-bromo-2-hydroxybenzoic acid.

¹H NMR (6274 (m, 3H) 6.98 (d, 1H) 6.90 (dd, 1H) MS (ESI) m/z 435, 433 [M−1]⁻

### EXAMPLE 83

### 4-(Benzofuran-2-yl)-2-fluoro-N-(2-sulfamoylphenylsulfonyl)benzamide

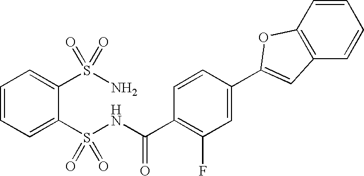

4-Bromo-2-fluoro-N-(2-sulfamoylphenylsulfonyl)benzamide (200 mg, 0.46 mmol), benzofuran-2-ylboronic acid (81 mg, 0.50 mmol), [1,1′-bis(diphenylphosphino)ferrocene]dichloropalladium (37 mg, 0.05 mmol) and potassium carbonate (379 mg, 2.74 mmol) were dissolved in tetrahydrofuran (5 mL) and water (1 mL) in a microwave vial. The reaction was heated at 150° C. for 60 min in a microwave, filtered through a plug of celite and concentrated in vacuo. Purification by preparative HPLC gave 84 mg (39% yield) of the title compound.

¹H NMR (DMSO-d₆) δ ppm 7.29 (t, 1H) 7.39-7.34 (m, 1H) 7.46 (s, 2H) 7.61 (s, 1H) 7.65 (d, 1H) 7.76-7.67 (m, 9H) 7.85 (t, 1H) 8.07 (d, 1H) 8.23 (d, 1H); MS (ESI) m/z 473 [M−1]⁻

### a) 4-Bromo-2-fluoro-N-(2-sulfamoylphenylsulfonyl)benzamide

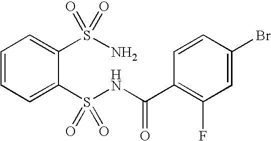

Benzene-1,2-disulfonamide (1.0 g, 4.23 mmol), 4-bromo-2-fluorobenzoic acid (0.93 g, 4.23 mmol), N-(3-dimethylaminopropyl)-N′-ethylcarbodiimide hydrochloride (1.14 g, 5.93 mmol) and 4-dimethylaminopyridine (1.29 g, 10.6 mmol) were dissolved in anhydrous N,N-dimethylformamide (15 mL) and the reaction was stirred at room temperature over night. Water was added and the solution was extracted with ethyl acetate. The aqueous phase was acidified using hydrochloric acid (2 M) and extracted with ethyl acetate. The combined organic phases were washed with water, dried over magnesium sulfate, filtered and concentrated in vacuo to give 1.69 g (91% yield) of the title compound.

MS (ESI) m/z 435, 437 [M−1]⁻.

### EXAMPLE 84

### 4-(3,3-Dimethylbut-1-ynyl)-2-fluoro-N-(2-sulfamoylphenylsulfonyl)benzamide

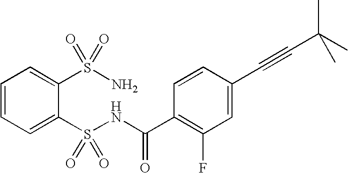

4-Bromo-2-fluoro-N-(2-sulfamoylphenylsulfonyl)benzamide (200 mg, 0.46 mmol), cuprous iodide (4 mg, 0.02 mmol), bis(triphenylphosphine)palladium(II) chloride (16 mg, 0.02 mmol), 3,3-dimethyl-1-butyne (0.169 mL, 1.37 mmol) and diisopropylamine (0.193 mL, 1.37 mmol) were slurried in anhydrous N,N-dimethylformamide (3 mL) in a microwave vial. The reaction was heated at 100° C. for 60 min in a microwave, filtered through a plug of celite and concentrated in vacuo. Purification by preparative HPLC gave 101 mg (50% yield) of the title compound.

¹H NMR (DMSO-d₆)₆ ppm 8.17-8.13 (m, 1H) 8.00 (dd, 1H) 7.72-7.52 (m, 6H) 7.10-7.04 (m, 2H) 1.28 (s, 9H); MS (ESI) m/z 437 [M−1]⁻

### EXAMPLE 85

### 4-(Cyclopentylethynyl)-2-fluoro-N-(2-sulfamoylphenylsulfonyl)benzamide

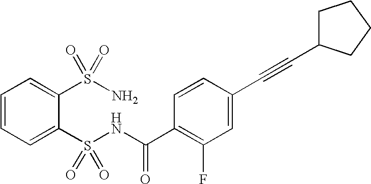

The title compound was synthesized as described for Example 84 in 42% yield, starting from 4-bromo-2-fluoro-N-(2-sulfamoylphenylsulfonyl)benzamide and ethynylcyclopentane.

¹H NMR (DMSO-d₆) δ ppm 8.15 (dd, 1H) 8.00 (dd, 1H) 7.71-7.52 (m, 6H) 7.14-7.00 (m, 2H) 2.86 (t, 1H) 2.02-1.91 (m, 2H) 1.70 (ddd, 2H) 1.49-1.65 (m, 4H); MS (ESI) m/z 449[M−1]⁻

### EXAMPLE 86

### 4-(Cyclopentylethynyl)-2-fluoro-3-methoxy-N-(2-sulfamoylphenylsulfonyl)benzamide

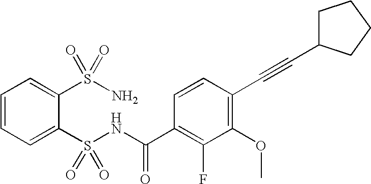

The title compound was synthesized as described for Example 84 in 9% yield, starting from 4-bromo-2-fluoro-3-methoxy-N-(2-sulfamoylphenylsulfonyl)benzamide and ethynylcyclopentane.

¹H NMR (CD₃OD) δ ppm 8.35 (dd, 1H) 8.21 (dd, 1H) 7.76-7.66 (m, 2H) 7.39 (t, 1H) 7.07 (d, 1H) 3.91 (s, 3H) 2.96-2.85 (m, 1H) 2.07-1.97 (m, 2H) 1.84-1.61 (m, 6H);

MS (ESI) m/z 479 [M−1]⁻

### a) 4-Bromo-2-fluoro-3-methoxy-N-(2-sulfamoylphenylsulfonyl)benzamide

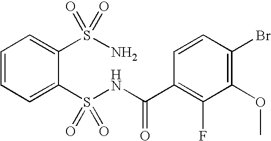

The title compound was synthesized as described for Example 83a) in 91% yield, starting from 4-bromo-2-fluoro-3-methoxybenzoic acid.

MS (ESI) m/z 465, 467 [M−1]⁻

### EXAMPLE 87

### 4-(Benzofuran-2-yl)-2-fluoro-3-methoxy-N-(2-sulfamoylphenylsulfonyl)benzamide

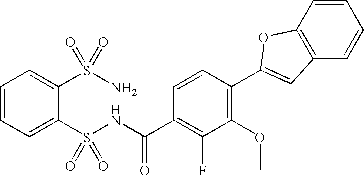

The title compound was synthesized as described for Example 83 in 14% yield, starting from 4-bromo-2-fluoro-3-methoxy-N-(2-sulfamoylphenylsulfonyl)benzamide.

¹H NMR (DMSO-d₆) δ ppm 12.78 (br. s., 1H) 8.35 (dd, 1H) 8.19 (dd, 1H) 7.97-7.88 (m, 2H) 7.77 (dd, 2H) 7.66 (d, 1H) 7.60 (s, 1H) 7.49 (dd, 1H) 7.45 (s, 2H) 7.42-7.37 (m, 1H) 7.31 (t, 1H) 4.01 (s, 3H); MS (ESI) m/z 503 [M+1]⁺

### EXAMPLE 88

### 5-(Cyclohexylethynyl)-N-(2-sulfamoylphenylsulfonyl)picolinamide

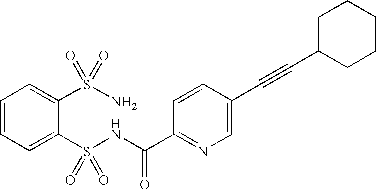

The title compound was synthesized as described for Example 84 in 4% yield, starting from 5-bromo-N-(2-sulfamoylphenylsulfonyl)picolinamide and ethynylcyclohexane.

¹H NMR (CDCl₃) δ ppm 8.21 (s, 1H) 8.12 (d, 1H) 7.90 (d, 1H) 7.73 (d, 1H) 7.46 (d, 1H) 7.38 (t, 1H) 7.33 (t, 1H) 2.39-2.27 (m, 1H) 1.56-1.63 (m, 2H) 1.50-1.40 (m, 2H) 1.29-1.20 (m, 2H) 1.13-1.02 (m, 4H); MS (ESI) m/z 446 [M−1]⁻

### a) 5-Bromo-N-(2-sulfamoylphenylsulfonyl)picolinamide

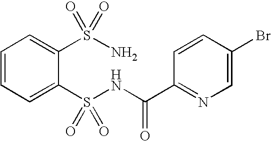

The title compound was synthesized as described for Example 83a) in 57% yield, starting from 5-bromopicolinic acid made acidic.

MS (ESI) m/z 418, 420 [M−1]⁻

### EXAMPLE 89

### 5-(3,3-Dimethylbut-1-ynyl)-N-(2-sulfamoylphenylsulfonyl)picolinamide

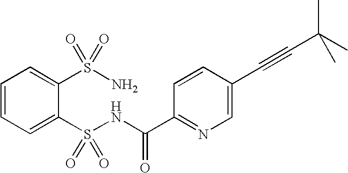

The title compound was synthesized as described for Example 84 in 4% yield, starting from 5-bromo-N-(2-sulfamoylphenylsulfonyl)picolinamide and 3,3-dimethylbut-1-yne.

¹H NMR (CDCl₃) δ ppm 8.24 (s, 1H) 8.16 (d, 1H) 7.92 (dl H) 7.69 (d, 1H) 7.51-7.41 (m, 3H) 1.02 (s, 9H); MS (ESI) m/z 420 [M−1]⁻

### EXAMPLE 90

### 4-(3,3-Dimethylbut-1-ynyl)-2-fluoro-3-methoxy-N-(2-sulfamoylphenylsuifonyl)benzamide

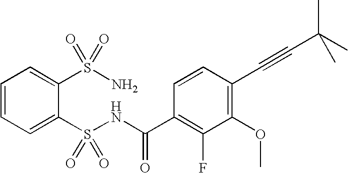

The title compound was synthesized as described for Example 84 in 8% yield, starting from 4-bromo-2-fluoro-3-methoxy-N-(2-sulfamoylphenylsulfonyl)benzamide and 3,3-dimethylbut-1-yne but was heated at 100° C. for 180 min in a microwave.

¹H NMR (CDCl₃) δ ppm 8.22-8.16 (m, 1H) 8.00-7.95 (m, 1H) 7.57-7.50 (m, 2H) 7.10 (t, 1H) 6.84 (d 1H) 3.66 (s, 3H) 1.02 (s, 9H); MS (ESI) m/z 467 [M−1]⁻

### EXAMPLE 91

### 4-(Benzofuran-2-yl)-2-chloro-N-(2-sulfamoylphenylsulfonyl)benzamide

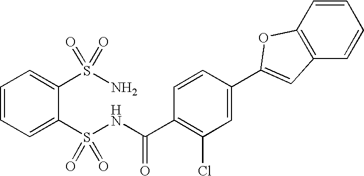

The title compound was synthesized as described for Example 83 in 8% yield, starting from 4-bromo-2-chloro-N-(2-sulfamoylphenylsulfonyl)benzamide but was heated at 150° C. for 15 min in a microwave.

¹H NMR (CD₃OD) δ ppm 8.53 (dd, 1H) 8.33 (dd, 1H) 8.01 (d, 1H) 7.93-7.88 (m, 3H) 7.70 (dl H) 7.66 (d, 1H) 7.58 (d, 1H) 7.37 (t, 1H) 7.28 (t, J=7.25 Hz, 1H); MS (ESI) m z 489 [M−1]⁻

### a) 4-Bromo-2-chloro-N-(2-sulfamoylphenylsulfonyl)benzamide

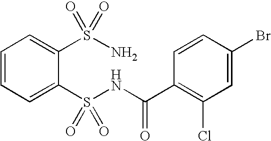

The title compound was synthesized as described for Example 83a) in 80% yield, starting from 4-bromo-2-chlorobenzoic acid.

MS (ESI) m/z 451, 453 [M−1]⁻

### EXAMPLE 92

### 4-(Cyclopentylethynyl)-2-hydroxy-N-(2-sulfamoylphenylsulfonyl)benzamide

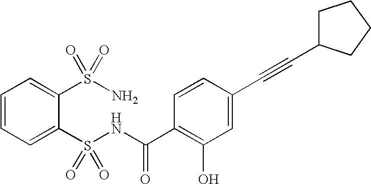

The title compound was synthesized as described for Example 83 in 35% yield, starting from 4-bromo-2-hydroxy-N-(2-sulfamoylphenylsulfonyl)benzamide and ethynylcyclopentane but was heated at 100° C. for 30 min in a microwave: ¹H NMR CD₃OD) δ ppm 8.33 (dd, 1H) 8.20 (dd, 1H) 7.75 (d, 1H) 7.69 (ddd, 2H) 6.78-6.72 (m, 2H) 2.88-2.82 (m, 1H) 2.05-1.98 (m, 2H) 1.835-1.75 (m, 2H) 1.71-1.62 (m, 4H);

MS (ESI) m/z 447 [M−1]⁻

### EXAMPLE 93

### 6-(Cyclopentylethynyl)-N-(2-sulfamoylphenylsulfonyl)nicotinamide

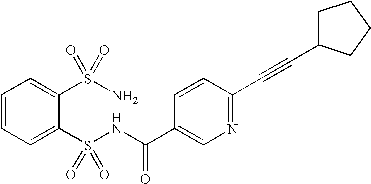

Copper(I) iodide (0.267 μL, 7.88 μmol) was added to a stirred solution of 6-bromo-N-(2-sulfamoylphenylsulfonyl)nicotinamide (0.177 g, 0.42 mmol), cyclopentylacetylene (0.050 mL, 0.43 mmol), tetrakis(triphenylphosphine)palladium(0) (0.0301 g, 0.03 mmol) and triethylamine (1 mL, 7.2 mmol) in N,N-dimethylformamide (5 mL) under an atmosphere of nitrogen. The resulting mixture was heated at 65° C. over night. Water and ethyl acetate was added and the aqueous phase was washed with ethyl acetate. The aqueous phase was acidified (pH ˜2) with 2 M hydrochloric acid and extracted with ethyl acetate. The organic phase was washed with water/brine (1:1) and brine, dried over magnesium sulfate and the solvent was evaporated. Dissolved in dichloromethane and the organic phase was washed with water and water/brine (1:1), dried over magnesium sulfate and the solvent was evaporated to give 0.090g (49% yield) of the title compound.

¹H NMR (400 MHz, DMSO-d₆) δ ppm 8.92 (d, 1H) 8.27-8.38 (m, 1H) 8.07-8.21 (m, 2H) 7.78-7.90 (m, 2H) 7.51 (d, 1H) 7.45 (br. s., 2H) 2.85-2.99 (m, 1H) 1.90-2.07 (m, 2H) 1.52-1.78 (m, 6H). MS (ESI) m/z 434.1 [M+H]⁺, 432.2 [M−H]⁻.

### a) 6-Bromo-N-(2-sulfamoylphenylsulfonyl)nicotinamide

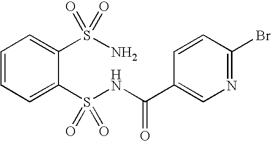

1-(3-Dimethylaminopropyl)-3-ethylcarbodiimide hydrochloride (0.508 g, 2.65 mmol) was added to a solution of 6-bromonicotinic acid (0.357 g, 1.77 mmol), benzene-1,2-disulfonamide (0.418 g, 1.77 mmol) and 4-dimethylaminopyridine (0.318 g, 2.60 mmol) in N,N-dimethylformamide (20 mL) at room temperature and the mixture was stirred over night. Water was added and the aqueous phase was washed with ethyl acetate. The aqueous phase was acidified (pH ˜2) with 2 M hydrochloric acid and extracted with ethyl acetate. The organic phase was washed with water and water/brine (1:1), dried over magnesium sulfate and the solvent was evaporated to give 0.677 g (91% yield) of the title compound.

¹H NMR (400 MHz, DMSO-d₆) δ ppm 8.80 (d, 1H) 8.29-8.37 (m, 1H) 8.08-8.16 (m, 2H) 7.81-7.92 (m, 2H) 7.78 (d, 1H) 7.46 (m, 1H); MS (ESI) m/z 420.0 [M+H], 421.8 [M−H]⁻.

### EXAMPLE 94

### 6-(Pyridin-2-ylethynyl)-N-(2-sulfamoylphenylsulfonyl)nicotinamide

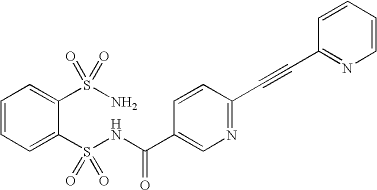

The title compound was synthesized as described for Example 93 in 46% yield, starting from 2-ethynylpyridine.

¹H NMR (400 MHz, DMSO-d₆) δ ppm 9.04 (d, 1H) 8.67 (d, 1H) 8.30-8.37 (m, 1H) 8.27 (dd, 1H) 8.09-8.16 (m, 1H) 7.87-7.97 (m, 1H) 7.73-7.88 (m, 4H) 7.41-7.53 (m, 3H); MS (ESI) m/z 443.0 [M+H]⁺, 441.2 [M−H]⁻.

### EXAMPLE 95

### 6-(Pyridin-3-ylethynyl)-N-(2-sulfamoylphenylsulfonyl)nicotinamide

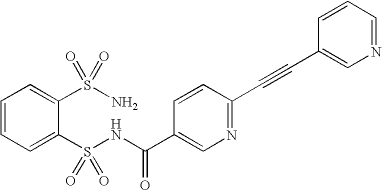

The title compound was synthesized as described for Example 93 in 17% yield, starting from 3-ethynylpyridine.

¹H NMR (400 MHz, DMSO-d₆) δ ppm 9.03 (d, 1H) 8.85 (d, 1H) 8.67 (dd, 1H) 8.31-8.38 (m, 1H) 8.27 (dd, 1H) 8.07-8.16 (m, 2H) 7.82-7.90 (m, 2H) 7.79 (d, 1H) 7.54 (dd, 1H) 7.47 (br. s., 2H); MS (ESI) m/z 443.0 [M+H]⁺, 441.2 [M−H]⁻.

### EXAMPLE 96

### 2-(3,3-Dimethylbut-1-ynyl)-N-(2-sulfamoylphenylsulfonyl)pyrimidine-5-carboxamide

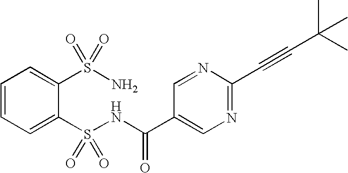

The title compound was synthesized as described for Example 93a) in 59% yield, starting from of 2-(3,3-dimethylbut-1-ynyl)pyrimidine-5-carboxylic acid. The residue was dissolved in warm dichloromethane/methanol (9:1), a small amount of dichloromethane was added and the mixture was allowed to cool down. The formed precipitate was removed by filtration, washed with dichloromethane and dried in vacuo.

¹H NMR (400 MHz, DMSO-d₆) δ ppm 8.99 (s, 2H) 8.18 (dd, 1H) 8.00 (dd, 1H) 7.57-7.72 (m, 2H) 7.39 (s, 2H) 1.31 (s, 9H); MS (ESI) m/z 423.0 [M+H]⁺, 421.2 [M−H]⁻.

### a) 2-(3,3-Dimethylbut-1-ynyl)pyrimidine-5-carboxylic Acid

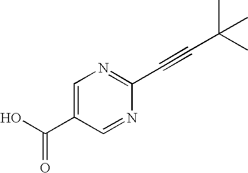

A solution of lithium hydroxide monohydrate (0.047 g, 1.13 mmol) in water (1 mL) was added to a solution of methyl 2-(3,3-dimethylbut-1-ynyl)pyrimidine-5-carboxylate (0.080 g, 0.37 mmol) in tetrahydrofuran (4 mL) and the mixture was stirred at room temperature over night. Water was added and the pH was set to ˜1 with 2 M hydrochloric acid. The aqueous phase was extracted with ethyl acetate and the combined organic phases were washed with water and brine, dried over magnesium sulfate and concentrated to give 0.061 g (82% yield) of the title compound.

¹H NMR (400 MHz, DMSO-d₆) δ ppm 13.65 (s, 1H) 8.85 (d, 1H) 8.16 (dd, 1H) 7.80 (d, 1H); MS (ESI) m/z 205.0 [M+H]⁺, 203.1 [M−H]⁻.

### b) Methyl 2-(3,3-dimethylbut-1-ynyl)pyrimidine-5-carboxylate

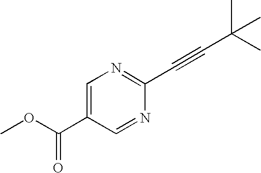

Water (2 mL) was added to a stirred suspension of methyl 2-chloropyrimidine-5-carboxylate (0.306 g, 1.77 mmol), (2-tert-butyl-1-ethynyl)diisopropoxyborane (0.45 mL, 1.91 mmol), [1,1′-bis(diphenylphosphino)ferrocene]palladium(II) chloride (0.111 g, 0.14 mmol) and potassium carbonate (0.770 g, 5.57 mmol) in tetrahydrofuran (8 mL) and the resulting mixture was heated at 60° C. over night. Water and ethyl acetate was added. The aqueous phase was extracted with ethyl acetate and the combined organic phases were washed with water and brine, dried over magnesium sulfate and the solvent was evaporated. Purification by column chromatography, using 0-10% methanol in dichloromethane as the eluent, gave 0.082 g (21% yield) of the title compound.

¹H NMR (400 MHz, DMSO-d₆) δ ppm 9.16 (s, 2H) 3.91 (s, 3H) 1.33 (s, 9H).

### EXAMPLE 97

### N-(2-Sulfamoylphenylsulfonyl)-4-((3,3,3-trifluoropropoxy)methyl)benzamide

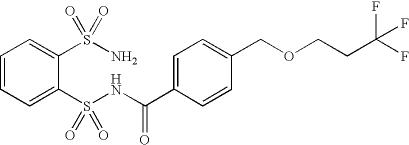

The title compound was synthesized as described for Example 93a) in 43% yield, starting from 4-((3,3,3-trifluoropropoxy)methyl)benzoic acid. Purification by column chromatography, using a gradient of 0-10% methanol in dichloromethane as the eluent.

¹H NMR (400 MHz, DMSO-d₆) δ ppm 8.35 (dd, 1H) 8.16 (dd, 1H) 7.85-7.96 (m, 4H) 7.36-7.46 (m, 4H) 4.57 (s, 2H) 3.66 (t, 2H) 2.53-2.68 (m, 2H); MS (ESI) m/z 465.2 [M−H]⁻.

### a) 4-((3,3,3-Trifluoropropoxy)methyl)benzoic acid

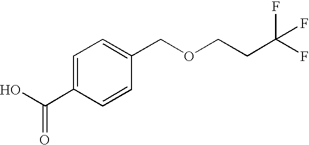

The title compound was synthesized as described for Example 96a) in 82% yield, starting from methyl 4-((3,3,3-trifluoropropoxy)methyl)benzoate.

¹H NMR (400 MHz, CDCl₃) δ ppm 8.12 (d, 2H) 7.45 (d, 2H) 4.63 (s, 2H) 3.74 (t, 2H) 2.38-2.61 (m, 2H); MS (ESI) m/z 247.2 [M−H]⁻.

### b) Methyl 4-((3,3,3-trifluoropropoxy)methyl)benzoate

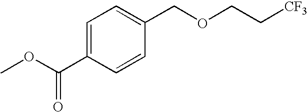

3,3,3-Trifluoropropan-1-ol (0.200 mL, 2.27 mmol) was added dropwise to a stirred suspension of sodium hydride (0.084 mL, 2.52 mmol, prewashed with heptane) in tetrahydrofuran (2 mL) and the resulting mixture was stirred at room temperature for 5 min. A solution of methyl 4-(bromomethyl)benzoate (0.519 g, 2.27 mmol) in tetrahydrofuran (2.5 mL) was added dropwise followed by addition of tetrabutylammonium iodide (0.083 g, 0.22 mmol). The mixture was heated at 65° C. for 2.5 hours and was then allowed to cool down to room temperature. Water was added and the aqueous phase was extracted with ethyl acetate. The combined organic phases were washed with water and brine, dried over magnesium sulfate and the solvent was evaporated. Purification by column chromatography, using 0-100% ethyl acetate in n-heptane as the eluent, gave 0.435 g (73% yield) of the title compound.

¹H NMR (400 MHz, CDCl₃) δ ppm 8.04 (d, 2H) 7.37-7.46 (m, 2H) 4.60 (s, 2H) 3.93 (s, 3H) 3.72 (t, 2H) 2.37-2.55 (m, 2H); MS (ESI) m/z 261.2 [M−H]⁻.

### EXAMPLE 98

### 4-(Cyclopentylethynyl)-3-(hydroxymethyl)-N-(2-sulfamoylphenylsulfonyl)benzamide

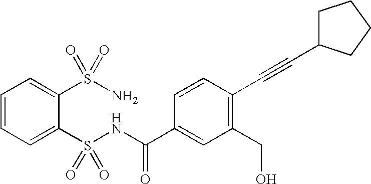

Copper(I) iodide (0.89 μL, 0.03 mmol) was added to a stirred solution of 4-bromo-3-(hydroxymethyl)-N-(2-sulfamoylphenylsulfonyl)benzamide (0.1970 g, 0.44 mmol), cyclopentylacetylene (0.050 mL, 0.43 mmol), tetrakis(triphenylphosphine)palladium(0) (0.0251 g, 0.02 mmol) and triethylamine (0.92 mL, 6.60 mmol) in N,N-dimethylformamide (6 mL) under an atmosphere of nitrogen. The resulting mixture was heated at 65° C. over night. Another portion of cyclopentylacetylene (0.050 mL, 0.43 mmol) was added, and the mixture was stirred at 65° C. over night. Water and ethyl acetate was added and the aqueous phase was washed with ethyl acetate. The aqueous phase was acidified (pH ˜2) with 2 M hydrochloric acid and extracted with ethyl acetate. The organic phase was washed with water/brine (1:1) and brine, dried over magnesium sulfate and the solvent was evaporated. Purification by column chromatography, using a gradient of 0-10% methanol in dichloromethane as the eluent, followed by purification by preparative HPLC gave 0.045 g (22% yield) of the title compound.

¹H NMR (400 MHz, DMSO-d₆) δ ppm 8.22-8.34 (m, 1H) 8.05-8.16 (m, 1H) 8.00 (s, 1H) 7.76-7.91 (m, 2H) 7.70-7.76 (m, 1H) 7.40 (s, 2H) 7.31-7.38 (m, 1H) 4.59 (s, 2H) 2.86-2.98 (m, 1H) 1.99 (s, 2H) 1.68-1.78 (m, 2H) 1.51-1.68 (m, 4H); MS (ESI) m/z 463.1 [M+H]⁺, 461.3 [M−H]⁻.

### a) 4-Bromo-3-(hydroxymethyl)benzoic acid

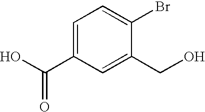

The title compound was synthesized as described for Example 96a) in 98% yield, starting from methyl 3-(acetoxymethyl)-4-bromobenzoate.

¹H NMR (400 MHz, DMSO-d₆) δ ppm 13.12 (br. s., 1H) 8.11 (d, 1H) 7.64-7.78 (m, 2H) 5.59 (br. s., 1H) 4.54 (br. s., 2H); MS (ESI) m/z 229 and 231 [M−H]⁻.

### b) Methyl 3-(acetoxymethyl)-4-bromobenzoate

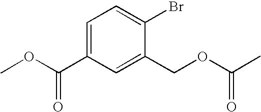

Potassium acetate (1.89 g, 19.3 mmol) was added to a solution of methyl 4-bromo-3-(bromomethyl)benzoate (3.015 g, 9.79 mmol) in acetic acid (12 mL) and the mixture was heated at 100° C. for 5 hours. Water and ethyl acetate was added. The aqueous phase was extracted with ethyl acetate. The combined organic phases were washed with water, saturated sodium hydrogen carbonate and brine, dried over magnesium sulfate and the solvent was evaporated. Purification by column chromatography, using 0-30% ethyl acetate in n-heptane as the eluent, gave 1.61 g (57% yield from methyl 4-bromo-3-methylbenzoate).

¹H NMR (400 MHz, CDCl₃) δ ppm 8.07 (d, 1H) 7.86 (dd, 1H) 7.67 (d, 1H) 5.23 (s, 2H) 3.94 (s, 3H) 2.18 (s, 3H).

### c) Methyl 4-bromo-3-(bromomethyl)benzoate

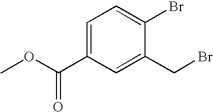

N-Bromosuccinimide (1.0 mL, 12 mmol) and 2,2′-azobisisobutyronitrile (0.005 g, 0.03 mmol) was added to a stirred solution of methyl 4-bromo-3-methylbenzoate (2.190 g, 9.56 mmol) in carbon tetrachloride (50 mL) and the resulting mixture was stirred at 70° C. for 2.5 days. Water and chloroform was added. The aqueous phase was extracted with chloroform and the combined organic phases were washed with water and 5% aqueous sodium hydrogen carbonate, dried over magnesium sulfate and the solvent was evaporated to give 3.015 g of the title compound.

GC MS (EI) m/z 308 [M]⁺.

### EXAMPLE 99

### 6-(3-Methylbut-1-ynyl)-N-(2-sulfamoylphenylsulfonyl)nicotinamide

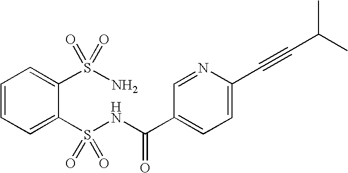

The title compound was synthesized as described for Example 93 in 40% yield, starting from 6-bromo-N-(2-sulfamoylphenylsulfonyl)nicotinamide and 3-methyl-1-butyne but the mixture was heated at 65° C. for 1.5 hours. Purification by column chromatography, using dichloromethane/methanol (85:15) as the eluent.

¹H NMR (400 MHz, DMSO-d₆) δ ppm 8.93 (d, 1H) 8.30-8.39 (m, 1H) 8.09-8.21 (m, 2H) 7.80-7.94 (m, 2H) 7.54 (d, 1H) 7.45 (br. s., 2H) 2.79-2.94 (m, 1H) 1.23 (d, 6H);

MS (ESI) m/z 408.1 [M+H]⁺, 406.3 [M−H]⁻.

### EXAMPLE 100

### 3-(Hydroxymethyl)-4-(phenylethynyl)-N-(2-sulfamoylphenylsulfonyl)benzamide

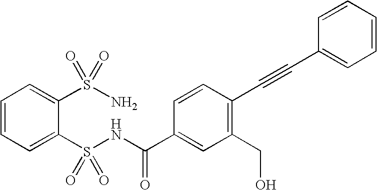

The title compound was synthesized as described for Example 93 in 29% yield, starting from 4-bromo-3-(hydroxymethyl)-N-(2-sulfamoylphenylsulfonyl)benzamide and phenylacetylene but was heated at 65° C. for 2 days. Purification by preparative HPLC.

¹H NMR (400 MHz, DMSO-d₆) δ ppm 8.29-8.40 (m, 1H) 8.11-8.19 (m, 1H) 8.05 (s, 1H) 7.86-7.93 (m, 2H) 7.84 (dd, 1H) 7.56-7.63 (m, 3H) 7.43-7.50 (m, 3H) 7.42 (br. s., 2H) 4.73 (s, 2H); MS (ESI) m/z 471.1 [M+H]⁺, 469.3 [M−H]⁻.

### EXAMPLE 101

### 4-(Cyclohexylethynyl)-3-(hydroxymethyl)-N-(2-sulfamoylphenylsulfonyl)benzamide

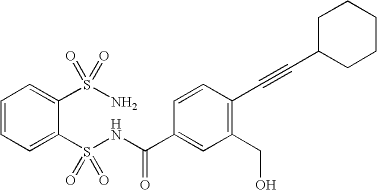

The title compound was synthesized as described for Example 93 in 32% yield, starting from 4-bromo-3-(hydroxymethyl)-N-(2-sulfamoylphenylsulfonyl)benzamide and cyclohexylacetylene but was heated at 65° C. for 3 days. Purification by preparative HPLC.

¹H NMR (400 MHz, DMSO-d₆) δ ppm 8.33 (dd, 1H) 8.10-8.20 (m, 1H) 7.99 (s, 1H) 7.82-7.95 (m, 2H) 7.76 (dd, 1H) 7.32-7.46 (m, 3H) 4.61 (s, 2H) 2.68-2.81 (m, 1H) 1.81 (dd, 2H) 1.59-1.74 (m, 2H) 1.44-1.59 (m, 3H) 1.28-1.44 (m, 3H); MS (ESI) m/z 477.1 [M+H]⁺, 475.3 [M−H]⁻.

### EXAMPLE 102

### 2-((4-chlorophenyl)ethynyl)-N-(2-sulfamoylphenylsulfonyl)pyrimidine-5-carboxamide

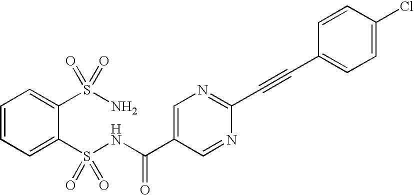

1-(3-Dimethylaminopropyl)-3-ethylcarbodiimide hydrochloride (0.0857 g, 0.45 mmol) was added to a solution of benzene-1,2-disulfonamide (0.0753 g, 0.32 mmol), 2-((4-chlorophenyl)ethynyl)pyrimidine-5-carboxylic acid (0.080 g, 0.31 mmol) and 4-dimethylaminopyridine (0.0567 g, 0.46 mmol) in N,N-dimethylformamide (15 mL) at room temperature and the mixture was stirred over night. Water was added and the aqueous phase was washed with ethyl acetate. The aqueous phase was acidified to pH ˜1 with 2 M hydrochloric acid and extracted with ethyl acetate. The organic phase was washed with water and brine, dried over magnesium sulfate and the solvent was evaporated. Purification by preparative HPLC gave 0.042 g (29% yield) of the title compound.

¹H NMR (400 MHz, DMSO-d₆) δ ppm 9.12 (s, 2H) 8.26 (dd, 1H) 8.06 (dd, 1H) 7.67-7.78 (m, 4H) 7.52-7.61 (m, 2H) 7.44 (br. s., 2H); MS (ESI) m/z 477.0 [M+H]⁺, 475.2 [M−H]⁻.

### a) 2-((4-Chlorophenyl)ethynyl)pyrimidine-5-carboxylic Acid

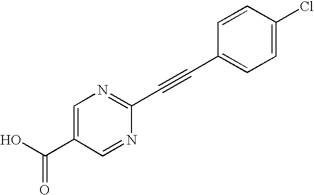

The title compound was synthesized as described for Example 96a) in 85% yield, starting from methyl 2-((4-chlorophenyl)ethynyl)pyrimidine-5-carboxylate.

¹H NMR (400 MHz, DMSO-d₆) δ ppm 13.71-14.20 (br. s., 1H) 9.22 (s, 2H) 7.68-7.85 (m, 2H) 7.49-7.67 (m, 2H); MS (ESI) m/z 259.0 [M+H]⁺, 257.1 [M−H]⁻.

### b) Methyl 2-((4-chlorophenyl)ethynyl)pyrimidine-5-carboxylate

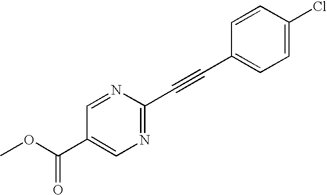

The title compound was synthesized as described for Example 93 in 26% yield, starting from methyl 2-chloropyrimidine-5-carboxylate and 1-chloro-4-ethynylbenzene but was heated at 65° C. for 3 hours. Purification by preparative HPLC.

¹H NMR (400 MHz, DMSO-d₆) δ ppm 9.26 (s, 2H) 7.68-7.82 (m, 2H) 7.53-7.65 (m, 2H) 3.93 (s, 3H); MS (ESI) m/z 273.0 [M+H]⁺.

### EXAMPLE 103

### 4-(Benzofuran-2-yl)-3-(hydroxymethyl)-N-(2-sulfamoylphenylsulfonyl)benzamide

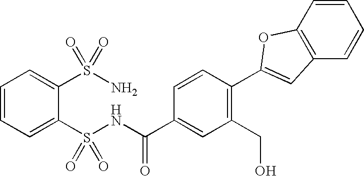

4-Bromo-3-(hydroxymethyl)-N-(2-sulfamoylphenylsulfonyl)benzamide (0.1912 g, 0.43 mmol), benzofuran-2-ylboronic acid (0.0783 g, 0.48 mmol), potassium carbonate (0.2428 g, 1.76 mmol) and [1,1′-bis(diphenylphosphino)ferrocene]palladium(II) chloride (0.0385 g, 0.05 mmol) in tetrahydrofuran (10 mL) and water (2 mL) was heated at 65° C. overnight. Water and ethyl acetate was added and the aqueous phase was acidified with hydrochloric acid (2 M). The aqueous phase was extracted with ethyl acetate. The combined organic phases were washed with water, water/brine (1:1) and brine, dried over magnesium sulfate and the solvent was evaporated. Purification by preparative HPLC gave 0.042 g (20% yield) of the title compound.

¹H NMR (400 MHz, DMSO-d₆) δ ppm 8.29-8.39 (m, 1H) 8.18 (s, 1H) 8.11-8.17 (m, 1H) 7.92-8.02 (m, 2H) 7.82-7.92 (m, 2H) 7.72 (s, 1H) 7.65 (s, 1H) 7.42 (d, 3H) 7.38 (s, 1H) 7.30 (s, 1H) 4.78 (s, 2H); MS (ESI) m/z 487.1 [M+H]⁺, 485.3 [M−H]⁻.

### EXAMPLE 104

### 4-(Benzofuran-2-yl)-N-(2-sulfamoylphenylsulfonyl)cyclohexanecarboxamide

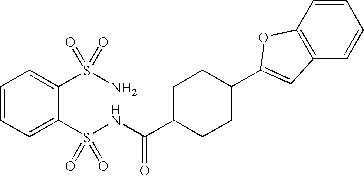

4-(Benzofuran-2-yl)cyclohexanecarboxylic acid (0.337 g, 1.38 mmol), N-(3-dimethylaminopropyl)-N′-ethylcarbodiimide hydrochloride (0.264 g, 1.38 mmol) and 4-(dimethylamino)pyridine (0.234 g, 1.92 mmol) were added to a solution of benzene-1,2-disulfonamide (0.181 g, 0.77 mmol) in N,N-dimethylformamide (10 mL) at room temperature. The reaction mixture was stirred for 3 hours and the solvent was evaporated. Purification by preparative HPLC gave 0.14 g (38% yield) of the title compound as a mixture of regioisomers.

### a) 4-(Benzofuran-2-yl)cyclohexanecarboxylic acid

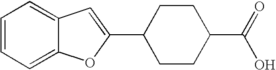

A solution of sodium hypochlorite (0.147 g, 1.97 mmol and sulfamic acid (0.191 g, 1.97 mmol) in water (5 mL) was added dropwise to a cooled (0° C.) solution of 4-(benzofuran-2-yl)cyclohexanecarbaldehyde (0.300 g, 1.31 mmol) in tetrahydrofuran (15 mL). The reaction mixture was stirred at 0° C. for 10 min and was then allowed to reach 10° C. before the reaction was quenched with solid sodium thiosulphate. The resulting mixture was partitioned between brine and ethyl acetate, the organic phase was dried over magnesium sulfate and the solvent was evaporated to give 0.38 g (quantitative yield) of the title compound.

### b) 4-(Benzofuran-2-yl)cyclohexanecarbaldehyde

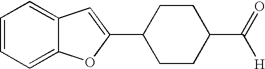

A solution of potassium tert-butoxide (1.006 g, 8.96 mmol) dissolved in tetrahydrofuran (15 mL) was added dropwise to a cooled (0° C.) solution of (methoxymethyl)triphenylphosphonium chloride (3.07 g, 8.96 mmol) in tetrahydrofuran (15 mL) under an atmosphere of argon. The reaction mixture was stirred for 15 min at 0° C. and was then allowed to reach room temperature. A solution of 4-(benzofuran-2-yl)cyclohexanone (0.960 g, 4.48 mmol, WO 2004099191 A2) in tetrahydrofuran (15 mL) was added dropwise and the mixture was stirred over night. The reaction mixture was cooled to 0° C. and water (10 mL) and 6 M aqueous hydrochloric acid (10 mL) were added dropwise. The resulting mixture was stirred for 1 hour at room temperature and was then extracted with ethyl acetate. The organic phase was washed with brine, dried over magnesium sulfate and the solvent was evaporated. Purification by column chromatography, using heptane/ethyl acetate (13:1-10:1) as the eluent, gave 0.31 g (30% yield) of the title compound.

GC MS (EI) m/z 228 [M]⁺.

### EXAMPLE 105

### (1s,4s)-4-(Benzofuran-2-yl)-N-(2-sulfamoylphenysulfonyl)cyclohexanecarboxamide

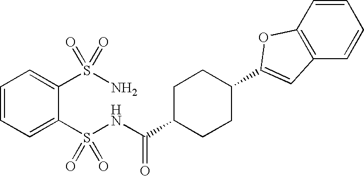

The regioisomers of 4-(benzofuran-2-yl)-N-(2-sulfamoylphenylsulfonyl)cyclohexanecarboxamide (0.125 g, 0.27 mmol) were separated by preparative chromatography was run on a SFC Berger Multigram system with a Knauer K-2501 UV detector. Column; Chiralcel AD 10 μm 21.2×250 mm. The column temperature was set to 35° C. An isocratic condition of 40% ethanol and 60% C₂₀ was applied at flow rate 50.0 mL/min. The UV detector scanned at 220 nm. The UV signal determined the fraction collection, to give 0.033 g (26% yield) of the title compound.

¹H NMR (400 MHz, CD₃OD) δ ppm 8.37 (dd, 1H), 8.09-8.30 (m, 1H), 7.72-7.92 (m, 2H), 7.40-7.54 (m, 1H), 7.34 (d, 1H), 7.03-7.27 (m, 2H), 6.39 (s, 1H), 2.82-3.07 (m, 1H), 2.53 (d, 1H), 1.87-2.12 (m, 2H), 1.73-1.89 (m, 4H), 1.56-1.74 (m, 2H); MS (ESI) m/z 461 [M−1]⁻

### EXAMPLE 106

### (1r,4r)-4-(Benzofuran-2-yl)-N-(2-sulfamoylphenylsulfonyl)cyclohexanecarboxamide

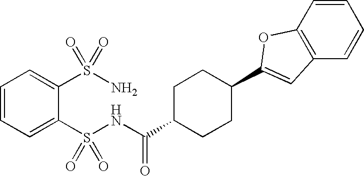

The regioisomers of 4-(benzofuran-2-yl)-N-(2-sulfamoylphenylsulfonyl)cyclohexanecarboxamide (0.125 g, 0.27 mmol were separated by preparative chromatography was run on a SFC Berger Multigram system with a Knauer K-2501 UV detector. Column; Chiralcel AD 10 μm 21.2×250 mm. The column temperature was set to 35° C. An isocratic condition of 40% ethanol and 60% C₂₀ was applied at flow rate 50.0 mL/min. The UV detector scanned at 220 nm. The UV signal determined the fraction collection, to give 0.065 g (52% yield) of the title compound.

¹H NMR (400 MHz, CD₃OD) δ ppm 8.41 (dd, 1H), 8.26 (dd, 1H), 7.71-7.96 (m, 2H), 7.40-7.57 (m, 1H), 7.35 (d, 1H), 7.03-7.28 (m, 2H), 6.41 (s, 1H), 2.54-2.86 (m, 1H), 2.28-2.47 (m, 1H), 2.18 (dd, 2H), 1.80-2.07 (m, 2H), 1.21-1.66 (m, 4H); MS (ESI) m/z 461 [M−1]⁻.

### EXAMPLE 107

### 4-(Benzofuran-2-yl)-1-methyl-N-(2-sulfamoylphenylsulfonyl)cyclohexanecarboxamide

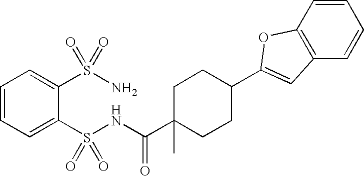

4-(Benzofuran-2-yl)-1-methylcyclohexanecarboxylic acid (0.158 g, 0.61 mmol), N-(3-dimethylaminopropyl)-N′-ethylcarbodiimide hydrochloride (0.176 g, 0.92 mmol) and 4-dimethylaminopyridine (0.156 g, 1.27 mmol) were added to a solution of benzene-1,2-disulfonamide (0.120 g, 0.51 mmol) in N,N-dimethylformamide (10 mL) at room temperature and stirred over night. More of N-(3-dimethylaminopropyl)-N′-ethylcarbodiimide hydrochloride (0.076 g, 0.40 mmol) and 4-dimethylaminopyridine (0.056 g, 0.46 mmol) were added. The reaction mixture was stirred for another 2 hours and was then partitioned between water and ethyl acetate. The organic phase was dried over magnesium sulfate and the solvent was evaporated. Purification by preparative HPLC gave 0.112 g (46% yield) of the title compound as a mixture of regioisomers.

MS (ESI) m/z 475 [M−1]⁻.

### a) 4-(Benzofuran-2-yl)-1-methylcyclohexanecarboxylic acid

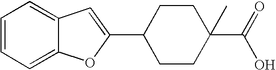

The title compound was synthesized as described for 104 b) in 86% yield, starting from 4-(benzofuran-2-yl)-1-methylcyclohexanecarbaldehyde.

MS (ES−) m/z 257 [M−1]⁻

### b) 4-(Benzofuran-2-yl)-1-methylcyclohexanecarbaldehyde

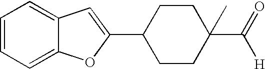

Potassium tert-butoxide (0.151 g, 1.34 mmol) was added to a cooled solution (0° C.) of 4-(benzofuran-2-yl)cyclohexanecarbaldehyde (0.236 g, 1.03 mmol) in dichloromethane (15 mL) followed by addition of iodomethane (0.193 mL, 3.10 mmol). The mixture was stirred at 0° C. for 30 min, the cooling was removed and the mixture was stirred at room temperature for another 1.5 hour. The reaction mixture was partitioned between brine and dichloromethane. The organic phase was dried over magnesium sulfate and the solvent was evaporated. Purification by column chromatography, using heptane/ethyl acetate (10:1) as the eluent, gave 0.173 g (69% yield) of the title compound.

GC MS (EI) m/z 242 [M]⁺.

### EXAMPLE 108

### (1 r,4r)-4-(Benzofuran-2-yl)-1-methyl-N-(2-sulfamoylphenylsulfonyl)cyclohexanecarboxamide

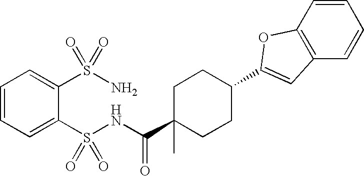

The regioisomers 4-(benzofuran-2-yl)-1-methyl-N-(2-sulfamoylphenylsulfonyl)cyclohexanecarboxamide (0.111 g, 0.23 mmol) were separated by preparative chromatography was run on a SFC Berger Multigram system with a Knauer K-2501 UV detector. Column; Chiralcel OD 10 μm 21.2×250 mm. The column temperature was set to 335° C. An isocratic condition of 40% methanol+0.1% DEA and 60% C₂₀ was applied at flow rate 50.0 mL/min. The UV detector scanned at 220 nm. The UV signal determined the fraction collection, to give 0.064g (58% yield) of the title compound.

¹H NMR (400 MHz, CD₃OD) δ ppm 8.20 (d, 1H), 8.15 (dd, 1H), 7.54-7.65 (m, 2H), 7.44-7.51 (m, 1H), 7.36 (d, 1H), 7.07-7.21 (m, 2H), 6.34 (s, 1H), 2.59-2.74 (m, 1H), 2.37 (d, 2H), 1.93 (d, 2H), 1.65 (d, 2H), 1.17-1.25 (m, 2H), 1.14 (s, 3H); MS (ESI) m/z 461 [M−1]⁻.

### EXAMPLE 109

### (1s,4s)-4-(Benzofuran-2-yl)-1-methyl-N-(2-sulfamoylphenylsulfonyl)cyclohexanecarboxamide

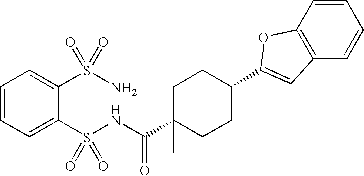

The regioisomers of 4-(benzofuran-2-yl)-1-methyl-N-(2-sulfamoylphenylsulfonyl)cyclohexanecarboxamide (0.111 g, 0.23 mmol) were separated by preparative chromatography was run on a SFC Berger Multigram system with a Knauer K-2501 UV detector. Column; Chiralcel OD 10 μm 21.2×250 mm. The column temperature was set to 35° C. An isocratic condition of 40% methanol+0.1% DEA and 60% C₂₀ was applied at flow rate 50.0 mL/min. The UV detector scanned at 220 nm. The UV signal determined the fraction collection, to give 0.011g (10% yield) of the title compound.

¹H NMR (400 MHz, CD₃OD) δ ppm 8.14-8.30 (m, 2H), 7.61-7.76 (m, 2H), 7.46-7.54 (m, 1H), 7.34-7.43 (m, 1H), 7.10-7.23 (m, 2H), 6.44-6.51 (m, 1H), 2.75 (br. s., 1H), 1.99 (br. s., 2H), 1.84-1.96 (m, 2H), 1.79 (br. s., 4H), 1.20-1.24 (m, 3H), MS (ESI) m/z 475 [M−1]⁻.

### EXAMPLE 110

### 4-(3,3-Dimethylbut-1-ynyl)-3-methoxy-N-(2-sulfamoylphenylsulfonyl)benzamide

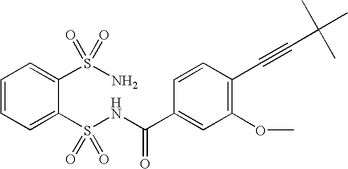

4-Bromo-3-methoxy-N-(2-sulfamoylphenylsulfonyl)benzamide (0.227 g, 0.51 mmol), diisopropyl 3,3-dimethylbut-1-ynylboronate (0.238 mL, 1.01 mmol) 1,1′-bis(diphenylphosphino)ferrocene-palladium dichloride (0.042 g, 0.05 mmol) were dissolved in N,N-dimethylformamide (3 mL) under an atmosphere of argon and a solution of aqueous sodium carbonate (0.758 mL, 1.52 mmol) was added. The reaction mixture was heated in a microwave at 120° C. for 20 min under argon atmosphere. The reaction mixture was partitioned between water and ethyl acetate. The organic phase was dried over magnesium sulfate and the solvent was evaporated. Purification by preparative HPLC and gave 0.019 g (8% yield) of the title compound. ¹H NMR (400 MHz, CD₃OD) δ ppm 8.35 (d, 1H), 8.16-8.28 (m, 1H), 7.67-7.79 (m, 2H), 7.53-7.63 (m, 1H), 7.46 (d, 1H), 7.27 (d, 1H), 3.87 (s, 3H), 1.27-1.37 (m, 9H); MS (ESI) m/z 449 [M−1]⁻.

### EXAMPLE 111

### 4-(Cyclopropylethynyl)-3-methoxy-N-(2-sulfamoylphenylsulfonyl)benzamide

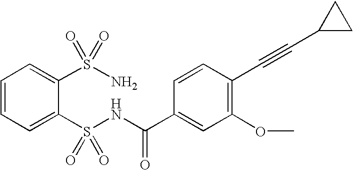

Ethynylcyclopropane (0.215 mL, 2.54 mmol), tetrakis(triphenylphosphine)palladium(0) (0.049 g, 0.04 mmol) and triethylamine (1.763 mL, 12.69 mmol) was added to a solution of 4-bromo-3-methoxy-N-(2-sulfamoylphenylsulfonyl)benzamide (0.190 g, 0.42 mmol) in N,N-dimethylformamide (13 mL) under an atmosphere of argon. The reaction mixture was stirred at room temperature for 5 min, copper(I) iodide (0.012 g, 0.06 mmol) was added and the reaction mixture was heated at 65° C. After 4 days was the reaction mixture filtered and the solvent was evaporated. Purification by preparative HPLC gave 0.088 g (48% yield) of the title compound.

¹H NMR (400 MHz, CD₃OD) δ ppm 8.30 (d, 1H), 8.19 (d, 1H), 7.57-7.74 (m, 3H), 7.47 (d, 1H), 7.24 (d, 1H), 3.87 (s, 3H), 1.42-1.56 (m, 1H), 0.83-0.94 (m, 2H), 0.69-0.80 (m, 2H); MS (ESI) m/z 433 [M−1]⁻.

### EXAMPLE 112

### 4-(3-Methoxy-3-methylbut-1-ynyl)-N-(2-sulfamoylphenylsulfonyl)benzamide

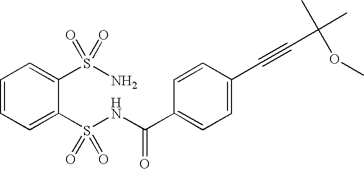

The title compound was synthesized as described for Example 111 in 36% yield, starting from 3-methoxy-3-methylbut-1-yne (Jackson, W. Roy et al., Aust. J. Chem., 1988, 41(2), 251-61) and 4-bromo-N-(2-sulfamoylphenylsulfonyl)benzamide.

¹H NMR (400 MHz, CD₃OD) δ ppm 8.29 (dd, 1H), 8.19 (dd, 1H), 7.98 (d, 2H), 7.58-7.73 (m, 2H), 7.39 (d, 2H), 3.41 (s, 3H), 1.52 (s, 6H); MS (ESI) m/z 435 [M−1]⁻.

### EXAMPLE 113

### 4-(3-Methylbut-1-ynyl)-N-(2-sulfamoylphenylsulfonyl)benzamide

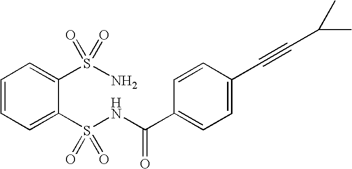

3-Methylbut-1-yne (0.085 g, 1.25 mmol), tetrakis(triphenylphosphine)palladium(0) (0.072 g, 0.06 mmol) and triethylamine (2.60 mL, 18.68 mmol) were added to a solution of 4-bromo-N-(2-sulfamoylphenylsulfonyl)benzamide (0.261 g, 0.62 mmol) in N,N-dimethylformamide (10 mL) under an atmosphere of argon. The reaction mixture was stirred at room temperature for 5 min, copper(I) iodide (0.018 g, 0.09 mmol) was added and the reaction mixture was heated at 65° C. over night. The reaction mixture was partitioned between water (set to pH-2 with aqueous 2 M hydrochloric acid) and ethyl acetate. The organic phase was dried over magnesium sulfate and the solvent was evaporated. Purification by preparative HPLC gave 0.058 g (23% yield) of the title compound.

¹H NMR (500 MHz, CD₃OD) δ ppm 8.38 (d, 1H) 8.17 (d, 1H) 7.73-7.80 (m, 2H) 7.70 (d, 2H) 7.32 (d, 2H) 2.61-2.79 (m, 1H) 1.16 (s, 3H) 1.15 (s, 3H); MS (ESI) m/z 405 [M−1]⁻.

### EXAMPLE 114

### 3-Methoxy-4-(3-methoxy-3-methylbut-1-ynyl)-N-(2-sulfamoylphenylsulfonyl)benzamide

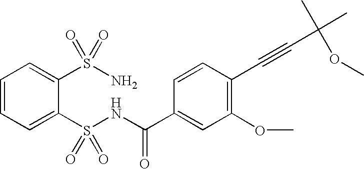

The title compound was synthesized as described for Example 111 in 33% yield, starting from 4-bromo-3-methoxy-N-(2-sulfamoylphenylsulfonyl)benzamide and 3-methoxy-3-methylbut-1-yne (Jackson, W. Roy et al., Aust. J. Chem., 1988, 41(2), 251-61).

¹H NMR (400 MHz, CD₃OD) δ ppm 8.29 (dd, 1H), 8.21 (dd, 1H), 7.62-7.76 (m, 3H), 7.55 (d, 1H), 7.30 (d, 1H), 3.88 (s, 3H), 3.43 (s, 3H), 1.52 (s, 6H); MS (ESI) m/z 465 [M−1]⁻.

### EXAMPLE 115

### 3-Hydroxy-4-(3-methoxy-3-methylbut-1-ynyl)-N-(2-sulfamoylphenylsulfonyl)benzamide

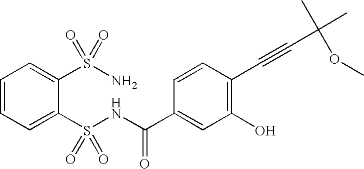

The title compound was synthesized as described for Example 111 in 31% yield, starting from 4-bromo-3-hydroxy-N-(2-sulfamoylphenylsulfonyl)benzamide and 3-methoxy-3-methylbut-1-yne (Jackson, W. Roy et al., Aust. J. Chem., 1988, 41(2), 251-61). Purification by preparative HPLC followed by column chromatography, using ethyl acetate/methanol (50:1-30:1+1% triethylamine) as the eluent.

¹H NMR (400 MHz, CD₃OD) δ ppm 8.28 (dd, 1H), 8.19 (dd, 1H), 7.57-7.75 (m, 2H), 7.48 (s, 1H), 7.43 (d, 1H), 7.24 (d, 1H), 3.44 (s, 3H), 1.53 (s, 6H); MS (ESI) m/z 451 [M−1]⁻.

### EXAMPLE 116

### 6-(3,3-Dimethylbut-1-ynyl)-N-(2-sulfamoylphenylsulfonyl)nicotinamide

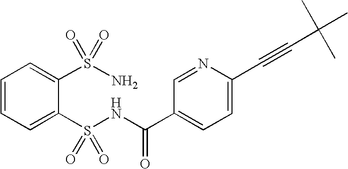

The title compound was synthesized as described for Example 110 in 25% yield, starting from 6-bromo-N-(2-sulfamoylphenylsulfonyl)nicotinamide and diisopropyl 3,3-dimethylbut-1-ynylboronate.

¹H NMR (400 MHz, CD₃OD) δ ppm 9.02 (d, 1H), 8.26-8.37 (m, 2H), 8.20 (dd, 1H), 7.60-7.75 (m, 2H), 7.42 (d, 1H), 1.31-1.44 (m, 9H); MS (ESI) m/z 420 [M−1]⁻.

### EXAMPLE 117

### 6-(Benzofuran-2-yl)-N-(2-sulfamoylphenylsulfonyl)nicotinamide

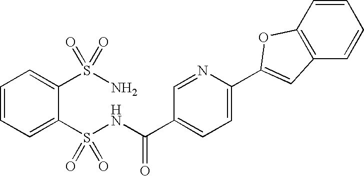

The title compound was synthesized as described for Example 110 in 25% yield, starting from 6-bromo-N-(2-sulfamoylphenylsulfonyl)nicotinamide and benzofuran-2-ylboronic acid.

¹H NMR (400 MHz, CD₃OD) δ ppm 9.20 (br. s., 1H), 8.47 (d, 1H), 8.35 (dd, 1H), 8.22 (dd, 1H), 7.99 (d, 1H), 7.63-7.78 (m, 3H), 7.54-7.62 (m, 2H), 7.33-7.45 (m, 1H), 7.28 (t, 1H); MS (ESI) m/z 456 [M−1]⁻.

### EXAMPLE 118

### 4-(3,3-Dimethylbut-1-ynyl)-3-(2-(2-methoxyethoxy)ethoxy)-N-(2-sulfamoylphenylsulfonyl)benzamide

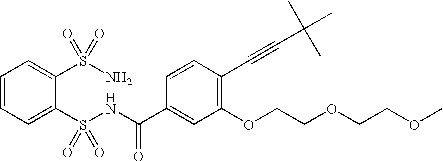

The title compound was synthesized as described for Example 110 in 28% yield, starting from 4-bromo-3-(2-(2-methoxyethoxy)ethoxy)-N-(2-sulfamoylphenylsulfonyl)benzamide and diisopropyl 3,3-dimethylbut-1-ynylboronate. ¹H NMR (500 MHz, DMSO-d₆) δ ppm 8.12 (dd, 1H) 7.98 (dd, 1H) 7.60-7.68 (m, 1H) 7.53-7.60 (m, 1H) 7.40-7.48 (m, 4H) 7.21 (d, 1H) 4.02-4.13 (m, 2H) 3.73-3.82 (m, 2H) 3.67 (dd, 2H) 3.46 (dd, 2H) 3.24 (s, 3H) 1.27 (s, 9H); MS (ESI) m/z 438 [M−1]⁻.

### a) 4-Bromo-3-(2-(2-methoxyethoxy)ethoxy)-N-(2-sulfamoylphenylsulfonyl)benzamide

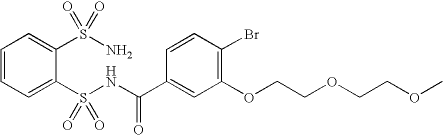

2-(2-Methoxyethoxy)ethanol (0.309 mL, 2.60 mmol), triphenylphosphine (0.681 g, 2.60 mmol) and diisopropyl azodicarboxylate (0.511 mL, 2.60 mmol) were added to a solution of methyl 4-bromo-3-hydroxybenzoate (0.4 g, 1.7 mmol) in tetrahydrofuran (20 mL) and the reaction mixture was stirred at room temperature for 2 days. A solution of lithium hydroxide monohydrate (0.124 g, 5.19 mmol) in water (2 mL) was added and the reaction mixture was stirred for another 4 days. The reaction mixture acidified with 2.0 M aqueous hydrochloric acid and partitioned between water and ethyl acetate. The organic phase was dried over magnesium sulfate and the solvent was evaporated. The product 4-bromo-3-(2-(2-methoxyethoxy)ethoxy)benzoic acid (0.562 g, 1.76 mmol), N-(3-dimethylaminopropyl)-N′-ethylcarbodiimide hydrochloride (0.506 g, 2.64 mmol) and 4-dimethylaminopyridine (0.323 g, 2.64 mmol) were added to a solution of benzene-1,2-disulfonamide (0.546 g, 2.31 mmol) in N,N-dimethylformamide (30 mL) at room temperature and stirred over night. Water was added and the solution was extracted with ethyl acetate. The aqueous phase was acidified with 2 M aqueous hydrochloric acid and extracted with ethyl acetate. The organic phase was dried over magnesium sulfate and the solvent was evaporated. Purification by column chromatography, using ethyl acetate/methanol (50:1+1% triethylamine) as the eluent, gave 0.55 g (60% yield) of the title compound.

¹H NMR (400 MHz, CD₃OD) δ ppm 8.45-8.55 (m, 1H), 8.22-8.31 (m, 1H), 7.81-7.89 (m, 2H), 7.62 (d, 1H), 7.53 (d, 1H), 7.35 (dd, 1H), 4.18-4.29 (m, 2H), 3.83-3.91 (m, 2H), 3.72 (dd, 2H), 3.51-3.58 (m, 2H), 3.27-3.35 (m, 3H); MS (ES) m/z 435 and 437 [M−1]⁻.

### EXAMPLE 119

### 4-(Benzofuran-2-yl)-3-(2-(2-methoxyethoxy)ethoxy)-N-(2-sulfamoylphenylsulfonyl)benzamide

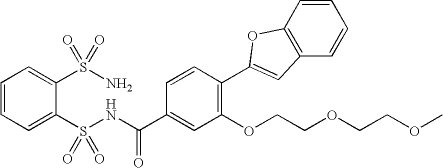

The title compound was synthesized as described for Example 110 in 21% yield, starting from 4-bromo-3-(2-(2-methoxyethoxy)ethoxy)-N-(2-sulfamoylphenylsulfonyl)benzamide and benzofuran-2-ylboronic acid.

¹H NMR (400 MHz, CD₃OD) δ ppm 8.43 (d, 1H), 8.20-8.30 (m, 1H), 8.06 (d, 1H), 7.73-7.84 (m, 3H), 7.59-7.71 (m, 3H), 7.53 (d, 1H), 7.32 (td, 1H), 7.15-7.27 (m, 1H), 4.42 (dd, 2H), 3.98-4.08 (m, 2H), 3.77-3.84 (m, 2H), 3.59-3.68 (m, 2H), 3.36-3.40 (m, 3H); MS (ESI) m/z 573 [M−1]⁻.

### EXAMPLE 120

### 2-(2-Methoxyphenyl)-N-(2-sulfamoylphenylsulfonyl)benzofuran-5-carboxamide

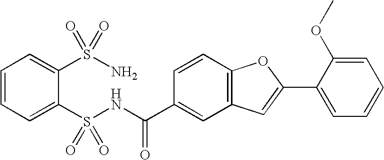

2-(2-Methoxyphenyl)benzofuran-5-carboxylic acid (0.058 g, 0.22 mmol) N-(3-dimethylaminopropyl)-N′-ethylcarbodiimide hydrochloride (0.062 g, 0.32 mmol) and 4-dimethylaminopyridine (0.026 g, 0.22 mmol) were added to a solution of benzene-1,2-disulfonamide (0.051 g, 0.22 mmol) in N,N-dimethylformamide (4 mL). The reaction mixture was stirred at room temperature over night and the solvent was evaporated. Purification by column chromatography, using ethyl acetate/methanol (40:1+1% triethylamine) as the eluent, gave 0.042 g (83% yield) of the title compound.

¹H NMR (400 MHz, DMSO-d₆) δ ppm 8.21 (d, 1H), 8.17 (dd, 1H), 8.00 (dd, 1H), 7.95 (dd, 1H), 7.90 (dd, 1H), 7.61-7.69 (m, 1H), 7.54-7.61 (m, 1H), 7.46-7.54 (m, 3H), 7.36-7.45 (m, 2H), 7.20 (d, 1H), 7.06-7.15 (m, 1H), 3.99 (s, 3H); MS (ESI) m/z 485 [M−1]⁻.

### a) 2-(2-Methoxyphenyl)benzofuran-5-carboxylic acid

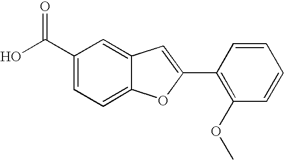

A solution of lithium hydroxide monohydrate (0.028 g, 0.67 mmol) in water (1 mL) was added to a solution of methyl 2-(2-methoxyphenyl)benzofuran-5-carboxylate (0.063 g, 0.22 mmol) in tetrahydrofuran (3 mL). The reaction mixture was stirred over night, acidified with 2.0 M aqueous hydrochloric acid and partitioned between water and ethyl acetate. The organic phase was dried over magnesium sulfate and the solvent was evaporated to give 0.058 g (97% yield) of the title compound.

¹H NMR (400 MHz, CD₃OD), δ ppm 13.68 (br. s., 1H), 9.11 (d, 1H), 8.76 (ddd, 2H), 8.50 (d, 1H), 8.31 (s, 1H), 8.19-8.29 (m, 1H), 8.03 (d, 1H), 7.87-7.98 (m, 1H), 4.76-4.87 (m, 3H); MS (ESI) m/z 267 [M−1]⁻.

### b) Methyl 2-(2-methoxyphenyl)benzofuran-5-carboxylate

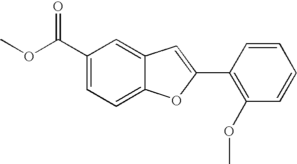

Methyl 4-hydroxy-3-iodobenzoate (0.111 g, 0.40 mmol, 2′-methoxyphenyl acetylene (0.052 ml, 0.40 mmol) 1,1,3,3-tetramethylguanidine (0.502 mL, 4.00 mmol) bis(triphenylphosphine)palladium(II) chloride (0.028 g, 0.04 mmol) and copper(I) iodide

(1.36 μL, 0.04 mmol) were dissolved in N,N-dimethylformamide (5 mL). The reaction mixture was heated at 50° C. under an atmosphere of argon over night and the solvent was evaporated. Purification by column chromatography, using heptane/ethyl acetate (9:1) as the eluent gave 0.064g (57% yield) of the title compound.

¹H NMR (400 MHz, CDCl₃), δ ppm 8.34 (d, 1H), 8.07 (dd, 1H), 8.01 (dd, 1H), 7.53 (d, 1H), 7.40 (s, 1H), 7.33-7.39 (m, 1H), 7.06-7.14 (m, 1H), 7.02 (d, 1H), 4.01 (s, 3H), 3.96 (s, 3H).

### EXAMPLE 121

### 2-(1-tert-Butoxyethyl)-N-(2-sulfamoylphenylsulfonyl)benzofuran-5-carboxamide

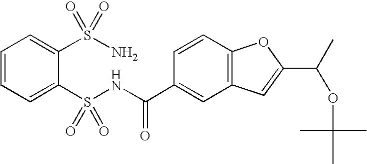

The title compound was synthesized as described for Example 120 in 64% yield, starting from 2-(1-tert-butoxyethyl)benzofuran-5-carboxylic acid.

¹H NMR (400 MHz, DMSO-d₆), δ ppm 8.12-8.17 (m, 2H), 7.99 (dd, 1H), 7.83 (dd, 1H), 7.53-7.67 (m, 2H), 7.41 (d, 1H), 6.76 (s, 1H) 4.88 (q, 1H), 1.41 (d, 3H), 1.16-1.22 (m, 9H); MS (ESI) m/z 479 [M−1]⁻.

### a) 2-(1-tert-Butoxyethyl)benzofuran-5-carboxylic acid

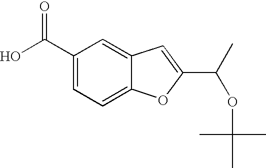

The title compound was synthesized as described for Example 120a) in 44% yield, starting from methyl 2-(1-tert-butoxyethyl)benzofuran-5-carboxylate.

¹H NMR (400 MHz, CD₃CD₂OD) δ ppm 13.61 (br. s., 1H) 9.02 (d, 1H) 8.67 (dd, 1H) 8.42 (d, 1H) 7.65 (s, 1H) 5.73 (q, 1H) 2.23 (dd, 3H) 2.00 (s, 9H); GC MS (ES) m/z 261 [M]⁺.

### b) Methyl 2-(1-tert-butoxyethyl)benzofuran-5-carboxylate

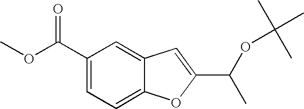

The title compound was synthesized as described for Example 120b) in 53% yield, starting from 3-tert-butoxybut-1-yne.

MS (ES) m/z 276 [M]⁺.

### EXAMPLE 122

### 2-(Pyridin-2-yl)-N-(2-sulfamoylphenylsulfonyl)benzofuran-5-carboxamide

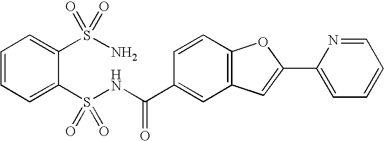

The title compound was synthesized as described for Example 120 in 35% yield, starting from 2-(pyridin-2-yl)benzofuran-5-carboxylic acid.

¹H NMR (500 MHz, CD₃OD), δ ppm 8.64 (dt, 1H), 8.46-8.54 (m, 1H), 8.30 (d, 1H), 8.23-8.29 (m, 1H), 8.00-8.07 (m, 1H), 7.97 (td, 1H), 7.92 (s, 1H), 7.79-7.89 (m, 2H), 7.66 (d, 1H), 7.60 (s, 1H,) 7.43 (ddd, 1H); MS (ESI) m/z 456 [M−1]⁻.

### a) 2-(Pyridin-2-yl)benzofuran-5-carboxylic acid

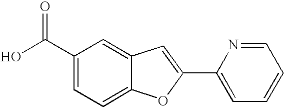

The title compound was synthesized as described for Example 120a) in 91% yield, starting from methyl 2-(pyridin-2-yl)benzofuran-5-carboxylate.

¹H NMR (400 MHz, CDCl₃) δ ppm 8.71 (d, 1H) 8.40 (d, 1H) 8.08 (dd, 1H) 7.93 (d, 1H) 7.83 (td, 1H) 7.60 (d, 1H) 7.51 (s, 1H) 7.30 (ddd, 1H); MS (ESI) m/z 239 [M−1]⁻.

### b) Methyl 2-(pyridin-2-yl)benzofuran-5-carboxylate

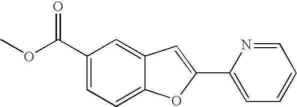

The title compound was synthesized as described for Example 120b) in 87% yield, starting from 2-ethynylpyridine.

¹H NMR (400 MHz, CDCl₃) δ ppm 8.71 (d, 1H) 8.40 (d, 1H) 8.08 (dd, 1H) 7.93 (d, 1H) 7.83 (td, 1H) 7.60 (d, 1H) 7.51 (s, 1H) 7.30 (ddd, 1H); GC MS (EI) m/z 253 [M]⁺.

### EXAMPLE 123

### 2-(Pyridin-3-yl)-N-(2-sulfamoylphenylsulfonyl)benzofuran-5-carboxamide

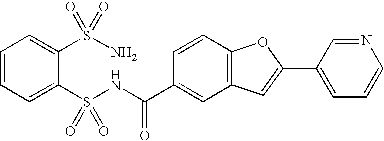

The title compound was synthesized as described for Example 120 in 24% yield, starting from 2-(pyridin-3-yl)benzofuran-5-carboxylic acid.

¹H NMR (500 MHz, CD₃OD), δ ppm 9.11 (s, 1H) 8.56 (d, 1H), 8.47-8.54 (m, 1H), 8.36 (dt, 1H), 8.22-8.30 (m, 2H), 7.91 (dd, 1H), 7.84 (dd, 2H), 7.65 (d, 1H), 7.57 (dd, 1H), 7.52 (s, 1H); MS (ESI) m/z 456 [M−1]⁻.

### a) 2-(Pyridin-2-yl)benzofuran-5-carboxylic Acid

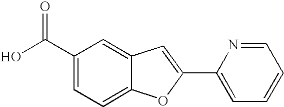

The title compound was synthesized as described for Example 120a) in 83% yield, starting from methyl 2-(pyridin-2-yl)benzofuran-5-carboxylate.

¹H NMR (400 MHz, DMSO-d₆) δ ppm 13.02 (br. s., 1H) 9.17 (d, 1H) 8.63 (dd, 1H) 8.31 (td, 2H) 7.96 (dd, 1H) 7.68-7.82 (m, 2H) 7.56 (dd, 1H); MS (ESI) m/z 238[M−1]⁻.

### b) Methyl 2-(pyridin-3-yl)benzofuran-5-carboxylate

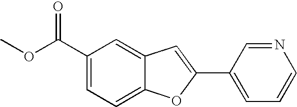

The title compound was synthesized as described for Example 120b) in 83% yield, starting from 3-ethynylpyridine.

¹H NMR (400 MHz, CDCl₃) δ ppm 9.14 (d, 1H) 8.63 (dd, 1H) 8.37 (d, 1H) 8.15 (dt, 1H) 8.07 (dd, 1H) 7.59 (d, 1H) 7.37-7.48 (m, 1H) 7.19 (d, 1H); GC MS (EI) m/z 253[M]⁺.

### EXAMPLE 124

### 2-(2-Hydroxypropan-2-yl)-N-(2-sulfamoylphenylsulfonyl)benzofuran-5-carboxamide

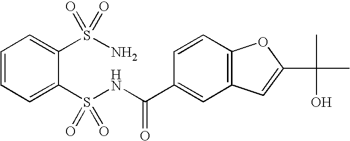

The title compound was synthesized as described for Example 120 in 85% yield, starting from 2-(2-hydroxypropan-2-yl)benzofuran-5-carboxylic acid.

¹H NMR (500 MHz, DMSO-d₆) δ ppm 8.10-8.19 (m, 2H) 7.99 (dd, 1H) 7.82 (dd, 1H) 7.60-7.67 (m, 1H) 7.54-7.60 (m, 1H) 7.42 (d, 1H) 6.71 (d, 1H) 1.51 (s, 6H); MS (ESI) m/z 437 [M−1]⁻.

### a) 2-(2-Hydroxypropan-2-yl)benzofuran-5-carboxylic Acid

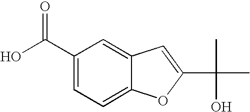

The title compound was synthesized as described for Example 120a) in 46% yield, starting from methyl 2-(2-hydroxypropan-2-yl)benzofuran-5-carboxylate.

¹H NMR (400 MHz, CD₃OD) δ ppm 8.26 (d, 1H) 7.96 (dd, 1H) 7.50 (d, 1H) 6.74 (s, 1H) 1.63 (s, 6H); MS (ESI) m/z 219 [M−1]⁻.

### b) Methyl 2-(2-hydroxypropan-2-yl)benzofuran-5-carboxylate

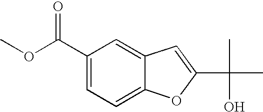

The title compound was synthesized as described for Example 120b) in 79% yield, starting from 2-methylbut-3-yn-2-ol.

GC MS (EI) m/z 234 [M]⁺.

### EXAMPLE 125

### 2-(2-Methoxypropan-2-yl)-N-(2-sulfamoylphenylsulfonyl)benzofuran-5-carboxamide

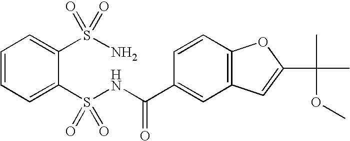

The title compound was synthesized as described for Example 120 in 85% yield, starting from 2-(2-methoxypropan-2-yl)benzofuran-5-carboxylic acid.

¹H NMR (500 MHz, DMSO-d₆), δ ppm 8.17 (d, 1H), 8.14 (dd, 1H), 7.98 (dd, 1H), 7.85 (dd, 1H), 7.62 (dd, 1H), 7.58 (dd, 1H), 7.49 (br. s., 2H), 7.46 (d, 1H), 6.91 (d, H), 2.98 (s, 3H) 1.51-1.58 (m, 6H); MS (ESI) m/z 451 [M−1]⁻.

### a) 2-(2-Methoxypropan-2-yl)benzofuran-5-carboxylic Acid

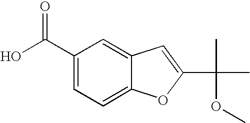

The title compound was synthesized as described for Example 120a) in 65% yield, starting from methyl 2-(2-methoxypropan-2-yl)benzofuran-5-carboxylate.

¹H NMR (400 MHz, CD₃OD) δ ppm 8.30 (d, 1H) 7.99 (dd, 1H) 7.53 (d, 1H) 6.87 (s, 1H) 3.12 (s, 3H) 1.62 (s, 6H); MS (ESI) m/z 233 [M−1]⁻.

### b) Methyl 2-(2-methoxypropan-2-yl)benzofuran-5-carboxylate

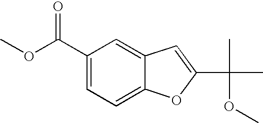

The title compound was synthesized as described for Example 120b) in 65% yield, starting from 3-methoxy-3-methylbut-1-yne.

GC MS (EI) m/z 248 [M]⁺.

### EXAMPLE 126

### 2-Cyclopropyl-N-(2-sulfamoylphenylsulfonyl)benzofuran-5-carboxamide

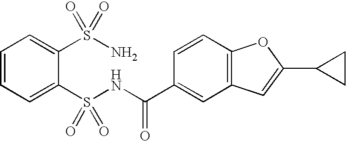

The title compound was synthesized as described for Example 120 in 36% yield, starting from 2-cyclopropylbenzofuran-5-carboxylic acid.

¹H NMR (500 MHz, CD₃OD) δ ppm 8.33-8.43 (m, 1H) 8.09-8.20 (m, 1H) 7.94 (d, 1H) 7.73 (dd, 2H) 7.64 (dd, 1H) 7.30 (dd, 1H) 6.36-6.47 (m, 1H) 1.95-2.05 (m, 1H) 0.90-1.00 (m, 2H) 0.80-0.90 (m, 2H); MS (ESI) m/z 419 [M−1]⁻.

### a) 2-Cyclopropylbenzofuran-5-carboxylic Acid

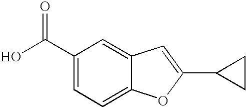

The title compound was synthesized as described for Example 120a) in 46% yield, starting from methyl 2-cyclopropylbenzofuran-5-carboxylate.

¹H NMR (400 MHz, CD₃OD) δ ppm 8.15 (d, 1H) 7.85-7.93 (m, 1H) 7.34-7.47 (m, 1H) 6.52 (s, 1H) 2.09 (tt, 1H) 0.99-1.07 (m, 2H) 0.90-0.99 (m, 2H); MS (ESI) m/z 201 [M−1]⁻.

### b) Methyl 2-cyclopropylbenzofuran-5-carboxylate

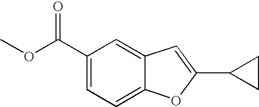

The title compound was synthesized as described for Example 120b) in 73% yield, starting from ethynylcyclopropane.

GC MS (EI) m/z 216 [M]⁺.

### EXAMPLE 127

### 4-(Benzofuran-2-yl)-3-isopropoxy-N-(2-sulfamoylphenylsulfonyl)benzamide

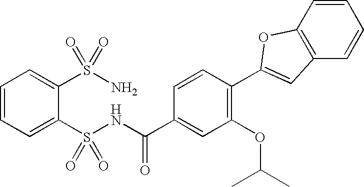

4-Bromo-3-isopropoxy-N-(2-sulfamoylphenylsulfonyl)benzamide (0.114 g, 0.24 mmol), benzofuran-2-ylboronic acid (0.077 g, 0.48 mmol) and 1,1′-bis(diphenylphosphino)ferrocene-palladium dichloride (0.020 g, 0.02 mmol) were dissolved in N,N-dimethylformamide under an atmosphere of argon followed by addition of aqueous sodium carbonate (0.358 mL, 0.72 mmol). The reaction mixture was heated in a microwave at 120° C. for 20 min under an atmosphere of argon and was then partitioned between water and ethyl acetate. The aqueous phase was acidified with aqueous hydrochloric acid (2 M) and extracted with ethyl acetate. The organic phase was dried over magnesium sulfate and the solvent was evaporated. Purification by preparative HPLC gave 0.064g (52% yield) of the title compound.

¹H NMR (500 MHz, DMSO-d₆), δ ppm 8.35 (d, 1H) 8.15 (d, 1H), 8.01 (d, 1H), 7.88 (br. s., 2H), 7.69-7.78 (m, 2H), 7.62 (d, 1H), 7.53-7.60 (m, 2H), 7.46 (s, 2H), 7.31-7.40 (m, 1H) 7.23-7.31 (m, 1H), 4.91-5.03 (m, 1H), 1.46 (s, 3H), 1.45 (s, 3H); MS (ESI) m/z 513 [M−1]⁻.

### a) 4-Bromo-3-isopropoxy-N-(2-sulfamoylphenylsulfonyl)benzamide

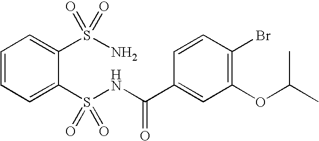

4-bromo-3-isopropoxybenzoic acid (0.621 g, 2.40 mmol), N-(3-dimethylaminopropyl)-N′-ethylcarbodiimide hydrochloride (0.689 g, 3.60 mmol) and 4-dimethylaminopyridine (0.439 g, 3.60 mmol) were added to a solution of benzene-1,2-disulfonamide (0.566 g, 2.40 mmol) in N,N-dimethylformamide (30 mL). The reaction mixture was stirred at room temperature over night and was then partitioned between water and ethyl acetate. The aqueous phase was acidified with aqueous hydrochloric acid (2 M) and extracted with ethyl acetate. The organic phase was dried over magnesium sulfate and the solvent was evaporated. Purification by column chromatography, using ethyl acetate as the eluent, gave 0.944g (83% yield) of the title compound.

¹H NMR (500 MHz, CD₃OD) δ ppm 8.39 (d, 1H), 8.20-8.28 (m, 1H), 7.73-7.79 (m, 2H), 7.61 (s, 1H), 7.56 (d, 1H), 7.39 (dd, 1H), 4.72 (dt, 1H), 1.37 (s, 3H), 1.35 (s, 3H);

MS (ESI) m/z 475, 477 [M−1]⁻.

### b) 4-Bromo-3-isopropoxybenzoic Acid

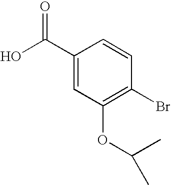

A solution of lithium hydroxide (0.355 g, 8.46 mmol) in water (3 mL) was added to a solution of methyl 4-bromo-3-isopropoxybenzoate (0.770 g, 2.82 mmol) in tetrahydrofuran (20 mL) and the reaction mixture was stirred at room temperature over night. The reaction mixture was acidified with 2.0 M aqueous hydrochloric acid and partitioned between water and ethyl acetate. The organic phase was dried over magnesium sulfate and the solvent was evaporated to give 0.62 1g (85% yield) of the title compound.

¹H NMR (500 MHz, CDCl₃) δ ppm 7.66 (d, 1H), 7.61 (d, 1H), 7.53-7.58 (m, 1H), 4.68 (dt, 1H), 1.43 (d, 6H); MS (ESI) m/z 257, 259 [M−1]⁻.

### c) Methyl 4-bromo-3-isopropoxybenzoate

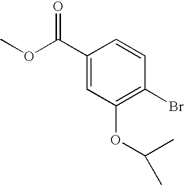

2-Propanol (0.348 mL, 4.54 mmol), triphenylphosphine (1.192 g, 4.54 mmol) and diisopropyl azodicarboxylate (0.895 mL, 4.54 mmol) were added to a solution of methyl 4-bromo-3-hydroxybenzoate (0.7 g, 3.03 mmol) in tetrahydrofuran (20 mL). The reaction mixture was stirred at room temperature over night and the solvent was evaporated. Purification by column chromatography, using heptane/ethyl acetate (8:1) as the eluent, gave 0.775 g (94% yield) of the title compound.

¹H NMR (500 MHz, CDCl₃) δ ppm 7.61 (d, 1H) 7.56 (d, 1H) 7.49 (dd, 1H) 4.67 (dt, 1H) 3.92 (s, 3H) 1.42 (s, 3H) 1.41 (s, 3H); GC MS (ES) m/z 272, 274 [M]⁺.

### EXAMPLE 128

### 4-(3,3-Dimethylbut-1-ynyl)-3-isopropoxy-N-(2-sulfamoylphenysulfonyl)benzamide

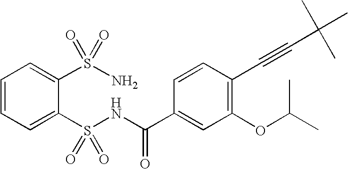

The title compound was synthesized as described for Example 127 in 30% yield, starting from 4-bromo-3-isopropoxy-N-(2-sulfamoylphenylsulfonyl)benzamide and diisopropyl 3,3-dimethylbut-1-ynylboronate.

¹H NMR (500 MHz, CD₃OD), δ ppm 8.34 (dd, 1H), 8.15 (dd, 1H), 7.67-7.80 (m, 2H), 7.38 (s, 1H), 7.29 (dd, 1H), 7.21 (d, 1H), 4.57 (dt, 1H), 1.24 (s, 3H), 1.23 (s, 3H), 1.18-1.22 (m, 9H); MS (ESI) m/z 477 [M−1]⁻.

### EXAMPLE 129

### 4-(3-Hydroxy-3-methylbut-1-ynyl)-3-isopropoxy-N-(2-sulfamoylphenylsulfonyl)benzamide

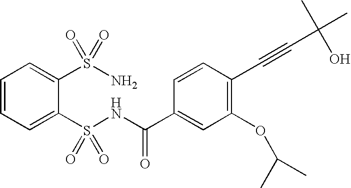

2-Methylbut-3-yn-2-ol (0.068 g, 0.81 mmol), tetrakis(triphenylphosphine)palladium(0) (0.047 g, 0.04 mmol) and triethylamine (1.699 ml, 12.19 mmol) were added to a solution of 4-bromo-3-isopropoxy-N-(2-sulfamoylphenylsulfonyl)benzamide (0.194 g, 0.41 mmol) in N,N-dimethylformamide (8 mL) under an atmosphere of argon. The reaction mixture was stirred at room temperature for 5 min, copper (I) iodide (0.012 g, 0.06 mmol) was added and the reaction mixture was heated at 65° C. over night. More 2-methylbut-3-yn-2-ol (0.068 g, 0.81 mmol) and tetrakis(triphenylphosphine)palladium(0) (0.047 g, 0.04 mmol) were added and the heating continued over the weekend. The reaction mixture was partitioned between water and ethyl acetate. The aqueous phase was acidified with aqueous hydrochloric acid (2 M) and extracted with ethyl acetate. The organic phase was dried over magnesium sulfate and the solvent was evaporated. Purification by preparative HPLC followed by column chromatography, using heptane/ethyl acetate (1:1) followed by ethyl acetate/methanol (100:1+1% triethylamine) as the eluent, gave 0.044g (23% yield) of the title compound.

¹H NMR (500 MHz, CD₃OD) δ ppm 8.28 (dd, 1H), 8.20 (dd, 1H), 7.61-7.74 (m, 3H), 7.50-7.58 (m, 1H), 7.30 (d, 1H), 4.60-4.74 (m, 1H), 1.56 (s, 6H), 1.34 (s, 3H), 1.33 (s, 3H); MS (ESI) m/z 479 [M−1]⁻.

### EXAMPLE 130

### 4-(Cyclopentylethynyl)-3-isopropoxy-N-(2-sulfamoylphenylsulfonyl)benzamide

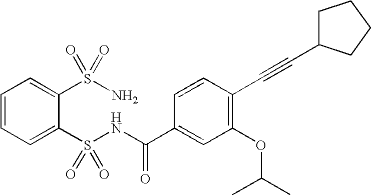

Ethynylcyclopentane (0.060 g, 0.64 mmol), tetrakis(triphenylphosphine)palladium(0) (0.049 g, 0.04 mmol) and triethylamine (1.787 mL, 12.82 mmol) were added to a solution of 4-bromo-3-isopropoxy-N-(2-sulfamoylphenylsulfonyl)benzamide (0.204 g, 0.43 mmol) in N,N-dimethylformamide (9 mL) under an atmosphere of argon. The reaction mixture was stirred at room temperature for 5 min, copper(I) iodide (0.012 g, 0.06 mmol) was added and the reaction mixture was heated at 65° C. over night. Ethynylcyclopentane (0.028 g, 0.3 mmol) was added and the reaction mixture was heated for an additional 24 hours. The reaction mixture was partitioned between water and ethyl acetate. The aqueous phase was acidified with aqueous hydrochloric acid (2 M) and extracted with ethyl acetate. The organic phase was dried over magnesium sulfate and the solvent was evaporated. Purification by preparative HPLC gave 0.023 g (11% yield) of the title compound.

¹H NMR (500 MHz, CD₃OD) δ ppm 1.31 (d, 6H) 1.58-1.68 (m, 2H) 1.68-1.77 (m, 2H) 1.76-1.87 (m, 2H) 1.92-2.08 (m, 2H) 2.89 (t, 1H) 4.55-4.71 (m, 1H) 7.24 (d, 1H) 7.55 (dd, 1H) 7.64 (d, 1H) 7.65-7.73 (m, 2H) 8.21 (d, 1H) 8.26 (d, 1H); MS (ESI) m/z 476 [M−1]⁻.

### EXAMPLE 131

### 4-(Cyclohexylethynyl)-3-isopropoxy-N-(2-sulfamoylphenylsulfonyl)benzamide

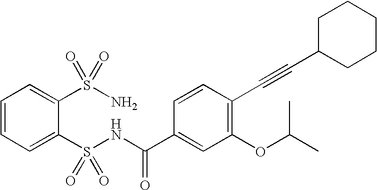

The title compound was synthesized as described for Example 130 in 16% yield, starting from 4-bromo-3-isopropoxy-N-(2-sulfamoylphenylsulfonyl)benzamide and ethynylcyclohexane.

¹H NMR (500 MHz, CD₃OD) δ ppm 8.27 (dd, 1H) 8.19-8.24 (m, 1H) 7.62-7.74 (m, 3H) 7.56 (dd, 1H) 7.26 (d, 1H) 4.59-4.73 (m, 1H) 2.67 (br. s., 1H) 1.73-1.94 (m, 4H) 1.49-1.67 (m, 3H) 1.36-1.49 (m, 3H) 1.32 (s, 3H) 1.31 (s, 3H); MS (ESI) m/z 503 [M−1]⁻.

### EXAMPLE 132

### 4-(Cyclopropylethynyl)-3-isopropoxy-N-(2-sulfamoylphenylsulfonyl)benzamide

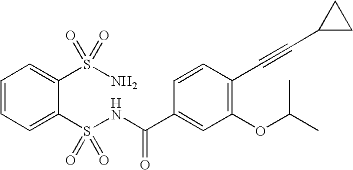

The title compound was synthesized as described for Example 130 in 16% yield, starting from 4-bromo-3-isopropoxy-N-(2-sulfamoylphenylsulfonyl)benzamide and ethynylcyclopropane.

¹H NMR (500 MHz, CD₃OD) δ ppm 8.39-8.53 (m, 1H) 8.16-8.34 (m, 1H) 7.72-7.93 (m, 2H) 7.42-7.52 (m, 1H) 7.34-7.41 (m, 1H) 7.24-7.34 (m, 1H) 4.57-4.76 (m, 1H) 1.43-1.56 (m, 1H) 1.33 (s, 3H) 1.32 (s, 3H) 0.86-0.94 (m, 2H) 0.71-0.78 (m, 2H);

MS (ESI) m/z 461 [M−1]⁻.

### EXAMPLE 133

### 4-((1-Hydroxycycloheptyl)ethynyl)-3-isopropoxy-N-(2-sulfamoylphenylsulfonyl)benzamide

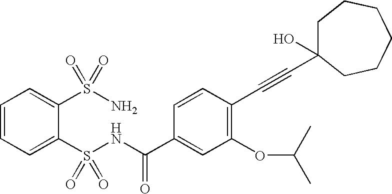

1-Ethynylcycloheptanol (0.105 g, 0.76 mmol, Verkruijsse, H D.; De Graaf, W.; Brandsma, L. Synth. Commun., 1988, 18(2), 131-4) tetrakis(triphenylphosphine)palladium(0) (0.044 g, 0.04 mmol) and triethylamine (1.594 mL, 11.44 mmol) was added to a solution of 4-bromo-3-isopropoxy-N-(2-sulfamoylphenylsulfonyl)benzamide (0.182g, 0.38 mmol) in N,N-dimethylformamide (8 mL) under an atmosphere of argon. The reaction mixture was stirred at room temperature for 5 min, copper(I) iodide (10.9 mg, 0.06 mmol) was added and the reaction mixture was heated at 65° C. for 2 days. The reaction mixture was partitioned between water and ethyl acetate. The aqueous phase was acidified with aqueous hydrochloric acid (2 M) and extracted with ethyl acetate. The organic phase was dried over magnesium sulfate and the solvent was evaporated. Purification by preparative HPLC gave 0.060g (29% yield) of the title compound.

¹H NMR (500 MHz, CD₃OD) δ ppm 8.43-8.51 (m, 1H) 8.23-8.31 (m, 1H) 7.80-7.90 (m, 2H) 7.50 (s, 1H) 7.39 (s, 2H) 4.69-4.79 (m, 1H) 2.03-2.16 (m, 2H) 1.80-1.92 (m, 2H) 1.66-1.79 (m, 6H) 1.55-1.66 (m, 2H) 1.36 (s, 3H) 1.34 (s, 3H); MS (ESI) m/z 533 [M−1]⁻.

### EXAMPLE 134

### 6-(3,3-Dimethylbut-1-ynyl)-5-(2-(2-methoxyethoxy)ethoxy)-N-(2-sulfamoylphenylsulfonyl)nicotinamide

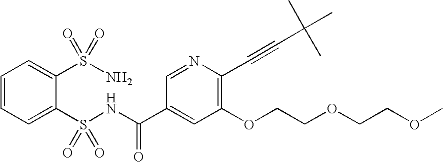

6-Chloro-5-(2-(2-methoxyethoxy)ethoxy)-N-(2-sulfamoylphenylsulfonyl)nicotinamide (0.162 g, 0.33 mmol), diisopropyl 3,3-dimethylbut-1-ynylboronate (0.155 mL, 0.66 mmol) and 1,1′-bis(diphenylphosphino)ferrocene-palladium dichloride (0.027 g, 0.03 mmol) were dissolved in N,N-dimethylformamide under an atmosphere of argon followed by addition of aqueous sodium carbonate (0.492 mL, 0.98 mmol). The reaction mixture was heated in a microwave at 120° C. for 40 min under an atmosphere of argon and was then partitioned between water and ethyl acetate. The aqueous phase was acidified with aqueous hydrochloric acid (2 M) and extracted with ethyl acetate. The organic phase were dried over magnesium sulfate and the solvent was evaporated. Purification by preparative HPLC gave 0.028 g (16% yield) of the title compound.

¹H NMR (500 MHz, CD₃OD), δ ppm 8.50 (s, 1H), 8.46 (dd, 1H), 8.25 (dd, 1H), 7.94 (s, 1H) 7.82 (dd, 2H), 4.24-4.30 (m, 2H), 3.90 (dd, 2H), 3.71-3.78 (m, 2H), 3.52-3.58 (m, 2H), 3.33 (s, 3H), 1.35 (s, 9H); MS (ESI) m/z 538 [M−1]⁻.

### a) 6-Chloro-5-(2-(2-methoxyethoxy)ethoxy)-N-(2-sulfamoylphenylsulfonyl)nicotinamide

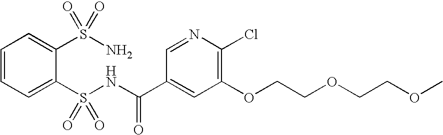

6-Chloro-5-(2-(2-methoxyethoxy)ethoxy)nicotinic acid (0.516 g, 1.87 mmol) N-(3-dimethylaminopropyl)-N′-ethylcarbodiimide hydrochloride (0.466 g, 2.43 mmol) and 4-dimethylaminopyridine (0.297 g, 2.43 mmol) were added to a solution of benzene-1,2-disulfonamide (0.420 g, 1.78 mmol)in N,N-dimethylformamide (20 mL) at room temperature and the reaction mixture was stirred over night. The reaction mixture was partitioned between water and ethyl acetate. The aqueous phase was acidified with aqueous hydrochloric acid (2 M) and extracted with ethyl acetate, the organic phase was dried over magnesium sulfate and the solvent was evaporated. Purification by column chromatography, using ethyl acetate/methanol (100:1+1% triethyamine) as the eluent, gave 0.74 g (81% yield) of the title compound.

MS (ESI) m/z 492, 494, 496 [M−1]⁻.

### b) 6-Chloro-5-(2-(2-methoxyethoxy)ethoxy)nicotinic Acid

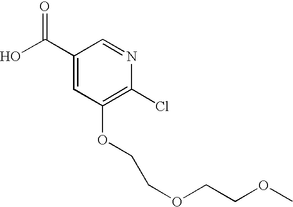

2-(2-Methoxyethoxy)ethanol (0.333 mL, 2.80 mmol), triphenylphosphine (0.734 g, 2.80 mmol) and diisopropyl azodicarboxylate (0.551 mL, 2.80 mmol) were added to a solution of methyl 6-chloro-5-hydroxynicotinate (0.350 g, 1.87 mmol) in tetrahydrofuran (15 mL). The reaction mixture was stirred at room temperature over night. A solution of lithium hydroxide monohydrate (0.134 g, 5.60 mmol) in water (2 mL) was added and the reaction mixture was stirred for 3 days at room temperature. The aqueous phase was acidified with aqueous hydrochloric acid (2 M) and extracted with ethyl acetate. The organic phase was dried over magnesium sulfate and the solvent was evaporated to give the title compound.

MS (ESI) m/z 276, 278, 280 [M+1]⁺.

### c) Methyl 6-chloro-5-hydroxynicotinate

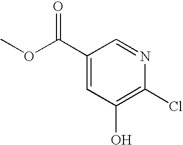

N-Chlorosuccinimide (2.093 g, 15.67 mmol) was added to a solution of methyl 5-hydroxynicotinate (2.0 g, 13.06 mmol) in N,N-dimethylformamide (20 mL). The reaction mixture was heated at 80° C. over night and the solvent was evaporated. Purification by column chromatography, using heptane/ethyl acetate (3: 1-1:1) as the eluent, gave 0.957 g of the title compound.

MS (ESI) m/z 186, 188, 190 [M−1]⁻.

### EXAMPLE 135

### 6-(Benzofuran-2-yl)-5-(2-(2-methoxyethoxy)ethoxy)-N-(2-sulfamoylphenylsulfonyl)nicotinamide

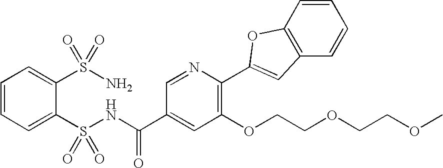

The title compound was synthesized as described for Example 134 in 31% yield, starting from 6-chloro-5-(2-(2-methoxyethoxy)ethoxy)-N-(2-sulfamoylphenylsulfonyl)nicotinamide and benzofuran-2-ylboronic acid. The reaction mixture was heated in a microwave at 120° C. for 20 min.

¹H NMR (500 MHz, CD₃OD) δ ppm 8.60 (d, 1H), 8.34-8.46 (m, 1H) 8.13-8.24 (m, 1H) 7.98 (d, 1H), 7.84 (d, 1H), 7.70-7.80 (m, 2H), 7.61 (d, 1H), 7.51 (d, 1H), 7.30 (td, 1H), 7.14-7.23 (m, 1H), 4.29-4.42 (m, 2H), 3.86-3.98 (m, 2H), 3.63-3.74 (m, 2H), 3.45-3.56 (m, 2H), 3.23-3.27 (m, 3H); MS (ESI) m/z 574 [M−1]⁻.

### EXAMPLE 136

### 6-(Cyclopentylethynyl)-5-(2-(2-methoxyethoxy)ethoxy)-N-(2-sulfamoylphenylsulfonyl)nicotinamide

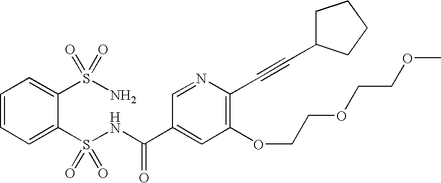

Ethynylcyclopentane (0.054 g, 0.58 mmol) tetrakis(triphenylphosphine)palladium(0) (0.044 g, 0.04 mmol) and triethylamine (1.608 mL, 11.54 mmol) were added to a solution of 6-chloro-5-(2-(2-methoxyethoxy)ethoxy)-N-(2-sulfamoylphenylsulfonyl)nicotinamide (0.190 g, 0.38 mmol) in N,N-dimethylformamide (8 mL) under an atmosphere of argon. The reaction mixture was stirred at room temperature for 5 min, copper(I) iodide (10.99 mg, 0.06 mmol) was added and the reaction mixture was heated at 65° C. over night. The reaction mixture was partitioned between water and ethyl acetate. The aqueous phase was acidified with aqueous hydrochloric acid (2 M) and extracted with ethyl acetate. The organic phase was dried over magnesium sulfate and the solvent was evaporated. Purification by preparative HPLC gave 0.063 g (30% yield) of the title compound.

¹H NMR (500 MHz, CD₃OD) δ ppm 8.43-8.55 (m, 2H), 8.21-8.31 (m, 1H), 7.93 (s, 1H), 7.77-7.89 (m, 2H), 4.23-4.35 (m, 2H), 3.86-3.96 (m, 2H), 3.74 (dd, 2H), 3.54 (dd, 2H), 3.33 (s, 3H), 2.91-3.01 (m, 1H), 1.96-2.08 (m, 2H), 1.71-1.87 (m, 4H), 1.59-1.70 (m, 2H); MS (ESI) m/z 550 [M−1]⁻.

### EXAMPLE 137

### 6-(Cyclopentylethynyl)-5-methoxy-N-(2-sulfamoylphenylsulfonyl)nicotinamide

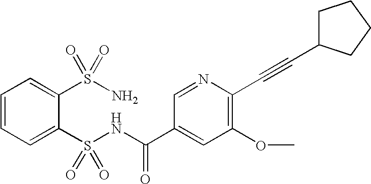

The title compound was synthesized as described for Example 136 in 34% yield, starting from 6-chloro-5-methoxy-N-(2-sulfamoylphenylsulfonyl)nicotinamide and ethynylcyclopentane.

¹H NMR (500 MHz, CD₃OD) δ ppm 8.31-8.46 (m, 2H) 8.12-8.20 (m, 1H) 7.81 (s, 1H) 7.70-7.78 (m, 2H) 3.84 (s, 3H) 2.84 (t, 1H) 1.82-2.03 (m, 2H) 1.59-1.79 (m, 4H) 1.44-1.59 (m, 2H); MS (ESI) m/z 462 [M−1]⁻.

### a) 6-Chloro-5-methoxy-N-(2-sulfamoylphenylsulfonyl)nicotinamide

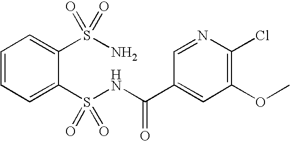

The title compound was synthesized as described for Example 127a) in 62% yield, starting from 6-chloro-5-methoxynicotinic acid.

¹H NMR (500 MHz, CD₃OD) δ ppm 8.45-8.57 (m, 1H) 8.38 (d, 1H) 8.20-8.34 (m, 1H) 7.81-7.98 (m, 3H) 3.98 (s, 3H); MS (ESI) m/z 404, 406, 408 [M−1]⁻.

### b) 6-Chloro-5-methoxynicotinic Acid

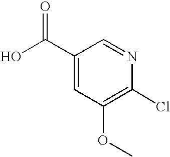

The title compound was synthesized as described for Example 127b) in 74% yield, starting from methyl 6-chloro-5-methoxynicotinate.

¹H NMR (500 MHz, CD₃OD), δ ppm 8.51 (d, 1H), 7.94 (d, 1H), 4.00 (s, 3H); MS (ESI) m/z 186, 188, 190 [M−1]⁻.

### c) Methyl 6-chloro-5-methoxynicotinate

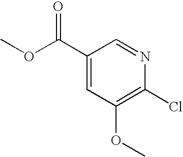

Potassium carbonate (2.59 g, 18.71 mmol) and iodomethane (1.031 mL, 16.55 mmol) were added to a solution of methyl 6-chloro-5-hydroxynicotinate (2.7 g, 14.4 mmol) in N,N-dimethylformamide (40 mL) at room temperature and the resulting mixture was stirred over night. The reaction mixture was partitioned between water and ethyl acetate. The organic phase was washed with water, dried over magnesium sulfate and the solvent was evaporated to give 2.48 g (85% yield) of the title compound.

MS (ESI) m/z 202, 204, 206 [M+1]⁺.

### EXAMPLE 138

### 6-(Cyclohexylethynyl)-5-methoxy-N-(2-sulfamoylphenylsulfonyl)nicotinamide

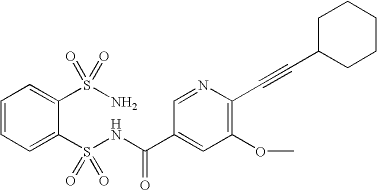

The title compound was synthesized as described for Example 136 in 11% yield, starting from 6-chloro-5-methoxy-N-(2-sulfamoylphenylsulfonyl)nicotinamide and ethynylcyclohexane.

¹H NMR (500 MHz, CD₃OD) δ ppm 1.36-1.48 (m, 3H) 1.51-1.67 (m, 3H) 1.76-1.86 (m, 2H) 1.86-1.97 (m, 2H) 2.63-2.78 (m, 1H) 3.92 (s, 3H) 7.63-7.75 (m, 2H) 8.00 (d, 1H) 8.21 (dd, 1H) 8.30 (dd, 1H) 8.59 (d, 1H); MS (ESI) m/z 476 [M−1]⁻.

### EXAMPLE 139

### 5-Methoxy-N-(2-sulfamoylphenylsulfonyl)-6-((4-(trifluoromethyl)phenyl)ethynyl)nicotinamide

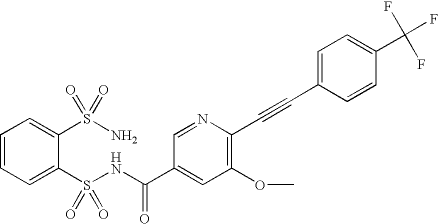

The title compound was synthesized as described for Example 136 in 28% yield, starting from 6-chloro-5-methoxy-N-(2-sulfamoylphenylsulfonyl)nicotinamide and 1-ethynyl-4-(trifluoromethyl)benzene.

¹H NMR (500 MHz, CD₃OD) δ ppm 8.67 (d, 1H) 8.37 (dd, 1H) 8.20 (dd, 1H) 8.10 (d, 1H) 7.76-7.84 (m, 2H) 7.63-7.76 (m, 4H) 4.01 (s, 3H); MS (ESI) m/z 538 [M−1]⁻.

### EXAMPLE 140

### N-(3-Dimethylaminopropyl)-N′-ethylcarbodiimide Hydrochloride

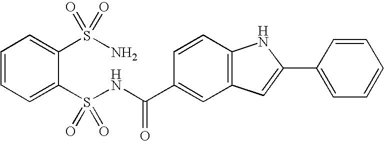

2-Phenyl-1H-indole-5-carboxylic acid (0.080 g, 0.34 mmol), N-(3-dimethylaminopropyl)-N′-ethylcarbodiimide hydrochloride (0.097 g, 0.51 mmol) and 4-dimethylaminopyridine (0.062 g, 0.51 mmol) were added to a solution of benzene-1,2-disulfonamide (0.080 g, 0.34 mmol) in N,N-dimethylformamide (30 mL) at room temperature and the reaction mixture was stirred over night. Water was added and the solution was extracted with ethyl acetate. The aqueous phase was acidified with 2 M aqueous hydrochloric acid and extracted with ethyl acetate. The organic phase was dried over magnesium sulfate and the solvent was evaporated. Purification by preparative HPLC gave 0.056 g (37% yield) of the title compound.

¹H NMR (500 MHz, CD₃OD) δ ppm 8.49 (dd, 1H) 8.27 (dd, 1H) 8.15-8.22 (m, 1H) 7.83-7.91 (m, 2H) 7.81 (d, 2H) 7.64 (dd, 1H) 7.39-7.50 (m, 3H) 7.27-7.39 (m, 1H) 6.95 (s, 1H); MS (ESI) m/z 454 [M−1]⁻.

### a) 2-Phenyl-1H-indole-5-carboxylic Acid

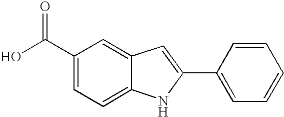

A solution of lithium hydroxide monohydrate (0.057 g, 2.36 mmol) in water (2 mL) was added to a solution of methyl 2-phenyl-1H-indole-5-carboxylate (0.198 g, 0.79 mmol) in tetrahydrofuran (10 mL) at room temperature and the resulting mixture was stirred for 5 days. Additional amounts of lithium hydroxide monohydrate (0.057 g, 2.36 mmol) dissolved in water (2 mL) was added and the reaction mixture was stirred over night. The reaction mixture was partitioned between water and ethyl acetate. The aqueous phase was acidified with aqueous hydrochloric acid (2 M) and extracted with ethyl acetate. The organic phase was dried over magnesium sulfate and the solvent was evaporated to give 0.085 g (46% yield) of the title compound.

MS (ESI) m/z 236 [M−1]⁻.

### b) Methyl 2-phenyl-1H-indole-5-carboxylate

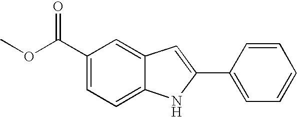

Methyl 3-iodo-4-(2,2,2-trifluoroacetamido)benzoate (0.600 g, 1.61 mmol), ethynylbenzene (0.265 mL, 2.41 mmol), 1,1,3,3-tetramethylguanidine (2.020 mL, 16.08 mmol), bis(triphenylphosphine)palladium(II) chloride (0.113 g, 0.16 mmol) and copper(I) iodide (0.03 1g, 0.16 mmol) were dissolved in N,N-dimethylformamide (15 mL), the resulting mixture was stirred at 50° C. under an atmosphere of argon over night and the solvent was evaporated. Purification by column chromatography, using heptane/ethyl acetate (7:1 to 4:1) as the eluent, gave 0.202 g (50% yield) of the title compound.

¹H NMR (400 MHz, CDCl₃) δ ppm 3.92-3.98 (m, 3H) 6.92 (dd, 1H) 7.33-7.40 (m, 1H) 7.42 (d, 1H) 7.48 (t, 2H) 7.69 (d, 2H) 7.92 (dd, 1H) 8.40 (d, 1H) 8.55 (br. s., 1H);

MS (ESI) m/z 250[M−1]⁻.

### c) Methyl 3-iodo-4-(2,2,2-trifluoroacetamido)benzoate

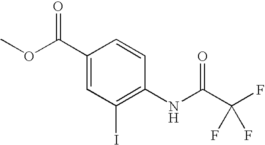

A solution of methyl 4-amino-3-iodobenzoate (1.0 g, 3.61 mmol) and triethylamine (1.003 mL, 7.22 mmol) in dichloromethane (20 mL) was added dropwise to a cooled (0° C.) solution of trifluoroacetic anhydride (1.275 mL, 9.02 mmol) in dichloromethane (5 mL). The cooling was removed, the mixture was stirred at room temperature for 3 hours, poured into ice-water and extracted with dichloromethane. The organic phase was dried over sodium sulfate and the solvent was evaporated. Purification by column chromatography, using heptane/ethyl acetate (4:1) as the eluent, gave 1.23 g (91% yield) of the title compound.

¹H NMR (500 MHz, CD₃OD) δ ppm 8.54 (d, 1H) 8.07 (dd, 1H) 7.57 (d, 1H) 3.93 (s, 3H); MS (ESI) m/z 372 [M−1]⁻.

### a) 1-(2-Methoxyethyl)-2-phenyl-1H-indole-5-carboxylic Acid

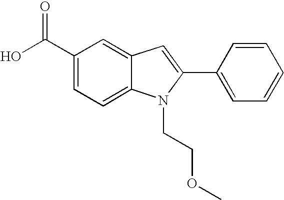

A solution of lithium hydroxide (0.024 g, 0.99 mmol) in water (2 mL) was added to a solution of methyl 1-(2-methoxyethyl)-2-phenyl-1H-indole-5-carboxylate (0.102 g, 0.33 mmol) in tetrahydrofuran (6 mL) at room temperature and the reaction mixture was stirred over the weekend. Another 16 equivalents of lithium hydroxide was added and the reaction was stirred for 3 days. The reaction was partitioned between water and ethyl acetate, the aqueous phase was acidified with 2 M aqueous hydrochloric acid and extracted with ethyl acetate. The organic phase was dried over magnesium sulfate and the solvent was evaporated to give 0.029 g (30% yield) of the title compound.

MS (ESI) m/z 294 [M−1]⁻.

### b) Methyl 1-(2-methoxyethyl)-2-phenyl-1H-indole-5-carboxylate

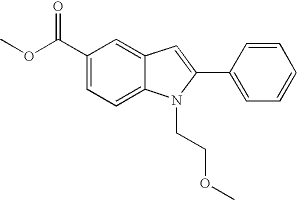

Potassium hydroxide (0.041 g, 0.74 mmol) was added to a solution of methyl 2-phenyl-1H-indole-5-carboxylate (0.084 g, 0.33 mmol) and 2-bromoethyl methyl ether (0.035 mL, 0.37 mmol) in N,N-dimethylformamide (5 mL) at room temperature and the reaction was stirred over night. 2-Bromoethyl methyl ether (0.035 mL, 0.37 mmol) was added and the reaction mixture was stirred for another 2 hours. More 2-bromoethyl methyl ether (0.035 mL, 0.37 mmol) was added and the mixture was stirred for another 1.5 hours. The reaction was partitioned between water and ethyl acetate, the organic phase was dried over magnesium sulfate and the solvent was evaporated to give the title compound.

MS (ESI) m/z 310 [M+1]⁺.

### EXAMPLE 141

### 1-(2-Methoxyethyl)-2-phenyl-N-(2-sulfamoylphenylsulfonyl)-1H-indole-5-carboxamide

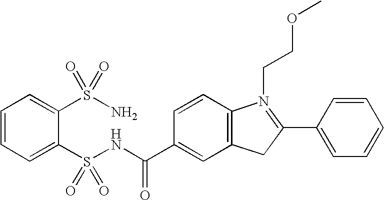

The title compound was synthesized as described for Example 140 in 26% yield, starting from 1-(2-methoxyethyl)-2-phenyl-1H-indole-5-carboxylic acid.

¹H NMR (500 MHz, CD₃OD) δ ppm 8.43-8.53 (m, 1H) 8.25-8.32 (m, 1H) 8.21 (d, 1H) 7.78-7.90 (m, 2H) 7.73 (dd, 1H) 7.53-7.60 (m, 3H) 7.47-7.53 (m, 2H) 7.39-7.47 (m, 1H) 6.62 (s, 1H) 4.39 (t, 2H) 3.57 (t, 2H) 3.11 (s, 3H); MS (ESI) m/z 512 [M−1]⁻.

### EXAMPLE 142

### 6-(cyclopropylethynyl)-5-isopropoxy-N-(2-sulfamoylphenylsulfonyl)nicotinamide

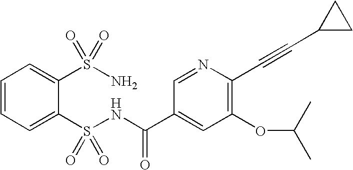

The title compound was synthesized as described for Example 130 in 37% yield, starting from 6-chloro-5-isopropoxy-N-(2-sulfamoylphenylsulfonyl)nicotinamide and ethynylcyclopropane.

¹H NMR (500 MHz, CD₃OD) δ ppm 8.41-8.49 (m, 2H) 8.25 (dd, 1H) 7.90 (s, 1H) 7.81 (dd, 2H) 4.68-4.78 (m, 1H) 1.53-1.61 (m, 1H) 1.38 (s, 3H) 1.36 (s, 3H) 0.96-1.03 (m, 2H) 0.81-0.89 (m, 2H); MS (ESI) m/z 462 [M−1]⁻.

### a) 6-Chloro-5-isopropoxy-N-(2-sulfamoylphenylsulfonyl)nicotinamide

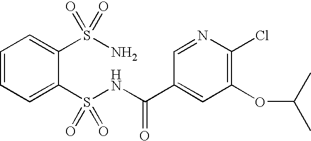

The title compound was synthesized as described for Example 127a in 54% yield, starting from 6-chloro-5-isopropoxynicotinic acid.

¹H NMR (500 MHz, CD₃OD) δ ppm 8.51 (dd, 1H) 8.36 (d, 1H) 8.26-8.32 (m, 1H) 7.84-7.94 (m, 3H) 4.74-4.85 (m, 1H) 1.41-1.45 (m, 3H) 1.40 (s, 3H); MS (ESI) m/z 432, 434, 436 [M−1]⁻.

### b) 6-Chloro-5-isopropoxynicotinic Acid

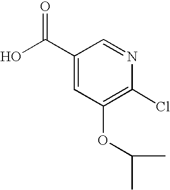

The title compound was synthesized as described for Example 127b) in 80% yield, starting from methyl 6-chloro-5-isopropoxynicotinate. ¹H NMR (500 MHz, CDCl₃) δ ppm 8.67 (d, 1H) 7.80 (d, 1H) 4.66-4.73 (m, 1H) 1.46 (s, 3H) 1.45 (s, 3H); MS (ES) m/z 214, 216, 218 [M−1]⁻.

### c) Methyl 6-chloro-5-isopropoxynicotinate

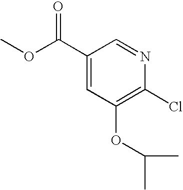

The title compound was synthesized as described for Example 127c) in 88% yield, starting from methyl 6-chloro-5-methoxynicotinate.

¹H NMR (500 MHz, CDCl₃) δ ppm 8.57 (d, 1H) 7.76 (d, 1H) 4.58-4.78 (m, 1H) 3.97 (s, 3H) 1.44 (s, 3H) 1.43 (s, 3H); GC MS (EI) m/z 229 [M]⁺.

### EXAMPLE 143

### 6-(Cyclopentylethynyl)-5-isopropoxy-N-(2-sulfamoylphenylsulfonyl)nicotinamide

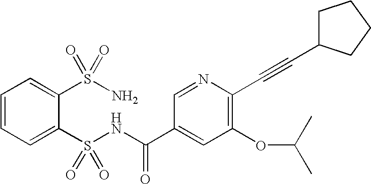

Ethynylcyclopentane (0.039 g, 0.41 mmol), tetrakis(triphenylphosphine)palladium(0) (0.048 g, 0.04 mmol) and triethylamine (1.735 mL, 12.45 mmol) was added to a solution of 6-chloro-5-isopropoxy-N-(2-sulfamoylphenylsulfonyl)nicotinamide (0.180 g, 0.41 mmol) in N,N-dimethylformamide (8 mL) under an atmosphere of argon. The reaction mixture was stirred at room temperature for 5 min, copper(I) iodide (0.012 g, 0.06 mmol) was added and the reaction mixture was heated at 65° C. over night. The reaction mixture was partitioned between water and ethyl acetate. The aqueous phase was acidified with aqueous hydrochloric acid (2 M) and extracted with ethyl acetate. The organic phase was dried over magnesium sulfate and the solvent was evaporated. Purification by preparative HPLC gave 0.033 g (16% yield) of the title compound.

¹H NMR (500 MHz, CD₃OD) δ ppm 8.50 (d, 1H) 8.39-8.45 (m, 1H) 8.20-8.26 (m, 1H) 7.94 (d, 1H) 7.72-7.82 (m, 2H) 4.69-4.77 (m, 1H) 2.90-3.02 (m, 1H) 1.97-2.07 (m, 2H) 1.73-1.89 (m, 4H) 1.62-1.73 (m, 2H) 1.38 (s, 3H) 1.37 (s, 3H); MS (ESI) m/z 490 [M−1]⁻.

### EXAMPLE 144

### 6-(Cyclohexylethynyl)-5-isopropoxy-N-(2-sulfamoylphenylsulfonyl)nicotinamide

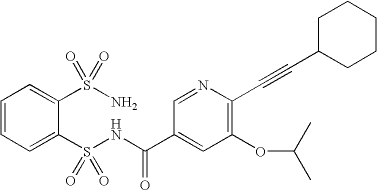

The title compound was synthesized as described for Example 127a) in 14% yield, starting from ethynylcyclohexane but the reaction mixture was heated at 65° C. over the weekend.

¹H NMR (500 MHz, CD₃OD) δ ppm 8.42-8.52 (m, 2H) 8.20-8.30 (m, 1H) 7.91 (s, 1H) 7.77-7.85 (m, 2H) 4.74 (dt, 1H) 2.69-2.81 (m, 1H) 1.83 (d, 4H) 1.50-1.68 (m, 3H) 1.40-1.48 (m, 3H) 1.38 (s, 3H) 1.36 (s, 3H); MS (ESI) m/z 504 [M−1]⁻.

### EXAMPLE 145

### 4-(Benzofuran-2-yl)-3-(3-methoxy-3-methylbutoxy)-N-(2-sulfamoylphenylsulfonyl)benzamide

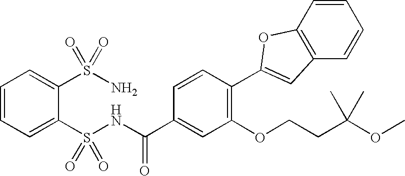

4-bromo-3-(3-methoxy-3-methylbutoxy)-N-(2-sulfamoylphenylsulfonyl)benzamide (0.250 g, 0.47 mmol),benzofuran-2-ylboronic acid (0.151 g, 0.93 mmol) and 1,1′-bis(diphenylphosphino)ferrocene-palladium dichloride (0.038 g, 0.05 mmol) were dissolved in N,N-dimethylformamide (3 mL) under an atmosphere of argon. Aqueous sodium carbonate (0.700 mL, 1.40 mmol) was added, the reaction mixture was heated in a microwave at 120° C. for 20 min under an atmosphere of argon and was then partitioned between water and ethyl acetate. The aqueous phase was acidified with aqueous hydrochloric acid (2 M) and extracted with ethyl acetate. The organic phase were dried over magnesium sulfate and the solvent was evaporated. Purification by preparative HPLC gave 0.181 g (68% yield) of the title compound.

¹H NMR (500 MHz, CD₃OD) δ ppm 8.45-8.53 (m, 1H) 8.28 (dd, 1H) 8.09 (d, 1H) 7.80-7.91 (m, 2H) 7.71 (s, 1H) 7.64 (d, 1H) 7.58 (dd, 1H) 7.55 (s, 1H) 7.52 (d, 1H) 7.28-7.36 (m, 1H) 7.23 (t, 1H) 4.38 (t, 2H) 3.29 (s, 3H) 2.24 (t, 2H) 1.33 (s, 6H); MS (ESI) m/z 571 [M−1]⁻.

### a) 4-Bromo-3-(3-methoxy-3-methylbutoxy)-N-(2-sulfamoylphenylsulfonyl)benzamide

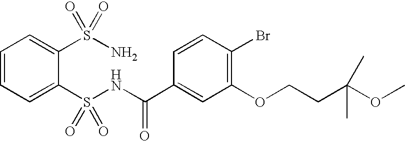

The title compound was synthesized as described for Example 127a) in 75% yield, starting from 4-bromo-3-(3-methoxy-3-methylbutoxy)benzoic acid.

¹H NMR (500 MHz, CD₃OD) δ ppm 8.33 (d, 1H) 8.21 (dd, 1H) 7.64-7.77 (m, 3H) 7.52 (d, 1H) 7.44 (dd, 1H) 4.19 (t, 2H) 3.24 (s, 3H) 2.06 (t, 2H) 1.22-1.35 (m, 6H); MS (ESI) m/z 533, 535 [M−1]⁻.

### b) 4-Bromo-3-(3-methoxy-3-methylbutoxy)benzoic Acid

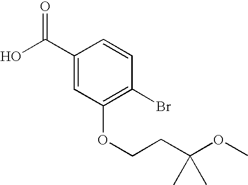

The title compound was synthesized as described for Example 127b) in 99% yield, starting from methyl 4-bromo-3-(3-methoxy-3-methylbutoxy)benzoate.

¹H NMR (500 MHz, CDCl₃) δ ppm 7.69 (d, 1H) 7.64 (d, 1H) 7.57 (dd, 1H) 4.25 (t, 2H) 3.27 (s, 3H) 2.13 (t, 2H) 1.31 (s, 6H); MS (ESI) m/z 315, 317 [M−1]⁻.

### c) Methyl 4-bromo-3-(3-methoxy-3-methylbutoxy)benzoate

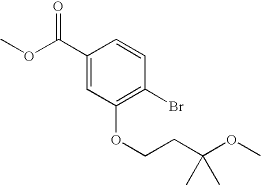

The title compound was synthesized as described for Example 127c) in 98% yield, starting from methyl 4-bromo-3-hydroxybenzoate.

¹H NMR (500 MHz, CDCl₃) δ ppm 7.61 (d, 1H) 7.56 (d, 1H) 7.50 (dd, 1H) 4.19 (t, 2H) 3.93 (s, 3H) 3.25 (s, 3H) 2.10 (t, 2H) 1.29 (s, 6H); GC MS (EI) m/z 330, 332 [M]⁺.

### EXAMPLE 146

### 4-(Cyclopentylethynyl)-3-fluoro-N-(2-sulfamoylphenylsulfonyl)benzamide

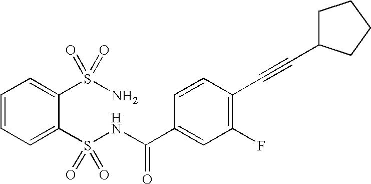

A mixture of 4-bromo-3-fluoro-N-(2-sulfamoylphenylsulfonyl)benzamide (131 mg, 0.30 mmol), cyclopentylacetylene (0.035 mL, 0.30 mmol), copper(I) iodide (5.7 mg, 0.030 mmol), bis(triphenylphosphine)palladium(II) chloride (21.1 mg, 0.030 mmol) and diisopropylamine (0.13 mL, 0.90 mmol) in N,N-dimethylformamide (2 mL) under an atmosphere of argon was heated at 100° C. for 2 hours in a microwave. The reaction mixture was partitioned between ethyl acetate and aqueous hydrochloric acid. The organic phase was dried over magnesium sulfate and the solvent was evaporated. Purification by preparative HPLC gave 0.070g (52% yield) of the title compound.

¹H NMR (CD₃OD) δ ppm 8.34-8.39 (m, 1H) 8.14-8.18 (m, 1H) 7.73-7.77 (m, 2H) 7.49-7.55 (m, 2H) 7.35 (t, 1H) 2.77-2.85 (m, 1H) 1.87-1.97 (m, 2H) 1.49-1.75 (m, 6H); MS (ESI) m/z 449 [M−1]⁻.

### EXAMPLE 147

### 6-(Benzofuran-2-yl)-5-chloro-N-(2-sulfamoylphenylsulfonyl)nicotinamide

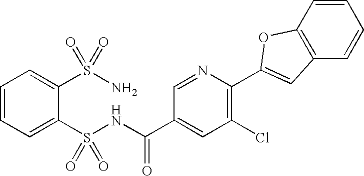

A mixture of 5,6-dichloro-N-(2-sulfamoylphenylsulfonyl)nicotinamide (164 mg, 0.40 mmol), 2-benzofuranboronic acid (84 mg, 0.52 mmol), 1,1′-bis(diphenylphosphino)ferrocene-palladium dichloride (32.9 mg, 0.040 mmol), N,N-dimethylformamide (4 mL) and sodium carbonate (2 M, 0.60 mL, 1.20 mmol) under an atmosphere of argon was heated at 120° C. for 0.5 hour in a microwave. The reaction mixture was partitioned between ethyl acetate and diluted hydrochloric acid, the organic phase was dried over magnesium sulfate and the solvent was evaporated. Purification by preparative HPLC gave 0.047 g (24% yield) of the title compound.

¹H NMR (DMSO-d₆) δ ppm 8.96 (d, 1H) 8.37 (s, 1H) 8.26 (dd, 3.70 Hz, 1H) 8.05 (dd, 3.39 Hz, 1H) 7.88 (s, 1H) 7.73-7.80 (m, 3H) 7.66 (d, 1H) 7.37-7.50 (m, 3H) 7.26-7.31 (m, 1H); MS (ESI) m/z 490 [M−1]⁻.

### EXAMPLE 148

### 5-Chloro-6-(cyclopentylethynyl)-N-(2-sulfamoylphenylsulfonyl)nicotinamide

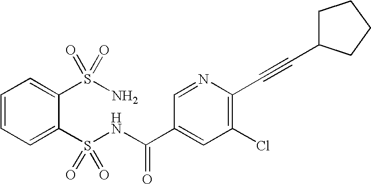

The title compound was synthesized as described for Example 146 in 34% yield, starting from 5,6-dichloro-N-(2-sulfamoylphenylsulfonyl)nicotinamide. Purification by preparative HPLC.

¹H NMR (DMSO-d₆) δ ppm 8.76 (d, 1H) 8.20-8.29 (m, 2H) 8.00-8.08 (m, 1H) 7.73-7.81 (m, 2H) 7.41 (br. s, 2H) 2.89-3.00 (m, 1H) 1.90-1.99 (m, 2H) 1.48-1.71 (m, 6H); MS (ESI) m/z 466 [M−1]⁻.

### a) 5,6-Dichloro-N-(2-sulfamoylphenylsulfonyl)nicotinamide

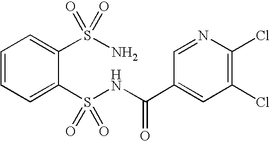

The title compound was synthesized as described for Example 73a) in 88% yield, starting from 5,6-dichloronicotinic acid.

¹H NMR (DMSO-d₆) δ ppm 8.71-8.77 (m, 1H) 8.36-8.43 (m, 1H) 8.23-8.31 (m, 1H) 8.05-8.11 (m, 1H) 7.72-7.81 (m, 2H) 7.43-7.50 (m, 2H); MS (ESI) m/z 408 [M−1]⁻.

### EXAMPLE 149

### 5-Chloro-6-(3,3-dimethylbut-1-ynyl)-N-(2-sulfamoylphenylsulfonyl)nicotinamide

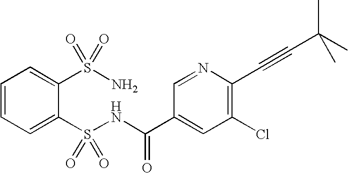

The title compound was synthesized as described for Example 146 in 34% yield, starting from 5,6-dichloro-N-(2-sulfamoylphenylsulfonyl)nicotinamide and 3,3-dimethylbut-1-yne. Purification by preparative HPLC.

¹H NMR (DMSO-d₆) δ ppm 8.83 (d, 1H) 8.27-8.35 (m, 2H) 8.07-8.15 (m, 1H) 7.79-7.88 (m, 2H) 7.48 (br. s., 2H) 1.34 (s, 9H); MS (ESI) m/z 454 [M−1]⁻.

### EXAMPLE 150

### 4-(Benzofuran-2-yl)-N-(2-sulfamoylphenylsulfonyl)-2-(trifluoromethyl)benzamide

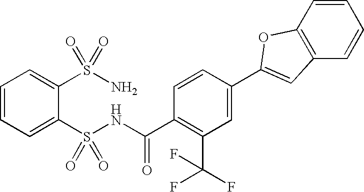

The title compound was synthesized as described for Example 147 in 39% yield, starting from 4-iodo-N-(2-sulfamoylphenylsulfonyl)-2-(trifluoromethyl)benzamide.

¹H NMR (DMSO-d₆) δ ppm 8.32-8.40 (m, 1H) 8.16-8.31 (m, 3H) 7.85-7.99 (m, 2H) 7.76-7.85 (m, 2H) 7.67-7.76 (m, 2H) 7.36-7.46 (m, 3H) 7.27-7.36 (m, 1H); MS (ESI) m/z 523 [M−1]⁻.

### EXAMPLE 151

### 4-(3,3-Dimethylbut-1-ynyl)-N-(2-sulfamoylphenylsulfonyl)-2-(trifluoromethyl)benzamide

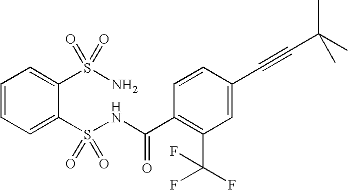

The title compound was synthesized as described for Example 146 in 22% yield, starting from 4-iodo-N-(2-sulfamoylphenylsulfonyl)-2-(trifluoromethyl)benzamide and 3,3-dimethylbut-1-yne (1.5 equiv.). Purification by preparative HPLC.

¹H NMR (DMSO-d₆) δ ppm 8.34 (d, 1H) 8.18 (d, 1H) 7.85-7.96 (m, 2H) 7.67-7.73 (m, 2H) 7.62-7.66 (m, 1H) 7.39 (s, 2H) 1.31 (s, 9H); MS (ESI) m/z 487 [M−1]⁻.

### a) 4-Iodo-N-(2-sulfamoylphenylsulfonyl)-2-(trifluoromethyl)benzamide

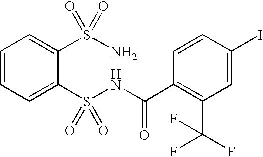

The title compound was synthesized as described for Example 73a) in 14% yield, starting from 4-iodo-2-(trifluoromethyl)benzoic acid.

MS (ESI) m/z 533 [M−1]⁻.

### b) 4-Iodo-2-(trifluoromethyl)benzoic Acid

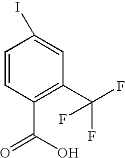

A solution of sodium nitrite (0.37 g, 5.36 mmol) in water (1.5 mL) was added dropwise to a cooled (0° C.) suspension of 4-amino-2-(trifluoromethyl)benzoic acid (1 g, 4.9 mmol) in hydrochloric acid (37%, 2 mL) and ice (3 g). After 20 min at 0° C. the reaction mixture was slowly added to a stirred solution of potassium iodide (8.09 g, 48.8 mmol) in water (8 mL) at 0° C. The resulting mixture was stirred at room temperature over night, dichloromethane and sodium sulfite (2.52 g, 20.0 mmol) was added, the organic phase was collected, dried over magnesium sulfate and the solvent was evaporated to give the title compound.

¹H NMR (DMSO-d₆) δ ppm 13.78 (s, 1H) 8.11-8.24 (m, 2H) 7.49-7.66 (m, 1H); MS (ESI) m/z 315 [M−1]⁻.

### EXAMPLE 152

### 4-(Benzofuran-2-yl)-2,6-difluoro-N-(2-sulfamoylphenylsulfonyl)benzamide

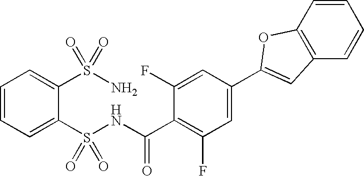

The title compound was synthesized as described for Example 147 in 26% yield, starting from 4-bromo-2,6-difluoro-N-(2-sulfamoylphenylsulfonyl)benzamide.

¹H NMR (DMSO-d₆) δ ppm 8.19-8.28 (m, 1H) 8.05-8.13 (m, 1H) 7.75-7.86 (m, 2H) 7.57-7.69 (m, 5H) 7.33 (dt, 1H) 7.20-7.30 (m, 3H); MS (ESI) m/z 491 [M−1]⁻.

### a) 4-Bromo-2,6-difluoro-N-(2-sulfamoylphenylsulfonyl)benzamide

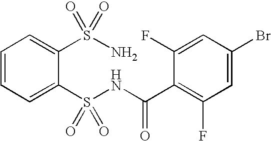

The title compound was synthesized as described for Example 73a) in 27% yield, starting from 4-bromo-2,6-difluorobenzoic acid.

MS (ESI) m/z 453, 455 [M−1]⁻.

### EXAMPLE 153

### 4-(Cyclopentylethynyl)-2,6-difluoro-N-(2-sulfamoylphenylsulfonyl)benzamide

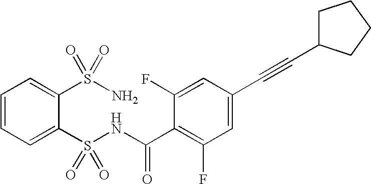

The title compound was synthesized as described for Example 146 in 43% yield, starting from 4-bromo-2,6-difluoro-N-(2-sulfamoylphenylsulfonyl)benzamide. Purification by preparative HPLC.

¹H NMR (DMSO-d₆) δ ppm 8.23-8.31 (m, 1H) 8.13-8.19 (m, 1H) 7.83-7.94 (m, 2H) 7.32 (s, 2H) 7.19 (d, 2H) 2.85-2.94 (m, 1H) 1.93-2.03 (m, 2H) 1.53-1.77 (m, 6H);

MS (ESI) m/z 467 [M−1]⁻.

### EXAMPLE 154

### 4-(Benzofuran-2-yl)-3-(3-hydroxy-3-methylbut-1-ynyl)-N-(2-sulfamoylphenylsulfonyl)benzamide

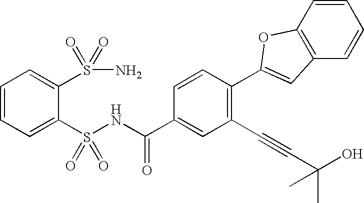

The title compound was synthesized as described for Example 146 in 34% yield, starting from 4-(benzofuran-2-yl)-3-bromo-N-(2-sulfamoylphenylsulfonyl)benzamide and 2-Methyl-3-butyn-2-ol (3 equiv.). Purification by preparative HPLC.

¹H NMR (DMSO-d₆) δ ppm 8.33 (br s, 1H) 8.04-8.20 (m, 3H) 7.93-8.01 (m, 2H) 7.87 (br s, 2H) 7.74 (d, 1H) 7.67 (d, 1H) 7.47 (s, 2H) 7.38-7.44 (m, 1H) 7.32 (t, 1H) 1.59 (s, 6H); MS (ESI) m/z 537 [M−1]⁻.

### EXAMPLE 155

### 4-(Benzofuran-2-yl)-3-bromo-N-(2-sulfamoylphenylsulfonyl)benzamide

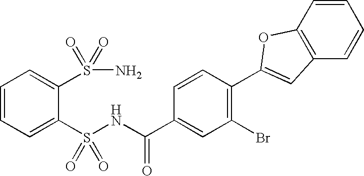

The title compound was synthesized as described for Example 147 in 33% yield, starting from 3-bromo-4-iodo-N-(2-sulfamoylphenylsulfonyl)benzamide and using 2-benzofuranboronic acid (1 equiv.).

¹H NMR (DMSO-d₆) δ ppm 8.30-8.39 (m, 2H) 8.11-8.18 (m, 1H) 7.97-8.07 (m, 2H) 7.86 (br s, 2H) 7.77-7.81 (m, 2H) 7.65-7.72 (m, 1H) 7.48 (s, 2H) 7.40-7.45 (m, 1H) 7.31-7.36 (m, 1H); MS (ESI) m/z 533, 535 [M−1]⁻.

### a) 3-Bromo-4-iodo-N-(2-sulfamoylphenylsulfonyl)benzamide

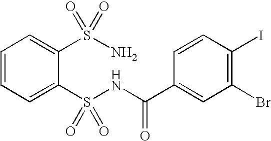

The title compound was synthesized as described for Example 73a) in 75% yield, starting from 3-bromo-4-iodobenzoic acid.

¹H NMR (DMSO-d₆) δ ppm 8.26-8.34 (m, 1H) 8.18 (br s, 1H) 8.09-8.15 (m, 1H) 8.01-8.07 (m, 1H) 7.85 (br s, 2H) 7.53 (dd, 1H) 7.46 (br s, 2H); MS (ESI) m/z 543, 545 [M-1]⁻.

### b) 3-Bromo-4-iodobenzoic Acid

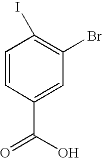

The title compound was synthesized as described for Example 74a) in 98% yield, starting from methyl 3-bromo-4-iodobenzoate.

¹H NMR (DMSO-d₆) δ ppm 13.46 (s, 1H) 8.06-8.20 (m, 2H) 7.61 (dd, 1H); MS (ESI) m/z 325, 327 [M−1]⁻.

### c) Methyl 3-bromo-4-iodobenzoate

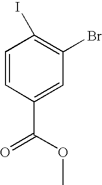

The title compound was synthesized as described for Example 151b) in 70% yield, starting from methyl 4-amino-3-bromobenzoate. Purification by column chromatography, using heptane/ethyl acetate (19:1) as the eluent.

¹H NMR (CDCl₃) δ ppm 8.18 (d, 1H) 7.88 (d, 1H) 7.55 (dd, 1H) 3.85 (s, 3H).

### EXAMPLE 156

### 4-(Benzyloxy)-3-(3-hydroxy-3-methylbut-1-ynyl)-N-(2-sulfamoylphenylsulfonyl)benzamide

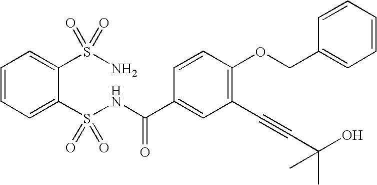

The title compound was synthesized as described for Example 154 in 37% yield, starting from 4-(benzyloxy)-3-iodo-N-(2-sulfamoylphenylsulfonyl)benzamide.

¹H NMR (DMSO-d₆) δ ppm 8.24 (br s, 1H) 8.01-8.10 (m, 1H) 7.90-7.94 (m, 1H) 7.72-7.86 (m, 3H) 7.41-7.46 (m, 2H) 7.30-7.39 (m, 4H) 7.22-7.29 (m, 1H) 7.10-7.18 (m, 1H) 5.19 (s, 2H) 1.39 (s, 6H); MS (ESI) m/z 527 [M−1]⁻.

### EXAMPLE 157

### 4-(Benzyloxy)-3-iodo-N-(2-sulfamoylphenylsulfonyl)benzamide

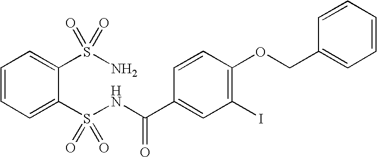

The title compound was synthesized as described for Example 73a) in 26% yield, starting from 4-(benzyloxy)-3-iodobenzoic acid. Purification by column chromatography, using a gradient of heptane/ethyl acetate (3:1-1:3) as the eluent.

MS (ESI) m/z 571 [M−1]⁻.

### a) 4-(Benzyloxy)-3-iodobenzoic acid

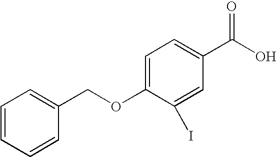

The title compound was synthesized as described for Example 74a), starting from benzyl 4-(benzyloxy)-3-iodobenzoate.

¹H NMR (DMSO-d₆) δ ppm 12.91 (s, 1H) 8.30 (d, 1H) 7.94 (dd, 1H) 7.48-7.55 (m, 2H) 7.40-7.47 (m, 2H) 7.33-7.39 (m, 1H) 7.19 (d, 1H) 5.30 (s, 2H); MS (ESI) m/z 353 [M-1]⁻

### b) Benzyl 4-(benzyloxy)-3-iodobenzoate

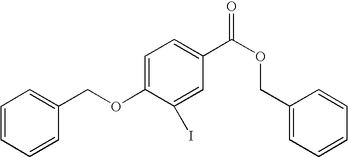

Sodium hydride (60% in mineral oil, 0.88 g, 22.0 mmol) was added in portions to a solution of 4-hydroxy-3-iodobenzoic acid (2.64 g, 10.0 mmol) in N,N-dimethylformamide (30 mL), after 0.5 hour benzyl bromide (3.56 mL, 30.0 mmol) was added and the reaction was stirred for 3 days. The reaction mixture was diluted with toluene and washed with water. The organic phase was dried over magnesium sulfate and the solvent was evaporated. Purification by column chromatography, using heptane/ethyl acetate (7:1) as the eluent, gave 1.91 g (43% yield) of the title compound.

¹H NMR (CDCl₃) δ ppm 8.56 (d, 1H) 8.07 (dd, 1H) 7.36-7.58 (m, 10H) 6.92 (d, 1H) 5.39 (s, 2H) 5.28 (s, 2H).

### EXAMPLE 158

### 2-Benzyl-N-(2-sulfamoylphenylsulfonyl)-1H-indole-5-carboxamide

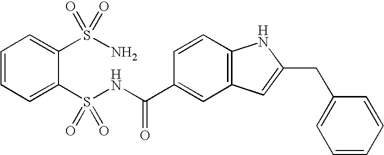

The title compound was synthesized as described for Example 73a) in 23% yield, starting from 2-benzyl-1H-indole-5-carboxylic acid. Purification by preparative HPLC.

¹H NMR (DMSO-d₆) δ ppm 12.15 (br s, 1H) 11.42 (br s, 1H) 8.28-8.38 (m, 1H) 8.09-8.19 (m, 2H) 7.90 (br s, 2H) 7.52-7.58 (m, 1H) 7.40 (br s, 2H) 7.27-7.35 (m, 5H) 7.20-7.26 (m, 1H) 6.30 (s, 1H) 4.09 (s, 2H); MS (ESI) m/z 468 [M−1]⁻.

### a) 2-Benzyl-1H-indole-5-carboxylic Acid

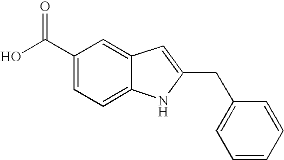

The title compound was synthesized as described for Example 74a), starting from methyl 2-benzyl-1H-indole-5-carboxylate.

MS (ESI) m/z 250 [M−1]⁻.

### b) Methyl 2-benzyl-1H-indole-5-carboxylate

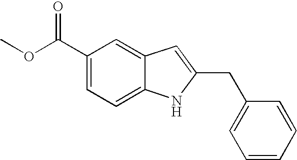

A mixture of methyl 3-iodo-4-(2,2,2-trifluoroacetamido)benzoate (0.60 g, 1.61 mmol), 3-phenyl-1-propyne (0.20 ml, 1.61 mmol) 1,1,3,3-tetramethylguanidine (2.02 ml, 16.08 mmol), bis(triphenylphosphine)palladium(II) chloride (0.113 g, 0.16 mmol) and copper(I) iodide (0.031 g, 0.16 mmol) in N,N-dimethylformamide (15 mL) was stirred under an atmosphere of argon at 50° C. over night. The reaction mixture was concentrated and purified by column chromatography, using heptane/ethyl acetate (4:1) as the eluent, to give 0.18 g (82% yield) of the title compound.

¹H NMR (DMSO-d₆) δ ppm 11.43 (br s, 1H) 8.14 (d, 1H) 7.66 (dd, 1H) 7.29-7.38 (m, 5H) 7.21-7.26 (m, 1H) 6.31 (s, 1H) 4.09 (s, 2H) 3.82 (s, 3H); MS (ESI) m/z 264 [M−1]⁻.

### EXAMPLE 159

### 7-(Cyclopropylethynyl)-2,2-difluoro-N-(2-sulfamoylphenylsulfonyl)benzo[d][1,3]dioxole-4-carboxamide

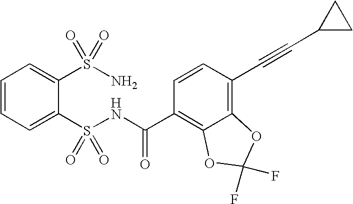

The title compound was synthesized as described for Example 146 in 20% yield, starting from 7-bromo-2,2-difluoro-N-(2-sulfamoylphenylsulfonyl)benzo[d][1,3]dioxole-4-carboxamide and 2-cyclopropylethyn-1-ylium. Purification by preparative HPLC.

¹H NMR (DMSO-d₆) δ ppm 8.20-8.28 (m, 1H) 8.03-8.11 (m, 1H) 7.70-7.82 (m, 2H) 7.56 (d, 1H) 7.44 (br s, 2H) 7.17-7.24 (m, 1H) 1.61-1.70 (m, 1H) 0.94-1.00 (m, 2H) 0.79-0.85 (m, 2H); MS (ESI) m/z 483 [M−1]⁻.

### a) 7-Bromo-2,2-difluorobenzo[d][1,3]dioxole-4-carboxylic Acid

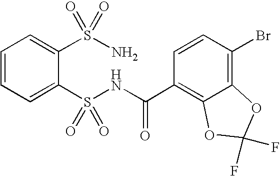

The title compound was synthesized as described for Example 73a), starting from 7-bromo-2,2-difluorobenzo[d][1,3]dioxole-4-carboxylic acid. Purification by column chromatography using chloroform/methanol (9:1) as the eluent.

MS (ESI) m/z 497, 499 [M−1]⁻.

### b) 7-Bromo-2,2-difluorobenzo[d][1,3]dioxole-4-carboxylic Acid

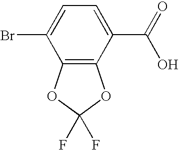

Diisopropylamine (1.18 mL, 8.44 mmol) and 4-bromo-2,2-difluoro-1,3-benzodioxole (2.0 g, 8.44 mmol) were added to a cooled (−100° C.) solution of n-butyllithium (1.6 M, in hexane, 5.27 mL, 8.44 mmol) in tetrahydrofuran (15 mL). The reaction mixture was stirred for 2 hours and was then poured onto freshly crushed dry-ice. When the mixture had reached room temperature, water was added and the mixture was washed with dichloromethane, the water phase was acidified with 2 M hydrochloric acid and extracted with diethyl ether. The organic phase was dried over magnesium sulfate and the solvent was evaporated to give the crude title compound (contains a des-bromo impurity that was present through the synthesis until the final purification step).

MS (ESI) m/z 279, 281 [M−1]⁻.

### EXAMPLE 160

### 4-(Cyclopropylethynyl)-N-(2-sulfamoylphenylsulfonyl)-3-(3,3,3-trifluoropropoxy)benzamide

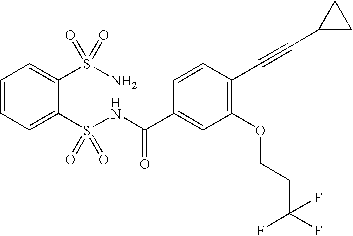

Triethylamine (1.296 mL, 9.30 mmol) was added to a mixture of 4-bromo-N-(2-sulfamoylphenylsulfonyl)-3-(3,3,3-trifluoropropoxy)benzamide (165 mg, 0.31 mmol), cyclopropylacetylene (0.079 mL, 0.93 mmol) and tetrakis(triphenylphosphine)palladium(0) (35.8 mg, 0.030 mmol) in N,N-dimethylformamide (2 mL). The mixture was stirred for 5 min, copper(I) iodide (8.9 mg, 0.050 mmol) was added and the reaction was heated at 65° C. over night. The reaction mixture was partitioned between ethyl acetate and aqueous hydrochloric acid, the organic phase was dried over magnesium sulfate and the solvent was evaporated. Purification by column chromatography, using chloroform/methanol (9:1) as the eluent, gave 37% yield of the title compound.

¹H NMR (DMSO-d₆) δ ppm 8.21-8.10 (m, 1H) 7.97-8.06 (m, 1H) 7.25-7.53 (m, 2H) 7.41-7.52 (m, 4H) 7.27 (d, 1H) 4.21 (t, 2H) 2.75-2.87 (m, 2H) 1.47-1.58 (m, 1H) 0.84-0.93 (m, 2H) 0.67-0.73 (m, 2H); MS (ESI) m/z 515 [M−1]⁻.

### a) 4-Bromo-N-(2-sulfamoylphenylsulfonyl)-3-(3,3,3-trifluoropropoxy)benzamide

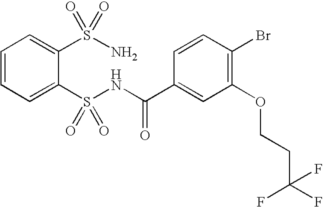

The title compound was synthesized as described for Example 73a), starting from 4-bromo-3-(3,3,3-trifluoropropoxy)benzoic acid.

MS (ESI) m/z 529, 531 [M−1]⁻.

### b) 4-Bromo-3-(3,3,3-trifluoropropoxy)benzoic Acid

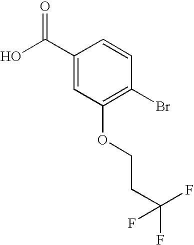

The title compound was synthesized as described for Example 74a) in 96% yield, starting from methyl 4-bromo-3-(3,3,3-trifluoropropoxy)benzoate.

¹H NMR (DMSO-d₆) δ ppm 13.28 (br s, 1H) 7.74 (d, 1H) 7.58 (d, 1H) 7.49 (dd, 1H) 4.37 (t, 2H) 2.78-2.91 (m, 2H); MS (ESI) m/z 311, 313 [M−1]⁻.

### c) Methyl 4-bromo-3-(3,3,3-trifluoropropoxy)benzoate

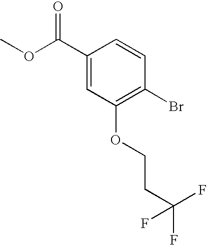

Triphenylphosphine (0.51 g, 1.95 mmol) and diisopropyl azodicarboxylate (0.38 mL, 1.95 mmol) were added to a solution of methyl 4-bromo-3-hydroxybenzoate (0.30 g, 1.30 mmol) and 3,3,3-trifluoro-1-propanol (0.17 mL, 1.95 mmol) in tetrahydrofuran (10 mL). The reaction was stirred over night, concentrated and the residue was purified by column chromatography, using heptane/ethyl acetate (9:1) as the eluent, to give 74% yield of the title compound.

¹H NMR (DMSO-d₆) δ ppm 7.71 (d, 1H) 7.52 (d, 1H) 7.44 (dd, 1H) 4.31 (t, 2H) 3.80 (s, 3H) 2.72-2.84 (m, 2H); MS (EI) m/z 326, 328 [M]⁺.

### EXAMPLE 161

### 4-(Benzofuran-2-yl)-N-(4-(hydroxymethyl)-2-sulfamoylphenylsulfonyl)benzamide

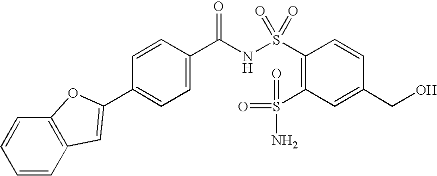

4-(Benzofuran-2-yl)-N-(4-((tert-butyldimethylsilyloxy)methyl)-2-(N-tert-butylsulfamoyl)phenylsulfonyl)benzamide (241 mg, 0.37 mmol) was dissolved in 2,2,2-trifluoroacetic acid (3 mL, 40.39 mmol) and heated at 90° C. for 1 hour. The 2,2,2-trifluoroacetic acid was evaporated, the residue was diluted in 1 M sodium hydroxide (5 mL) and methanol (5 mL) and was stirred at 60° C. for 10 min. The resulting mixture was concentrated in vacuo and purified using preparative HPLC to give 137 mg (76% yield) of the title compound.

¹H NMR (CD₃OD) δ ppm 8.29 (d, 1H) 8.20 (d, 1H) 8.09 (d, 2H) 7.89 (d, 2H) 7.67-7.60 (m, 2H) 7.53 (d, 1H) 7.30 (td, 1H) 7.27 (s, 1H) 7.25-7.21 (m, 1H) 4.70 (s, 2H); MS (ESI) m/z 485 [M−1]⁻

### a) 4-(Benzofuran-2-yl)-N-(4-((tert-butyldimethylsilyloxy)methyl)-2-(N-tert-butylsulfamoyl)phenylsulfonyl)benzamide

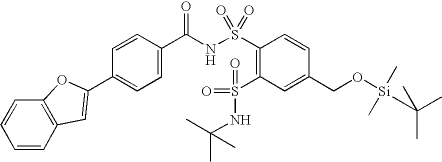

4-bromo-N-(4-((tert-butyldimethylsilyloxy)methyl)-2-(N-tert-butylsulfamoyl)phenylsulfonyl)benzamide (1.0 g, 1.61 mmol), [1,1′-bis(diphenylphosphino)ferrocene]dichloropalladium (0.130g, 0.16 mmol), benzofuran-2-ylboronic acid (0.287 g, 1.78 mmol) and potassium carbonate (1.338 g, 9.68 mmol) were dissolved in tetrahydrofurane (14 mL) and water (1 mL). The reaction was irradiated for 15 min at 150° C. in a microwave, filtered through a plug of celite and concentrated in vacuo. Purification by column chromatography, using a gradient with increasing polarity (0 to 100% ethyl acetate in heptane) as the eluent, gave 0.266 g (25% yield) of the title compound.

MS (ESI) m/z 655 [M−1]⁻

### b) 4-Bromo-N-(4-((tert-butyldimethylsilyloxy)methyl)-2-(N-tert-butylsulfamoyl)phenylsulfonyl)benzamide

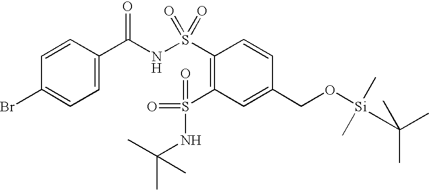

N1-tert-butyl-5-((tert-butyldimethylsilyloxy)methyl)benzene-1,2-disulfonamide (600 mg, 1.37 mmol), 4-bromobenzoic acid (276 mg, 1.37 mmol), N-(3-dimethylaminopropyl)-N′-ethylcarbodiimide hydrochloride (369 mg, 1.92 mmol) and 4-dimethylaminopyridine (420 mg, 3.44 mmol) were dissolved in anhydrous N,N-dimethylformamide (15 mL) and the reaction was stirred at room temperature over night. Water was added and the solution was extracted with ethyl acetate. The aqueous phase was acidified using hydrochloric acid (2 M) and extracted with ethyl acetate. The combined organic phases were washed with water, dried over magnesium sulfate and concentrated in vacuo to give 895 mg (quantitative yield) of the title compound.

MS (ESI) m/z 617, 619 [M−1]⁻

### c) N1-tert-Butyl-5-((tert-butyldimethylsilyloxy)methyl)benzene-1,2-disulfonamide

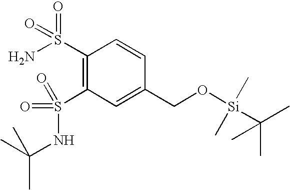

2-(Benzylthio)-N-tert-butyl-5-((tert-butyldimethylsilyloxy)methyl)benzenesulfonamide (500 mg, 1.04 mmol) was dissolved in dichloromethane (5 mL), water (5 mL) and formic acid (5 mL). Chlorine gas was bubbled through the vigorously stirred mixture for 1 min at 0° C. The reaction was allowed to reach room temperature and was stirred for 15 min. Ammonium hydroxide (33%) was added dropwise at 0° C. to the mixture until it became basic. The mixture was extracted with dichloromethane and ethyl acetate and the combined organic phases were dried over magnesium sulfate, filtered and concentrated in vacuo. Purification by column chromatography, using a gradient with increasing polarity (0 to 100% ethyl acetate in heptane) as the eluent, gave 172 mg (38% yield) of the title compound.

MS (ESI) m/z 435 [M−1]⁻

### d) 2-(Benzylthio)-N-tert-butyl-5-((tert-butyldimethylsilyloxy)methyl)benzenesulfonamide

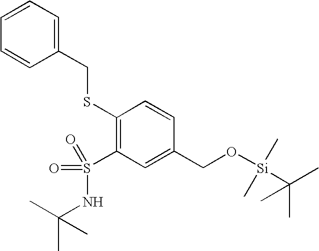

2-Bromo-N-tert-butyl-5-((tert-butyldimethylsilyloxy)methyl)benzenesulfonamide (7.7 g, 17.64 mmol), phenylmethanethiol (2.326 mL, 19.41 mmol), N-ethyldiisopropylamine (5.83 mL, 35.28 mmol), 9,9-dimethyl-4,5-bis(diphenylphosphino)xanthene (0.510 g, 0.88 mmol) and tris(dibenzylideneacetone)palladium(0) (0.404 g, 0.44 mmol) were dissolved in anhydrous N,N-dimethylformamide (22 mL). The reaction was split into two 20-mL microwave vials each were run in a microwave at 180° C. for 30 min. The combined vials were dissolved in 1 M sodium hydroxide (100 mL) and extracted with dichloromethane. The combined organic phases were dried over magnesium sulfate and concentrated in vacuo. Purification by column chromatography, using a gradient with increasing polarity (0 to 100% ethyl acetate in heptane) as the eluent, gave 7.30 g (86% yield) of the title compound.

MS (ESI) m/z 478 [M−1]⁻

### e) 2-Bromo-N-tert-butyl-5-((tert-butyldimethylsilyloxy)methyl)benzenesulfonamide

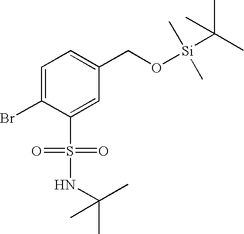

2-Bromo-N-tert-butyl-5-(hydroxymethyl)benzenesulfonamide (5.9 g, 18.31 mmol), tert-butylchlorodimethylsilane (5.52 g, 36.62 mmol) and 1H-imidazole (2.493 g, 36.62 mmol) were dissolved in anhydrous acetonitrile (100 mL). The reaction was stirred at room temperature over night, diluted with water (100 mL) and extracted with ethyl acetate. The combined organic phases were dried through a plug of celite and concentrated in vacuo to give 7.70 g (96% yield) of the title compound.

MS (ESI) m/z 434, 436 [M−1]⁻

### f) 2-Bromo-N-tert-butyl-5-(hydroxymethyl)benzenesulfonamide

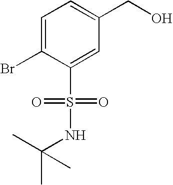

Aluminum(III) lithium hydride (47.1 mL, 47.11 mmol) was slowly added dropwise to a solution of methyl 4-bromo-3-(N-tert-butylsulfamoyl)benzoate (11 g, 31.41 mmol) in anhydrous tetrahydrofuran (50 mL) at 0° C. The reaction was allowed to reach room temperature and was stirred at room temperature for 15 min. Water (5 mL) was added dropwise, followed by 25% aqueous sodium hydroxide (5 mL) and followed by water (15 mL). The reaction was stirred for 5 min and filtered. The filtrate was diluted with water, extracted with dichloromethane and the solvent was evaporated to give 4.10 g (40.5% yield) of the title compound.

MS (ESI) m/z 320, 322 [M−1]⁻

### g) Methyl 4-bromo-3-(N-tert-butylsulfamoyl)benzoate

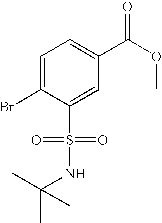

2-Methylpropan-2-amine (28.7 mL, 272.10 mmol) followed by triethylamine (37.7 mL, 272.10 mmol) was added to a solution of 4-bromo-3-(chlorosulfonyl)benzoic acid (40.75 g, 136.05 mmol in dichloromethane (100 mL). The reaction was stirred at room temperature for 2 hours and was acidified using hydrochloric acid (2 M). The mixture was extracted with ethyl acetate, silica was added and the solvent was evaporated. The silica was placed in a glass filter funnel and rinsed with a mobile phase consisting of ethyl acetate, methanol and formic acid (2:2:1). The resulting mixture was concentrated in vacuo, the residue was dissolved in methanol (50 mL), sulfuric acid (1.213 mL, 12.12 mmol) was added and the reaction was refluxed over night. The solution was concentrated under vacuum until half of the volume remained and water (5 mL) was added. The mixture was extracted with dichloromethane, the combined organic phases were dried over magnesium sulfate, filtered and concentrated in vacuo. Purification by column chromatography, using a gradient with increasing polarity (0 to 100% ethyl acetate in heptane) as the eluent, gave 3 1.0 g (65% yield) of the title compound.

MS (ESI) m/z 348, 350 [M−1]⁻

### EXAMPLE 162

### Benzene-1,2-disulfonic acid 1-amide 2-[(quinoline-3-carbonyl)-amide]

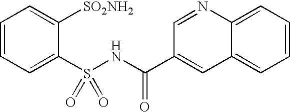

A mixture of benzene-1,2-disulfonamide (0.20 g, 0.85 mmol), 3-quinoline carboxylic acid (0.15 g, 0.85 mmol), N-(3-dimethylaminopropyl)-NA-ethylcarbodiimide hydrochloride (0.16 g, 0.85 mmol) and 4-dimethylaminopyridine (0.10 g, 0.85 mmol) in anhydrous N,N-dimethylformamide (5 mL) was stirred at room temperature for 3.5 days. Water (20 mL) and ethyl acetate (10 mL) were added, and the layers were separated. The aqueous phase was concentrated under reduced pressure and the resulting solid was washed with methanol and dried. Purification by preparative HPLC gave 35.1 mg (11% yield) of the title compound.

¹H NMR (400 MHz, DMSO-d₆) δ (ppm) 9.28 (s, 1H), 9.05 (s, 1H), 8.41-8.32 (m, 1H), 8.21-8.08 (m, 3H), 7.95 (t, 1H), 7.90-7.81 (m, 2H), 7.76 (t, 1H), 7.48 (br.s., 2H);

MS (ESI) m/z 392.0 [M+1]⁺

### Assays for Determining Biological Activity

### Inhibition of Prostaglandin E Synthase Activity

Compounds were tested as inhibitors of microsomal prostaglandin E synthase activity in microsomal prostaglandin E synthase assays and whole cell assays. These assays measure prostaglandin E2 (PGE2) synthesis which is taken as a measure of prostaglandin E synthase activity. Microsomal prostaglandin E synthase biochemical assays used microsomal prostaglandin E synthase-1 in microsomal preparations. The source of the microsomes can be for example interleukin-1β-stimulated human A549 cells (which express human mPGES-1) or Sf9 cells transfected with plasmids encoding human mPGES-1 cDNA.

The whole blood assay [described by Patrignani, P. et al., Journal of Pharmacology and Experimental Therapeutics, 1994, vol. 271, pp 1705-1712] was used as the whole cell assay for testing the compounds. Whole blood provides a protein and cell rich milieu for the study of biochemical efficacy of anti-inflammatory compounds such as prostaglandin synthase inhibitors. To study the inhibitory activities of these compounds, human blood was stimulated with lipopolysaccharide (LPS) for typically 16 hours to induce mPGES-1 expression, after which the concentration of produced PGE2 was measured by competitive-immuno assay (homogeneous time-resolved fluorescence, HTRF) as read out for effectiveness against mPGES-1-dependent PGE2 production.

### Microsomal Prostaglandin E Synthase Biochemical Assay

A solution of test compound was added to a diluted microsome preparation containing human mPGES-1 and pre-incubated for 15 minutes in potassium phosphate buffer pH 6.8 with cofactor glutathione (GSH). Corresponding solutions without test compound were used as positive controls, and corresponding solutions without test compound and without microsomes were used as negative controls. The enzymatic reaction was then started by addition of the substrate PGH2 in an organic solution (dry acetonitrile).

The typical reaction conditions of the enzymatic reaction were thus: Test compound: ranging from 60 μM to 0.002 μM, or zero in positive and negative controls; potassium phosphate buffer pH 6.8: 50 mM; GSH: 2.5 mM; mPGES-1-containing microsomes: 2 μg/mL (sample and positive controls) or 0 μg/mL (negative control); PGH2: 10.8 μM; Acetonitrile: 7.7% (v/v); DMSO: 0.6% (v/v). The reaction was stopped after one minute by adding an acidic solution (pH 1.9) of ferric chloride and citrate (final concentrations 7 mM and 47 mM respectively), by which the PGH2 was sequestered (the PGH2 is reduced to mainly 12-hydroxy heptadecatrineoic acid (12-HHT) which is not detected by the subsequent PGE2 detection step). The resulting solution was then pH neutralized by addition of potassium phosphate buffer, prior to diluting an aliquot of the resulting solution in a weak potassium phosphate buffer (50 mM, pH 6.8) containing 0.2% BSA (w/v). [Adapted from Jacobsson et al., Proc. Natl. Acad. Sci. USA, 1999, vol. 96, pp. 7220-7225] The PGE2 formed was quantified by use of a commercial HTRF based kit (catalogue #62PG2PEC or #62P2APEC from Cisbio International). 100% activity was defined as the PGE2 production in positive controls subtracted by the PGE2 production in the negative controls. IC50 values were then determined using standard procedures.

Data from this assay for representative compounds is shown in the Table below. The potency is expressed as IC50 and the value indicated is an average of at least n=2. The data indicate that the compounds of the invention are expected to possess useful therapeutic properties.

| Example No. | IC₅₀ (μM) |
|---|---|
| 1 | 0.24 |
| 2 | 2 |
| 3 | 0.0058 |
| 4 | 0.04 |
| 5 | 0.023 |
| 6 | 1.1 |
| 7 | 1 |
| 8 | 0.086 |
| 9 | 0.078 |
| 10 | 0.44 |
| 11 | 5.5 |
| 12 | 0.17 |
| 13 | 0.29 |
| 14 | 1.4 |
| 15 | 2 |
| 16 | 5.2 |
| 17 | 9.8 |
| 18 | 0.1 |
| 19 | 8.7 |
| 20 | 0.59 |
| 21 | 2.2 |
| 22 | 0.03 |
| 23 | 1 |
| 24 | 5.4 |
| 25 | 0.02 |
| 26 | 0.12 |
| 27 | 0.14 |
| 28 | 0.044 |
| 29 | 0.29 |
| 30 | 0.16 |
| 31 | 0.32 |
| 32 | 1.5 |
| 33 | 4.6 |
| 34 | 1.6 |
| 35 | 0.53 |
| 36 | 0.28 |
| 37 | 1.1 |
| 38 | 1.5 |
| 39 | 0.082 |
| 40 | 2.2 |
| 41 | 5.4 |
| 42 | 0.11 |
| 43 | 0.028 |
| 44 | 0.24 |
| 45 | 0.0055 |
| 46 | 0.046 |
| 47 | 0.14 |
| 48 | 0.15 |
| 49 | 0.0081 |
| 49a | 0.54 |
| 50 | 0.0032 |
| 51 | 0.0034 |
| 52 | 0.45 |
| 53 | 1.6 |
| 54 | 0.062 |
| 55 | 0.12 |
| 56 | 2.3 |
| 57 | 8.8 |
| 58 | 1.9 |
| 59 | 0.056 |
| 60 | 0.27 |
| 61 | 0.099 |
| 62 | 0.02 |
| 63 | 0.096 |
| 64 | 6.2 |
| 65 | 0.014 |
| 66 | 0.22 |
| 67 | 0.085 |
| 68 | 2 |
| 69 | 0.079 |
| 70 | 0.32 |
| 71 | 1 |
| 72 | 0.01 |
| 73 | 0.06 |
| 74 | 0.024 |
| 75 | 0.029 |
| 76 | 0.11 |
| 77 | 0.72 |
| 78 | 5.7 |
| 79 | 0.07 |
| 80 | 0.13 |
| 81 | 1 |
| 82 | 0.54 |
| 83 | 0.042 |
| 84 | 0.17 |
| 85 | 0.049 |
| 86 | 0.071 |
| 87 | 0.016 |
| 88 | 0.14 |
| 89 | 1.2 |
| 90 | 0.26 |
| 91 | 0.12 |
| 92 | 0.019 |
| 93 | 0.058 |
| 94 | 13 |
| 95 | 2 |
| 96 | 1.7 |
| 97 | 5.1 |
| 98 | 0.11 |
| 99 | 0.4 |
| 100 | 0.07 |
| 101 | 0.048 |
| 102 | 0.053 |
| 103 | 0.015 |
| 104 | Not tested |
| 105 | 2.1 |
| 106 | 0.14 |
| 107 | Not tested |
| 108 | 7 |
| 109 | 0.27 |
| 110 | 0.27 |
| 111 | 0.34 |
| 112 | 1.4 |
| 113 | 0.08 |
| 114 | 1.6 |
| 115 | 4.3 |
| 116 | 0.35 |
| 117 | 0.18 |
| 118 | 0.62 |
| 119 | 0.017 |
| 120 | 0.028 |
| 121 | 2.1 |
| 122 | 0.65 |
| 123 | 2 |
| 124 | 21 |
| 125 | 12 |
| 126 | 0.26 |
| 127 | 0.0095 |
| 128 | 0.045 |
| 129 | 7 |
| 130 | 0.02 |
| 131 | 0.014 |
| 132 | 0.11 |
| 133 | 0.56 |
| 134 | 0.18 |
| 135 | 0.081 |
| 136 | 0.065 |
| 137 | 0.02 |
| 138 | 0.012 |
| 139 | 0.0068 |
| 140 | 0.14 |
| 141 | 0.3 |
| 142 | 0.049 |
| 143 | 0.014 |
| 144 | 0.011 |
| 145 | 0.023 |
| 146 | 0.015 |
| 147 | 0.054 |
| 148 | 0.022 |
| 149 | 0.064 |
| 150 | 0.36 |
| 151 | 0.38 |
| 152 | 0.57 |
| 153 | 0.33 |
| 154 | 0.0099 |
| 155 | Not tested |
| 156 | 0.11 |
| 157 | Not tested |
| 158 | 0.58 |
| 159 | 0.063 |
| 160 | 0.032 |
| 161 | 0.32 |
| 162 | 11 |

### Whole Blood Assay

Human blood collected from human volunteers in heparinized tubes was incubated with 100 μM acetyl salicylic acid, in order to inhibit the constitutively expressed cyclooxygenase (COX)-1/COX-2 enzymes, and then stimulated with 0.1 μg/ml LPS to induce the expression of enzymes along the COX-2 pathway, e.g., COX-2 and mPGES-1. 100 μL of this blood was added to the wells of a 384-well plate containing 1 μL DMSO solutions of compounds typically in the final concentration range 316 μM to 0.01 μM. Naproxen was used as reference compound. The mix was incubated at 37° C. for 16 hours. Plasma was harvested by centrifugation and stored at −70° C. until further analysis of PGE2 levels. For the calculations, the 0%-activity value was represented by blood treated with acetyl salicylic acid, LPS and the reference compound (1 mM Naproxen). The 100%-activity value was represented by blood treated with aspirin, LPS and DMSO. [Reference: Patrignani, P. et al., Journal of Pharmacology and Experimental Therapeutics, 1994, vol. 271, pp 1705-1712]. The PGE2 formed was quantified, after dilution in a weak potassium phosphate buffer (50 mM, pH 6.8) containing 0.2% BSA (w/v), by use of a commercial HTRF based kit (catalogue #62PG2PEC or #62P2APEC from Cisbio International). IC50 values were then determined using standard procedures.

1. A compound of formula (I) or a pharmaceutically acceptable salt thereof 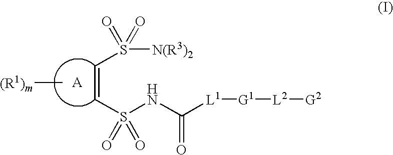
wherein:
A is selected from phenyl or a 5- or 6-membered heteroaryl moiety; said phenyl or a 5- or 6-membered heteroaryl moiety in group A being optionally fused to a phenyl, a 5- or 6-membered heteroaryl, C₅₋₆-carbocyclyl or C₅₋₆heterocyclyl ring;
R¹ is independently selected from halogen, nitro, SF₅, OH, CHO, CO₂R⁴, CONR⁵R⁶, C₁₋₄alkyl, C₁₋₄alkoxy, G³, OG³ or OCH₂G³; said C₁₋₄alkyl or C₁₋₄alkoxy being optionally substituted by OH or by one or more F atoms;
m represents an integer 0, 1 or 2;
R³ is hydrogen;
L¹ represents a direct bond, C₁₋₄alkylene, C₂₋₄alkenylene or C₂₋₄alkynylene;
L² represents a direct bond, —O—, —OCH₂—, C₁₋₂alkylene or —C≡C—;
G¹ represents phenyl, 5- or 6-membered heteroaryl, C₃₋₁₀carbocyclyl or C₅₋₈heterocyclyl;
G² represents H, C₁₋₆alkyl, C₁₋₆alkenyl, phenyl, 5- or 6-membered heteroaryl, C₃₋₁₀carbocyclyl or
C₅₋₈heterocyclyl; said C₁₋₆alkyl being optionally further substituted by one or more groups selected from OH, C₁₋₆alkoxy and halogen;
the phenyl, heteroaryl, carbocyclyl or heterocyclyl moieties in G¹ and G² being optionally fused to one or two further rings independently selected from phenyl, a 5- or 6-membered heteroaryl, C₅₋₆-carbocyclyl or C₅₋₆heterocyclyl ring;
any phenyl, heteroaryl, carbocyclyl or heterocyclyl moieties in G¹ and G² being optionally substituted by one or more substituents independently selected from halogen, OH, CN, NO₂, CO₂R⁹, C₁₋₆alkyl, C₁₋₆alkoxy, C₁₋₄thioalkoxy, SO₂NR¹⁰R¹¹,NR¹²R¹³, —O(CH₂)₂—O—(CH₂)₂—C₁₋₆alkoxy, —NHCOC(OH)(CH₃)CF₃, —CH₂OCH₂CF₂CHF₂ or —CH₂OCH₂CH₂CF₃; said C₁₋₆alkyl or C₁₋₆alkoxy being optionally substituted by OH, C₁₋₆alkoxy, phenyl or by one or more F atoms;
G³ represents phenyl or 5- or 6-membered heteroaryl; and
each R⁴, R⁵, R⁶, R⁹, R¹⁰, R¹¹, R¹² and R¹³ is independently selected from H or C₁₋₄alkyl;
provided that the compounds
1,2-Benzenedisulfonamide, N1-[[(4,6-dimethyl-2-pyrimidinyl)amino]carbonyl];
1,2-Benzenedisulfonamide, N1-[[(4,6-dimethoxy-1,3,5-triazin-2-yl)amino]carbonyl]; 1,2-Benzenedisulfonamide, N1-[[(4-methoxy-6-methyl-2-pyrimidinyl)amino]carbonyl]; 1,2-Benzenedisulfonamide, N1-[[(4,6-dimethoxy-2-pyrimidinyl)amino]carbonyl] are excluded.

2. A compound according to claim 1 wherein
G¹ represents phenyl, 5- or 6-membered heteroaryl, C₃₋₁₀carbocyclyl or C₅₋₈heterocyclyl;
G² represents H, C₁₋₆alkyl, phenyl, 5- or 6-membered heteroaryl, C₃₋₁₀carbocyclyl or C₅₋₈heterocyclyl; said C₁₋₆alkyl being optionally further substituted by one or more groups selected from OH, C₁₋₆alkoxy and halogen;
any phenyl, heteroaryl, carbocyclyl or heterocyclyl moieties in G¹ and G² being optionally substituted by one or more substituents independently selected from halogen, OH, CN, NO₂, CO₂R⁹, C₁₋₆alkyl, C₁₋₆alkoxy, C₁₋₄thioalkoxy, SO₂NR¹⁰R¹¹,NR¹²R¹³, —NHCOC(OH)(CH₃)CF₃ or —CH₂OCH₂CF₂CHF₂; said C₁₋₆alkyl or C₁₋₆alkoxy being optionally substituted by OH or by one or more F atoms.

3. A compound according to claim 1 wherein A represents phenyl.

4. A compound according to claim 1 wherein R¹ is independently selected from halogen, C₁₋₄alkyl or C₁₋₄alkoxy; said C₁₋₄alkyl or C₁₋₄alkoxy being optionally substituted by OH or by one or more F atoms;

5. A compound according to claim 1 wherein m is 0 or 1.

6. A compound according to claim 1 wherein m is 0.

7. A compound according to claim 1 wherein L¹ is a direct bond or C₁₋₄alkylene.

8. A compound according to claim 1 wherein L² is a direct bond, —OCH₂— or —C≡C—.

9. A compound according to claim 1 wherein any phenyl, heteroaryl, carbocyclyl or heterocyclyl moieties in G¹ and G² is optionally substituted by one or more substituents independently selected from halogen, CO₂R⁹, C₁₋₆alkyl, C₁₋₆alkoxy, —O(CH₂)₂—O—(CH₂)₂—C₁₋₆alkoxy, —CH₂OCH₂CF₂CHF₂ or —CH₂OCH₂CH₂CF₃; said C₁₋₆alkyl or C₁₋₆alkoxy being optionally substituted by OH, C₁₋₆alkoxy, phenyl or by one or more F atoms.

10. A compound according to claim 1 wherein any phenyl, heteroaryl, carbocyclyl or heterocyclyl moieties in G¹ and G² being optionally substituted by one or more substituents independently selected from halogen, CO₂R⁹, C₁₋₆alkyl, C₁₋₆alkoxy or —CH₂OCH₂CF₂CHF₂; said C₁₋₆alkyl or C₁₋₆alkoxy being optionally substituted by OH or by one or more F atoms.

11. A compound according to claim 1 wherein G¹ is phenyl, pyridyl, thiazolyl, thienyl, furanyl, pyrimidinyl, cyclohexyl, adamantyl or bicycloheptyl.

12. A compound according to claim 1 wherein G² is phenyl, benzofuranyl, benzothienyl, benzthiazolyl, [1,3]oxazolo[4,5-c]pyridyl, [1,3]oxazolo[5,4-c]pyridyl, benzoxazolyl, 2,3-dihydro-1-benzofuranyl, indolyl, pyridyl, quinolyl, cyclopropyl, cyclopentyl, cyclohexyl, or cycloheptyl.

13. A compound according to claim 1 wherein G² represents C₂₋₄alkenylene.

14. A compound according to claim 1 wherein A is selected from phenyl or pyridyl;
R¹ is independently selected from halogen, C₁₋₄alkyl or C₁₋₄alkoxy; said C₁₋₄alkyl or C₁₋₄alkoxy being optionally substituted by OH or by one or more F atoms;
m represents an integer 0 or 1;
R³ is hydrogen;
L¹ represents a direct bond or C₁₋₄alkylene;
L² represents a direct bond, —OCH₂—, C₁₋₂alkylene or —C≡C—;
G¹ represents phenyl, 5- or 6-membered heteroaryl or C₃₋₁₀carbocyclyl; optionally fused to one further ring selected from phenyl or 5- or 6-membered heteroaryl;
G² represents H, C₁₋₆alkyl, C₂₋₄alkenylene, phenyl, 5- or 6-membered heteroaryl, C₃₋₁₀carbocyclyl or C₅₋₈heterocyclyl; said C₁₋₆alkyl being optionally further substituted by one or more groups selected from OH, C₁₋₆alkoxy or halogen;
the phenyl, heteroaryl, carbocyclyl or heterocyclyl moieties in G¹ and G² being optionally fused to one or two further rings independently selected from phenyl, a 5- or 6-membered heteroaryl, C₅₋₆-carbocyclyl or C₅₋₆heterocyclyl ring;
any phenyl, heteroaryl, carbocyclyl or heterocyclyl moieties in G¹ and G² being optionally substituted by one or more substituents independently selected from halogen, OH, CN, NO₂, CO₂R⁹, C₁₋₆alkyl, C₁₋₆alkoxy, C₁₋₄thioalkoxy, SO₂NR¹⁰R¹¹,NR¹²R¹³, —O(CH₂)₂—O—(CH₂)₂—C₁₋₆alkoxy, —NHCOC(OH)(CH₃)CF₃ or —CH₂OCH₂CF₂CHF₂; said C₁₋₆alkyl or C₁₋₆alkoxy being optionally substituted by OH, C₁₋₆alkoxy, phenyl or by one or more F atoms;
G³ represents phenyl or 5- or 6-membered heteroaryl; and
each R⁴, R⁵, R⁶, R⁹, R¹⁰, R¹¹, R¹² and R¹³ is independently selected from H or C₁₋₄alkyl.

15. A compound according to claim 1 wherein
A is selected from phenyl;
R¹ is independently selected from halogen, C₁₋₄alkyl or C₁₋₄alkoxy; said C₁₋₄alkyl or C₁₋₄alkoxy being optionally substituted by OH or by one or more F atoms;
m represents an integer 0 or 1;
R³ is hydrogen;
L¹ represents a direct bond or C₁₋₄alkylene;
L² represents a direct bond, —OCH₂—, C₁₋₂alkylene or —C≡C—;
G¹ represents phenyl or 5- or 6-membered heteroaryl; optionally fused to one further ring selected from phenyl or 5- or 6-membered heteroaryl;
G² represents H, C₁₋₆alkyl, C₁₋₆alkenylene, phenyl, 5- or 6-membered heteroaryl, C₃₋₁₀carbocyclyl or
C₅₋₈heterocyclyl; said C₁₋₆alkyl being optionally further substituted by one or more groups selected from OH, C₁₋₆alkoxy or halogen;
the phenyl, heteroaryl, carbocyclyl or heterocyclyl moieties in G¹ and G² being optionally fused to one or two further rings independently selected from phenyl, a 5- or 6-membered heteroaryl, C₅₋₆-carbocyclyl or C₅₋₆heterocyclyl ring;
any phenyl, heteroaryl, carbocyclyl or heterocyclyl moieties in G¹ and G² being optionally substituted by one or more substituents independently selected from halogen, OH, CN, NO₂, CO₂R⁹, C₁₋₆alkyl, C₁₋₆alkoxy, C₁₋₄thioalkoxy, SO₂NR¹⁰R¹¹,NR¹²R¹³, —O(CH₂)₂—O—(CH₂)₂—C₁₋₆alkoxy, —NHCOC(OH)(CH₃)CF₃, —CH₂OCH₂CF₂CHF₂ or —CH₂OCH₂CH₂CF₃; said C₁₋₆alkyl or C₁₋₆alkoxy being optionally substituted by OH, C₁₋₆alkoxy, phenyl or by one or more F atoms.

16. A compound selected from the group consisting of:
5-Benzofuran-2-yl-N-(2-sulfamoylphenyl)sulfonyl-pyridine-2-carboxamide
5-(2,3-Dichlorophenyl)-N-(2-sulfamoylphenyl)sulfonyl-pyridine-2-carboxamide
4-Benzofuran-2-yl-N-(2-sulfamoylphenyl)sulfonyl-benzamide
4-Benzothiophen-2-yl-N-(2-sulfamoylphenyl)sulfonyl-benzamide
4-Benzothiazol-2-yl-N-(2-sulfamoylphenyl)sulfonyl-benzamide
4-(7-Oxa-3,9-diazabicyclo[4.3.0]nona-2,4,8,10-tetraen-8-yl)-N-(2-sulfamoylphenyl)sulfonyl-benzamide
4-(7-Oxa-5,9-diazabicyclo[4.3.0]nona-2,4,8,10-tetraen-8-yl)-N-(2-sulfamoylphenyl)sulfonyl-benzamide
4-Benzooxazol-2-yl-N-(2-sulfamoylphenyl)sulfonyl-benzamide
2-Phenyl-N-(2-sulfamoylphenyl)sulfonyl-benzofuran-6-carboxamide
4-Bromo-N-(2-sulfamoylphenyl)sulfonyl-benzamide
4-Bromo-2-chloro-N-(2-sulfamoylphenyl)sulfonyl-benzamide
4-Bromo-3-methyl-N-(2-sulfamoylphenyl)sulfonyl-benzamide
4-Bromo-3-fluoro-N-(2-sulfamoylphenyl)sulfonyl-benzamide
4-Bromo-2-fluoro-N-(2-sulfamoylphenyl)sulfonyl-benzamide
4-Bromo-2-methyl-N-(2-sulfamoylphenyl)sulfonyl-benzamide
2-(1-Adamantyl)-N-(2-sulfamoylphenyl)sulfonyl-acetamide
N-(2-Sulfamoylphenyl)sulfonylnorbornane-2-carboxamide
1-Phenyl-N-(2-sulfamoylphenyl)sulfonyl-cyclohexane-1-carboxamide
3-(Difluoromethoxy)-N-(2-sulfamoylphenyl)sulfonyl-benzamide
3-Bromo-4-fluoro-N-(2-sulfamoylphenyl)sulfonyl-benzamide
N-(2-Sulfamoylphenyl)sulfonyl-3-(2,2,3,3-tetrafluoropropoxymethyl)benzamide
4-Methyl-N-(2-sulfamoylphenyl)sulfonyl-2-[3-(trifluoromethyl)phenyl]1,3-thiazole-5-carboxamide
4-Chloro-2-fluoro-N-(2-sulfamoylphenyl)sulfonyl-benzamide
2-Benzyl-4-chloro-N-(2-sulfamoylphenyl)sulfonyl-benzamide
2-Phenyl-N-(2-sulfamoylphenyl)sulfonyl-benzofuran-5-carboxamide
4-Methyl-N-(2-sulfamoylphenyl)sulfonyl-2-[4-(trifluoromethyl)phenyl]1,3-thiazole-5-carboxamide
2-(2,3-Dihydrobenzofuran-5-yl)-4-methyl-N-(2-sulfamoylphenyl)sulfonyl-1,3-thiazole-5-carboxamide
2-(4-Chlorophenyl)-4-methyl-N-(2-sulfamoylphenyl)sulfonyl-1,3-thiazole-5-carboxamide
4-Methyl-2-phenyl-N-(2-sulfamoylphenyl)sulfonyl-1,3-thiazole-5-carboxamide
4-Phenylmethoxy-N-(2-sulfamoylphenyl)sulfonyl-benzamide
4-Phenyl-N-(2-sulfamoylphenyl)sulfonyl-benzamide
N-(2-Sulfamoylphenyl)sulfonyl-4-tert-butyl-benzamide
1-Methyl-N-(2-sulfamoylphenyl)sulfonyl-indole-2-carboxamide
5-Pyridin-2-yl-N-(2-sulfamoylphenyl)sulfonyl-thiophene-2-carboxamide
5-Phenyl-N-(2-sulfamoylphenyl)sulfonyl-thiophene-2-carboxamide
5-(3,4-Dichlorophenyl)-N-(2-sulfamoylphenyl)sulfonyl-furan-2-carboxamide
N-(2-Sulfamoylphenyl)sulfonyl-5-[3-(trifluoromethyl)phenyl]furan-2-carboxamide
1-(3,5-Dichlorophenyl)-5-propyl-N-(2-sulfamoylphenyl)sulfonyl-pyrazole-4-carboxamide
3,6-Dichloro-N-(2-sulfamoylphenyl)sulfonyl-benzothiophene-2-carboxamide
N-(2-Sulfamoylphenyl)sulfonylbenzothiophene-3-carboxamide
Ethyl 4-[5-[(2-Sulfamoylphenyl)sulfonylcarbamoyl]-2-furyl]benzoate
2-(3-Chlorophenyl)-4-methyl-N-(2-sulfamoylphenyl)sulfonyl-1,3-thiazole-5-carboxamide
4-(3,3-Dimethylbut-1-ynyl)-N-(2-sulfamoylphenylsulfonyl)benzamide
4-(3-Hydroxy-3-methylbut-1-ynyl)-N-(2-sulfamoylphenylsulfonyl)benzamide
4-(Benzofuran-2-yl)-2-methyl-N-(2-sulfamoylphenylsulfonyl)benzamide;
4-(Benzofuran-2-yl)-2-methyl-N-(2-sulfamoylphenylsulfonyl)benzamide;
4-(Benzofuran-2-yl)-3,5-dimethoxy-N-(2-sulfamoylphenylsulfonyl)benzamide;
4-(Benzofuran-2-yl)-2-methoxy-N-(2-sulfamoylphenylsulfonyl)benzamide;
4-(Benzofuran-2-yl)-2-hydroxy-N-(2-sulfamoylphenylsulfonyl)benzamide;
4-(Benzofuran-2-yl)-3-methoxy-N-(2-sulfamoylphenylsulfonyl)benzamide;
4-(Benzofuran-2-yl)-3-hydroxy-N-(2-sulfamoylphenylsulfonyl)benzamide;
4-(Benzofuran-2-yl)-2,6-dimethyl-N-(2-sulfamoylphenylsulfonyl)benzamide;
4-(3-Methoxyprop-1-ynyl)-N-(2-sulfamoylphenylsulfonyl)benzamide;
4-(3-Methylbut-3-en-1-ynyl)-N-(2-sulfamoylphenylsulfonyl)benzamide;
6-(Phenylethynyl)-N-(2-sulfamoylphenylsulfonyl)nicotinamide;
4-(3-Ethyl-3-hydroxypent-1-ynyl)-N-(2-sulfamoylphenylsulfonyl)benzamide;
4-(3-Hydroxy-3-methylpent-1-ynyl)-N-(2-sulfamoylphenylsulfonyl)benzamide;
4-((1-Hydroxycyclopentyl)ethynyl)-N-(2-sulfamoylphenylsulfonyl)benzamide;
3-(3-Hydroxy-3-methylbut-1-ynyl)-N-(2-sulfamoylphenylsulfonyl)benzamide;
3-(3,3-Dimethylbut-1-ynyl)-N-(2-sulfamoylphenylsulfonyl)benzamide;
4-(3,3-Dimethylbut-1-ynyl)-N-(2-sulfamoylphenylsulfonyl)-1-naphthamide;
4-(Benzofuran-2-yl)-N-(2-sulfamoylphenylsulfonyl)-1-naphthamide;
2-(Benzofuran-2-yl)-4-methyl-N-(2-sulfamoylphenylsulfonyl)thiazole-5-carboxamide;
3′-(3-Hydroxy-3-methylbut-1-ynyl)-N-(2-sulfamoylphenylsulfonyl)biphenyl-2-carboxamide;
4-(Cyclopentylethynyl)-N-(2-sulfamoylphenylsulfonyl)benzamide;
3-(Cyclopentylethynyl)-N-(2-sulfamoylphenylsulfonyl)benzamide;
4-(Cyclopentylethynyl)-2-methyl-N-(2-sulfamoylphenylsulfonyl)benzamide;
4-(3,3-Dimethylbut-1-ynyl)-3-methoxy-2-methyl-N-(2-sulfamoylphenylsulfonyl)benzamide;
4-(Benzofuran-2-yl)-3-methoxy-2-methyl-N-(2-sulfamoylphenylsulfonyl)benzamide;
4-(Pyridin-3-ylethynyl)-N-(2-sulfamoylphenylsulfonyl)benzamide;
4-(Pyridin-2-ylethynyl)-N-(2-sulfamoylphenylsulfonyl)benzamide;
4-(Phenylethynyl)-N-(2-sulfamoylphenylsulfonyl)benzamide;
4-(3,3-Dimethylbut-1-ynyl)-3-fluoro-N-(2-sulfamoylphenylsulfonyl)benzamide;
2-(3-Methoxyphenyl)-N-(2-sulfamoylphenylsulfonyl)benzofuran-5-carboxamide;
2-(4-Methoxyphenyl)-N-(2-sulfamoylphenylsulfonyl)benzofuran-5-carboxamide;
2-tert-Butyl-N-(2-sulfamoylphenylsulfonyl)benzofuran-5-carboxamide;
2-(1-Hydroxycyclopentyl)-N-(2-sulfamoylphenylsulfonyl)benzofuran-5-carboxamide;
2-Cyclopentyl-N-(2-sulfamoylphenylsulfonyl)benzofuran-5-carboxamide;
3-Cyano-4-(3,3-dimethylbut-1-ynyl)-N-(2-sulfamoylphenylsulfonyl)benzamide;
4-(Benzofuran-2-yl)-3-cyano-N-(2-sulfamoylphenylsulfonyl)benzamide;
4-Chloro-2-hydroxy-N-(2-sulfamoylphenylsulfonyl)benzamide;
4-Bromo-2-hydroxy-N-(2-sulfamoylphenylsulfonyl)benzamide;
4-(Benzofuran-2-yl)-2-fluoro-N-(2-sulfamoylphenylsulfonyl)benzamide;
4-(3,3-Dimethylbut-1-ynyl)-2-fluoro-N-(2-sulfamoylphenylsulfonyl)benzamide;
4-(Cyclopentylethynyl)-2-fluoro-N-(2-sulfamoylphenylsulfonyl)benzamide;
4-(Cyclopentylethynyl)-2-fluoro-3-methoxy-N-(2-sulfamoylphenylsulfonyl)benzamide;
4-(Benzofuran-2-yl)-2-fluoro-3-methoxy-N-(2-sulfamoylphenylsulfonyl)benzamide;
5-(Cyclohexylethynyl)-N-(2-sulfamoylphenylsulfonyl)picolinamide;
5-(3,3-Dimethylbut-1-ynyl)-N-(2-sulfamoylphenylsulfonyl)picolinamide;
4-(3,3-Dimethylbut-1-ynyl)-2-fluoro-3-methoxy-N-(2-sulfamoylphenylsulfonyl)benzamide;
4-(Benzofuran-2-yl)-2-chloro-N-(2-sulfamoylphenylsulfonyl)benzamide;
4-(Cyclopentylethynyl)-2-hydroxy-N-(2-sulfamoylphenylsulfonyl)benzamide;
6-(Cyclopentylethynyl)-N-(2-sulfamoylphenylsulfonyl)nicotinamide;
6-(Pyridin-2-ylethynyl)-N-(2-sulfamoylphenylsulfonyl)nicotinamide;
6-(Pyridin-3-ylethynyl)-N-(2-sulfamoylphenylsulfonyl)nicotinamide;
2-(3,3-Dimethylbut-1-ynyl)-N-(2-sulfamoylphenylsulfonyl)pyrimidine-5-carboxamide;
N-(2-Sulfamoylphenylsulfonyl)-4-((3,3,3-trifluoropropoxy)methyl)benzamide;
4-(Cyclopentylethynyl)-3-(hydroxymethyl)-N-(2-sulfamoylphenylsulfonyl)benzamide;
6-(3-Methylbut-1-ynyl)-N-(2-sulfamoylphenylsulfonyl)nicotinamide;
3-(Hydroxymethyl)-4-(phenylethynyl)-N-(2-sulfamoylphenylsulfonyl)benzamide;
4-(Cyclohexylethynyl)-3-(hydroxymethyl)-N-(2-sulfamoylphenylsulfonyl)benzamide;
2-((4-chlorophenyl)ethynyl)-N-(2-sulfamoylphenylsulfonyl)pyrimidine-5-carboxamide;
4-(Benzofuran-2-yl)-3-(hydroxymethyl)-N-(2-sulfamoylphenylsulfonyl)benzamide;
4-(Benzofuran-2-yl)-N-(2-sulfamoylphenylsulfonyl)cyclohexanecarboxamide;
(1S,4S)-4-(Benzofuran-2-yl)-N-(2-sulfamoylphenylsulfonyl)cyclohexanecarboxamide;
(1R,4R)-4-(Benzofuran-2-yl)-N-(2-sulfamoylphenylsulfonyl)cyclohexanecarboxamide;
4-(Benzofuran-2-yl)-1-methyl-N-(2-sulfamoylphenylsulfonyl)cyclohexanecarboxamide;
(1R,4R)-4-(Benzofuran-2-yl)-1-methyl-N-(2-sulfamoylphenylsulfonyl)cyclohexanecarboxamide;
(1S,4S)-4-(Benzofuran-2-yl)-1-methyl-N-(2-sulfamoylphenylsulfonyl)cyclohexanecarboxamide;
4-(3,3-Dimethylbut-1-ynyl)-3-methoxy-N-(2-sulfamoylphenylsulfonyl)benzamide;
4-(Cyclopropylethynyl)-3-methoxy-N-(2-sulfamoylphenylsulfonyl)benzamide;
4-(3-Methoxy-3-methylbut-1-ynyl)-N-(2-sulfamoylphenylsulfonyl)benzamide;
4-(3-Methylbut-1-ynyl)-N-(2-sulfamoylphenylsulfonyl)benzamide;
3-Methoxy-4-(3-methoxy-3-methylbut-1-ynyl)-N-(2-sulfamoylphenylsulfonyl)-benzamide;
3-Hydroxy-4-(3-methoxy-3-methylbut-1-ynyl)-N-(2-sulfamoylphenylsulfonyl)-benzamide;
6-(3,3-Dimethylbut-1-ynyl)-N-(2-sulfamoylphenylsulfonyl)nicotinamide;
6-(Benzofuran-2-yl)-N-(2-sulfamoylphenylsulfonyl)nicotinamide;
4-(3,3-Dimethylbut-1-ynyl)-3-(2-(2-methoxyethoxy)ethoxy)-N-(2-sulfamoylphenylsulfonyl)benzamide;
4-(Benzofuran-2-yl)-3-(2-(2-methoxyethoxy)ethoxy)-N-(2-sulfamoylphenylsulfonyl)benzamide;
2-(2-Methoxyphenyl)-N-(2-sulfamoylphenylsulfonyl)benzofuran-5-carboxamide;
2-(1-tert-Butoxyethyl)-N-(2-sulfamoylphenylsulfonyl)benzofuran-5-carboxamide;
2-(Pyridin-2-yl)-N-(2-sulfamoylphenylsulfonyl)benzofuran-5-carboxamide;
2-(Pyridin-3-yl)-N-(2-sulfamoylphenylsulfonyl)benzo furan-5-carboxamide;
2-(2-Hydroxypropan-2-yl)-N-(2-sulfamoylphenylsulfonyl)benzofuran-5-carboxamide;
2-(2-Methoxypropan-2-yl)-N-(2-sulfamoylphenylsulfonyl)benzofuran-5-carboxamide;
2-Cyclopropyl-N-(2-sulfamoylphenylsulfonyl)benzofuran-5-carboxamide;
4-(Benzofuran-2-yl)-3-isopropoxy-N-(2-sulfamoylphenylsulfonyl)benzamide;
4-(3,3-Dimethylbut-1-ynyl)-3-isopropoxy-N-(2-sulfamoylphenylsulfonyl)benzamide;
4-(3-Hydroxy-3-methylbut-1-ynyl)-3-isopropoxy-N-(2-sulfamoylphenylsulfonyl)benzamide;
4-(Cyclopentylethynyl)-3-isopropoxy-N-(2-sulfamoylphenylsulfonyl)benzamide;
4-(Cyclohexylethynyl)-3-isopropoxy-N-(2-sulfamoylphenylsulfonyl)benzamide;
4-(Cyclopropylethynyl)-3-isopropoxy-N-(2-sulfamoylphenylsulfonyl)benzamide;
4-((1-Hydroxycycloheptyl)ethynyl)-3-isopropoxy-N-(2-sulfamoylphenylsulfonyl)benzamide;
6-(3,3-Dimethylbut-1-ynyl)-5-(2-(2-methoxyethoxy)ethoxy)-N-(2-sulfamoylphenylsulfonyl)nicotinamide;
6-(Benzofuran-2-yl)-5-(2-(2-methoxyethoxy)ethoxy)-N-(2-sulfamoylphenylsulfonyl)nicotinamide;
6-(Cyclopentylethynyl)-5-(2-(2-methoxyethoxy)ethoxy)-N-(2-sulfamoylphenylsulfonyl)nicotinamide;
6-(Cyclopentylethynyl)-5-methoxy-N-(2-sulfamoylphenylsulfonyl)nicotinamide;
6-(Cyclohexylethynyl)-5-methoxy-N-(2-sulfamoylphenylsulfonyl)nicotinamide;
5-Methoxy-N-(2-sulfamoylphenylsulfonyl)-6-((4-(trifluoromethyl)phenyl)ethynyl)nicotinamide;
N-(3-Dimethylaminopropyl)-N′-ethylcarbodiimide hydrochloride;
1-(2-Methoxyethyl)-2-phenyl-N-(2-sulfamoylphenylsulfonyl)-1H-indole-5-carboxamide;
6-(Cyclopropylethynyl)-5-isopropoxy-N-(2-sulfamoylphenylsulfonyl)nicotinamide;
6-(Cyclopentylethynyl)-5-isopropoxy-N-(2-sulfamoylphenylsulfonyl)nicotinamide;
6-(Cyclohexylethynyl)-5-isopropoxy-N-(2-sulfamoylphenylsulfonyl)nicotinamide-4-(Benzofuran-2-yl)-3-(3-methoxy-3-methylbutoxy)-N-(2-sulfamoylphenylsulfonyl)benzamide;
4-(Cyclopentylethynyl)-3-fluoro-N-(2-sulfamoylphenylsulfonyl)benzamide;
6-(Benzofuran-2-yl)-5-chloro-N-(2-sulfamoylphenylsulfonyl)nicotinamide;
5-Chloro-6-(cyclopentylethynyl)-N-(2-sulfamoylphenylsulfonyl)nicotinamide;
5-Chloro-6-(3,3-dimethylbut-1-ynyl)-N-(2-sulfamoylphenylsulfonyl)nicotinamide;
4-(Benzofuran-2-yl)-N-(2-sulfamoylphenylsulfonyl)-2-(trifluoromethyl)benzamide;
4-(3,3-Dimethylbut-1-ynyl)-N-(2-sulfamoylphenylsulfonyl)-2-(trifluoromethyl)benzamide;
4-(Benzofuran-2-yl)-2,6-difluoro-N-(2-sulfamoylphenylsulfonyl)benzamide;
4-(Cyclopentylethynyl)-2,6-difluoro-N-(2-sulfamoylphenylsulfonyl)benzamide;
4-(Benzofuran-2-yl)-3-(3-hydroxy-3-methylbut-1-ynyl)-N-(2-sulfamoylphenylsulfonyl)benzamide;
4-(Benzofuran-2-yl)-3-bromo-N-(2-sulfamoylphenylsulfonyl)benzamide;
4-(Benzyloxy)-3-(3-hydroxy-3-methylbut-1-ynyl)-N-(2-sulfamoylphenylsulfonyl)benzamide;
4-(Benzyloxy)-3-iodo-N-(2-sulfamoylphenylsulfonyl)benzamide;
2-Benzyl-N-(2-sulfamoylphenylsulfonyl)-1H-indole-5-carboxamide;
7-(Cyclopropylethynyl)-2,2-difluoro-N-(2-sulfamoylphenylsulfonyl)benzo[d][1,3]dioxole-4-carboxamide;
4-(Cyclopropylethynyl)-N-(2-sulfamoylphenylsulfonyl)-3-(3,3,3-trifluoropropoxy)benzamide;
4-(Benzofuran-2-yl)-N-(4-(hydroxymethyl)-2-sulfamoylphenylsulfonyl)benzamide; and
Benzene-1,2-disulfonic acid 1-amide 2[(quinoline-3-carbonyl)-amide]
or a pharmaceutically acceptable salt thereof.

17. A process for the preparation of a compound of formula (I) or a pharmaceutically acceptable salt thereof as defined in claim 1 which comprises,
(a) reacting a compound of formula (II) 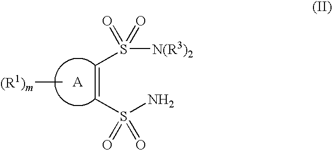
wherein R¹, R³, A and m are as defined in formula (I),
with a compound of formula (III) 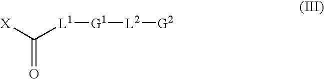
wherein L¹, L², G¹ and G² are as defined in formula (I) and X represents a leaving group such as OH or halogen; or
(b) when L² represents a direct bond and G¹ and G² are both aromatic moieties, reacting a compound of formula (IV) 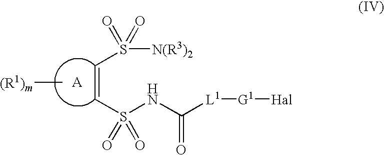
wherein Hal represents a halogen atom and R¹, R³, A, m and L¹ are as defined in formula (I),
with a nucleophile G²-M wherein M represents an organo-tin or organo boronic acid group;
and optionally after (a) or (b) carrying out one or more of the following:
converting the compound obtained to a further compound of the invention
forming a pharmaceutically acceptable salt of the compound.

18. A pharmaceutical composition comprising a compound of formula (I) or a pharmaceutically acceptable salt thereof as claimed in claim 1 in association with a pharmaceutically acceptable adjuvant, diluent or carrier.

19. A method for the treatment of human diseases or conditions in which modulation of microsomal prostaglandin E synthase-1 activity is beneficial comprising administering to a patient in need thereof a therapeutically effective amount of the compound of claim 1 or a pharmaceutically acceptable salt thereof.

20. A method for treating osteoarthritis, rheumatoid arthritis, benign or malignant neoplasias or acute or chronic pain comprising administering to a patient in need thereof a therapeutically effective amount of the compound of claim 1 or a pharmaceutically acceptable salt thereof.

21. A method for treating acute or chronic pain, nociceptive pain, neuropathic pain, apnea, sudden infant death (SID), atherosclerosis, cancer, aneurysm, hyperthermia, myositis, Alzheimer's disease or arthritis comprising administering to a patient in need thereof a therapeutically effective amount of the compound of claim 1 or a pharmaceutically acceptable salt thereof.

22. A method of treating, or reducing the risk of, an inflammatory disease or condition which comprises administering to a patient in need thereof a therapeutically effective amount of a compound of claim 1 or a pharmaceutically acceptable salt thereof.
